(19) United States
(12) Patent Application Publication
(10) Pub. No.: US 2026/0022377 A1
(43) Pub. Date: Jan. 22, 2026

(54) POLYNUCLEOTIDE COMPOSITIONS AND METHODS FOR TREATMENT OF NEURODEGENERATIVE DISEASES

(71) Applicant: APTAH BIO, INC., San Carlos, CA (US)

(72) Inventor: Caio BRUNO QUINTA DE SOUZA LEAL, Florianópolis - SC (BR)

(21) Appl. No.: 19/272,413

(22) Filed: Jul. 17, 2025

Related U.S. Application Data
(63) Continuation of application No. PCT/US2024/011890, filed on Jan. 17, 2024.
(60) Provisional application No. 63/480,464, filed on Jan. 18, 2023.

(51) Int. Cl. C12N 15/113; A61K 9/00; A61K 31/712; A61K 31/7125; A61P 25/28

(52) U.S. Cl. CPC C12N 15/113; A61K 9/0085; A61K 31/712; A61K 31/7125; A61P 25/28; C12N 2310/14; C12N 2310/315; C12N 2310/322; C12N 2310/3519; C12N 2310/531; C12N 2320/33

(57) ABSTRACT

This disclosure concerns an engineered polynucleotide that interacts with a pre-mRNA and a spliceosome to regulate gene expression. The engineered polynucleotide may have stem-loop structure that recruits the spliceosome and targeting sequences that are complementary to a target sequence at an exon-intron splice junction and may include nucleotides with 2′ modifications and phosphorothioate linkages. The engineered polynucleotide can be administered to a subject to treat a neurodegenerative disease.

### CROSS REFERENCE

This application is a continuation application of International Application No. PCT/US2024/011890, filed Jan. 17, 2024, which claims the benefit of U.S. Provisional Application No. 63/480,464, filed Jan. 18, 2023, which applications are incorporated herein by reference in their entirety.

### SEQUENCE LISTING

The instant application contains a Sequence Listing which has been submitted electronically in XML format and is hereby incorporated by reference in its entirety. Said XML copy, created on Oct. 5, 2025, is named 61572-703.301.xml and is 52,541 bytes in size.

### BACKGROUND

Aberrant splicing and premature polyadenylation have been implicated in many disease states. Proper splicing of pre-messenger RNA (pre-mRNA) and premature polyadenylation suppression are important processes for proper translation of proteins. Efficiency of splicing and premature polyadenylation suppression must be regulated in order to ensure proper splicing of pre-mRNA to avoid the production of misfolded and possibly disease-causing proteins. Efficiency of gene regulations at ribonucleic acid (RNA) levels remain limited. Accordingly, there is a need for developing polynucleotide compositions and methods for regulating gene expression and activity, e.g., at therapeutically efficacious and safe levels.

### SUMMARY

Described herein, in some aspects, is a method of treating a neurodegenerative disease in a subject in need thereof comprising administering to the subject a pharmaceutical composition comprising an engineered polynucleotide comprising: (1) one or more targeting moieties configured to specifically bind a pre-messenger ribonucleic acid (pre-mRNA) at a target sequence therein; and (2) a recruiting moiety configured to recruit a spliceosomal moiety, wherein, when associated with the pre-mRNA and the engineered polynucleotide, the spliceosomal moiety alters the pre-mRNA in or in proximity to the target sequence.

Described herein, in some aspects, is a method of treating a neurodegenerative disease in a subject in need thereof comprising administering to the subject a pharmaceutical composition comprising an engineered polynucleotide comprising: one or more targeting moieties configured to specifically bind a pre-messenger ribonucleic acid (pre-mRNA) at a target sequence therein; and a recruiting moiety configured to recruit a spliceosomal moiety, wherein, when associated with the pre-mRNA and the engineered polynucleotide, the spliceosomal moiety alters the pre-mRNA in or in proximity to the target sequence. In some embodiments, the neurodegenerative disease is selected from the group consisting of: Alzheimer's disease. Pick's disease, chronic traumatic encephalopathy, progressive supranuclear palsy, argyrophilic grain disease, Amyotrophic Lateral Sclerosis (ALS), and Frontotemporal Dementia. In some embodiments, the neurodegenerative disease is a neurodegenerative disease associated with presence of Tau in a subject's brain. In some embodiments, the expression of Tau is altered. In some embodiments, the expression of Tau is reduced. In some embodiments, the total amount of Tau in a subject's brain is reduced. In some embodiments, the neurodegenerative disease is a neurodegenerative disease associated with presence of amyloid β in a subject's brain. In some embodiments, the method alters the expression of amyloid β. In some embodiments, the method reduces the expression of amyloid β. In some embodiments, the method reduces the total amount of amyloid β in a subject's brain. In some embodiments, the method reduces the total amount of amyloid plaques in a subject's brain. In some embodiments, the engineered polynucleotide is administered at a concentration of at least 5 nM. In some embodiments, the engineered polynucleotide is administered at a concentration of at least 10 nM. In some embodiments, the engineered polynucleotide is administered at a concentration of about 5 nM to about 20 nM.

Described herein, in some aspects, is a method of reducing Tau expression by a neuron, the method comprising: administering to the neuron an engineered polynucleotide comprising: one or more targeting moieties configured to specifically bind a pre-messenger ribonucleic acid (pre-mRNA) at a target sequence therein; and a recruiting moiety configured to recruit a spliceosomal moiety, wherein, when associated with the pre-mRNA and the engineered polynucleotide, the spliceosomal moiety alters the pre-mRNA in or in proximity to the target sequence. In some embodiments, the neuron is from an individual afflicted with a neurodegenerative disorder. In some embodiments, the neurodegenerative disorder is Alzheimer's disease. In some embodiments, the neuron is a healthy neuron. In some embodiments, the neuron is a healthy neuron. In some embodiments, the neuron is from a healthy subject. In some embodiments, the engineered polynucleotide is administered at a concentration of at least 5 nM. In some embodiments, the engineered polynucleotide is administered at a concentration of at least 10 nM. In some embodiments, the engineered polynucleotide is administered at a concentration of about 5 nM to about 20 nM.

Described herein, in some aspects, is method of increasing fiber breadth in a neuron, comprising administering to the neuron an engineered polynucleotide comprising: one or more targeting moieties configured to specifically bind a pre-messenger ribonucleic acid (pre-mRNA) at a target sequence therein; and a recruiting moiety configured to recruit a spliceosomal moiety, wherein, when associated with the pre-mRNA and the engineered polynucleotide, the spliceosomal moiety alters the pre-mRNA in or in proximity to the target sequence. In some embodiments, the neuron is from an individual afflicted with a neurodegenerative disorder. In some embodiments, the neurodegenerative disorder is Alzheimer's disease. In some embodiments, the neuron is a healthy neuron. In some embodiments, the neuron is a healthy neuron. In some embodiments, the neuron is from a healthy subject. In some embodiments, the engineered polynucleotide is administered at a concentration of at least 5 nM. In some embodiments, the engineered polynucleotide is administered at a concentration of at least 10 nM. In some embodiments, the engineered polynucleotide is administered at a concentration of about 5 nM to about 20 nM.

Described herein, in some aspects, is a method of increasing branching number in a neuron, comprising administering to the neuron an engineered polynucleotide comprising: one or more targeting moieties configured to specifically bind a pre-messenger ribonucleic acid (pre-mRNA) at a target sequence therein; and a recruiting moiety configured to recruit a spliceosomal moiety, wherein, when associated with the pre-mRNA and the engineered polynucleotide, the spliceosomal moiety alters the pre-mRNA in or in proximity to the target sequence. In some embodiments, the neuron is from an individual afflicted with a neurodegenerative disorder. In some embodiments, the neurodegenerative disorder is Alzheimer's disease. In some embodiments, the neuron is a healthy neuron. In some embodiments, the neuron is a healthy neuron. In some embodiments, the neuron is from a healthy subject.

Described herein, in some aspects, is a method of increasing electrical activity in a neuron, the method comprising administering to the neuron an engineered polynucleotide comprising: one or more targeting moieties configured to specifically bind a pre-messenger ribonucleic acid (pre-mRNA) at a target sequence therein; and a recruiting moiety configured to recruit a spliceosomal moiety, wherein, when associated with the pre-mRNA and the engineered polynucleotide, the spliceosomal moiety alters the pre-mRNA in or in proximity to the target sequence.

In some embodiments, the neuron is from an individual afflicted with a neurodegenerative disorder. In some embodiments, the neurodegenerative disorder is selected from the group consisting of: Alzheimer's disease. Pick's disease, chronic traumatic encephalopathy, progressive supranuclear palsy, argyrophilic grain disease, and Amyotrophic Lateral Sclerosis (ALS), and Frontotemporal Dementia. In some embodiments, the neuron is a healthy neuron. In some embodiments, the neuron is from a healthy subject.

In some embodiments, the method alters the expression of genes in the neuron. In some embodiments, the method alters the expression of genes that are part of the MAPK signaling pathway. In some embodiments, the method alters the expression of genes that are part of the cholesterol metabolism pathway. In some embodiments, the method alters the expression of genes that are part of the steroid biosynthesis pathway. In some embodiments, the method alters the expression of genes that are part of the P13K-Akt signaling pathway. In some embodiments, the method alters the expression of genes that are part of the cell cycle regulation or cellular senescence pathway. In some embodiments, the method alters the expression of genes that are part of the pyruvate metabolism pathway. In some embodiments, the method alters the expression of genes that are part of the Ras signaling pathway. In some embodiments, the method alters the expression of genes that are part of the AMPK signaling pathway. In some embodiments, the method alters the expression of genes that are part of the fatty acid metabolism pathway. In some embodiments, the method alters the expression of genes that are part of the fatty acid synthesis pathway. In some embodiments, the method alters the expression of genes that are part of the PPAR signaling pathway. In some embodiments the method alters the expression of genes that are related to mRNA transport. In some embodiments, the method increases mRNA transport of the neuron. In some embodiments, the method decreases excitotoxicity of the neuron. In some embodiments, method increases synaptic integrity of the neuron. In some embodiments, the method decreases premature polyadenylation of one or more transcripts of the neuron.

Described herein, in some aspects, is a method of treating a subject with Alzheimer's Disease comprising administering to the subject a pharmaceutical composition comprising an engineered polynucleotide having the nucleotide sequence of SEQ ID NO: 3, thereby modulating pre-mRNAs affected by U1 snRNP dysfunction in the brain of the subject. In some embodiments, one or more, e.g., all internucleotide bonds of the engineered polynucleotide comprise a phosphorothioate linkage. In some embodiments, nucleotides at positions 1-5 and 20-24 of the engineered polynucleotide comprise a 2′-O-methyl moiety. In some embodiments, the pre-mRNA comprise tau, and the method reduces tau proteins levels in the brain.

Described herein, in some aspects, is a method of reducing amyloid β expression and/or aggregation in a neuron, the method comprising: administering to the neuron an engineered polynucleotide comprising: (i) one or more targeting moieties configured to specifically bind a pre-messenger ribonucleic acid (pre-mRNA) at a target sequence therein that could be a conserved splice site; and (ii) a recruiting moiety configured to recruit a spliceosomal moiety, wherein, when associated with the pre-mRNA and the engineered polynucleotide, the spliceosomal moiety alters the pre-mRNA in or in proximity to the target sequence.

Described herein, in some aspects, is a method of reducing U1-70K aggregation and/or mislocalization in a neuron, the method comprising: administering to the neuron an engineered polynucleotide comprising: (i) one or more targeting moieties configured to specifically bind a pre-messenger ribonucleic acid (pre-mRNA) at a target sequence therein; and (ii) a recruiting moiety configured to recruit a spliceosomal moiety, wherein, when associated with the pre-mRNA and the engineered polynucleotide, the spliceosomal moiety alters the pre-mRNA in or in proximity to the target sequence.

Described herein, in some aspects, is a method of modulating astrogliosis, the method comprising: administering to the neuron an engineered polynucleotide comprising: (i) one or more targeting moieties configured to specifically bind a pre-messenger ribonucleic acid (pre-mRNA) at a target sequence therein; and (ii) a recruiting moiety configured to recruit a spliceosomal moiety, wherein, when associated with the pre-mRNA and the engineered polynucleotide, the spliceosomal moiety alters the pre-mRNA in or in proximity to the target sequence.

In some embodiments, the engineered polynucleotide is administered intratumorally. In some embodiments the engineered polynucleotide is administered intravenously. In some embodiments the engineered polynucleotide is administered intrathecally. In some embodiments the engineered polynucleotide is administered via subcutaneous injection, intramuscular injection, intradermal injection, transdermal injection percutaneous administration, intranasal administration, intralymphatic injection, intrathecal administration, pulmonary administration, rectal administration intragastric administration, or any other suitable parenteral administration.

Described herein, in some aspects, are engineered polynucleotides for use in the methods disclosed throughout this disclosure. In some embodiments, the engineered polynucleotide comprises a nucleotide sequence that is at least 70%, 80%, 85%, or 90% identical or complementary to any one of SEQ ID NOs: 1-4. In some embodiments, the engineered polynucleotide comprises a nucleotide sequence that is identical or complementary to any one of SEQ ID NOs: 3 or 4

Described herein, in some aspects, is an engineered polynucleotide comprising: one or more targeting moiety configured to specifically bind a pre-messenger ribonucleic acid (pre-mRNA) at a target sequence therein; and a recruiting moiety configured to recruit a post-transcriptional regulating moiety (e.g., a spliceosomal moiety), wherein, when associated with the pre-mRNA and the engineered polynucleotide, the post-transcriptional regulating moiety alters the pre-mRNA in or in proximity to the target sequence.

In some embodiments, a targeting moiety of the one or more targeting moiety is sufficiently identical or complementary to a consensus sequence in the target sequence of a target gene. In some embodiments, a targeting moiety is complementary to and/or hybridizes with the target sequence. In some embodiments, a targeting moiety is complementary to and/or hybridizes with the consensus sequence of the target sequence. In some embodiment, the target sequence comprises a splice site. In some embodiment, splice sites is a conserved splice site. In some embodiment, splice site comprises 5′-GU-3′ In some embodiment, pre-mRNA is encoded by the target gene In some embodiment, the method alters an expression or activity of the target gene.

In some embodiments, the one or more targeting moiety comprises a first targeting moiety configured to specifically bind a first targeted sequence in the target sequence of the pre-mRNA, and a second targeting moiety configured to specifically bind a second targeted sequence in the target sequence of the pre-mRNA. In some embodiments, the first targeted sequence comprises a consensus sequence in the target sequence. In some embodiments, the second targeted sequence comprises a consensus sequence in the target sequence. In some embodiments, the first and second targeted sequences are apart in the target sequence by a spacing sequence of no more than five nucleotides (e.g., one or two nucleotides). In some embodiments, the target sequence comprises an exon-intron boundary in the pre-mRNA. In some embodiments, the first and second targeted sequences are both 5′ or 3′ with respect to the exon-intron boundary. In some embodiments, one of the first and second targeted sequences is 5′ with respect to the exon-intron boundary; and wherein the other of the first and second targeted sequences is 3′ with respect to the exon-intron boundary. In some embodiments, the target sequence comprises a splice site in the pre-mRNA. In some embodiments, the first or second targeted sequence comprises a splice site (e.g., 5′ ss) in the pre-mRNA. In some embodiments, one of the first and second targeting moieties is 5′ with respect to the recruiting moiety, and the other of the first and second targeting moieties is 3′ with respect to the recruiting moiety. In some embodiments, the targeting moiety comprises a sequence at least 80%, 90%, or identical to a ribosome binding site of a spliceosome snRNA, e.g., U1 snRNA. In some embodiments, the first targeting moiety comprises a sequence at least 80%, 90%, or identical to a ribosome binding site of a spliceosome snRNA, e.g., U1 snRNA. In some embodiments, the second targeting moiety comprises a sequence at least 80%, 90%, or identical to a ribosome binding site of a spliceosome snRNA, e.g., U1 snRNA. The sequence at least 80%, 90%, or identical to the ribosome binding site may be about 2 nucleotides to about 10 nucleotides. In some embodiments, the recruiting nucleotide sequence comprising: (i) a nucleotide sequence complementary to at least 4 nucleotides of the Stem-Loop II (SL2) of an U1 snRNA.

In some embodiments, the first targeting moiety or the second targeting moiety comprises a sequence identical or complementary to a sequence set forth in Table 1. In some embodiments, the first targeting moiety comprises a sequence identical or complementary to a sequence selected from the 5′-targeting moiety sequences column of Table 1; and wherein the second targeting moiety comprises a sequence identical or complementary to a sequence set forth in the 3′-targeting moiety sequences column of Table 1. In some embodiments, the first targeting moiety comprises a sequence identical or complementary to a sequence set forth in the 3′-targeting moiety sequences column of Table 1; and wherein the second targeting moiety comprises a sequence identical or complementary to a sequence set forth in the 5′-targeting moiety sequences column of Table 1. In some embodiments, the first targeting moiety or the second targeting moiety comprises a sequence identical or complementary to a consensus sequence of an intron donor site (e.g., selected from GU, GT, GC, and CA). In some embodiments, the first targeting moiety or the second targeting moiety comprises a sequence identical or complementary to a consensus sequence of an exon donor site (e.g., G). In some embodiments, the first targeting moiety or the second targeting moiety comprises a sequence identical or complementary to a consensus sequence selected from GU, GC, G, and CA. In some embodiments, the spliceosomal moiety is selected from a spliceosomal ribonucleoprotein complex, a spliceosomal small nuclear ribonucleic acid (snRNA), a spliceosomal protein, a functional variant thereof, or a functional fragment thereof. In some embodiments, the spliceosomal moiety comprises U1 snRNA and a spliceosomal protein. In some embodiments, the spliceosomal snRNA is selected from U1, U2, U4, U5, U6, U11, U12, U14atac, U6atac, and combinations thereof. In some embodiments, the spliceosomal protein is selected from Sm, U1-70k, U1A, U1C, and combinations thereof. In some embodiments, the recruiting moiety comprises a nucleotide sequence that is at least 70%, 80%, 85%, or 90% identical or complementary to a sequence set forth in Tables 2-3. In some embodiments, the recruiting moiety is complementary to Stem loop-II region of snRNA, e.g., U1 snRNA. In some embodiments, the recruiting moiety hybridizes with Stem loop-II region of snRNA, e.g., U1 snRNA. In some embodiments, the recruiting moiety comprises AGGCC. In some embodiments, the recruiting moiety comprises a nucleotide sequence that is at least 80%, 90%, or identical to at least five contiguous nucleotides of a sequence set forth in Tables 2-3. In some embodiments, the recruiting moiety comprises a nucleotide sequence that is at least 80%, 90%, or identical to about 5 to about 10 contiguous nucleotides of a sequence set forth in Tables 2-3. In some embodiments, the recruiting moiety comprises a nucleotide sequence that is identical or complementary to a sequence set forth in Tables 2-3. In some embodiments, the engineered polynucleotide comprises a (e.g., secondary) structural feature. In some embodiments, the engineered polynucleotide comprises an apical loop, an upper stem, an internal loop, a lower stem, or a combination thereof. In some embodiments, the engineered polynucleotide comprises a loop (e.g., an internal loop) adjacent to a stem (e.g., a lower stem or an upper stem) comprising two complementary stem sequences. In some embodiments, a stem sequence of the stem (e.g., the lower stem or the upper stem) comprises no more than about five, four, or three nucleotides. In some embodiments, the loop is an internal loop adjacent to the stem (e.g., the lower stem) and a further stem (e.g., an upper stem) comprising two complementary stem sequences. In some embodiments, the internal loop comprises a nucleic acid sequence of no more than 10, 9, or 8 nucleotides. In some embodiments, a stem sequence of the further stem (e.g., the upper stem) comprises no more than about five, four, or three nucleotides. In some embodiments, the engineered polynucleotide further comprises an apical loop. In some embodiments, the apical loop comprises a nucleic acid sequence of no more than 10, 9, 8, 7, 6, or 5 nucleotides. In some embodiments, the engineered polynucleotide does not comprise any intramolecular disulfide bond. In some embodiments, the engineered polynucleotide, when associated with the spliceosomal moiety, the pre-mRNA exhibits substantially no base pairing with an RNA binding domain (RBD) of U1 snRNA. In some embodiments, the engineered polynucleotide, when associated with the spliceosomal moiety, the pre-mRNA exhibits substantially no base-specific interaction with U1-C protein. In some embodiments, the engineered polynucleotide is configured to specifically interact with zinc-finger of U1-C protein. In some embodiments, a 5′-targeting moiety of the engineered polynucleotide is configured to specifically interact with zinc-finger of U1-C protein. In some embodiments, the engineered polynucleotide (is configured to covalently interact (e.g., via disulfide bonding) with zinc-finger of U1-C protein. In some embodiments, the engineered polynucleotide is configured to non-covalently interact (e.g., via hydrogen bonding) with zinc-finger of U1-C protein. In some embodiments, the engineered polynucleotide comprises a nucleotide sequence complementary to a partial sequence of Stem-Loop II (SL2) of U1 snRNA. In some embodiments, a side of a stem-loop structure of the engineered polynucleotide comprises a nucleotide sequence complementary to a partial sequence of Stem-Loop II (SL2) of U1 snRNA. In some embodiments, the partial sequence comprises the sequence corresponding to 5′-GGCCU-3′ of SL2 of U1 snRNA. In some embodiments, the partial sequence does not comprise the sequence corresponding to 5′-CACGUUA-3′ of SL2 of U1 snRNA. In some embodiments, the engineered polynucleotide exhibits substantially no base pairing with an anchoring sequence of SL2 of U1 snRNA. In some embodiments, an internal loop of the engineered polynucleotide exhibits substantially no base pairing with the anchoring sequence of the SL2 of U1 snRNA. In some embodiments, a lower stem of the engineered polynucleotide exhibits substantially no base pairing with the anchoring sequence of the SL2 of U1 snRNA. In some embodiments, the anchoring sequence comprises the sequence corresponding to 5′-CACGUUA-3′. In some embodiments, the engineered polynucleotide exhibits substantially no base pairing with H helix of U1 snRNA. In some embodiments, the engineered polynucleotide comprises at least one chemical modification. In some embodiments, the engineered polynucleotide comprises at least one 2′-modified (e.g., 2′-methoxy, 2′-methoxymethyl, or 2′-methoxyethyl) nucleotides. In some embodiments, at least about 50%, 60%, 70%, 80%, or 90% nucleotides of the engineered polynucleotide are chemically modified nucleotides. In some embodiments, at least about 50%, 60%, 70%, 80%, or 90% nucleotides of the engineered polynucleotide are 2′-modified (e.g., 2′-methoxy, 2′-methoxymethyl, or 2′-methoxyethyl) nucleotides. In some embodiments, the 2′-modified nucleotide comprises a 2′-methoxy, 2′-methoxymethyl, 2′-methoxyethyl, 2′ fluoro, or 2′-aminoethyl nucleotide. In some embodiments, the engineered polynucleotide comprises nucleotides connected by internucleotide linkages and at least one of the internucleotide linkages does not comprise a phosphate. In some embodiments, engineered polynucleotide comprises nucleotides connected by internucleotide linkages, and at least one of the internucleotide linkages comprises a sulfur (S); selenium (Se); BR3, wherein each R is independently selected from the group consisting of hydrogen, alkyl, and aryl; Carbon (C); or NR2, wherein each R is independently selected from the group consisting of a hydrogen, alkyl, and aryl.

In some embodiments, the engineered polynucleotide comprises at least one phosphorothioate internucleotide bond. In some embodiments, at least about 50%, 60%, 70%, 80%, or 90% internucleotide linkages of the engineered polynucleotide are chemically modified. In some embodiments, at least about 50%, 60%, 70%, 80%, or 90% internucleotide linkages are phosphorathioate. In some embodiments, the internucleotide linkages comprises a methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo, or methyleneoxymethylimino. In some embodiments, the engineered polynucleotide comprises about 10 to about 40 nucleotides, about 10) to about 35 nucleotides, about 10 to about 30 nucleotides, or about 10 to about 25 nucleotides. In some embodiments, the recruiting moiety comprises about 10 to about 30 nucleotides, or about 10 to about 20 nucleotides. In some embodiments, the one or more targeting moiety each independently comprises about 2 to about 15 nucleotides, about 2 nucleotides to about 10 nucleotides, or about 2 nucleotides to about 8 nucleotides. In some embodiments, one of the first and second targeting moieties comprises about 2 nucleotides, and the other of the first and second targeting moieties comprises about 5 or 6 nucleotides. In some embodiments, when associated with the engineered polynucleotide and the pre-mRNA, the spliceosomal moiety cleaves or splices the pre-mRNA in the target sequence. In some embodiments, the spliceosomal moiety further facilitates modification of a cleaved pre-mRNA.

Described herein, in some aspects, is an engineered polynucleotide comprising a nucleotide sequence that is at least 70%, 80%, 85%, or 90% identical or complementary to a sequence set forth in Tables 2-3, which engineered polynucleotide is characterized by a (e.g., secondary) structural feature. In some embodiments, the nucleotide sequence is identical or complementary to a sequence set forth in Tables 2-3. In some embodiments, the structural feature comprises one or more stem-loop structures. In some embodiments, the structural feature comprises an apical loop, an upper stem, an internal loop, a lower stem, or a combination thereof. In some embodiments, the engineered polynucleotide comprises a loop (e.g., an internal loop) adjacent to a stem (e.g., a lower stem or an upper stem) comprising two complementary stem sequences. In some embodiments, a stem sequence of the stem (e.g., the lower stem or the upper stem) comprises no more than about five, four, or three nucleotides. In some embodiments, the loop is an internal loop adjacent to the stem (e.g., the lower stem) and a further stem (e.g., an upper stem) comprising two complementary stem sequences. In some embodiments, the internal loop comprises a nucleic acid sequence of no more than 10, 9, or 8 nucleotides. In some embodiments, a stem sequence of the further stem (e.g., the upper stem) comprises no more than about five, four, or three nucleotides. In some embodiments, the engineered polynucleotide further comprises an apical loop. In some embodiments, the apical loop comprises a nucleic acid sequence of no more than 10, 9, 8, 7, 6, or 5 nucleotides. In some embodiments, the engineered polynucleotide further comprises one or more targeting moiety sufficiently identical or complementary to a target sequence of a target gene. In some embodiments, a targeting moiety of the one or more targeting moiety is sufficiently identical or complementary to a consensus sequence in the target sequence of the target gene. In some embodiments, the target gene comprises microtubule associated protein tau (MAPT). In some embodiments, the engineered polynucleotide comprises at least one chemical modification. In some embodiments, the engineered polynucleotide comprises at least one phosphorothioate internucleotide bond. In some embodiments, at least about 50%, 60%, 70%, 80%, or 90% internucleotide linkages of the engineered polynucleotide are chemically modified. In some embodiments, at least about 50%, 60%, 70%, 80%, or 90% internucleotide linkages are phosphorathioate. In some embodiments, the engineered polynucleotide comprises at least one 2′-modified (e.g., 2′-methoxy, 2′-methoxymethyl, or 2′-methoxyethyl) nucleotides. In some embodiments, at least about 50%, 60%, 70%, 80%, or 90% nucleotides of the engineered polynucleotide are chemically modified nucleotides. In some embodiments, at least about 50%, 60%, 70%, 80%, or 90% nucleotides of the engineered polynucleotide are 2′-modified (e.g., 2′-methoxy, 2′-methoxymethyl, or 2′-methoxyethyl) nucleotides. In some embodiments, the engineered polynucleotide is about 10 to about 40 nucleotides, about 10) to about 35 nucleotides, about 10 to about 30 nucleotides, or about 10) to about 25 nucleotides.

Described herein, in some aspects, is a method for altering a pre-messenger ribonucleic acid (pre-mRNA) in a cell, the method comprising contacting the cell with an engineered polynucleotide that comprises one or more targeting moiety and a recruiting moiety, wherein the one or more targeting moiety binds to the pre-mRNA at a target sequence therein, and the recruiting moiety recruits a post-transcriptional regulating moiety (e.g., a spliceosomal moiety) within proximity of the target sequence of the pre-mRNA to alter the pre-mRNA in the cell, thereby yielding one or more altered pre-mRNA. In some embodiments, a targeting moiety of the one or more targeting moiety is sufficiently identical or complementary to a consensus sequence in the target sequence of a target gene. In some embodiments, the pre-mRNA corresponds to a target gene. In some embodiments, the target gene comprises microtubule associated protein tau (MAPT). In some embodiments, the method alters an expression or activity of the target gene. In some embodiments, prior to the contacting, the cell exhibits an aberrant messenger ribonucleic acid (mRNA) or protein corresponding to the target gene.

In some embodiments, the engineered polynucleotide comprises: (i) a first targeting moiety configured to specifically bind a pre-messenger ribonucleic acid (pre-mRNA) at a first targeted sequence therein, wherein the first targeting moiety comprises a sequence identical or complementary to 5′-GTCCA-3′, (ii) a recruiting moiety comprising a sequence that is at least 90% similar or complementary to SEQ ID NO. 1 and is configured to recruit a spliceosomal moiety that comprises U1 snRNA and a U1-C protein, wherein the recruiting moiety comprises an apical loop, an upper stem adjacent to the apical loop, a lower stem, and an internal loop situated between the upper stem and the lower stem, and (iii) a second targeting moiety configured to specifically bind the pre-mRNA at a second targeted sequence therein, wherein the second targeting moiety comprises a sequence identical or complementary to 5′-CG-3″.

Described herein, in some aspects, is a set of engineered polynucleotides each independently comprise: one or more targeting moiety configured to bind a pre-messenger ribonucleic acid (pre-mRNA) at a target sequence; and a recruiting moiety configured to recruit a post-transcriptional regulating moiety (e.g., a spliceosomal moiety), wherein the set of engineered polynucleotides are configured to specifically bind the pre-mRNA at a plurality of target sequences comprising the target sequence.

Another aspect described herein is an engineered polynucleotide comprising a first targeting moiety configured to specifically bind a pre-messenger ribonucleic acid (pre-mRNA) at a first targeted sequence therein, a recruiting moiety configured to recruit a spliceosomal moiety, and a second targeting moiety configured to specifically bind the pre-mRNA at a second targeted sequence therein; wherein the recruiting moiety comprises an apical loop, an upper stem adjacent to the apical loop, a lower stem, and an internal loop situated between the upper stem and the lower stem, and wherein the spliceosomal moiety alters the pre-mRNA in a target sequence comprising the first targeted sequence and the second targeted sequence when associated with the pre-mRNA and the engineered polynucleotide. In some embodiments, the first targeting moiety is complementary to and/or hybridizes with the first target sequence. In some embodiments, the second targeting moiety is complementary to and/or hybridizes with the second target sequence. In some aspects, the first targeted sequence and second targeted sequences are separated in the target sequence by a spacing sequence of no more than five nucleotides. In some aspects, the target sequence comprises an exon-intron boundary in the pre-mRNA. In some aspects, the first targeted sequences is 5′ of the exon-intron boundary and the second targeted sequence is 3′ of the exon-intron boundary. In some aspects, the first targeting moiety comprises a sequence identical to or complementary to a sequence set forth in the exon sequence column of Table 1; and the second targeting moiety comprises a sequence identical to or complementary to a sequence set forth in the intron sequence column of Table 1. In some embodiments, the first targeting moiety comprises a sequence at least 80%, 90%, or identical to a ribosome binding site of a spliceosome snRNA, e.g., U1 snRNA. In some embodiments, the second targeting moiety comprises a sequence at least 80%, 90%, or identical to a ribosome binding site of a spliceosome snRNA, e.g., U1 snRNA. In some embodiments, the sequence at least 80%, 90%, or identical to the ribosome binding site may be about 2 nucleotides to about 10 nucleotides. In some embodiments, the recruiting moiety is complementary to Stem loop-II region of snRNA, e.g., U1 snRNA. In some embodiments, the recruiting moiety hybridizes with Stem loop-II region of snRNA, e.g., U1 snRNA. In some embodiments, the recruiting moiety comprises AGGCC. In some embodiments, the recruiting moiety comprises a nucleotide sequence that is at least 80%, 90%, or identical to at least five contiguous nucleotides of a sequence set forth in Tables 2-3. In some embodiments, the recruiting moiety comprises a nucleotide sequence that is at least 80%, 90%, or identical to about 5 to about 10 contiguous nucleotides of a sequence set forth in Tables 2-3. In some aspects, the spliceosomal moiety comprises a U1 snRNA and a U1-C protein. In some aspects, the upper stem or the lower stem comprises two complementary sequences and each of the two complementary sequences comprises no more than 5 nucleotides; the internal loop comprises two nucleic acid sequences and each of the two nucleic acid sequence comprises no more than 5 nucleotides; and the apical loop comprises a nucleic acid sequence of no more than 8 nucleotides. In other aspects the pre-mRNA exhibits substantially no base pairing with an RNA binding domain (RBD) of U1 snRNA when associated with the engineered polynucleotide and the spliceosomal moiety exhibits substantially no base-specific interaction with a U1-C protein when associated with the engineered polynucleotide and the spliceosomal moiety. In another aspect, a 5′-targeting moiety of the engineered polynucleotide is configured to specifically interact with a zinc-finger of U1-C protein. In another aspect, the recruiting moiety comprises a nucleotide sequence complementary to at least 4 nucleotides of a sequence of Stem-Loop II (SL2) of U1 snRNA. In another aspect, sequence of SL2 of U1 snRNA comprises 5′-GGCCU-3′. The engineered polynucleotide nay have a 2′-modified nucleotide. At least 50% of the nucleotides of the engineered polynucleotide may be 2′-modified nucleotides. The 2′-modified nucleotides may be 2′-methoxy nucleotides. In another aspect, the engineered polynucleotide comprises nucleotides connected by internucleotide linkages and at least one of the internucleotide linkages does not comprise a phosphate. At least one or 50%, 60%, 70% 80% or 90% of the internucleotide linkages may be a phosphorothioate.

### BRIEF DESCRIPTION OF THE DRAWINGS

The patent or application file contains at least one drawing executed in color. Copies of this patent or patent application with color drawing(s) will be provided by the Office upon request and payment of the necessary fee.

FIG. 1 illustrates a schematic diagram for identification of splice-donor and splice-acceptor sites. An example consensus sequence for messenger ribonucleic acid (mRNA) splicing in animals and plants is “GU_AG,” where “GU” is an example splice donor sequence and “AG” is an example splice acceptor sequence. A longer splice donor consensus sequence in mammals may be “GUrAGU,” where “r” represents either “G” or “A.” Usually, an expression of “GU_AG” means that only the 5′ and 3′ terminal two nucleotides of the sequence are invariable as “GU” and “AG,” respectively, and that a sequence represented by the underscore can be any sequences. However, described herein this expression indicates that the sequence represented by the underscore can be any sequences except for sequences that do not match any of the other consensus sequences. The splice acceptor consensus sequence is preceded by a branch point sequence, which contains an adenine, which is ligated to the 5′ splice site ribonucleotide to form the intron lariat, and a polypyrimidine tract (C or U), which is between the branch point and the splice acceptor sequence. While the short GU_AG consensus sequence of introns is clearly not sufficient to differentiate amongst the multitude of alternative splicing events, surprisingly little is known about what other sequence information is required to regulate alternative RNA splicing. The flanking one or two nucleotides on either side of the intron are also often conserved, and they are included in our supplementary tables, but they will not be discussed further in this paper so that we can focus our analyses on consensus sequences at the ends of the introns. In this sense, the rational design of an engineered polynucleotide logically identifies the splice's intronic consensus sequences (GU_AG). Then, making it possible to determine conserved regions of the donor site (5′ exon and intron downstream) and acceptor (3′ exon and intron downstream). It is important to note that the conserved and consensus regions are located within the same site of constitutive splice donor or acceptor. The recognition of the consensus regions determines a 5′ splicing site, that is, the limit of junction between the exon and intron. Meanwhile, the recognition of the conserved regions identifies the identity of the transcript chosen for modulation.

FIGS. 2A-2B illustrate an example engineered polynucleotide described herein comprising: (1) a 3′-targeting moiety; 3′-GC-5′; (2) a lower stem; 3′-GA-5′/5′-CT3′; (3) an internal loop; 3′-CC-5′/5′-AA-3″; (4) an upper stem; 3′-GGA-5′/5′-CCT-3′; (5) an apical loop; 3′-CTT-5′; and (6) a 5′-targeting moiety; 5′-GTCCA-3′. FIG. 2C illustrates interactions of the example engineered polynucleotide with a target pre-mRNA sequence. FIG. 2D illustrates interactions of the example engineered polynucleotide with various components of a U1 RNP complex.

FIGS. 3A-3B illustrate the anchoring taking place through the engineered polynucleotide “Stem 5′/3” (a.k.a., 5′-targeting moiety or/and 3′-targeting moiety), which is designed to interact with conserved moieties present in the constitutive donor site. FIG. 3A. Stem 5′/3′ (GTCCA and CG) such as phosphorothioate internucleotide bonds and substitutions of 2′ O-methyl (2′ O-ME) for molecular sugar increases resistance to endonucleases and increases molecular strength of interaction between the bases of the Stem 5′/3′ with conserved regions from constitutive donor. FIG. 3B. Interaction of an engineered polynucleotide described herein with constitutive donor splicing and silencing of the RNA binding moiety (RBD) of U1 snRNA with constitutive donor splicing-exon.

FIG. 4 illustrates a diagram of a human U1 snRNP. U1 snRNP is composed of one U1 snRNA, seven common Sm proteins and three U1 snRNP-specific proteins (U1-70K, U1A, and U1C). Secondary structure of U1 snRNA consists of four stem-loops (SL) and an H helix highlighted. Nucleotides forming H helix are shown. In addition, U1 snRNA sequences relevant to RNA:protein or RNA:5′ss interactions are given as well. The loop portion of SL1 is drawn according to crystal structure. It is closed by a trans WC/Hoogsteen base pair formed between A29 and A36. Protein components of U1 snRNP, their sizes and their approximate locations are shown as well. The Sm ring formed by the Sm proteins shown as green circles bind to the Sm site, which is boxed. U1-70K shown in red recognizes SL1. U1A shown in yellow binds SL2. U1C shown in blue is recruited to U1 snRNP through protein:protein interactions with U1-70 K and Sm proteins. Note signifies interactions between U1C and Sm ring.

FIG. 5A-5C illustrates U1 snRNP binding to the 5′ exon-intron junction of pre-mRNA and thus playing a crucial role at an early stage of pre-mRNA splicing. Two crystal structures of engineered U1 sub-structures are shown, which together reveal at atomic resolution an almost complete network of protein-protein and RNA-protein interactions within U1 snRNP and show how the 5′ splice site of pre-mRNA is recognized by U1 snRNP. The zinc-finger of U1-C interacts with the duplex between pre-mRNA and the 5′-end of U1 snRNA. The binding of the RNA duplex is stabilized by hydrogen bonds and electrostatic interactions between U1-C and the RNA backbone around the splice junction, but U1-C makes no base-specific contacts with pre-mRNA. The structure, together with RNA binding assays, shows that the selection of 5′-splice site nucleotides by U1 snRNP is achieved predominantly through base pairing with U1 snRNA whilst U1-C fine-tunes relative affinities of mismatched 5′-splice sites. FIG. 5A. U1-70K in complex with U1 snRNA stem-loops and U1-A RRM in complex with stem-loop 2. FIG. 5B. U1 snRNA stem-loops 1 and 2 (55-MER). FIG. 5C. U1 small nuclear ribonucleoprotein A and 70 kDa.

FIG. 6 illustrates U1-70K in complex with U1 snRNA stem-loops and U1-A RRM in complex with stem-loop 2, stabilized through the U1-C zinc finger.

FIG. 7 illustrates a diagrammatic representation of the modulation of spliceosome machinery by an engineered polynucleotide (ASMO1, also known as APT20TTMG) described herein. The anchoring of the targeting moieties (“Stem 5′/3”) (5′-GTCCA-3′ and 5′-CG-3′) allows interaction with the conserved site of constitutive donor, through the silencing of the RNA binding moiety (RBD) of U1 snRNA. Stabilization of the U1 snRNP complex can be observed through the strong ionic attraction of the Zinc Finger of U1-C, induced by disulfide bridges with thiol of the ASMO1 Stem 5′/3′. The pre-mRNA/ASMO1 duplex bond is stabilized by hydrogen bonds and electrostatic interactions between U1-C and the backbone of the pre-mRNA around the seam joint, but U1-C does not make base-specific contacts with pre-mRNA. The structure demonstrates that the selection of nucleotides of 5″-splices by U1 snRNP is achieved predominantly through the interaction between Stem 5′/3′ with pre-mRNA. Meanwhile, U1-C adjusts relative affinities of incompatible sites of 5′-splices and stabilizes the central core of spliceosome machinery by the interaction bridge between U1-70K and the Sm ring. An electrostatic interaction and hydrogen bridges of Stem-Loop II with specific base (5″-AGGCC-3″) of an upper stem (a.k.a. “Hairpin-2”; see FIG. 2B) (3′-GGA-5′/5′-CCT-3′) and Internal loop: (3′-mCC-5′/5′-AA-3′); can be observed, associated with modulation of the polyadenylation signal and acetylation by U1-A. It is noted that the anchoring moiety of U1-A in Stem-Loop II (5′-CAACGUUA-3′) is not silenced by the upper stem, inducing the modulation of the levels of genic expression and acetylation. In addition, the presence of 2′-OME groups induces a change in the molecular dynamics of the medium facilitating the conformational alteration of U1-snRNA and approximation of Stem-loop II to ASMO1, where the ASMO1 targeting or recruiting moiety are distinct from the U1-A protein decreasing the likelihood of premature interruption of the reading frame by deregulation of the polyadenylation signal.

FIG. 8 illustrates U1-70K in complex with U1 snRNA stem-loops and U1-A RRM in complex with stem-loop 2, stabilized through the U1-C zinc finger. An electrostatic interaction and hydrogen bridges of Stem-Loop II with specific base (3′-CCGGA-5′) of an upper stem (3′-GGA-5′/5′-CCT-3′) and Internal loop: (3′-mCC-5′/5′-AA-3′); can be observed, associated with modulation of the polyadenylation signal and acetylation by U1-A. It is noted that the anchoring moiety of U1-A in Stem-Loop II (5′-CAACGUUA-3′) is not silenced by the upper stem, inducing the modulation of the levels of genic expression and acetylation. In addition, the presence of 2′-OME groups induces a change in the molecular dynamics of the medium facilitating the conformational alteration of U1-snRNA and approximation of Stem-loop II to the engineered polynucleotide described herein (ASMO1).

FIG. 9A illustrates U1-C sitting on SmD3 and its binding can be stabilized by the N-terminus of U1-70K. FIG. 9B illustrates U1-C forming hydrogen bonds with the sugar-phosphate backbone atoms but making no contact with RNA bases. On the 5′SS strand, nucleotides are colored teal for exonic and fawn for intronic sequence. FIG. 9C illustrates schematic representation of the 5′-splice site recognition. Red dotted lines: hydrogen bonds made by amino acid side chains of U1-C Zinc Finger. Blue dotted lines: hydrogen bonds made by main chain atoms of U1-C Zinc Finger. Green dotted lines: disulfide bonds made by amino acid side chains of the U1-C zinc finger. Orange dotted lines: disulfide bonds made by atoms in the main chain of the U1-C zinc finger. The 5′SS nucleotides are encoded by nuclei as in FIG. 9B.

FIG. 10A illustrates Fingerprint Z1 U1-C snRNP, represented by 36 amino acid residues in blue color. FIG. 10B illustrates Z1 finger moiety of U1-C snRNP, presenting the main residues that interact with the pre-mRNA/ASMO1 duplex in the 5′ constitutive donor region. FIG. 10C illustrates representative sequence of U1-C snRNP containing 145-aa, with the 36-aa highlighted in green refer to the Zinc Finger moiety.

FIG. 11A illustrates mitochondrial activity in neurons derived from induced pluripotent stem cells (iPSCs) from healthy patient (HDC) after incubation with ASMO1. FIG. 11B illustrates mitochondrial activity in neurons derived from induced pluripotent stem cells (iPSCs) derived from patient diagnosed with Alzheimer's disease (AD) (ADC) after incubation with ASMO1.

FIG. 12 illustrates mitochondrial activity in neurons from healthy donor (HDC) after incubation with ASMO1.

FIG. 13A illustrates mitochondrial activity in microglia derived from iPSCs from healthy patient (HDC) after incubation with ASMO1. FIG. 13B illustrates mitochondrial activity in microglia derived from iPSCs from patient diagnosed with AD (ADC) after incubation with ASMO1.

FIG. 14 illustrates mitochondrial activity in astrocytes after incubation with ASMO1.

FIG. 15A illustrates extracellular glutamate levels in neurons derived from iPSCs from healthy patient (HDC) after incubation with ASMO1. FIG. 15B illustrates extracellular glutamate levels in neurons derived from iPSCs from patient diagnosed with AD (ADC) after incubation with ASMO1.

FIG. 16 illustrates a representation of a well containing neurons derived from iPSCs in a plate from NeuroHTS™ technology as well as the parameters that are able to be evaluated in three distinct regions of the neurons using this system. In the upper compartment of the well, cell number and nuclear aggregate are evaluated in soma and dendrites, in the middle compartment of the channel (axons) the thickness of the axonal fiber is verified and, in the bottom compartment, where the axons and dendrites are located, four parameters are evaluated: number of branching, number of branching junctions, neurite straightness, and axonal material.

FIG. 17A illustrates the number of cultured neurons derived from iPSCs from healthy patient (HDC) after incubation with ASMO1. FIG. 17B illustrates the number of cultured neurons derived from iPSCs from patient diagnosed with AD (ADC) after incubation with ASMO1.

FIG. 18A illustrates the formation of cell aggregates in neurons derived from iPSCs from healthy patient (HDC) after incubation with ASMO1. FIG. 18B illustrates the formation of cell aggregates in neurons derived from patient diagnosed with AD (ADC) after incubation with ASMO1.

FIG. 19A illustrates fiber breadth in neurons derived from iPSCs from healthy patient (HDC) after incubation with ASMO1. FIG. 19B illustrates fiber breadth in neurons derived from patient diagnosed with AD (ADC) after incubation with ASMO1.

FIG. 20A illustrates number of branching in neurons derived from iPSCs from healthy patient (HDC) after incubation with ASMO1. FIG. 20B illustrates number of branching in neurons derived from iPSCs from patient diagnosed with AD (ADC) after incubation with ASMO1. FIG. 20C illustrates number of branching per cell in neurons derived from iPSCs from healthy patient (HDC) after incubation with ASMO1. FIG. 20D illustrates number of branching per cell in neurons derived from iPSCs from patient diagnosed with AD (ADC) after incubation with ASMO1.

FIG. 21A illustrates branching junction in neurons derived from iPSCs from healthy patient (HDC) after incubation with ASMO1. FIG. 21B illustrates branching junction in neurons derived from iPSCs from patient diagnosed with AD (ADC) after incubation with ASMO1. FIG. 21C illustrates branching junction per cell in neurons derived from iPSCs from healthy patient (HDC) after incubation with ASMO1. FIG. 21D illustrates branching junction per cell in neurons derived from iPSCs from patient diagnosed with AD (ADC) after incubation with ASMO1.

FIG. 22A illustrates neurite straightness in neurons derived from iPSCs from healthy patient (HDC) after incubation with ASMO1. FIG. 22B illustrates neurite straightness in neurons derived from iPSCs from patient diagnosed with AD (ADC) after incubation with ASMO1.

FIG. 23A illustrates total axonal material in neurons derived from iPSCs from healthy patient (HDC) after incubation with ASMO1. FIG. 23B illustrates total axonal material in neurons derived from iPSCs from patient diagnosed with AD (ADC) after incubation with ASMO1. FIG. 23C illustrates axonal material per cell in neurons derived from iPSCs from healthy patient (HDC) after incubation with ASMO1. FIG. 23D illustrates axonal material per cell in neurons derived from iPSCs from patient diagnosed with AD (ADC) after incubation with ASMO1.

FIG. 24 illustrates a representative image of morphological changes in neurons derived from iPSCs of healthy patient (HDC) after incubation in ASMO1.

FIG. 25 illustrates a representative image of morphological changes in neurons derived from iPSCs from patient diagnosed with AD (ADC) after incubation in ASMO1.

FIG. 26A illustrates the binding of ASMO1 to U1-70K protein in a neuroblastoma cell line using Western blotting. FIG. 26B illustrates the binding of ASMO1 to U1-C protein in a neuroblastoma cell line using Western blotting. FIG. 26C illustrates the binding of ASMO1 to U1-A protein in a neuroblastoma cell line using Western blotting. FIG. 26D illustrates the binding of ASMO1 to GAPDH protein in a neuroblastoma cell line using Western blotting.

FIG. 27A-27C shows the binding of ASMO1 to U1 snRNA and pre-mRNAs. FIG. 27A U1 snRNA. FIG. 27B Tau pre-mRNA, and FIG. 27C GAPDH.

FIG. 28A illustrates the difference in the tau levels normalized with negative control of neurons derived from iPSCs of healthy patient (HDC) after incubation in ASMO1. FIG. 28B illustrates the difference in the tau levels normalized with negative control of neurons derived from iPSCs from patient diagnosed with AD (ADC) after incubation in ASMO1.

FIG. 29 illustrates a plot of the mapping profile (in percentage) of reads to the human reference genome using STAR RNA-seq aligner for the RNA-seq differential gene expression analysis.

FIG. 30 illustrates the percentage of reads mapped in genomic features such as genes, exons, promoters, gene bodies, genomic bins, and chromosomal locations using featureCounts.

FIG. 31A illustrates a principal component analysis (PCA) plot of individual samples. FIG. 31B illustrates a PCA plot with gray circles to indicate the first three clusters identified in the PCA results.

FIG. 32A illustrates a volcano plot of differentially expressed (DE) genes (DEGs) of ADC cells in 18.52 nM ASMO1 treated conditions in relation to non-treated ADC neurons. FIG. 32B illustrates a volcano plot of differentially expressed genes ADC cells in 500 nM ASMO1 treated conditions in relation to non-treated ADC neurons.

FIG. 33A illustrates a volcano plot of differentially expressed genes of HDC cells in 18.52 nM ASMO1 treated conditions in relation to non-treated HDC neurons. FIG. 33B illustrates a volcano plot of differentially expressed genes HDC cells in 500 nM ASMO1 treated conditions in relation to non-treated HDC neurons.

FIG. 34 illustrates a volcano plot of expressed genes of HDC non-treated neurons in relation to non-treated ADC neurons.

FIG. 35 illustrates top ranked terms of Gene Ontology biological processes obtained in the enrichment analysis of DE genes (DEGs) from ADC neurons treated with 18.52 nM ASMO1 in relation to non-treated ADC (medium only).

FIG. 36 illustrates top ranked terms of Gene Ontology biological processes obtained in the enrichment analysis of DE genes (DEGs) from HDC neurons treated with 18.52 nM ASMO1 in relation to non-treated HDC (medium only).

FIG. 37 illustrates top ranked terms of Gene Ontology biological processes obtained in the enrichment analysis of DE genes (DEGs) from HDC neurons treated with 500 nM ASMO1 in relation to non-treated HDC (medium only).

FIG. 38 illustrates all terms related to Gene Ontology biological processes (GO:BP) obtained for DEGs of ADC neurons treated with 500 nM ASMO1 in relation to non-treated ADC (medium only).

FIG. 39A illustrates all terms of Kyoto Encyclopedia of Genes and Genomes (KEGG) obtained in the enrichment analysis for DEGs of ADC neurons treated with 18.52 nM ASMO1 in relation to non-treated ADC (medium only). FIG. 39B illustrates all terms of Kyoto Encyclopedia of Genes and Genomes (KEGG) obtained in the enrichment analysis for DEGs of ADC neurons treated with 500 nM ASMO1 in relation to non-treated ADC (medium only).

FIG. 40A illustrates all terms of KEGG obtained in the enrichment analysis for DEGs of HDC neurons treated with 18.52 nM ASMO1 in relation to non-treated HDC (medium only).

FIG. 40B illustrates all terms of KEGG obtained in the enrichment analysis for DEGs of HDC neurons treated with 500 nM ASMO1 in relation to non-treated HDC (medium only). FIG. 40C illustrates all terms of KEGG obtained in the enrichment analysis for DEGs of HDC non-treated neurons in relation to non-treated ADC (medium only).

FIG. 41 illustrates a protein-protein interaction network and clustering for DEGs of treated ADC with 18.52 nM ASMO1.

FIG. 42 shows the 3′UTR/CDS expression ratio of genes associated with aging, indicating the potential premature polyadenylation profile and consequently the size of the expressed transcripts, aging.

FIG. 43 illustrates DE genes (DEGs) in ADC neurons treated with 18.52 nM ASMO1 and their proposed main roles in the mechanism of action of ASMO1.

FIG. 44A-44B shows spontaneous electrophysiological activity in neurons derived from iPSCs from HDC after incubation with APT20TTMG (ASMO1).

FIG. 45A-45B shows spontaneous electrophysiological activity in neurons derived from iPSCs from ADC after incubation with APT20TTMG (ASMO1).

FIG. 46A-46D shows levels of soluble Aβ40 and Aβ42 in cortex in a senescence animal model (SAMP-8) measured by ELISA. Graphs depict levels of (A) soluble Aβ40, (B) soluble Aβ42 (C) insoluble Aβ40) (D) insoluble Aβ42 in cortex samples from n=7 animals per treatment. Biological outliers as identified by histology were excluded from analysis. Data are given as pg/mg protein (Aβ40) or Aβ42).

FIG. 47A-47D shows levels of soluble Aβ40 and Aβ42 in hippocampus in a senescence animal model (SAMP-8) measured by ELISA. Graphs depict levels of (A) soluble Aβ40, prB) soluble Aβ42 (C) insoluble Aβ40) (D) insoluble Aβ42 in cortex samples from n=7 animals per treatment. Biological outliers as identified by histology were excluded from analysis. Data are given as pg/mg protein (Aβ40 or Aβ42).

FIG. 48A-48D shows quantification of Amyloid immunofluorescence in the cerebral cortex in a senescence animal model (SAMP-8).

FIG. 49A-49D shows quantification of Amyloid immunofluorescence in the hippocampus in a senescence animal model (SAMP-8).

FIG. 50A-50D shows levels of soluble and insoluble tau and ptau (T231) in cortex in a senescence animal model (SAMP-8) measured by ELISA.

FIG. 51A-51D shows levels of soluble and insoluble tau and ptau (T231) in the hippocampus in a senescence animal model (SAMP-8) measured by ELISA.

FIG. 52A-52D shows quantification of pSer202/pThr205-tau immunofluorescence in the cerebral cortex in a senescence animal model (SAMP-8).

FIG. 53A-53D shows quantification of pSer202/pThr205-tau immunofluorescence in the hippocampus in a senescence animal model (SAMP-8).

FIG. 54A-54D shows quantification of U1-70K immunofluorescence in the cerebral cortex in a senescence animal model (SAMP-8).

FIG. 55A-55D quantification of U1-70K immunofluorescence in the hippocampus in a senescence animal model (SAMP-8).

FIG. 56A-56C shows quantification of GFAP in the cerebral cortex in a senescence animal model (SAMP-8) by ELISA (58A) and immunofluorescence (58 B and 58C).

FIG. 57A-57C shows quantification of GFAP in the hippocampus in a senescence animal model (SAMP-8) by ELISA (58A) and immunofluorescence (58 B and 58C).

FIG. 58A-58H shows quantification of proinflammatory cytokines in plasma.

FIG. 59A-59B shows immunofluorescence signal in a senescence animal model (SAMP-8).

FIG. 60 shows the 3′untranslated region to coding sequence (3′UTR/CDS) ratios of TARDBP and STMN2 for HDC and ADC cells.

The novel features of the disclosure are set forth with particularity in the appended claims. A better understanding of the features and advantages of the present disclosure will be obtained by reference to the following detailed description that sets forth illustrative embodiments.

### DETAILED DESCRIPTION

Described herein are (e.g., engineered) polynucleotides, and (e.g., pharmaceutical) compositions and methods for utilizing the same, e.g., for modulating gene expression or activity.

Described herein are methods of treating neurodegenerative diseases and Alzheimer's disease using in the engineered polynucleotides described in this disclosure. The engineered polynucleotides described herein may be effective at modulating splicing of pre-mRNA by the recruitment of components of the spliceosome or at suppressing premature polyadenylation by the recruitment of components of the spliceosome involved in the telescripting process. Without being bound by a particular mechanism, the improved recruitment of components of the spliceosome to a pre-mRNA may be effective at modulating the cellular mechanisms that are causative of neurodegenerative diseases. The engineered polynucleotides may be administered to a subject that is suffering from a neurodegenerative disease, and may treat the neurodegenerative disease, for example by decreasing the number of protein aggregates, fibroids, or plaques in a subjects brain or preventing the proliferation or fibroids, protein aggregates, plaques, or other structures associated with neurodegenerative diseases.

Neurodegenerative diseases are diseases that affect the brain and nervous system and can often result in the progressive deterioration of neurons and the cells that support them. Progressive deterioration of cells in the nervous system, commonly exacerbated by the aging process, can impair an individual's ability to interact with the environment and function independently. Alzheimer's disease (AD) is a neurodegenerative disorder characterized by memory loss and the progressive loss of the ability to carry out daily activities. Millions of people worldwide are affected by different manifestations of neurodegenerative disease. Although many environmental and genetic causes of neurodegenerative disease have been identified, these disorders have no cure.

AD is one of many diseases classified as a tauopathy. AD is characterized by the presence of amyloid-β plaques and hyperphosphorylated Tau aggregation in neurofibrillary tangles, neuropil threads, and neuritic plaques in the brain. Progressive loss of white matter in regions displaying tau pathology has been observed Furthermore, it has been shown that AD is can also be characterized by U1 small nuclear ribonucleoprotein (snRNP) nuclei depletion, accumulation, and aggregation in the cytoplasm along with splicing disorders. Additionally, the basic-acidic dipeptide domain of U1-70K was demonstrated to interact with Tau from AD brains, and both U1-70K and Tau co-localize to neurofibrillary tangles in late-onset sporadic and familial case of AD).

In a study integrating data from human postmortem brain tissue and Drosophila melanogaster models. AD Tau neurofibrillary tangle pathology has been shown to disrupt spliceosome activity leading to transcriptome failure and ultimately CNS dysfunction and neurodegeneration. Tau may be involved in spliceosome cytoplasmic sequestration and disrupting snRNP assembly and/or stability. For example, some spliceosome components (including U1-70K) can physically associate with Tau in human brains with AD pathology, and in Drosophila, genetic manipulation of these factors can enhance Tau neurotoxicity. Additionally, an increase of cryptic splicing load has been observed in human postmortem brains with Tau pathology.

Additionally, the molecular events involved in the conversion from Alzheimer's Disease initiation, related to Aβ accumulation, to mild cognitive impairment (MCI) and symptomatic Alzheimer's Disease, mainly associated with Tau aggregation, remain poorly understood (Hales et al., 2016a). U1 small nuclear ribonucleoprotein particle (snRNP), particularly U1-70K and U1-A proteins are enriched in asymptomatic Alzheimer's Disease. MCI, and Alzheimer's Disease insoluble proteomes of postmortem brain tissues. Moreover, insoluble U1-A and U1-70K strongly correlate with both insoluble Aβ and Tau quantifications (Hales et al., 2016b). This evidence not only suggests a potential involvement of U1 snRNP aggregation in the mechanisms linking plaque development to NFT formation and, consequently. Alzheimer's Disease pathogenesis, but also proposes that dysfunction of snRNPs—marked by aggregation, overactivation, and mislocalization—could represent an upstream event that might significantly contribute to the initial development of Alzheimer's Disease.

The U1 snRNP complex is one of five complexes (named U1, U2, U4, U5, and U6) that comprise the human spliceosome, which is responsible for processing pre-messenger RNA (pre-mRNA), thereby, removing the intronic region and producing mature mRNA within the nucleus. The U1 snRNP complex specifically plays a role in the recognition of pre-mRNA splicing sites in the initial steps of spliceosome assembly. Additionally, U1 snRNP performs a vital global function in 3′ end pre-mRNA processing by actively suppressing premature 3′-end cleavage and polyadenylation, particularly within intronic GU-rich regions. Due to this important physiologic role, U1 snRNP dysfunction in Alzheimer's disease (AD) can result in RNA splicing deficiency as well as the suppression of premature cleavage and polyadenylation in RNA transcripts and autophagy-lysosome system. Moreover, U1 mislocalization and depletion from the nucleus and translocation to cytoplasm can cause a cell cycle-reentry (CCR) in neurons, causing a mitotic catastrophe and potentially neuron death. Besides, as described previously, U1 snRNP aggregation may be involved in the mechanisms that link plaque development to neurofibrillary tangle (NFT) formation and, therefore. AD pathogenesis. The engineered polynucleotides disclosed (e.g., ASMO1) can target pre-mRNAs in general, like the Tau pre-mRNA, recruiting and helping the U1 complex to stabilize the initial spliceosome assembly at splicing sites, enhancing the accuracy of the splice site selection. Moreover, interactions with key domains of the U1 complex can occur, like with the U1-C zinc finger, which was already proposed to be relevant, if not essential, for the binding of the U1-C snRNP to the U1 complex. These features may allow the engineered polynucleotides (e.g., ASMO1) to facilitate proper recognition of the 5′ end and initiation of the splicing process, even in pathological conditions, preventing also U1 nuclear depletion, cytoplasmic aggregation associated with reduced 3′ processing/premature polyadenylation (in other words, reduction of U1 telescript function).

Accordingly, the present disclosure provides engineered polynucleotides that function as a neurodegenerative disease therapy, and corresponding methods of using the engineered polynucleotides. The engineered polynucleotides may function through a mechanism of homeostatic modulation of U1 snRNPs function, regulation of tau expression, a combination (e.g., synergistically) of both, allowing for a broad application for neurodegenerative disease therapy. To allow this U1 modulation, the engineered polynucleotides can be designed to have size, conformation, and strategic chemical modifications that allow the attraction and interaction with U1C directly, and U1-70K indirectly. The engineered polynucleotides (e.g., ASMO1) can have a sequence complementary to highly conserved regions present pre-mRNAs in general at exon-intron junctions at the 5′ end of introns-called donor splicing sites. The U1 modulation can ensure the correct assembly (with the right distance, position, and behavior) of the snRNPs that make up the U1 complex, which is highly relevant in the splicing process. As splicing could control patterns of expression at a post-transcriptional level, in cells with abnormal gene expression caused by U1 dysfunction, a U1 modulation triggered by the engineered polynucleotides (e.g., ASMO1) could help protein expression normalization. The regulation of tau expression upon treatment with the engineered polynucleotides (e.g., ASMO1) could be a response to this splicing modulation in dysregulated cells, what could be useful for neurodegenerative disease therapy. Altogether, the key roles of U1 snRNPs and tau protein in neurodegenerative disease progression, and our in vitro and in vivo findings indicate that the engineered polynucleotides contemplated in this disclosure can act as a therapeutic agent for the treatment of neurodegenerative diseases.

### Engineered Polynucleotide(s)

The present disclosure provides engineered polynucleotides and methods of using the engineered polynucleotides. Engineered polynucleotides generally refer to polynucleotides that are not naturally occurring. These engineered polynucleotides are not limited to any form of synthesis and can be generated via any method of synthesis (e.g. recombinant technologies, or solid phase synthesis). In some embodiments described herein, the engineered polynucleotide comprises: (i) one or more targeting moiety configured to (e.g., specifically) bind a ribonucleic acid (RNA) (e.g., a messenger ribonucleic acid (mRNA), such as a pre-messenger ribonucleic acid (pre-mRNA)) at a target sequence therein. The engineered polynucleotide may further comprise (ii) a recruiting moiety configured to recruit a post-transcriptional regulating moiety (e.g., a spliceosomal moiety) such that, when associated with the RNA (e.g., the mRNA, such as the pre-mRNA) and the engineered polynucleotide, the post-transcriptional regulating moiety alters the RNA (e.g., the mRNA, such as the pre-mRNA) in or in proximity to the target sequence. In some embodiment, “configured to specifically bind to” refers to hybridizes to. In some embodiments, a moiety configured to specifically bind to a pre-mRNA refers to a moiety that is at least 80%, 90%, or 100% complementary to the pre-mRNA sequence to which it specifically binds. In some embodiments, the RNA (e.g., the mRNA, such as the pre-mRNA) encodes a target gene. In some embodiments, a pre-mRNA associated with an engineered polynucleotide refers to a pre-mRNA hybridized with at least one nucleotide (and up to about 100%) of the engineered polynucleotide. In some embodiments, “alters the RNA” refers to modifying a nucleic acid molecule by cleaving a nucleic acid, reacting a nucleotide of the nucleic acid, or splicing the nucleic acid. In some embodiments, “proximity” refers to a distance of no more than 5 nucleotides away.

An engineered polynucleotide or an engineered polynucleotide as described herein can include various moieties. A “moiety” can refer to a region of an engineered polynucleotide. In some cases, a moiety can be described in terms of a function of the moiety. For instance, a “targeting moiety” can refer to a region of the engineered polynucleotide that can be at least partially complementary to a target RNA; a “recruiting moiety” can refer to a moiety that can recruit any one of the regulating moiety described herein; and a “spacing sequence” can refer to a moiety that provides space between other moieties. In some instances, recitation of a moiety name does not limit the moiety to a particular function. For example, a “targeting moiety” that can be at least partially complementary to a target RNA can in some instances recruit a regulating moiety. The various moieties as described throughout this disclosure can be combined to generate engineered polynucleotides, which can perform a particular function. For example, an engineered polynucleotides can comprise a targeting moiety disclosed herein with a recruiting moiety disclosed herein.

### Targeting Moieties

In various aspects, the engineered polynucleotides comprise one or more targeting moiety. The targeting moiety may allow the engineered polynucleotide to interact with ribonucleic acid (e.g., a pre-mRNA). The targeting moiety may comprise sequences that are complementary or identical to sequence of the ribonucleic acid (e.g., a pre-mRNA) such that the engineered polynucleotide and ribonucleic acid can interact or hybridize. The targeting moiety may be complementary or identical to a target sequence. In some embodiments of the engineered polynucleotide described herein, a targeting moiety of the one or more targeting moiety is sufficiently identical or complementary to a consensus sequence in the target sequence. The target sequence may be in a target gene. The target sequence may be a conserved or consensus sequence that is present in multiple different pre-mRNA. For example, the target sequence may allow the engineered polynucleotides to interact with conserved regions present in a first pre-mRNA that encodes for one gene and in a second pre-mRNA for another gene. Targeting moieties can be found at the 5′ end and 3′ end of an engineered polynucleotide and may also be referred to as lower stems or legs. Without wishing to be bound by theory, stable binding of one or two targeting moieties to a constitutive donor 5′ of a target pre-mRNA may allow the interaction with the conserved site of the constitutive donor and may silence the U1 snRNA RNA-binding domain (RBD). The targeting moieties may comprise sequences that are substantially identical to a sequence of the U1 snRNA RBD. For example, the RBD may comprises the following sequence: 3′-GUCCAUUCAUA-5′ (SEQ ID NO: 6), and the targeting moiety may comprise GTCCA (or GUCCA). The targeting moieties may effectively displace the U1 snRNA RBD from bind the pre-mRNA or prevent binding of the U1 snRNA RBD to the pre-mRNA, based on the on the sequence similarity of the U1 snRNA RBD and the targeting moiety.

A consensus sequence can be determined based on identification of genetic variants of unknown significance (VUS). Any exonic or intronic VUS can be spliceogenic by disrupting the cis DNA sequences that define exons, introns, and regulatory sequences necessary for a correct RNA splicing process. The cis DNA elements can include: exon-intron boundary core consensus nucleotides (e.g., GT at +1 and +2 of the 5′donor site and AG at −1 and −2 of the 3′ acceptor site); or intronic and exonic nucleotides adjacent to these invariable nucleotides that are also highly conserved and have been found to be involved for splice site selection (e.g., CAG/GUAAGU in donor sites and NYAG/G in acceptor sites). A nucleotide change in any of these elements can lead to incorrect splice site recognition, creating a new splice site or activating a cryptic splice site, resulting in aberrant transcripts or non-functional proteins associated with a disease or condition. In some embodiments, at least one targeting moiety of the one or more targeting moiety is sufficiently identical or complementary to a consensus sequence in the target sequence of a target ribonucleic acid or target gene. In some embodiments, the consensus sequence comprises about 2 to about 15 nucleotides, about 2 nucleotides to about 10) nucleotides, or about 2 nucleotides to about 8 nucleotides. In some embodiments, at least two targeting moieties of the one or more targeting moiety are sufficiently identical or complementary to at least two consensus sequences in the target sequence of a target gene. In some embodiments, the one or more targeting moiety are each independently sufficiently identical or complementary to a consensus sequence in the target sequence of a target gene.

In some embodiments of the engineered polynucleotide described herein, the one or more targeting moiety each independently comprises about 2 to about 15 nucleotides, about 2 nucleotides to about 10 nucleotides, or about 2 nucleotides to about 8 nucleotides. In some embodiments of the engineered polynucleotide described herein, the one or more targeting moiety each independently comprises 2, 3, 4, 5, 6, 7, 8, 9, or 10 nucleotides

In some embodiments of the engineered polynucleotide described herein, the one or more targeting moiety comprises (1) a first targeting moiety configured to specifically bind a first targeted sequence in the target sequence of the RNA (e.g., the mRNA, such as the pre-mRNA), and (2) a second targeting moiety configured to specifically bind a second targeted sequence in the target sequence of the RNA (e.g., the mRNA, such as the pre-mRNA). In some embodiments, the first targeted sequence comprises a consensus sequence in the target sequence. In some embodiments, the consensus sequence of the first targeted sequence comprises about 2 to about 15 nucleotides, about 2 nucleotides to about 10 nucleotides, or about 2 nucleotides to about 8 nucleotides. In some embodiments, the consensus sequence of the first targeted sequence comprises 2-3, 3-4, 4-5, 5-6, 6-7, 7-8, or 8-10 nucleotides. In some embodiments, the second targeted sequence comprises a consensus sequence in the target sequence. In some embodiments, the consensus sequence of the second targeted sequence comprises about 2 to about 15 nucleotides, about 2 nucleotides to about 10 nucleotides, or about 2 nucleotides to about 8 nucleotides. In some embodiments, the consensus sequence of the second targeted sequence comprises 2-3, 3-4, 4-5, 5-6, 6-7, 7-8, or 8-10) nucleotides. In some embodiments, the consensus sequence of the first targeted sequence and the consensus sequence of the second targeted sequence are of different nucleotide lengths. In some embodiments, one of the consensus sequences of the first and second targeted sequences comprises about 1 to about 5 nucleotides, and the other of the consensus sequences of the first and second targeted sequences comprises about 4 to about 8 nucleotides. In some embodiments, one of the consensus sequences of the first and second targeted sequences comprises at least about 2 nucleotides, and the other of the consensus sequences of the first and second targeted sequences comprises at least about 5 or 6 nucleotides. In some embodiments, one of the consensus sequences of the first and second targeted sequences comprises about 2 nucleotides, and the other of the consensus sequences of the first and second targeted sequences comprises about 5 or 6 nucleotides.

In some embodiments of the engineered polynucleotide described herein, a first targeting moiety and a second targeting moiety are of different nucleotide lengths. In some embodiments, one of a first targeting moiety and a second targeting moiety comprises about 1 to about 5 nucleotides, and the other of a first targeting moiety and a second targeting moiety comprises about 4 to about 8 nucleotides. In some embodiments, one of a first targeting moiety and a second targeting moiety comprises at least about 2 nucleotides, and the other of a first targeting moiety and a second targeting moiety comprises at least about 5 or 6 nucleotides. In some embodiments, one of a first targeting moiety and a second targeting moiety comprises about 2 nucleotides, and the other of a first targeting moiety and a second targeting moiety comprises about 5 or 6 nucleotides.

In some embodiments, a (e.g., first or second) targeting moiety comprises a nucleic acid sequence having at least one, two, three, four, five, six, seven, eight, nine, ten, or more nucleotides. In some embodiments, a (e.g., first or second) targeting moiety comprises a nucleic acid sequence having at most ten, nine, eight, seven, six, five, four, three, or two nucleotides. In some embodiments, a (e.g., first or second) targeting moiety comprises a nucleic acid sequence having one, two, three, four, five, six, seven, eight, nine, or ten nucleotides, or a range between any two foregoing values.

In some embodiments, the (e.g., first or second) targeting moiety comprises a nucleic acid sequence targeting the nucleotides in an exon. In some embodiments, the (e.g., first or second) targeting moiety comprises a nucleic acid sequence targeting the nucleotides in an exon that are immediately adjacent to an intron. In some embodiments, the (e.g., first or second) targeting moiety comprises a nucleic acid sequence targeting the nucleotides in an exon that are adjacent to or immediately adjacent to an intron that is at the 3′ end of the exon. In some embodiments, the (e.g., first or second) targeting moiety comprises a nucleic acid sequence targeting the nucleotides in an exon that are adjacent to or immediately adjacent to an intron that is at the 5′ end of the exon. In some embodiments, the (e.g., first or second) targeting moiety comprises a nucleic acid sequence comprising 5′-AA-3′, 5′-AT-3′, 5′-AC-3′, 5′-AG-3′, 5′-TA-3′, 5′-TT-3′, 5′-TC-3′, 5′-TG-3′, 5′-CA-3′, 5′-CT-3′, 5′-CC-3′, 5′-CG-3′, 5′-GA-3′, 5′-GT-3′, 5″-GC-3′, or 5′-GG-3′ targeting the nucleotides that are in the exon. In some embodiments, the (e.g., first or second) targeting moiety comprises a nucleic acid sequence targeting the nucleotides in an exon that is immediately adjacent to an intron that is at the 5′ end of the exon. In some embodiments, the (e.g., first or second) targeting moiety comprises a nucleic acid sequence comprising 5′-AA-3′, 5′-AT-3′, 5′-AC-3′, 5′-AG-3′, 5′-TA-3′, 5′-TT-3′, 5′-TC-3′, 5′-TG-3′, 5′-CA-3′, 5′-CT-3′, 5′-CC-3′, 5′-CG-3′, 5′-GA-3′, 5′-GT-3′, 5′-GC-3′, or 5′-GG-3′ targeting the nucleotides that are in the exon that is adjacent to the an intron. In some embodiments, the (e.g., first or second) targeting moiety comprises a nucleic acid sequence targeting the nucleotides in an exon that is immediate adjacent to an intron that is at the 5′ end of the exon. In some embodiments, the (e.g., first or second) targeting moiety comprises a nucleic acid sequence comprising 5′-AA-3′, 5′-AT-3′, 5′-AC-3′, 5′-AG-3′, 5′-TA-3′, 5′-TT-3′, 5′-TC-3′, 5′-TG-3′, 5′-CA-3′, 5′-CT-3′, 5′-CC-3′, 5′-CG-3′, 5′-GA-3′, 5′-GT-3′, 5′-GC-3′, or 5′-GG-3′ targeting the nucleotides that are in the exon that is at the 5′ of an intron. In some embodiments, the (e.g., first or second) targeting moiety comprises a nucleic acid sequence comprising 5′-AA-3′, 5′-AT-3′, 5′-AC-3′, 5′-AG-3′, 5′-TA-3′, 5′-TT-3′, 5′-TC-3′, 5′-TG-3′, 5′-CA-3′, 5′-CT-3′, 5′-CC-3′, 5′-CG-3′, 5′-GA-3′, 5′-GT-3′, 5′-GC-3′, or 5′-GG-3′ targeting the nucleotides that are in the exon that is at the 3′ of an intron. In some embodiments, the (e.g., first or second) targeting moiety comprises a nucleic acid sequence comprising 5′-AA-3′, 5′-AT-3′, 5′-AC-3′, 5′-AG-3′, 5′-TA-3′, 5′-TT-3′, 5′-TC-3′, 5′-TG-3′, 5′-CA-3′, 5′-CT-3′, 5′-CC-3′, 5′-CG-3′, 5′-GA-3′, 5′-GT-3′, 5′-GC-3′, or 5′-GG-3′ targeting the nucleotides that are in the exon that is not immediately adjacent to an intron.

In some embodiments, the (e.g., first or second) targeting moiety comprises a nucleic acid sequence targeting the nucleotides in intron. In some embodiments, the (e.g., first or second) targeting moiety comprises a nucleic acid sequence targeting the nucleotides in an intron that are adjacent to or immediately adjacent to the exon. In some embodiments, the (e.g., first or second) targeting moiety comprises a nucleic acid sequence targeting the nucleotides in an intron that are adjacent to or immediately adjacent to the exon that is at the 5′ end of the intron. In some embodiments, the (e.g., first or second) targeting moiety comprises a nucleic acid sequence targeting the nucleotides in an intron that are adjacent to or immediately adjacent to the exon that is at the 5′ end of the intron. In some embodiments, the (e.g., first or second) targeting moiety comprises a nucleic acid sequence targeting the nucleotides that are located entirely within an intron. In some embodiments, the (e.g., first or second) targeting moiety comprises a nucleic acid sequence comprising 5′-AA-3′, 5′-AT-3′, 5′-AC-3′, 5′-AG-3′, 5′-TA-3′. 5′-TT-3′, 5′-TC-3′, 5′-TG-3′, 5′-CA-3′, 5′-CT-3′, 5′-CC-3′, 5′-CG-3′, 5′-GA-3′, 5′-GT-3′, 5′-GC-3′, or 5′-GG-3′ targeting the nucleotides that are in an intron that is adjacent to the exon. In some embodiments, the (e.g., first or second) targeting moiety comprises a nucleic acid sequence comprising 5′-AA-3′, 5′-AT-3′, 5′-AC-3′, 5′-AG-3′, 5′-TA-3′, 5′-TT-3′, 5′-TC-3′, 5′-TG-3′, 5′-CA-3′, 5′-CT-3′, 5′-CC-3′, 5′-CG-3′, 5′-GA-3′, 5′-GT-3′, 5′-GC-3′, or 5′-GG-3′ targeting the nucleotides that are in the intron and immediately adjacent to the exon that is at the 5′ end of the intron. In some embodiments, the (e.g., first or second) targeting moiety comprises a nucleic acid sequence comprising 5′-AA-3′, 5′-AT-3′, 5′-AC-3′, 5′-AG-3′, 5′-TA-3′, 5′-TT-3′, 5′-TC-3′, 5′-TG-3′, 5′-CA-3′, 5′-CT-3′, 5′-CC-3′, 5′-CG-3′, 5′-GA-3′, 5′-GT-3′, 5′-GC-3′, or 5′-GG-3′ targeting the nucleotides that are in the intron and immediately adjacent to the exon that is at the 3′ end of the intron. In some embodiments, the (e.g., first or second) targeting moiety comprises a nucleic acid sequence comprising 5′-AA-3′, 5′-AT-3′, 5′-AC-3′, 5′-AG-3′, 5′-TA-3′, 5′-TT-3′, 5′-TC-3′, 5′-TG-3′, 5′-CA-3′, 5′-CT-3′, 5′-CC-3′, 5′-CG-3′, 5′-GA-3′, 5′-GT-3′, 5′-GC-3′, or 5′-GG-3′ targeting the nucleotides that are in the intron that is not adjacent to an exon.

As described, a targeting moiety (e.g., first or second) may be gene agnostic such that it can target conserved regions that are shared (or are substantially similar) in pre-mRNA of multiple different genes. Additionally, a targeting moiety (e.g., first or second) may target a specific gene, or specific exon and intron of a gene. As an example. Table 1 provides the MAPT gene Exon-Intron junction sequences for multiple exons-introns junctions. For example using Table 1 (or other sequences from a gene or exon-intron junctions of a gene of interest), engineered polynucleotides can be designed such to target a specific exon-intron junction, or may be designed to target more than one exon-intron junction. In some embodiments of the engineered polynucleotide described herein, a (e.g., first or second) targeting moiety comprises a sequence at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical or complementary to a sequence set forth in Table 1. In some embodiments of the engineered polynucleotide described herein, a (e.g., first or second) targeting moiety comprises a sequence identical or complementary to a sequence set forth in Table 1. In some embodiments, a (e.g., first or second) targeting moiety comprises a sequence at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical or complementary to a sequence selected from the “Exon sequence” column of Table 1 and the “Intron sequence” column of Table 1. In some embodiments, a (e.g., first or second) targeting moiety comprises a sequence identical or complementary to a sequence selected from the “Exon sequence” column of Table 1 and the “Intron sequence” column of Table 1.

In some embodiments, a first targeting moiety comprises a sequence at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical or complementary to a sequence selected from the “Exon sequence” column of Table 1; and a second targeting moiety comprises a sequence at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical or complementary to a sequence set forth in the “Intron sequence” column of Table 1. In some embodiments, a first targeting moiety comprises a sequence identical or complementary to a sequence selected from the “Exon sequence” of Table 1; and a second targeting moiety comprises a sequence identical or complementary to a sequence set forth in the “Intron sequence” column of Table 1. In some embodiments, a first targeting moiety comprises a sequence at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical or complementary to a sequence set forth in the “Intron sequence” column of Table 1; and a second targeting moiety comprises a sequence at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical or complementary to a sequence set forth in the “Exon sequence” of Table 1. In some embodiments, a first targeting moiety comprises a sequence identical or complementary to a sequence set forth in the “Intron sequence” column of Table 1; and a second targeting moiety comprises a sequence identical or complementary to a sequence set forth in the “Exon sequence” column of Table 1. In some embodiments, a (e.g., first or second) targeting moiety comprises a sequence identical or complementary to a consensus sequence of an intron donor site (e.g., selected from GU, GT, GC, and CA). In some embodiments, a (e.g., first or second) targeting moiety comprises a sequence identical or complementary to a consensus sequence of an exon donor site (e.g., G). In some embodiments, a (e.g., first or second) targeting moiety comprises a sequence identical or complementary to a consensus sequence selected from GU, GC, G, and CA.

TABLE 1
Examples of MAPT gene Exon-Intron junction
sequences targeted by the 5′ and 3′-targeting
moieties of engineered polynucleotides and the
distance between the two target sequences

| | Exon | | Intron |
|---|---|---|---|
| Exon | sequence | Spacer | sequence |
| Exon 1 | 5′-CAGGT-3′ | 2 nt | 3′-CG-5′ |
| Exon 2 | 5′-AAGGT-3′ | 2 nt | 3′-TG-5′ |
| Exon 3 | 5′-AAGGT-3′ | 2 nt | 3′-CG-5′ |
| Exon 4 | 5′-CAGGT-3′ | 2 nt | 3′-GG-5′ |
| Exon 5 | 5′-AAGGT-3′ | 2 nt | 3′-TG-5′ |
| Exon 6a | 5′-AAGGT-3′ | 2 nt | 3′-TG-5′ |
| Exon 6b | 5′-AAGGT-3′ | 2 nt | 3′-TG-5′ |
| Exon 7 | 5′-AAGGT-3′ | 2 nt | 3′-CG-5′ |
| Exon 8 | 5′-AAGGT-3′ | 2 nt | 3′-GG-5′ |
| Exon 9 | 5′-CTGGT-3′ | 1 nt | 3′-AG-5′ |
| Exon 10 | 5′-CTGGT-3′ | 2 nt | 3′-AG-5′ |
| Exon 11 | 5′-AGTGT-3′ | 2 nt | 3′-TG-5′ |
| Exon 12 | 5′-CAGGT-3′ | 1 nt | 3′-CG-5′ |
| Exon 13 | 5′-AAGGT-3′ | 3 nt | 3′-GG-5′ |
| Exon 14 | 5′-AAGGT-3′ | 2 nt | 3′-CC-5′ |
| Exon 1 | 5′-AAAAG-3′ | 3 nt | 3′-CGA-5′ |
| Exon 2 | 5′-AAAAG-3′ | 3 nt | 3′-TGA-′5 |
| Exon 3 | 5′-GGAAG-3′ | 3 nt | 3′-GGG-′5 |
| Exon 4 | 5′-CACAG-3′ | 3 nt | 3′-GGA-5′ |
| Exon 5 | 5′-CCAAG-3′ | 3 nt | 3′-TGA-5′ |
| Exon 6a | 5′-CAAAG-3′ | 3 nt | 3′-TGT-5′ |
| Exon 6b | 5′-CAAAG-3′ | 3 nt | 3′-TGT-5′ |
| Exon 7 | 5′-CCAAG-3′ | 3 nt | 3′-CGT-5′ |
| Exon 8 | 5′-TCAAG-3′ | 3 nt | 3′-GGA-5′ |
| Exon 9 | 5′-CTCTG-3′ | 3 nt | 3′-AGA-5′ |
| Exon 10 | 5′-GGAAG-3′ | 3 nt | 3′-AGA-5′ |
| Exon 11 | 5′-GGCAG-3′ | 3 nt | 3′-AGA-5′ |
| Exon 12 | 5′-AACAG-3′ | 2 nt | 3′-CGA-5′ |
| Exon 13 | 5′-AAAAG-3′ | 3 nt | 3′-GAA-5′ |
| Exon 14 | 5′-TTAAA-3′ | 2 nt | 3′-GGA-5′ |

Example consensus sequences include: (e.g., 5′-) intron donor site #1: GU; (e.g., 5′-) intron donor site #2: GC; (e.g., 5′-) exon donor site #1: G; and (e.g., 5′-) intron donor site #3: CA.

A targeting moiety (e.g., first or second) may be specific to a gene, or specific exon and intron of a gene. For example, the targeting moiety may be specific to an exon-intron junction and a portion of the exon or intron. In some embodiments, the targeting moiety is specific to a portion of the gene that is outside of the splice site. In some embodiments, the targeting moiety targets a specific gene. For example, the targeting moiety may comprise a section that is specific to the splice site and a specific to the gene. As described elsewhere herein, the targeting moiety may comprise sequences of consensus splice site. By having a targeting moiety of specific to consensus splice sites, the targeting moiety may be gene agnostic and target multiple different mRNA of different genes. Alternatively, the targeting moieties may comprise a portion that is specific to a gene. The engineered polynucleotide may then be able to modulate the splicing of a specific target gene (as opposed to any gene/mRNA that comprises a consensus splice site). For example, the targeting moieties may comprise sequences complementary to sequences unique to MAPT.

### Target Sequence

As described in this disclosure, the engineered polynucleotides may comprise one or more targeting moieties. The targeting moieties may target (e.g., bind to, hybridize to, or otherwise be configured to interact with) a target sequence, for example, where the targeting moieties target adjacent or nearby regions in a target sequence. The targeting moieties may be complementary to a targeted sequence. In some embodiments of the engineered polynucleotide described herein, a first targeted sequence and a second targeted sequence are apart in the target sequence by a spacing sequence of no more than five, four, or three nucleotides (e.g., one or two nucleotides).

In some embodiments of the engineered polynucleotide described herein, a first targeted sequence and a second targeted sequence are contiguous or adjacent to each other.

In some embodiments, when a spacing sequence in the target sequence is adjacent to a 5′- or 3′-end of a targeted sequence of the target sequence, the spacing sequence may not be complementary to a targeting moiety of the engineered polynucleotide. In some embodiments, when a spacing sequence in the target sequence is adjacent to a 5′- or 3′-end of a targeted sequence of the target sequence, the spacing sequence may not be complementary to any targeting moiety of the engineered polynucleotide.

In some embodiments, the spacing sequence separate a first targeted sequence and a second targeted sequence described herein. In some embodiments, the spacing sequence is not complementary and does not bind to a targeting moiety of the engineered polynucleotide. In some embodiments, the spacing sequence is not complementary and does not bind to all targeting moieties of the engineered polynucleotide.

In some embodiments of the engineered polynucleotide described herein, the target sequence may comprise an exon-intron boundary in the RNA (e.g., the mRNA, such as the pre-mRNA). In some embodiments, a first targeted sequence and a second targeted sequence are both 5′ or 3′ with respect to the exon-intron boundary. In some embodiments, one of a first targeted sequence and a second targeted sequence is 5′ with respect to the exon-intron boundary, and the other of a first targeted sequence and a second targeted sequence is 3′ with respect to the exon-intron boundary.

In some embodiments of the engineered polynucleotide described herein, the target sequence comprises a splice site in the RNA (e.g., the mRNA, such as the pre-mRNA). In some embodiments, a (e.g., first or second) targeted sequence comprises a splice site (e.g., 5′ ss) in the RNA (e.g., the mRNA, such as the pre-mRNA).

In some embodiments, two targeted sequences (e.g., a first targeted sequences and a second targeted sequence) are part of a single nucleic acid molecule (i.e., the RNA, e.g., the mRNA, such as the pre-mRNA). In some embodiments, the first and second targeted sequences are spanned apart on the single nucleic acid molecule. In some embodiments, the first and second targeted sequences span an exon-intron boundary of the single nucleic acid molecule. In some embodiments, the first and second targeted sequences do not span an exon-intron boundary of the single nucleic acid molecule. In some embodiments, the first and second targeted sequences are adjacent to an exon-intron boundary of the single nucleic acid molecule. In some embodiments, the first and second targeted sequences both target an intron of the single nucleic acid molecule. In some embodiments, the first and second targeted sequences span a splice site in the single nucleic acid molecule. In some embodiments, the first and second targeted nucleic acid sequences do not span a splice site in the single nucleic acid molecule.

In some embodiments of the engineered polynucleotide described herein, a consensus sequence in the target sequence comprises about 2 to about 15 nucleotides, about 2 nucleotides to about 10 nucleotides, or about 2 nucleotides to about 8 nucleotides.

In some embodiments, the engineered polynucleated in complementary and binds to a target sequence. In some embodiments, at least a portion of the engineered polynucleotide binds to a target sequence. In some embodiments, the target sequence encodes for a gene (e.g., a target gene). Non-limiting example of the gene can include microtubule associated protein tau (MAPT).

In some embodiments, the target sequence comprises an RNA sequence. In some embodiments, the RNA is a nuclear RNA, a cytoplasmic RNA, or a mitochondrial RNA. In some embodiments, the target RNA sequence comprises a messenger RNA (mRNA), a pre-messenger RNA (pre-mRNA), a transfer RNA (tRNA), a ribosomal RNA (rRNA), a ribozyme, a recombinant polynucleotide, a branched polynucleotide, an isolated RNA, an guide RNA, an oligonucleotide, a nucleic acid probe, a primer, an snRNA, a long non-coding RNA, a small RNA, a snoRNA, a siRNA, a miRNA, a tRNA-derived small RNA (tsRNA), an antisense RNA, an shRNA, or a small rDNA-derived RNA (srRNA). In some embodiments, the target RNA sequence is a pre-mRNA. In some embodiments, the engineered polynucleotide is not an antisense oligonucleotide.

In some embodiments, the target RNA sequence comprises at least one exon. In some embodiments, the target RNA sequence comprises at least one intron. In some embodiments, the target RNA sequence comprises at least one exon or at least one intron. In some embodiments, the target RNA sequence comprises at least one exon-intron boundary. The target RNA sequence may comprise a sequence specific to a gene. For example, the target RNA sequence may comprise sequences encoding for a polypeptide. For example, the target RNA sequence may comprise sequences encoding for a Tau. The target sequence may comprise a sequence corresponding to an exon-intron boundary and a sequence corresponding to a specific gene or a consensus/conserved sequence present in many genes.

In some embodiments, the target sequence is an endogenous nucleic acid molecule. In some embodiments, the binding of the engineered polynucleotide to the target sequence by base pairing such as Watson-Crick base pairing.

### Recruiting Moiety

As described in this disclosure, the engineered polynucleotides can comprise a recruiting moiety. The recruiting moiety can recruit one or more components of the spliceosome. In some embodiments, a recruiting moiety configured to recruit a spliceosomal moiety refers to a recruiting moiety that hybridizes to the spliceosomal moiety. In some embodiments, a recruiting moiety configured to recruit a spliceosomal moiety refers to a recruiting moiety that is at least 80%, 90%, or 100% complementary to the spliceosomal moiety or a sequence of the spliceosomal moiety. In some embodiments, “recruit” refers to formation of at least one hydrogen bond between the recruiting moiety and the spliceosomal moiety. In some embodiments, “recruit” refers to hybridization between at least one nucleotide of the recruiting moiety and at least one nucleotide of the spliceosomal moiety. A recruiting moiety comprises a hairpin structure. The hairpin may be a full hairpin or may be intercalated by internal loops. The hairpin structure may consist of 13 to 17 nucleotides. The recruiting moiety may interact with stem-loop II of the U1 snRNA. The recruiting moiety can comprise a sequence complementary to a sequence or portion of stem-loop II. For example, the stem-loop II of the U1 snRNA comprise the sequence 5′-GUAGGCCUCACGUUACCUAU-3′, and the recruiting moiety may comprises 5′-CCGGA-3′. U1-A also can bind to stem-loop II of the U1 snRNA. Hydrogen bridges of stem-loop II with the hairpin/internal loop region can indirectly modulate polyadenylation and acetylation signaling by U1-A. The recruiting moiety may not silence the anchoring domain of U1-A in stem-loop II. This interaction can modulate gene expression and acetylation.

In some embodiments of the engineered polynucleotide described herein, the recruiting moiety comprises a nucleotide sequence that is at least 70%, 80%, 85%, or 90% identical or complementary to a sequence set forth in Table 2. In some embodiments, the recruiting moiety comprises a nucleotide sequence that is identical or complementary to a sequence set forth in Table 2. In some embodiments, the engineered polynucleotide comprises a nucleotide sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical or complementary to any sequence selected from SEQ ID NOs: 1-2 In some embodiments, the engineered polynucleotide comprises a nucleotide sequence that is at least 70%, 80%, 85%, or 90% identical or complementary to any sequence selected from SEQ ID NOs: 1-2. In some embodiments, the engineered polynucleotide comprises a nucleotide sequence that is identical or complementary to any sequence selected from SEQ ID NOs: 1-2.

TABLE 2
Example recruiting moieties of engineered
polynucleotides described herein

| | | Nucleo- | |
|---|---|---|---|
| SEQ ID | | tide | |
| No. | Sequence | length | |
| 1 | ctaacctttcaggccag | 17 nt | ASMO1 (DNA) |
| 2 | cuaaccuuucaggccag | 17 nt | ASMO1 (RNA) |

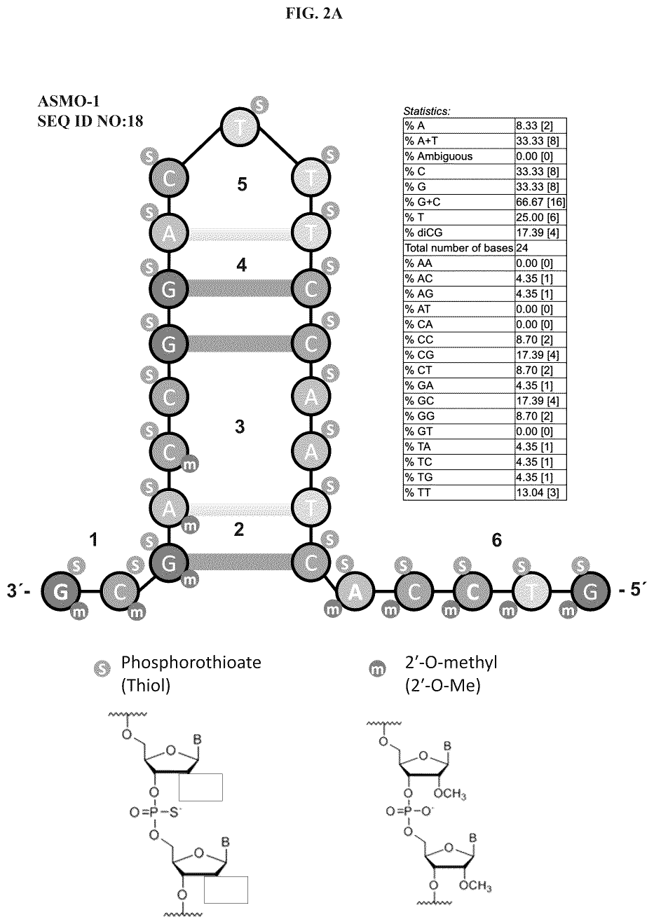

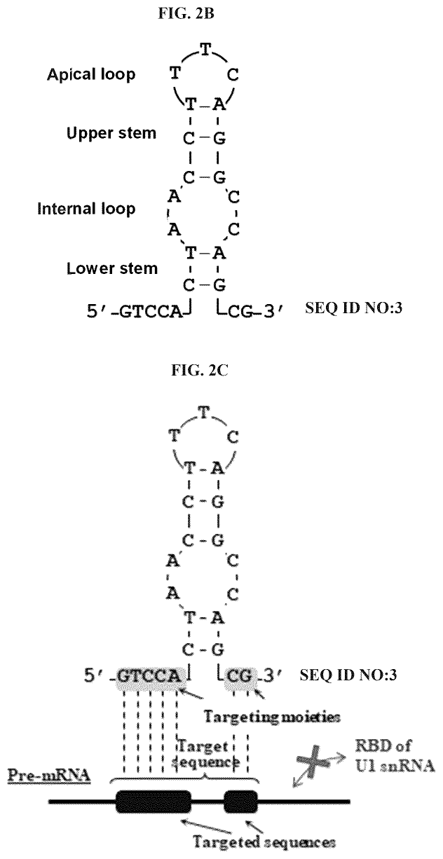

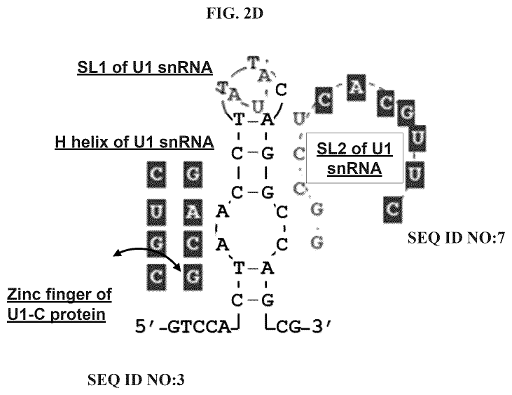

In some embodiments described herein, the engineered polynucleotide (e.g., the recruiting moiety) comprises a (e.g., secondary) structural feature (see FIGS. 2A-2D). In some embodiments, the engineered polynucleotide (e.g., the recruiting moiety) comprises an apical loop, an upper stem, an internal loop, a lower stem, or a combination thereof. In some embodiments, the engineered polynucleotide (e.g., the recruiting moiety) comprises a loop (e.g., an internal loop) adjacent to a stem (e.g., a lower stem or an upper stem) comprising two complementary stem sequences. In some embodiments, a stem sequence of the stem (e.g., the lower stem or the upper stem) comprises no more than about five, four, or three nucleotides. In some embodiments, the loop is an internal loop adjacent to the stem (e.g., the lower stem) and a further stem (e.g., an upper stem) comprising two complementary stem sequences. In some embodiments, the internal loop comprises a nucleic acid sequence of no more than 10, 9, or 8 nucleotides. In some embodiments, a stem sequence of the further stem (e.g., the upper stem) comprises no more than about five, four, or three nucleotides. In some embodiments, the engineered polynucleotide (e.g., the recruiting moiety) further comprises an apical loop. In some embodiments, the apical loop comprises a nucleic acid sequence of no more than 10, 9, 8, 7, 6, or 5 nucleotides.

In some embodiments of the engineered polynucleotide described herein, the recruiting moiety comprises about 10 to about 30 nucleotides, about 10 to about 25 nucleotides, or about 10 to about 20 nucleotides.

In some embodiments, the recruiting moiety is partially complementary to a post-transcriptional regulating moiety (or a regulating moiety) (e.g., a spliceosomal moiety) comprising a ribonucleoprotein complex. For example, the recruiting moiety can be partially complementary to a regulating moiety comprising a spliceosomal ribonucleoprotein complex, where the spliceosomal ribonucleoprotein complex comprises a small nuclear ribonucleic acid (snRNA). In some embodiments, the recruiting moiety is not complementary and does not bind to a target sequence described herein. For example, the recruiting moiety is not complementary and does not bind to a pre-mRNA described herein.

### Structural Configurations

In various aspects, the engineered polynucleotides comprise one or more moieties that can perform a function. These one or more moieties may be present in the engineered polypeptide in various structural configurations to allow for the engineered polynucleotide to perform a given function (e.g., recruitment of the regulating moiety or component of a spliceosome, or binding to a pre-mRNA). In some embodiments of the engineered polynucleotide described herein, one of the first and second targeting moieties is 5′ with respect to the recruiting moiety, and the other of the first and second targeting moieties is 3′ with respect to the recruiting moiety.

In some embodiments, the engineered polynucleotide has a structural arrangement from 5′-terminus to 3′-terminus as follows: a first targeting moiety, a recruiting moiety, and a second targeting moiety. In some embodiments, the engineered polynucleotide has a structural arrangement from 5′-terminus to 3′-terminus as follows: a second targeting moiety, a recruiting moiety, and a first targeting moiety.

### Example Polynucleotides

In some embodiments of the engineered polynucleotide described herein, the engineered polynucleotide comprises about 10 to about 40 nucleotides, about 10 to about 35 nucleotides, about 10 to about 30 nucleotides, or about 10 to about 25 nucleotides. In some embodiments, the engineered polynucleotide comprises a length of at least about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, or more nucleotides. In some embodiments, the engineered polynucleotide comprises a length of at least about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, or less nucleotides. In some embodiments, the engineered polynucleotide comprises a length of at least about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50 nucleotides, or a range between any two foregoing values.

In some embodiments of the engineered polynucleotide described herein, the recruiting moiety comprises a nucleotide sequence that is at least 70%, 80%, 85%, or 90% identical or complementary to a sequence set forth in Table 3. In some embodiments, the recruiting moiety comprises a nucleotide sequence that is identical or complementary to a sequence set forth in Table 3.

TABLE 3
Example sequences of engineered polynucleotides comprising
recruiting moieties and targeting moieties

| SEQ ID | | Nucleotide | |
|---|---|---|---|
| No. | Sequence | length | |
| 3 | gtccactaacctttcaggccagcg | 24 nt | ASMO1 |
| 4 | guccacuaaccuuucaggccagcg | 24 nt | ASMO1 |

In some embodiments, the engineered polynucleotide can be produced from a precursor of the engineered polynucleotide. In some cases, a precursor of the engineered polynucleotide can be linear. For example, a precursor of the engineered polynucleotide can be a linear polynucleotide transcribed from a plasmid. In another example, a precursor of the engineered polynucleotide can be constructed to be a linear polynucleotide with moieties such as a ribozyme moiety and a ligation moiety that allow for circularization of the engineered polynucleotide in a cell. The linear engineered polynucleotide with the ligation and ribozyme moieties can be transfected into a cell, where it can be circularized. In some cases, the engineered polynucleotide can be circular. In some cases, the engineered polynucleotide comprises DNA, RNA or both. In some cases, a precursor of the engineered polynucleotide comprises a precursor of the engineered polynucleotide. In some cases, a precursor of the engineered polynucleotide can be used to produce an engineered polynucleotide.

In some embodiments, the engineered polynucleotide comprises at least one secondary structure (such as those described anywhere herein). For example, the engineered polynucleotide comprises at least one, two, three, four, or more secondary structures, where the secondary structures can be any one or any combination of an apical loop, a stem, a stem loop, or an internal loop. The one or more secondary structures may perform a variety of different functions. For example, the secondary structure may provide stability, or otherwise help stabilize the engineered polynucleotide. The secondary structure may bind to, or interact with polypeptide or other polynucleotide. For example, the secondary structure may interact with a component of the spliceosome. In some embodiments, the recruiting moiety of the polynucleotide comprises at least one, two, three, four, or more secondary structures. In some embodiments, the targeting moiety does not have a secondary structure. In some embodiments, the secondary structure is an apical loop comprising at least two, three, four, five, six, seven, eight, nine, 10, or more nucleotides. In some embodiments, the apical loop is complementary and binds to the regulating moiety. In some embodiments, the apical loop is not complementary and does not bind to the regulating moiety. In some embodiments, the secondary structure is at least one stem. In some embodiments, the engineered polynucleotide comprises two stems, where one is an upper stem that is close to the apical loop and the other is a lower stem closer to the targeting moiety. In some embodiments, the upper stem comprises at least two, four, six, eight, 10, or more nucleotides, where the nucleotides are paired to form the upper stem. In some embodiments, the upper stem is complementary and binds to the regulating moiety. In some embodiments, the upper stem is not complementary and does not bind to the regulating moiety. In some embodiments, the secondary structure is a lower stem, where the lower stem comprises at least two, four, six, eight, 10, or more nucleotides, where the nucleotides are paired to form the lower stem. In some embodiments, the lower stem is complementary and binds to the regulating moiety. In some embodiments, the lower stem is not complementary and does not bind to the regulating moiety. In some embodiments, the engineered polynucleotide comprises an internal loop between the upper stem and the lower stem. In some embodiments, the internal loop comprises at least two, three, four, five, six, seven, eight, nine, 10, or more nucleotides. In some embodiments, the internal loop is complementary and binds to the regulating moiety. In some embodiments, the internal loop is not complementary and does not bind to the regulating moiety. In some embodiments, the engineered polynucleotide comprises secondary structures comprising an apical loop, an upper stem, an internal loop, and a lower stem, where the upper stem and the internal loop are at least partially complementary and bind to the regulating moiety. In some embodiments, the upper stem and the internal loop are complementary and bind to the regulating moiety comprising snRNA. In some embodiments, the snRNA is U1 snRNA such as US-A snRNA. In some embodiments, the snRNA is U2 snRNA.

In some embodiments, the nucleic acid sequence of the at least one secondary structure is partially complementary to the regulating moiety comprising the ribonucleoprotein complex. In some embodiments, the nucleic acid sequence of the at least one secondary structure is not complementary to the targeted nucleic acid sequence. In some embodiments, the engineered polynucleotide comprises at least one secondary structure of the nucleic acid. In some embodiments, the engineered polynucleotide comprises at least one, two, three, four, or more secondary structure of the nucleic acid. In some embodiments, the at least one secondary structure increases the binding between the recruiting moiety and the regulating moiety. In some embodiments, the at least one secondary structure stabilizes the assembly of the regulating moiety. In some embodiments, the at least one secondary structure stabilizes the assembly of the regulating moiety with other additional moiety. In some embodiments, the at least one secondary structure increases the efficiency of modulating the expression or activity of the gene encoding by the target sequence. In some embodiments, the at least one secondary structure increases the specificity of modulating the expression or activity of the gene encoding by the target sequence. In some embodiments, the at least one secondary structure increases the resistance of the engineered polynucleotide to degradation by hydrolysis. In some embodiments, the at least one secondary structure increases the resistance of the engineered polynucleotide to degradation by nuclease digestion. In some embodiments, the at least one secondary structure increases half-life of the engineered polynucleotide. In some embodiments, the at least one secondary structure decreases immunogenicity induced by the engineered polynucleotide.

In some embodiments, the engineered polynucleotide is characterized by a secondary structure. In some embodiments, the secondary structure comprises one or more stem-loop structures. In some embodiments, the secondary structure comprises an apical loop, an upper stem, an internal loop, and a lower stem. In some embodiments, the engineered polynucleotide comprises at least one secondary structure. In some embodiments, the first or second targeting moiety is not part of the secondary structure. In some embodiments, the engineered polynucleotide comprises at least one, two, three, four, or more secondary structure. In some embodiments, the engineered polynucleotide comprises a secondary structure comprising a stem-loop, a cruciform, a toe hold, a mismatch bulge, or any combination thereof. In some embodiments, the engineered polynucleotide comprises a secondary structure comprising an apical loop, an upper stem, an internal loop, or a lower stem. In some embodiments, the engineered polynucleotide comprises a secondary structure comprising an apical loop, an upper stem, an internal loop, and a lower stem. In some cases, a secondary structure can comprise a stem, a hairpin loop, a pseudoknot, a bulge, an internal loop, a multiloop, a G-quadruplex, or any combination thereof. In some embodiments, an engineered polynucleotide can adopt an A-form, a B-form, a Z-form, or any combination thereof. In some embodiments, the secondary structure is formed based on, at least partially, the nucleotide sequence of the engineered polynucleotide. In some embodiments, the secondary structure is formed within the nucleotide sequence of the engineered polynucleotide.

In some embodiments, the at least one secondary structure increases the binding between the recruiting moiety and the regulating moiety. In some embodiments, the at least one secondary structure increases the binding between the recruiting moiety and the regulating moiety by at least 10%, 20%, 30%, 40%, 50%, 60%, 70%, 80%, 90%, 2 fold, 3 fold, 4 fold, 5 fold, 10 fold, or more compared to the binding between the recruiting moiety without the secondary structure to the regulating moiety.

In some embodiments, the at least one chemical modification increases the binding between the recruiting moiety and the regulating moiety. In some embodiments, the at least one chemical modification increases the binding between the recruiting moiety and the regulating moiety by at least 10%, 20%, 30%, 40%, 50%, 60%, 70%, 80%, 90%, 2 fold, 3 fold, 4 fold, 5 fold, 10 fold, or more compared to the binding between the recruiting moiety without the chemical modification to the regulating moiety.

In some embodiments, the at least one chemical modification of the engineered polynucleotide stabilizes the assembly of the spliceosome comprising the regulating moiety, when the regulating moiety is associated with the target sequence. In some embodiments, the assembly of the spliceosome comprising the regulating moiety is at least 10%, 20%, 30%, 40%, 50%, 60%, 70%, 80%, 90%, 2 fold, 3 fold, 4 fold, 5 fold, 10 fold, or more stabilized by the engineered polynucleotide comprising the chemical modification compared to a comparable polynucleotide without the chemical modification. In some embodiments, the regulating moiety is U1 (U1SNP), U2 (U2SNP), U4, U5, U6, U11, U12, U14, or U16 of the spliceosome. In some embodiments, the regulating moiety is U1SNP of the spliceosome. In some embodiments, the regulating moiety is U1-A of the spliceosome. In some embodiments, the regulating moiety is U2SNP of the spliceosome. In some embodiments, the at least one chemical modification of the engineered polynucleotide stabilizes the assembly of the spliceosome comprising the regulating moiety and at least one additional moiety. For example, the at least one chemical modification of the engineered polynucleotide stabilizes the assembly of the spliceosome comprising the regulating moiety comprising U1-A and at least one additional moiety comprising U1-70K, UC-1, SmD1, SmD2, SmD3, SmE, SmF, or SmG. In some embodiments, the at least one additional moiety is U4, U5, U6, U11, U12, U14, or U16 of the spliceosome.

In some embodiments, the at least one chemical modification increases the efficiency of the engineered polynucleotide modulating the expression or activity of the gene encoding by the target sequence compared to a comparable polynucleotide without the chemical modification. In some embodiments, the efficiency of the engineered polynucleotide modulating the expression or activity of the gene encoding by the target sequence is increased by at least 10%, 20%, 30%, 40%, 50%, 60%, 70%, 80%, 90%, 2 fold, 3 fold, 4 fold, 5 fold, 10 fold, or more compared to the efficient of the comparable polynucleotide without the chemical modification modulating the expression or activity of the gene encoding by the target sequence.

In some embodiments, the at least one chemical modification increases the specificity of the engineered polynucleotide modulating the expression or activity of the gene encoding by the target sequence compared to a comparable polynucleotide without the chemical modification. In some embodiments, the specificity of the engineered polynucleotide modulating the expression or activity of the gene encoding by the target sequence is increased by at least 10%, 20%, 30%, 40%, 50%, 60%, 70%, 80%, 90%, 2 fold, 3 fold, 4 fold, 5 fold, 10 fold, or more compared to the efficient of the comparable polynucleotide without the chemical modification modulating the expression or activity of the gene encoding by the target sequence.

In some embodiments, the at least one chemical modification increases the resistance of the engineered polynucleotide to degradation by hydrolysis (e.g., degradation via an endonuclease). In some embodiments, the at least one chemical modification increases the resistance of the engineered polynucleotide to degradation by hydrolysis compared to a resistance of a comparable engineered polynucleotide without the chemical modification. In some embodiments, the resistance to degradation by hydrolysis of the engineered polynucleotide comprising the at least one chemical modification is increased by at least 10%, 20%, 30%, 40%, 50%, 60%, 70%, 80%, 90%, 2 fold, 3 fold, 4 fold, 5 fold, 10 fold, or more compared to the resistance of the comparable engineered polynucleotide without the chemical modification.

In some embodiments, the at least one chemical modification increases the resistance of the engineered polynucleotide to degradation by nuclease digestion. In some embodiments, the at least one chemical modification increases the resistance of the engineered polynucleotide to degradation by nuclease digestion compared to a resistance of a comparable engineered polynucleotide without the chemical modification. In some embodiments, the resistance to degradation by nuclease digestion of the engineered polynucleotide comprising the at least one chemical modification is increased by at least 10%, 20%, 30%, 40%, 50%, 60%, 70%, 80%, 90%, 2 fold, 3 fold, 4 fold, 5 fold, 10 fold, or more compared to the resistance of the comparable engineered polynucleotide without the chemical modification.

In some embodiments, the at least one chemical modification increases half-life of the engineered polynucleotide compared to a half-life of a comparable engineered polynucleotide without the chemical modification. In some embodiments, the half-life of the engineered polynucleotide comprising the at least one chemical modification is increased by at least 10%, 20%, 30%, 40%, 50%, 60%, 70%, 80%, 90%, 2 fold, 3 fold, 4 fold, 5 fold, 10 fold, or more compared to the half-life of the comparable engineered polynucleotide without the chemical modification.ne chemical modification increases half-life of the engineered polynucleotide.

In some embodiments, the at least one chemical modification decreases immunogenicity induced by the engineered polynucleotide. In some embodiments, the at least one chemical modification decreases immunogenicity induced by the engineered polynucleotide compared to an immunogenicity of a comparable engineered polynucleotide without the chemical modification. In some embodiments, the immunogenicity of the engineered polynucleotide comprising the at least one chemical modification is decreased by at least 10%, 20%, 30%, 40%, 50%, 60%, 70%, 80%, 90%, 2 fold, 3 fold, 4 fold, 5 fold, 10 fold, or more compared to the immunogenicity of the comparable engineered polynucleotide without the chemical modification.

### Chemical Modification(s)

In some embodiments described herein, the engineered polynucleotide comprises at least one chemical modification. As described in this disclosure, a chemical modification may confer structural or functional advantages to the engineered polynucleotides (e.g., increased half-life), decreased immunogenicity, increased resistance to hydrolysis or enzymatic degradation, improved reactivity or binding to a polypeptide (e.g., components of a spliceosome) or polynucleotide (e.g. pre-mRNA, or components of a spliceosome)

In some embodiments, all nucleotides of a targeting moiety are linked by phosphorothioate bonds. In some embodiments, all nucleotides of a targeting moiety comprise a 2′ O-methyl modification, 2′ modifications can prevent nuclease degradation and/or increase the affinity of the targeting moiety to a pre-mRNA target.

In some embodiments, all nucleotides of a recruiting moiety are linked by phosphorothioate bonds. In some embodiments, three nucleotides of a recruiting moiety comprise a 2′ O-methyl modification. The 2′ modifications can induce a change in the molecular dynamics of the recruiting moiety, thereby facilitating a conformational alteration of stem-loop II of U1-snRNA and binding of the recruiting moiety to U1-snRNA.

In some embodiments, the engineered polynucleotide comprises at least one 2′-modified (e.g., 2′-methoxy, 2′-methoxymethyl, or 2′-methoxyethyl) nucleotides. In some embodiments, at least about 50%, 60%, 70%, 80%, or 90% nucleotides of the engineered polynucleotide are chemically modified nucleotides. In some embodiments, at least about 50%, 60%, 70%, 80%, or 90% nucleotides of the engineered polynucleotide are 2′-modified (e.g., 2′-methoxy, 2′-methoxymethyl, or 2′-methoxyethyl) nucleotides. In some embodiments, the engineered polynucleotide comprises at least one phosphorothioate internucleotide bond. In some embodiments, at least about 50%, 60%, 70%, 80%, or 90% internucleotide linkages of the engineered polynucleotide are chemically modified. In some embodiments, at least about 50%, 60%, 70%, 80%, or 90% internucleotide linkages are phosphorathioate.

In some embodiments, the engineered polynucleotide comprises at least one chemical modification of the nucleic acid. In some embodiments, the engineered polynucleotide comprises at least one, two, three, four, or more chemical modification of the nucleic acid. In some embodiments, the at least one chemical modification increases the binding between the recruiting moiety and the regulating moiety. In some embodiments, the at least one chemical modification stabilizes the assembly of the regulating moiety. In some embodiments, the at least one chemical modification stabilizes the assembly of the regulating moiety with other additional moiety. In some embodiments, the at least one chemical modification increases the efficiency of modulating the expression or activity of the gene encoding by the target sequence. In some embodiments, the at least one chemical modification increases the specificity of modulating the expression or activity of the gene encoding by the target sequence. In some embodiments, the at least one chemical modification increases the resistance of the engineered polynucleotide to degradation by hydrolysis. In some embodiments, the at least one chemical modification increases the resistance of the engineered polynucleotide to degradation by nuclease digestion. In some embodiments, the at least one chemical modification increases half-life of the engineered polynucleotide. In some embodiments, the at least one chemical modification decreases immunogenicity induced by the engineered polynucleotide.

In some embodiments, the chemical modifications of the engineered polynucleotide comprise at least one substitution of one or both of non-linking phosphate oxygen atoms in a phosphodiester backbone linkage of the engineered polynucleotide. In some embodiments, the at least one chemical modification of the engineered polynucleotide comprises a substitution of one or more of linking phosphate oxygen atoms in a phosphodiester backbone linkage of the engineered polynucleotide. A non-limiting example of a chemical modification of a phosphate oxygen atom is a sulfur atom. Additional non-limiting examples are included in Table 3. In some embodiments, the chemical modifications of the engineered polynucleotide comprise at least one chemical modification to a sugar of a nucleotide of the engineered polynucleotide. In some embodiments, the chemical modifications of the engineered polynucleotide comprise at least one chemical modification to the sugar of the nucleotide, where the chemical modification comprises at least one locked nucleic acid (LNA). In some embodiments, the chemical modifications of the engineered polynucleotide comprise at least one chemical modification to the sugar of the nucleotide of the engineered polynucleotide comprising at least one unlocked nucleic acid (UNA). In some embodiments, the chemical modifications of the engineered polynucleotide comprise at least one chemical modification to the sugar comprising a modification of a constituent of the sugar, where the sugar is a ribose sugar. In some embodiments, the chemical modifications of the engineered polynucleotide comprise at least one chemical modification to the constituent of the ribose sugar of the nucleotide of the engineered polynucleotide comprising a 2′-O-Methyl group. In some embodiments, the chemical modification, instead of 2′-O-Methyl group modification, comprises a 2′-F-RNA. In such case, the 2′-F-RNA and pre-mRNA duplexes does not activate RNase H (degradation by nuclease digestion) and are more stable as determined by higher melting temperature (Tm) than 2′-O-methyl-RNA and pre-mRNA duplex.

In some embodiments, the chemical modifications of the engineered polynucleotide comprise at least one chemical modification comprising replacement of a phosphate moiety of the engineered polynucleotide with a dephospho linker. In some embodiments, the chemical modifications of the engineered polynucleotide comprise at least one chemical modification of a phosphate backbone of the engineered polynucleotide. In some embodiments, the engineered polynucleotide comprises a phosphothioate group. In some embodiments, the chemical modifications of the engineered polynucleotide comprise at least one chemical modification comprising a modification to a base of a nucleotide of the engineered polynucleotide. In some embodiments, the chemical modifications of the engineered polynucleotide comprise at least one chemical modification comprising an unnatural base of a nucleotide. In some embodiments, the chemical modifications of the engineered polynucleotide comprise at least one chemical modification comprising a morpholino group, a cyclobutyl group, pyrrolidine group, or peptide nucleic acid (PNA) nucleoside surrogate. In some embodiments, the chemical modifications of the engineered polynucleotide comprise at least one chemical modification comprising at least one stereopure nucleic acid. In some embodiments, the at least one chemical modification can be positioned proximal to a 5′ end of the engineered polynucleotide. In some embodiments, the at least one chemical modification can be positioned proximal to a 3′ end of the engineered polynucleotide. In some embodiments, the at least one chemical modification can be positioned proximal to both 5′ and 3′ ends of the engineered polynucleotide.

In some embodiments, the at least one chemical modification of the engineered polynucleotide comprises a modification of any one of or any combination of: modification of one or both of the non-linking phosphate oxygens in the phosphodiester backbone linkage; modification of one or more of the linking phosphate oxygens in the phosphodiester backbone linkage; modification of a constituent of the ribose sugar; Replacement of the phosphate moiety with “dephospho” linkers; modification or replacement of a naturally occurring nucleobase; modification of the ribose-phosphate backbone; modification of 5′ end of polynucleotide; modification of 3′ end of polynucleotide; modification of the deoxyribose phosphate backbone; substitution of the phosphate group; modification of the ribophosphate backbone; modifications to the sugar of a nucleotide; modifications to the base of a nucleotide; or stereopure of nucleotide. Example chemical modification to the engineered polynucleotide can be seen in Table 4.

TABLE 4
Example Chemical Modification

| Modification of | |
|---|---|
| engineered polynucleotide | Example(s) |
| Modification of one or both | sulfur (S), selenium (Se), BR3 (wherein R can be, e.g., hydrogen, |
| of the non-linking | alkyl, or aryl), C (e.g., an alkyl group, an aryl group, and the like), |
| phosphate oxygens in the | H, NR2, wherein R can be, e.g., hydrogen, alkyl, or aryl, or |
| phosphodiester backbone | wherein R can be, e.g., alkyl or aryl |
| linkage | |
| Modification of one or more | sulfur (S), selenium (Se), BR3 (wherein R can be, e.g., hydrogen, |
| of the linking phosphate | alkyl, or aryl), C (e.g., an alkyl group, an aryl group, and the like), |
| oxygens in the | H, NR2, wherein R can be, e.g., hydrogen, alkyl, or aryl, or |
| phosphodiester backbone | wherein R can be, e.g., alkyl or aryl |
| linkage | |
| Replacement of the | methyl phosphonate, hydroxylamino, siloxane, carbonate, |
| phosphate moiety with | carboxymethyl, carbamate, amide, thioether, ethylene oxide linker, |
| “dephospho” linkers | sulfonate, sulfonamide, thioformacetal, formacetal, oxime, |
| | methyleneimino, methylenemethylimino, methylenehydrazo, |
| | methylenedimethylhydrazo, or methyleneoxymethylimino |
| Modification or replacement | Nucleic acid analog |
| of a naturally occurring | |
| nucleobase | |
| Modification of the ribose- | phosphorothioate, phosphonothioacetate, phosphoroselenates, |
| phosphate backbone | boranophosphates, borano phosphate esters, hydrogen |
| | phosphonates, phosphonocarboxylate, phosphoroamidates, alkyl or |
| | aryl phosphonates, phosphonoacetate, or phosphotriesters |
| Modification of 5′ end of | 5′ cap or modification of 5′ cap -OH |
| polynucleotide | |
| Modification of 3′ end of | 3′ tail or modification of 3′ end -OH |
| polynucleotide | |
| Modification of the | phosphorothioate, phosphonothioacetate, phosphoroselenates, |
| deoxyribose phosphate | borano phosphates, borano phosphate esters, hydrogen |
| backbone | phosphonates, phosphoroamidates, alkyl or aryl phosphonates, or |
| | phosphotriesters |
| Substitution of the | methyl phosphonate, hydroxylamino, siloxane, carbonate, |
| phosphate group | carboxymethyl, carbamate, amide, thioether, ethylene oxide linker, |
| | sulfonate, sulfonamide, thioformacetal, formacetal, oxime, |
| | methyleneimino, methylenemethylimino, methylenehydrazo, |
| | methylenedimethylhydrazo, or methyleneoxymethylimino. |
| Modification of the | morpholino, cyclobutyl, pyrrolidine, or peptide nucleic acid (PNA) |
| ribophosphate backbone | nucleoside surrogates |
| Modifications to the sugar | Locked nucleic acid (LNA), unlocked nucleic acid (UNA), or |
| of a nucleotide | bridged nucleic acid (BNA) |
| Modification of a | 2′-O-methyl, 2′-O-methoxy-ethyl (2′-MOE), 2′-fluoro, 2′- |
| constituent of the ribose | aminoethyl, 2′-deoxy-2′-fuloarabinou-cleic acid, 2′-deoxy, 2′-O- |
| sugar | methyl, 3′-phosphorothioate, 3′-phosphonoacetate (PACE), or 3′- |
| | phosphonothioacetate (thioPACE) |
| Modifications to the base of | Modification of A, T, C, G, or U |
| a nucleotide | |
| Stereopure of nucleotide | S conformation of phosphorothioate or R conformation of |
| | phosphorothioate |

### Modification of Phosphate Backbone

In some embodiments, the chemical modification comprises modification of one or both of the non-linking phosphate oxygens in the phosphodiester backbone linkage or modification of one or more of the linking phosphate oxygens in the phosphodiester backbone linkage. As used herein, “alkyl” is meant to refer to a saturated hydrocarbon group which is straight-chained or branched. Example alkyl groups include methyl (Me), ethyl (Et), propyl (e.g., n-propyl or isopropyl), butyl (e.g., n-butyl, isobutyl, or t-butyl), or pentyl (e.g., n-pentyl, isopentyl, or neopentyl). An alkyl group can contain from 1 to about 20, from 2 to about 20, from 1 to about 12, from 1 to about 8, from 1 to about 6, from 1 to about 4, or from 1 to about 3 carbon atoms. As used herein, “aryl” refers to monocyclic or polycyclic (e.g., having 2, 3, or 4 fused rings) aromatic hydrocarbons such as, for example, phenyl, naphthyl, anthracenyl, phenanthrenyl, indanyl, or indenyl. In some embodiments, aryl groups have from 6 to about 20 carbon atoms. As used herein, “alkenyl” refers to an aliphatic group containing at least one double bond. As used herein, “alkynyl” refers to a straight or branched hydrocarbon chain containing 2-12 carbon atoms and characterized in having one or more triple bonds. Examples of alkynyl groups can include ethynyl, propargyl, or 3-hexynyl, “Arylalkyl” or “aralkyl” refers to an alkyl moiety in which an alkyl hydrogen atom is replaced by an aryl group. Aralkyl includes groups in which more than one hydrogen atom has been replaced by an aryl group. Examples of “arylalkyl” or “aralkyl” include benzyl, 2-phenylethyl, 3-phenylpropyl, 9-fluorenyl, benzhydryl, and trityl groups, “Cycloalkyl” refers to a cyclic, bicyclic, tricyclic, or polycyclic non-aromatic hydrocarbon groups having 3 to 12 carbons. Examples of cycloalkyl moieties include, but are not limited to, cyclopropyl, cyclopentyl, and cyclohexyl, “Heterocyclyl” refers to a monovalent radical of a heterocyclic ring system. Representative heterocyclyls include, without limitation, tetrahydrofuranyl, tetrahydrothienyl, pyrrolidinyl, pyrrolidonyl, piperidinyl, pyrrolinyl, piperazinyl, dioxanyl, dioxolanyl, diazepinyl, oxazepinyl, thiazepinyl, and morpholinyl. “Heteroaryl” refers to a monovalent radical of a heteroaromatic ring system. Examples of heteroaryl moieties can include imidazolyl, oxazolyl, thiazolyl, triazolyl, pyrrolyl, furanyl, indolyl, thiophenyl pyrazolyl, pyridinyl, pyrazinyl, pyridazinyl, pyrimidinyl, indolizinyl, purinyl, naphthyridinyl, quinolyl, and pteridinyl.

In some embodiments, the phosphate group of a chemically modified nucleotide can be modified by replacing one or more of the oxygens with a different substituent. In some embodiments, the chemically modified nucleotide can include replacement of an unmodified phosphate moiety with a modified phosphate as described herein. In some embodiments, the modification of the phosphate backbone can include alterations that result in either an uncharged linker or a charged linker with unsymmetrical charge distribution. Examples of modified phosphate groups can include phosphorothioate, phosphonothioacetate, phosphoroselenates, boranophosphates, boranophosphate esters, hydrogen phosphonates, phosphoroamidates, alkyl or aryl phosphonates and phosphotriesters. In some embodiments, one of the non-bridging phosphate oxygen atoms in the phosphate backbone moiety can be replaced by any of the following groups: sulfur(S), selenium (Se), BR3 (wherein R can be, e.g., hydrogen, alkyl, or aryl), C (e.g., an alkyl group, an aryl group, and the like), H, NR2 (wherein R can be, e.g., hydrogen, alkyl, or aryl), or (wherein R can be, e.g., alkyl or aryl). The phosphorous atom in an unmodified phosphate group can be achiral. However, replacement of one of the non-bridging oxygens with one of the above atoms or groups of atoms can render the phosphorous atom chiral. A phosphorous atom in a phosphate group modified in this way is a stereogenic center. The stereogenic phosphorous atom can possess either the “R” configuration (herein Rp) or the “S” configuration (herein Sp). In some cases, the engineered polynucleotide comprises stereopure nucleotides comprising S conformation of phosphorothioate or R conformation of phosphorothioate. In some embodiments, the chiral phosphate product is present in a diastereomeric excess of 50%, 60%, 70%, 80%, 90%, or more. In some embodiments, the chiral phosphate product is present in a diastereomeric excess of 95%. In some embodiments, the chiral phosphate product is present in a diastereomeric excess of 96%. In some embodiments, the chiral phosphate product is present in a diastereomeric excess of 97%. In some embodiments, the chiral phosphate product is present in a diastereomeric excess of 98%. In some embodiments, the chiral phosphate product is present in a diastereomeric excess of 99%.

In some embodiments, both non-bridging oxygens of phosphorodithioates can be replaced by sulfur. The phosphorus center in the phosphorodithioates can be achiral which precludes the formation of oligoribonucleotide diastereomers. In some embodiments, modifications to one or both non-bridging oxygens can also include the replacement of the non-bridging oxygens with a group independently selected from S, Se, B, C, H, N, and OR (R can be, e.g., alkyl or aryl). In some embodiments, the phosphate linker can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either or both of the linking oxygens.

In certain embodiments, nucleic acids comprise linked nucleic acids. Nucleic acids can be linked together using any inter nucleic acid linkage. The two main classes of inter nucleic acid linking groups are defined by the presence or absence of a phosphorus atom. Representative phosphorus containing inter nucleic acid linkages include, but are not limited to, phosphodiesters, phosphotriesters, methylphosphonates, phosphoramidate, and phosphorothioates (P═S). Representative non-phosphorus containing inter nucleic acid linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N′-dimethylhydrazine (—CH₂—N(CH₃)—N(CH₃)). In certain embodiments, inter nucleic acids linkages having a chiral atom can be prepared as a racemic mixture, as separate enantiomers. e.g., alkylphosphonates and phosphorothioates. Unnatural nucleic acids can contain a single modification. Unnatural nucleic acids can contain multiple modifications within one of the moieties or between different moieties.

Backbone phosphate modifications to nucleic acid include, but are not limited to, methyl phosphonate, phosphorothioate, phosphoramidate (bridging or non-bridging), phosphotriester, phosphorodithioate, phosphodithioate, and boranophosphate, and can be used in any combination. Other non-phosphate linkages may also be used.

In some embodiments, backbone modifications (e.g., methylphosphonate, phosphorothioate, phosphoroamidate and phosphorodithioate internucleotide linkages) can confer immunomodulatory activity on the modified nucleic acid and/or enhance their stability in vivo.

In some instances, a phosphorous derivative (or modified phosphate group) is attached to the sugar or sugar analog moiety in and can be a monophosphate, diphosphate, triphosphate, alkylphosphonate, phosphorothioate, phosphorodithioate, phosphoramidate or the like.

In some cases, backbone modification comprises replacing the phosphodiester linkage with an alternative moiety such as an anionic, neutral or cationic group. Examples of such modifications include: anionic internucleotide linkage; N3′ to P5′ phosphoramidate modification; boranophosphate DNA; prooligonucleotides; neutral internucleotide linkages such as methylphosphonates; amide linked DNA; methylene(methylimino) linkages; formacetal and thioformacetal linkages; backbones containing sulfonyl groups; morpholino oligos; peptide nucleic acids (PNA); and positively charged deoxyribonucleic guanidine (DNG) oligos. A modified nucleic acid may comprise a chimeric or mixed backbone comprising one or more modifications, e.g. a combination of phosphate linkages such as a combination of phosphodiester and phosphorothioate linkages.

Substitutes for the phosphate include, for example, short chain alkyl or cycloalkyl internucleotide linkages, mixed heteroatom and alkyl or cycloalkyl internucleotide linkages, or one or more short chain heteroatomic or heterocyclic internucleotide linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. It is also understood in a nucleotide substitute that both the sugar and the phosphate moieties of the nucleotide can be replaced, by for example an amide type linkage (aminoethylglycine)(PNA). It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethylammonium 1-di-O-hexadecyl-rac-glycero-S—H-phosphonate, a polyamine or a polyethylene glycol chain, or adamantane acetic acid, a palmityl moiety, or an octadecylamine or hexylamino-carbonyl-oxycholesterol moiety.

In some embodiments, the chemical modification described herein comprises modification of a phosphate backbone. In some embodiments, the engineered polynucleotide described herein comprises at least one chemically modified phosphate backbone. Example chemically modification of the phosphate group or backbone can include replacing one or more of the oxygens with a different substituent. Furthermore, the modified nucleotide present in the engineered polynucleotide can include the replacement of an unmodified phosphate moiety with a modified phosphate as described herein. In some embodiments, the modification of the phosphate backbone can include alterations resulting in either an uncharged linker or a charged linker with unsymmetrical charge distribution. Example modified phosphate groups can include, phosphorothioate, phosphonothioacetate, phosphoroselenates, borano phosphates, borano phosphate esters, hydrogen phosphonates, phosphoroamidates, alkyl or aryl phosphonates and phosphotriesters. In some embodiments, one of the non-bridging phosphate oxygen atoms in the phosphate backbone moiety can be replaced by any of the following groups: sulfur(S), selenium (Se), BR₃ (wherein R can be, e.g., hydrogen, alkyl, or aryl), C (e.g., an alkyl group, an aryl group, and the like), H, NR₂ (wherein R can be, e.g., hydrogen, alkyl, or aryl), or (wherein R can be, e.g., alkyl or aryl). The phosphorous atom in an unmodified phosphate group is achiral. However, replacement of one of the non-bridging oxygens with one of the above atoms or groups of atoms can render the phosphorous atom chiral; that is to say that a phosphorous atom in a phosphate group modified in this way is a stereogenic center. The stereogenic phosphorous atom can possess either the “R” configuration (herein Rp) or the “S” configuration (herein Sp). In such case, the chemically modified engineered polynucleotide can be stereopure (e.g. S or R confirmation). In some cases, the chemically modified engineered polynucleotide comprises stereopure phosphate modification. For example, the chemically modified engineered polynucleotide comprises S conformation of phosphorothioate or R conformation of phosphorothioate.

Phosphorodithioates have both non-bridging oxygens replaced by sulfur. The phosphorus center in the phosphorodithioates is achiral which precludes the formation of oligoribonucleotide diastereomers. In some embodiments, modifications to one or both non-bridging oxygens can also include the replacement of the non-bridging oxygens with a group independently selected from S, Se, B, C, H, N, and OR (R can be, e.g., alkyl or aryl).

The phosphate linker can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens.

### Replacement of Phosphate Moiety

In some embodiments, at least one phosphate group of the engineered polynucleotide can be chemically modified. In some embodiments, the phosphate group can be replaced by non-phosphorus containing connectors. In some embodiments, the phosphate moiety can be replaced by dephospho linker. In some embodiments, the charge phosphate group can be replaced by a neutral group. In some cases, the phosphate group can be replaced by methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino. In some embodiments, nucleotide analogs described herein can also be modified at the phosphate group. Modified phosphate group can include modification at the linkage between two nucleotides with phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates (e.g. 3′-amino phosphoramidate and aminoalkylphosphoramidates), thionophosphoramidates, thionoalkylphosphonates, thionoalkylphosphotriesters, and boranophosphates. The phosphate or modified phosphate linkage between two nucleotides can be through a 3-5′ linkage or a 2′-5′ linkage, and the linkage contains inverted polarity such as 3′-5′ to 5′-3′ or 2′-5′ to 5′-2′.

### Substitution of Phosphate Group

In some embodiments, the chemical modification described herein comprises modification by replacement of a phosphate group. In some embodiments, the engineered polynucleotide described herein comprises at least one chemically modification comprising a phosphate group substitution or replacement. Example phosphate group replacement can include non-phosphorus containing connectors. In some embodiments, the phosphate group substitution or replacement can include replacing charged phosphate group can by a neutral moiety. Example moieties which can replace the phosphate group can include methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino.

### Modification of the Ribophosphate Backbone

In some embodiments, the chemical modification described herein comprises modifying ribophosphate backbone of the engineered polynucleotide. In some embodiments, the engineered polynucleotide described herein comprises at least one chemically modified ribophosphate backbone. Example chemically modified ribophosphate backbone can include scaffolds that can mimic nucleic acids can also be constructed wherein the phosphate linker and ribose sugar are replaced by nuclease resistant nucleoside or nucleotide surrogates. In some embodiments, the nucleobases can be tethered by a surrogate backbone. Examples can include morpholino, cyclobutyl, pyrrolidine and peptide nucleic acid (PNA) nucleoside surrogates.

### Modification of Sugar

In some embodiments, the chemical modification described herein comprises modifying of sugar. In some embodiments, the engineered polynucleotide described herein comprises at least one chemically modified sugar. Example chemically modified sugar can include 2′ hydroxyl group (OH) modified or replaced with a number of different “oxy” or “deoxy” substituents. In some embodiments, modifications to the 2′ hydroxyl group can enhance the stability of the nucleic acid since the hydroxyl can no longer be deprotonated to form a 2′-alkoxide ion. The 2′-alkoxide can catalyze degradation by intramolecular nucleophilic attack on the linker phosphorus atom. Examples of “oxy”-2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH₂CH₂O)ₙCH₂CH₂OR, wherein R can be, e.g., H or optionally substituted alkyl, and n can be an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20). In some embodiments, the “oxy”-2″ hydroxyl group modification can include (LNA, in which the 2′ hydroxyl can be connected, e.g., by a Ci-₆ alkylene or Cj-6 heteroalkylene bridge, to the 4′ carbon of the same ribose sugar, where example bridges can include methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy, O(CH₂)ₙ-amino, (wherein amino can be, e.g., NH₂; alkylamino, dialkylamino, heterocyclyl, arylamino, diarylamino, heteroarylamino, or diheteroarylamino, ethylenediamine, or polyamino). In some embodiments, the “oxy”-2″ hydroxyl group modification can include the methoxyethyl group (MOE), (OCH₂CH₂OCH₃, e.g., a PEG derivative). In some cases, the deoxy modifications can include hydrogen (i.e. deoxyribose sugars, e.g., at the overhang portions of partially dsRNA); halo (e.g., bromo, chloro, fluoro, or iodo); amino (wherein amino can be, e.g., NH₂; alkylamino, dialkylamino, heterocyclyl, arylamino, diarylamino, heteroarylamino, diheteroarylamino, or amino acid); NH(CH₂CH₂NH)ₙCH₂CH₂-amino (wherein amino can be, e.g., as described herein), NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which can be optionally substituted with e.g., an amino as described herein. In some instances, the sugar group can also contain one or more carbons that possess the opposite stereochemical configuration than that of the corresponding carbon in ribose. Thus, a modified nucleic acid can include nucleotides containing e.g., arabinose, as the sugar. The nucleotide “monomer” can have an alpha linkage at the Γ position on the sugar, e.g., alpha-nucleosides. The modified nucleic acids can also include “abasic” sugars, which lack a nucleobase at C—. The abasic sugars can also be further modified at one or more of the constituent sugar atoms. The modified nucleic acids can also include one or more sugars that are in the L form, e.g. L-nucleosides. In some aspects, the engineered polynucleotide described herein includes the sugar group ribose, which is a 5-membered ring having an oxygen. Example modified nucleosides and modified nucleotides can include replacement of the oxygen in ribose (e.g., with sulfur(S), selenium (Se), or alkylene, such as, e.g., methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for example, anhydrohexitol, altritol, mannitol, cyclohexanyl, cyclohexenyl, and morpholino that also has a phosphoramidate backbone). In some embodiments, the modified nucleotides can include multicyclic forms (e.g., tricyclo; and “unlocked” forms, such as glycol nucleic acid (GNA) (e.g., R-GNA or S-GNA, where ribose is replaced by glycol units attached to phosphodiester bonds), threose nucleic acid. In some embodiments, the modifications to the sugar of the engineered polynucleotide comprises modifying the engineered polynucleotide to include locked nucleic acid (LNA), unlocked nucleic acid (UNA), or bridged nucleic acid (BNA).

### Modification of a Constituent of the Ribose Sugar

In some embodiments, the engineered polynucleotide described herein comprises at least one chemical modification of a constituent of the ribose sugar. In some embodiments, the chemical modification of the constituent of the ribose sugar can include 2′-O-methyl, 2′-O-methoxy-ethyl (2′-MOE), 2′-fluoro, 2-aminoethyl, 2′-deoxy-2′-fuloarabinou-cleic acid, 2′-deoxy, 2′-O-methyl, 3′-phosphorothioate, 3′-phosphonoacetate (PACE), or 3′-phosphonothioacetate (thioPACE). In some embodiments, the chemical modification of the constituent of the ribose sugar comprises unnatural nucleic acid. In some instances, the unnatural nucleic acids include modifications at the 5′-position and the 2-position of the sugar ring, such as 5′-CH₂-substituted 2-O-protected nucleosides. In some cases, unnatural nucleic acids include amide linked nucleoside dimers have been prepared for incorporation into oligonucleotides wherein the 3′ linked nucleoside in the dimer (5′ to 3″) comprises a 2′-OCH₃ and a 5′-(S)—CH₃. Unnatural nucleic acids can include 2′-substituted 5′-CH₂ (or O) modified nucleosides. Unnatural nucleic acids can include 5′-methylenephosphonate DNA and RNA monomers, and dimers. Unnatural nucleic acids can include 5′-phosphonate monomers having a 2-substitution and other modified 5′-phosphonate monomers. Unnatural nucleic acids can include 5′-modified methylenephosphonate monomers. Unnatural nucleic acids can include analogs of 5′ or 6′-phosphonate ribonucleosides comprising a hydroxyl group at the 5′ and/or 6′-position. Unnatural nucleic acids can include 5′-phosphonate deoxyribonucleoside monomers and dimers having a 5′-phosphate group. Unnatural nucleic acids can include nucleosides having a 6′-phosphonate group wherein the 5′ or/and 6′-position is unsubstituted or substituted with a thio-tert-butyl group (SC(CH₃)ₙ) (and analogs thereof); a methyleneamino group (CH₂NH₂) (and analogs thereof) or a cyano group (CN) (and analogs thereof).

In some embodiments, unnatural nucleic acids also include modifications of the sugar moiety. In some cases, nucleic acids contain one or more nucleosides wherein the sugar group has been modified. Such sugar modified nucleosides may impart enhanced nuclease stability, increased binding affinity, or some other beneficial biological property. In certain embodiments, nucleic acids comprise a chemically modified ribofuranose ring moiety. Examples of chemically modified ribofuranose rings include, without limitation, addition of substituent groups (including 5′ and/or 2′ substituent groups; bridging of two ring atoms to form bicyclic nucleic acids; replacement of the ribosyl ring oxygen atom with S, N(R), or C(R₁)(R₂) (R=H, C₁-C₁₂ alkyl or a protecting group); and combinations thereof.

In some instances, the engineered polynucleotide described herein comprises modified sugars or sugar analogs. Thus, in addition to ribose and deoxyribose, the sugar moiety can be pentose, deoxypentose, hexose, deoxyhexose, glucose, arabinose, xylose, lyxose, or a sugar “analog” cyclopentyl group. The sugar can be in a pyranosyl or furanosyl form. The sugar moiety can be the furanoside of ribose, deoxyribose, arabinose or 2′-O-alkylribose, and the sugar can be attached to the respective heterocyclic bases either in [alpha] or [beta] anomeric configuration. Sugar modifications include, but are not limited to, 2′-alkoxy-RNA analogs, 2′-amino-RNA analogs, 2″-fluoro-DNA, and 2-alkoxy- or amino-RNA/DNA chimeras. For example, a sugar modification may include 2′-O-methyl-uridine or 2′-O-methyl-cytidine. Sugar modifications include 2-O-alkyl-substituted deoxyribonucleosides and 2′-O-ethyleneglycol-like ribonucleosides.

Modifications to the sugar moiety include natural modifications of the ribose and deoxy ribose as well as unnatural modifications. Sugar modifications include, but are not limited to, the following modifications at the 2′ position: OH; F; O—, S—, or N-alkyl; O—, S—, or N-alkenyl; O—, S— or N-alkynyl; or O-alkyl-O-alkyl, wherein the alkyl, alkenyl and alkynyl can be substituted or unsubstituted C₁ to C₁₀, alkyl or C₂ to C₁₀ alkenyl and alkynyl, 2′ sugar modifications also include but are not limited to —O[(CH₂)ₙO]ₘCH₃, —O(CH₂)ₙOCH₃, —O(CH₂)ₙNH₂, —O(CH₂)ₙCH₃, —O(CH₂)ₙONH₂, and —O(CH₂)ₙON[(CH₂)ₙCH₃)]₂, where n and m are from 1 to about 10. Other chemical modifications at the 2′ position include but are not limited to: C₁ to C₁₀ lower alkyl, substituted lower alkyl, alkaryl, aralkyl, O-alkaryl, O-aralkyl, SH, SCH₃, OCN, Cl, Br, CN, CF₃, OCF₃, SOCH₃, SO₂ CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of the 5′ terminal nucleotide. Chemically modified sugars also include those that contain modifications at the bridging ring oxygen, such as CH₂ and S. Nucleotide sugar analogs can also have sugar mimetics such as cyclobutyl moieties in place of the pentofuranosyl sugar. Examples of nucleic acids having modified sugar moieties include, without limitation, nucleic acids comprising 5′-vinyl, 5′-methyl (R or S), 4′-S, 2′-F, 2′-OCH₃, and 2′-O(CH₂)₂OCH₃ substituent groups. The substituent at the 2′ position can also be selected from allyl, amino, azido, thio, O-allyl, O—(C₁-C₁₀ alkyl), OCF₃, O(CH₂)₂SCH₃, O(CH₂)₂—O—N(Rₘ)(Rₙ), and O—CH₂—C(═O)—N(Rₘ)(Rₙ), where each Rₘ and Rₙ is, independently. H or substituted or unsubstituted C₁-C₁₀ alkyl.

In certain embodiments, nucleic acids described herein include one or more bicyclic nucleic acids. In certain such embodiments, the bicyclic nucleic acid comprises a bridge between the 4′ and the 2′ ribosyl ring atoms. In certain embodiments, nucleic acids provided herein include one or more bicyclic nucleic acids wherein the bridge comprises a 4′ to 2′ bicyclic nucleic acid. Examples of such 4′ to 2′ bicyclic nucleic acids include, but are not limited to, one of the formulae: 4′-(CH₂)—O-2′ (LNA); 4-(CH₂)—S-2; 4′-(CH₂)₂—O-2′ (ENA); 4-CH(CH₃)—O-2′ and 4′-CH(CH₂OCH₃)—O-2′, and analogs thereof; 4′-C(CH₃)(CH₃)—O-2′ and analogs thereof.

### Modifications on the Base of Nucleotide

In some embodiments, the chemical modification described herein comprises modification of the base of nucleotide (e.g. the nucleobase). Example nucleobases can include adenine (A), thymine (T), guanine (G), cytosine (C), and uracil (U). These nucleobases can be modified or replaced to in the engineered polynucleotide described herein. The nucleobase of the nucleotide can be independently selected from a purine, a pyrimidine, a purine or pyrimidine analog. In some embodiments, the nucleobase can be naturally occurring or synthetic derivatives of a base.

In some embodiments, the chemical modification described herein comprises modifying an uracil. In some embodiments, the engineered polynucleotide described herein comprises at least one chemically modified uracil. Example chemically modified uracil can include pseudouridine, pyridin-4-one ribonucleoside, 5-aza-uridine, 6-aza-uridine, 2-thio-5-aza-uridine, 2-thio-uridine, 4-thio-uridine, 4-thio-pseudouridine, 2-thio-pseudouridine, 5-hydroxy-uridine, 5-aminoallyl-uridine, 5-halo-uridine (e.g., 5-iodo-uridine or 5-bromo-uridine), 3-methyl-uridine, 5-methoxy-uridine, uridine 5-oxyacetic acid, uridine 5-oxyacetic acid methyl ester, 5-carboxymethyl-uridine, 1-carboxymethyl-pseudouridine, 5-carboxyhydroxymethyl-uridine, 5-carboxyhydroxymethyl-uridine methyl ester, 5-methoxy carbonylmethyl-uridine, 5-methoxycarbonylmethyl-2-thio-uridine, 5-aminomethyl-2-thio-uridine, 5-methylaminomethyl-uridine, 5-methylaminomethyl-2-thio-uridine, 5-methylaminomethyl-2-seleno-uridine, 5-carbamoylmethyl-uridine, 5-carboxymethylaminomethyl-uridine, 5-carboxymethylaminomethyl-2-thio-uridine, 5-propynyl-uridine, 1-propynyl-pseudouridine, 5-taurinomethyl-uridine, 1-taurinomethyl-pseudouridine, 5-taurinomethyl-2-thio-uridine, 1-taurinomethyl-4-thio-pseudouridine, 5-methyl-uridine, 1 methyl-pseudouridine, 5-methyl-2-thio-uridine, 1-methyl-4-thio-pseudouridine, 4-thio-1-methyl-pseudouridine, 3-methyl-pseudouridine, 2-thio-1-methyl-pseudouridine, 1-methyl-1-deaza-pseudouridine, 2-thio-1-methyl-1-deaza-pseudouridine, dihydroundine, dihydropseudoundine, 5,6-dihydrouridine, 5-methyl-dihydrouridine, 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl) uridine, 1-methyl-3-(3-amino-3-carboxypropy pseudouridine, 5-(isopentenylaminomethyl) uridine, 5-(isopentenylaminomethy])-2-thio-uridine, a-thio-uridine, 2′-O-methyl-uridine, 5,2″-O-dimethyl-uridine, 2-O-methyl-pseudouridine, 2-thio-2′-O-methyl-uridine, 5-methoxycarbonylmethyl-2′-O-methyl-uridine, 5-carbamoylmethyl-2′-O-methyl-uridine, 5-carboxymethylaminomethyl-2-O-methyl-uridine, 3,2′-O-dimethyl-uridine, 5-(isopentenylaminomethyl)-2′-O-methyl-uridine, 1-thio-uridine, deoxy thymidine, 2-F-ara-uridine, 2′-F-uridine, 2′-OH-ara-uridine, 5-(2-carbomethoxyvinyl) uridine, 5-[3-(1-E-propenylamino) uridine, pyrazolo[3,4-d]pyrimidines, xanthine, and hypoxanthine.

In some embodiments, the chemical modification described herein comprises modifying a cytosine. In some embodiments, the engineered polynucleotide described herein comprises at least one chemically modified cytosine. Example chemically modified cytosine can include 5-aza-cytidine, 6-aza-cytidine, pseudoisocytidine, 3-methyl-cytidine, N4-acetyl-cytidine, 5-formyl-cytidine, N4-methyl-cytidine, 5-methyl-cytidine, 5-halo-cytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine, a-thio-cytidine, 2′-O-methyl-cytidine, 5,2′-O-dimethyl-cytidine, N4-acetyl-2′-O-methyl-cytidine, N4,2-O-dimethyl-cytidine, 5-formyl-2-O-methyl-cytidine, N4,N4,2′-O-trimethyl-cytidine, 1-thio-cytidine, 2′-F-ara-cytidine, 2′-F-cytidine, and 2′-OH-ara-cytidine.

In some embodiments, the chemical modification described herein comprises modifying a adenine. In some embodiments, the engineered polynucleotide described herein comprises at least one chemically modified adenine. Example chemically modified adenine can include 2-amino-purine, 2,6-diaminopurine, 2-amino-6-halo-purine (e.g., 2-amino-6-chloro-purine), 6-halo-purine (e.g., 6-chloi-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine, 2-methyl-adenine, N6-methyl-adenosine, 2-methylthio-N6-methyl-adenosine, N6-isopentenyl-adenosine, 2-methylthio-N6-isopentenyl-adenosine, N6-(cis-hydroxyisopentenyl) adenosine, 2-methylthio-N6-(cis-hydroxyisopentenyl) adenosine, N6-glycinylcarbamoyl-adenosine, N6-threonylcarbamoyl-adenosine, N6-methyl-N6-threonylcarbamoyl-adenosine, 2-methylthio-N6-threonylcarbamoyl-adenosine, N6,N6-dimethyl-adenosine, N6-hydroxynorvalylcarbamoyl-adenosine, 2-methylthio-N6-hydroxynorvalylcarbamoyl-adenosine, N6-acetyl-adenosine, 7-methyl-adenine, 2-methylthio-adenine, 2-methoxy-adenine, a-thio-adenosine, 2-O-methyl-adenosine, N6,2-O-dimethyl-adenosine, N6-Methyl-2′-deoxyadenosine, N6,N6,2-O-trimethyl-adenosine, 1,2′-O-dimethyl-adenosine, 2′-O-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

In some embodiments, the chemical modification described herein comprises modifying a guanine. In some embodiments, the engineered polynucleotide described herein comprises at least one chemically modified guanine. Example chemically modified guanine can include inosine, 1-methyl-inosine, wyosine, methylwyosine, 4-demethyl-wyosine, isowyosine, wybutosine, peroxywybutosine, hydroxywybutosine, undermodified hydroxywybutosine, 7-deaza-guanosine, queuosine, epoxyqueuosine, galactosyl-queuosine, mannosyl-queuosine, 7-cyano-7-deaza-guanosine, 7-aminomethyl-7-deaza-guanosine, archaeosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methyl-inosine, 6-methoxy-guanosine, 1-methyl-guanosine, N2-methyl-guanosine, N2,N2-dimethyl-guanosine, N2,7-dimethyl-guanosine, N2,N2,7-dimethyl-guanosine, 8-oxo-guanosine, 7-methyl-8-oxo-guanosine, 1-meththio-guanosine,N2-methyl-6-thio-guanosine, N2,N2-dimethyl-6-thio-guanosine, a-thio-guanosine, 2′-O-methyl-guanosine, N2-methyl-2″-O-methyl-guanosine, N2,N2-dimethyl-2′-O-methyl-guanosine, 1-methyl-2-O-methyl-guanosine, N2,7-dimethyl-2′-O-methyl-guanosine, 2′-O-methyl-inosine, 1,2′-O-dimethyl-inosine, 6-O-phenyl-2″-deoxyinosine, 2′-O-ribosylguanosine, 1-thio-guanosine, 6-O-methyguanosine, 06-Methyl-2′-deoxyguanosine, 2′-F-ara-guanosine, and 2-F-guanosine.

In some cases, the chemical modification of the engineered polynucleotide can include introducing or substituting a nucleic acid analog or an unnatural nucleic acid into the engineered polynucleotide. In some embodiments, nucleic acid analog can be any one of the chemically modified nucleic acid described herein. Example nucleic acid analog can be found in PCT/US2015/025175, PCT/US2014/050423, PCT/US2016/067353, PCT/US2018/041503. PCT/US18/041509, PCT/US2004/011786, or PCT/US2004/011833, all of which are expressly incorporated by reference in their entireties. The chemically modified nucleotide described herein can include a variant of guanosine, uridine, adenosine, thymidine, and cytosine, including any natively occurring or non-natively occurring guanosine, uridine, adenosine, thymidine or cytidine that has been altered chemically, for example by acetylation, methylation, hydroxylation. Example chemically modified nucleotide can include 1-methyl-adenosine, 1-methyl-guanosine, 1-methyl-inosine, 2,2-dimethyl-guanosine, 2,6-diaminopurine, 2′-amino-2′-deoxyadenosine, 2′-amino-2-deoxycytidine, 2-amino-2-deoxyguanosine, 2′-amino-2′-deoxyuridine, 2-amino-6-chloropurineriboside, 2-aminopurine-riboside, 2-araadenosine, 2′-aracytidine, 2′-arauridine, 2′-azido-2′-deoxyadenosine, 2-azido-2′-deoxycytidine, 2′-azido-2′-deoxyguanosine, 2′-azido-2′-deoxyuridine, 2-chloroadenosine, 2″-fluoro-2′-deoxyadenosine, 2′-fluoro-2-deoxycytidine, 2-fluoro-2-deoxyguanosine, 2″-fluoro-2′-deoxyuridine, 2′-fluorothymidine, 2-methyl-adenosine, 2-methyl-guanosine, 2-methyl-thio-N6-isopenenyl-adenosine, 2-O-methyl-2-aminoadenosine, 2-O-methyl-2″-deoxyadenosine, 2′-O-methyl-2′-deoxycytidine, 2-O-methyl-2-deoxyguanosine, 2-O-methyl-2′-deoxyuridine, 2′-O-methyl-5-methyluridine, 2-O-methylinosine, 2′-O-methylpseudouridine, 2-thiocytidine, 2-thio-cytidine, 3-methyl-cytidine, 4-acetyl-cytidine, 4-thiouridine, 5-(carboxyhydroxymethyl)-uridine, 5,6-dihydrouridine, 5-aminoallylcytidine, 5-aminoallyl-deoxyuridine, 5-bromouridine, 5-carboxymethylaminomethyl-2-thio-uracil, 5-carboxymethylamonomethyl-uracil, 5-chloro-ara-cytosine, 5-fluoro-uridine, 5-iodouridine, 5-methoxy carbonylmethyl-uridine, 5-methoxy-uridine, 5-methyl-2-thio-uridine, 6-Azacytidine, 6-azauridine, 6-chloro-7-deaza-guanosine, 6-chloropurineriboside, 6-mercapto-guanosine, 6-methyl-mercaptopurine-riboside, 7-deaza-2′-deoxy-guanosine, 7-deazaadenosine, 7-methyl-guanosine, 8-azaadenosine, 8-bromo-adenosine, 8-bromo-guanosine, 8-mercapto-guanosine, 8-oxoguanosine, benzimidazole-riboside, beta-D-mannosyl-queosine, dihydro-uridine, inosine, N1-methyladenosine, N6-([6-aminohexyl]carbamoylmethyl)-adenosine, N6-isopentenyl-adenosine, N6-methyl-adenosine, N7-methyl-xanthosine, N-uracil-5-oxyacetic acid methyl ester, puromycin, queosine, uracil-5-oxyacetic acid, uracil-5-oxyacetic acid methyl ester, wybutoxosine, xanthosine, and xylo-adenosine. In some embodiments, the chemically modified nucleic acid as described herein comprises at least one chemically modified nucleotide selected from 2-amino-6-chloropurineriboside-5′-triphosphate, 2-aminopurine-riboside-5′-triphosphate, 2-aminoadenosine-5′-triphosphate, 2′-amino-2′-deoxy cytidine-triphosphate, 2-thiocytidine-5-triphosphate, 2-thiouridine-5′-triphosphate, 2′-fluorothymidine-5″-triphosphate, 2′-O-methyl-inosine-5′-triphosphate, 4-thiouridine-5′-triphosphate, 5-aminoallylcytidine-5′-triphosphate, 5-aminoallyluridine-5′-triphosphate, 5-bromocytidine-5″-triphosphate, 5-bromouridine-5′-triphosphate, 5-bromo-2′-deoxy cytidine-5′-triphosphate, 5-bromo-2-deoxyuridine-5′-triphosphate, 5-iodocytidine-5′-triphosphate, 5-iodo-2′-deoxycytidine-5′-triphosphate, 5-iodouridine-5-triphosphate, 5-iodo-2′-deoxyuridine-5″-triphosphate, 5-methylcytidine-5′-triphosphate, 5-methyluridine-5′-triphosphate, 5-propynyl-2′-deoxycytidine-5′-triphosphate, 5-propynyl-2″-deoxyuridine-5″-triphosphate, 6-azacytidine-5′-triphosphate, 6-azauridine-5″-triphosphate, 6-chloropurineriboside-5-triphosphate, 7-deazaadenosine-5″-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5″-triphosphate, N1-methylguanosine-5′-triphosphate, N6-methyladenosine-5′-triphosphate, 6-methylguanosine-5′-triphosphate, pseudouridine-5-triphosphate, puromycin-5′-triphosphate, or xanthosine-5′-triphosphate. In some embodiments, the chemically modified nucleic acid as described herein comprises at least one chemically modified nucleotide selected from pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine. In some embodiments, the artificial nucleic acid as described herein comprises at least one chemically modified nucleotide selected from 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine. In some embodiments, the chemically modified nucleic acid as described herein comprises at least one chemically modified nucleotide selected from 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl) adenosine, 2-methylthio-N6-(cis-hydroxyisopentenyl) adenosine, N6-glycinylcarbamoyladenosine, N6-threonylcarbamoyladenosine, 2-methylthio-N6-threonylcarbamoyladenosine, N6,N6-dimethyladenosine, 7-methyladenine, 2-methylthio-adenine, and 2-methoxy-adenine. In other embodiments, the chemically modified nucleic acid as described herein comprises at least one chemically modified nucleotide selected from inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine. In certain embodiments, the chemically modified nucleic acid as described herein comprises at least one chemically modified nucleotide selected from 6-aza-cytidine, 2-thio-cytidine, alpha-thio-cytidine, pseudo-iso-cytidine, 5-aminoallyl-uridine, 5-iodo-uridine, N1-methyl-pseudouridine, 5,6-dihydrouridine, alpha-thio-uridine, 4-thio-uridine, 6-aza-uridine, 5-hydroxy-uridine, deoxy-thymidine, 5-methyl-uridine, pyrrolo-cytidine, inosine, alpha-thio-guanosine, 6-methyl-guanosine, 5-methyl-cytidine, 8-oxo-guanosine, 7-deaza-guanosine, N1-methyl-adenosine, 2-amino-6-chloro-purine, N6-methyl-2-amino-purine, pseudo-iso-cytidine, 6-chloro-purine, N6-methyl-adenosine, alpha-thio-adenosine, 8-azido-adenosine, 7-deaza-adenosine.

A modified base of a unnatural nucleic acid includes, but is not limited to, uracil-5-yl, hypoxanthin-9-yl (I), 2-aminoadenin-9-yl, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Certain unnatural nucleic acids, such as 5-substituted pyrimidines, 6-azapyrimidines and N-2 substituted purines, N-6 substituted purines, 0-6 substituted purines, 2-aminopropyladenine, 5-propynyluracil, 5-propynylcytosine, 5-methylcytosine, those that increase the stability of duplex formation, universal nucleic acids, hydrophobic nucleic acids, promiscuous nucleic acids, size-expanded nucleic acids, fluorinated nucleic acids, 5-substituted pyrimidines, 6-azapyrimidines and N-2, N-6 and O-6 substituted purines, including 2-aminopropyladenine, 5-propynyluracil and 5-propynylcytosine, 5-methylcytosine (5-me-C), 5-hydroxymethyl cytosine, xanthine, hypoxanthine, 2-aminoadenine, 6-methyl, other alkyl derivatives of adenine and guanine, 2-propyl and other alkyl derivatives of adenine and guanine, 2-thiouracil, 2-thiothymine and 2-thiocytosine, 5-halouracil, 5-halocytosine, 5-propynyl (—C≡C—CH₃) uracil, 5-propynyl cytosine, other alkynyl derivatives of pyrimidine nucleic acids, 6-azo uracil, 6-azo cytosine, 6-azo thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl, other 5-substituted uracils and cytosines, 7-methylguanine, 7-methyladenine, 2-F-adenine, 2-amino-adenine, 8-azaguanine, 8-azaadenine, 7-deazaguanine, 7-deazaadenine, 3-deazaguanine, 3-deazaadenine, tricyclic pyrimidines, phenoxazine cytidine([5,4-b][1,4]benzoxazin-2(3H)-one), phenothiazine cytidine (1H-pyrimido[5,4-b][1,4]benzothiazin-2(3H)-one), G-clamps, phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4.5]pyrrolo[2,3-d]pyrimidin-2-one), those in which the purine or pyrimidine base is replaced with other heterocycles, 7-deaza-adenine, 7-deazaguanosine, 2-aminopyridine, 2-pyridone, azacytosine, 5-bromocytosine, bromouracil, 5-chlorocytosine, chlorinated cytosine, cyclocytosine, cytosine arabinoside, 5-fluorocytosine, fluoropyrimidine, fluorouracil, 5,6-dihydrocytosine, 5-iodocytosine, hydroxyurea, iodouracil, 5-nitrocytosine, 5-bromouracil, 5-chlorouracil, 5-fluorouracil, and 5-iodouracil, 2-amino-adenine, 6-thio-guanine, 2-thio-thymine, 4-thio-thymine, 5-propynyl-uracil, 4-thio-uracil, N4-ethylcytosine, 7-deazaguanine, 7-deaza-8-azaguanine, 5-hydroxycytosine, 2′-deoxyuridine, or 2-amino-2-deoxyadenosine.

In some cases, the at least one chemical modification can comprise chemically modifying the 5′ or 3′ end such as 5′ cap or 3′ tail of the engineered polynucleotide. In some embodiments, the engineered polynucleotide comprises a chemical modification comprising 3″ nucleotides which can be stabilized against degradation, e.g., by incorporating one or more of the modified nucleotides described herein. In this embodiment, uridines can be replaced with modified uridines, e.g., 5-(2-amino) propyl uridine, and 5-bromo uridine, or with any of the modified uridines described herein; adenosines and guanosines can be replaced with modified adenosines and guanosines, e.g., with modifications at the 8-position, e.g., 8-bromo guanosine, or with any of the modified adenosines or guanosines described herein. In some embodiments, deaza nucleotides, e.g., 7-deaza-adenosine, can be incorporated into the gRNA. In some embodiments, O- and N-alkylated nucleotides, e.g., N6-methyladenosine, can be incorporated into the gRNA. In some embodiments, sugar-modified ribonucleotides can be incorporated, e.g., wherein the 2′ OH-group is replaced by a group selected from H, —OR, —R (wherein R can be, e.g., alkyl, cycloalkyl, aryl, aralkyl, heteroaryl or sugar), halo, —SH, —SR (wherein R can be, e.g., alkyl, cycloalkyl, aryl, aralkyl, heteroaryl or sugar), amino (wherein amino can be, e.g., NH₂; alkylamino, dialkylamino, heterocyclyl, arylamino, diarylamino, heteroarylamino, diheteroarylamino, or amino acid); or cyano (—CN). In some embodiments, the phosphate backbone can be modified as described herein, e.g., with a phosphothioate group. In some embodiments, the nucleotides in the overhang region of the gRNA can each independently be a modified or unmodified nucleotide including, but not limited to 2′-sugar modified, such as, 2-F 2′-O-methyl, thymidine (T), 2-O-methoxyethyl-5-methyluridine (Teo), 2′-O-methoxyethyladenosine (Aeo), 2′-O-methoxyethyl-5-methylcytidine (m5Ceo), or any combinations thereof.

In some embodiments, all nucleotides of a targeting moiety have 2′ O-methyl modifications. The 2′ O-methyl modification is thought to increase the affinity of an engineered polynucleotide for its pre-mRNA targets and/or prevent degradation of an engineered polynucleotide by nucleases. In some embodiments, all nucleotides of a targeting moiety have phosphorothioate modifications.

### Regulating Moiety

In some embodiments of the engineered polynucleotide described herein, the post-transcriptional regulating moiety (or the regulating moiety) (e.g., the spliceosomal moiety) is selected from a spliceosomal ribonucleoprotein complex, a spliceosomal small nuclear ribonucleic acid (snRNA), a spliceosomal protein, a functional variant thereof, or a functional fragment thereof. In some embodiments, the spliceosomal moiety comprises U1 snRNA and a spliceosomal protein. In some embodiments, the spliceosomal moiety comprises U2 snRNA and a spliceosomal protein. In some embodiments, the spliceosomal snRNA is selected from U1, U2, U4, U5, U6, U11, U12, U14atac, U6atac, and combinations thereof. In some embodiments, the spliceosomal snRNA is U1 or U2. In some embodiments, the spliceosomal protein is selected from Sm, U1-70k, U1A, U1C, and combinations thereof. Non-limiting example of the spliceosomal moiety includes SmD1, SmD2, SmD3, SmE, SmF, SmG, U1, U2, U4, U5, U6, U11, U12, U14, or U16.

In some embodiments described herein, when associated with the engineered polynucleotide and the RNA (e.g., the mRNA, such as the pre-mRNA), the spliceosomal moiety cleaves or splices the RNA (e.g., the mRNA, such as the pre-mRNA) in the target sequence. In some embodiments, the spliceosomal moiety further facilitates modification of a cleaved RNA (e.g., a cleaved mRNA, such as a cleaved pre-mRNA).

In some embodiments, the binding of the engineered polynucleotide to the target sequence by base pairing such as Watson-Crick base pairing. The binding of the engineered polynucleotide to the recruiting moiety provided herein can be utilized to modulate expression or activity of a target gene. In some embodiments, the binding of the engineered polynucleotide to the recruiting moiety enables the recruiting moiety to splice the pre-mRNA encoding the target gene with increased specificity, thus modulating the target gene. In some embodiments, the binding of the engineered polynucleotide to the recruiting moiety enables the recruiting moiety to splice the pre-mRNA encoding the target gene with increased efficiency, thus modulating the target gene. Modulation can refer to increasing or decreasing the expression or activity of the target gene. Non-limiting example of the target gene can include microtubule associated protein tau (MAPT). In some embodiments, the expression or activity of the target gene, when the engineered polynucleotide binds to the recruiting moiety, is increased by at least 10%, 20%, 30%, 40%, 50%, 60%, 70%, 80%, 90%, 2 fold, 3 fold, 4 fold, 5 fold, 10 fold, or more compared to the expression or activity of the target gene in the absence of the engineered polynucleotide binding to the recruiting moiety. In some embodiments, the expression or activity of the target gene, when the engineered polynucleotide binds to the recruiting moiety, is decreased by at least 10%, 20%, 30%, 40%, 50%, 60%, 70%, 80%, 90%, 2 fold, 3 fold, 4 fold, 5 fold, 10 fold, or more compared to the expression or activity of the target gene in the absence of the engineered polynucleotide binding to the recruiting moiety.

In some embodiments, the modulating of the expression or activity of the target comprises correcting aberrant expression of the target gene due to splice variant. In some embodiments, the expression or activity of misfolded target gene or protein due to aberrant splice variant, when the engineered polynucleotide binds to the recruiting moiety, is decreased by at least 10%, 20%, 30%, 40%, 50%, 60%, 70%, 80%, 90%, 2 fold, 3 fold, 4 fold, 5 fold, 10 fold, or more compared to the expression or activity of misfolded target gene or protein due to aberrant splice variant in the absence of the engineered polynucleotide binding to the recruiting moiety. In some embodiments, the amount of misfolded protein aggregate due to aberrant splice variant, when the engineered polynucleotide binds to the recruiting moiety, is decreased by at least 10%, 20%, 30%, 40%, 50%, 60%, 70%, 80%, 90%, 2 fold, 3 fold, 4 fold, 5 fold, 10 fold, or more compared to the amount of misfolded protein aggregate due to aberrant splice variant in the absence of the engineered polynucleotide binding to the recruiting moiety. In some embodiments, the amount of plaques comprising the misfolded protein due to aberrant splice variant, when the engineered polynucleotide binds to the recruiting moiety, is decreased by at least 10%, 20%, 30%, 40%, 50%, 60%, 70%, 80%, 90%, 2 fold, 3 fold, 4 fold, 5 fold, 10 fold, or more compared to the amount of plaques comprising the misfolded protein due to aberrant splice variant in the absence of the engineered polynucleotide binding to the recruiting moiety.

### Molecular Interaction(s)

In various aspects of the disclosure, engineered polynucleotides may be able to participate in molecular interactions with polypeptides (e.g., U1-C) or other polynucleotides (e.g., pre-mRNAs, U1 snRNA). The engineered polynucleotides may be configured to, or otherwise able to interact with other polypeptides or polynucleotides via one or more targeting moieties or recruiting moieties. In some embodiments described herein, a targeting moiety comprises free 5′ and 3′ ends that interact with a conserved site of a constitutive splice donor. This interaction can silence the U1 snRNA RNA-binding domain. In some embodiments, the targeting moiety comprises 2′ modified nucleotides that increase affinity to pre-mRNA targets.

In some embodiments, binding of a targeting moiety to a pre-mRNA is stabilized by hydrogen bonds and electrostatic interactions between U1-C and the pre-mRNA backbone around a splice junction region for the pre-mRNA. In such embodiments, U1-C may not make specific base contacts with the pre-mRNA. 2′ nucleotide modifications can favor hydrogen bonding between a targeting moiety and U1-C. Accordingly binding of a targeting moiety (free 5′ and 3′ ends) to a pre-mRNA duplex enables recognition and stabilization of the targeting interaction by U1-C

U1-C can stabilize the spliceosome central nucleus. U1-C enhances the affinities of incompatible 5′-splices and stabilizes the central core of the spliceosome machinery through an interaction bridge between U1-70KD and the Sm ring.

In some embodiments, a targeting moiety can interact with the zinc finger of U1-C. Phosphorothioate internucleotide linkages in the targeting moiety can promote interaction of a targeting moiety and the zinc finger. The engineered polynucleotides may comprise phosphorothioate internucleotide linkages at a specific or particular position that interacts with a zinc finger

In some embodiments, a recruiting moiety forms hydrogen bonds to stem-loop II of U1-A. Such interactions can modulate polyadenylation and acetylation signaling by U1-A because stem-loop II of U1-A may not be silenced by the recruiting moiety.

In some embodiments described herein, the engineered polynucleotide does not comprise any intramolecular disulfide bond.

In some embodiments of the engineered polynucleotide described herein, when associated with the engineered polynucleotide and the spliceosomal moiety, the RNA (e.g., the mRNA, such as the pre-mRNA) exhibits substantially no base pairing with an RNA binding domain (RBD) of U1 snRNA.

In some embodiments of the engineered polynucleotide described herein, when associated with the engineered polynucleotide and the spliceosomal moiety, the RNA (e.g., the mRNA, such as the pre-mRNA) exhibits substantially no base-specific interaction with U1-C protein.

In some embodiments of the engineered polynucleotide described herein, the engineered polynucleotide is configured to specifically interact with zinc-finger of U1-C protein. e.g., comprising an amino acid sequence of: YYCDYCDTYLTHDSPSVRKTHCTGRKHRDNVKF (SEQ ID NO: 5). In some embodiments, a 5′-targeting moiety of the engineered polynucleotide is configured to specifically interact with zinc-finger of U1-C protein. In some embodiments, (e.g., the 5′-targeting moiety of) the engineered polynucleotide is configured to covalently interact (e.g., via disulfide bonding) with zinc-finger of U1-C protein. In some embodiments, (e.g., the 5′-targeting moiety of) the engineered polynucleotide is configured to non-covalently interact (e.g., via hydrogen bonding) with zinc-finger of U1-C protein.

In some embodiments, the engineered polynucleotide (e.g., ASMO1 described herein) comprises a nucleotide sequence complementary to U1 snRNA. In some embodiments of the engineered polynucleotide described herein, the engineered polynucleotide comprises a nucleotide sequence complementary to a partial sequence of Stem-Loop II (SL2) of U1 snRNA. In some embodiments, a side of a stem-loop structure of the engineered polynucleotide comprises a nucleotide sequence complementary to a partial sequence of Stem-Loop II (SL2) of U1 snRNA. In some embodiments, the partial sequence comprises the sequence corresponding to 5′-GGCCU-3′ of SL2 of U1 snRNA. In some embodiments, the partial sequence does not comprise the sequence corresponding to 5′-CACGUUA-3′ of SL2 of U1 snRNA.

In some embodiments of the engineered polynucleotide described herein, the engineered polynucleotide exhibits substantially no base pairing with an anchoring sequence of SL2 of U1 snRNA. In some embodiments, the anchoring sequence comprises the sequence corresponding to 5′-CACGUUA-3′. In some embodiments, an internal loop of the engineered polynucleotide exhibits substantially no base pairing with the anchoring sequence of the SL2 of U1 snRNA. In some embodiments, a lower stem of the engineered polynucleotide exhibits substantially no base pairing with the anchoring sequence of the SL2 of U1 snRNA.

In some embodiments of the engineered polynucleotide described herein, the engineered polynucleotide exhibits substantially no base pairing with H helix of U1 snRNA.

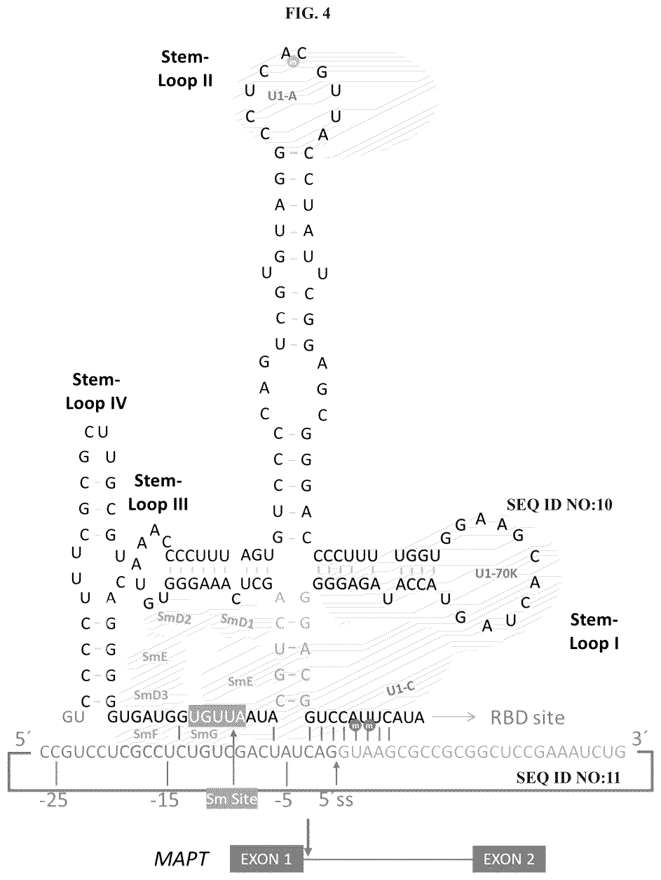

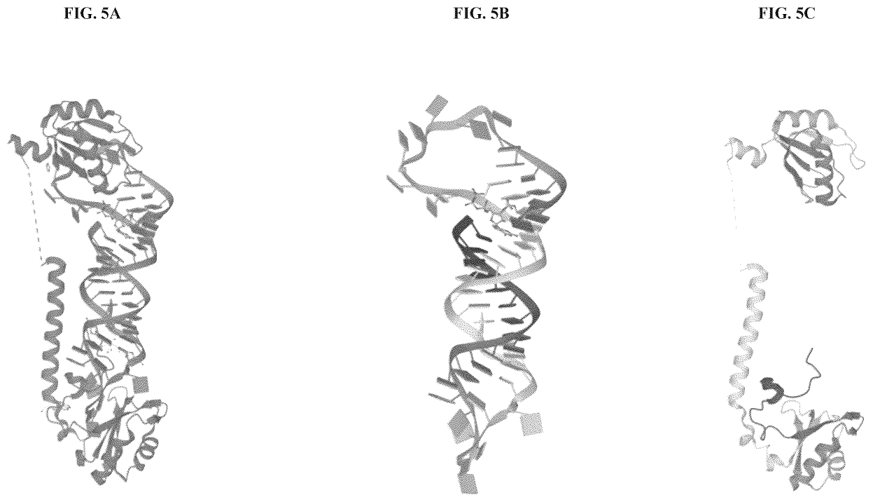

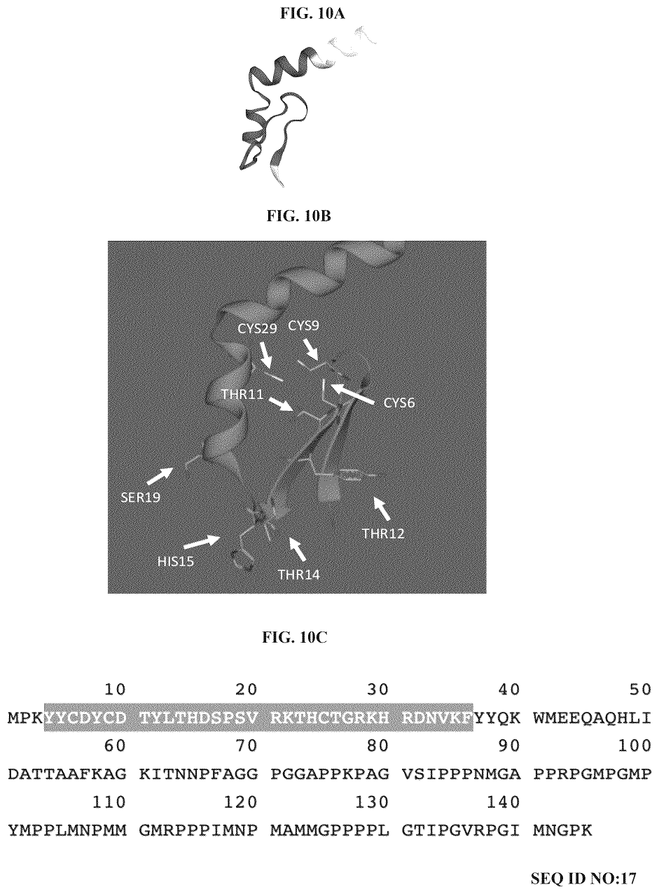

In some embodiments, the engineered polynucleotide does not comprise any intramolecular disulfide bond. In some embodiments, the engineered polynucleotide, when recruiting the spliceosomal moiety described to the target sequence such as target pre-mRNA, the engineered polynucleotide does not exhibit base pairing with an RNA binding domain (RBD) of the spliceosomal moiety such as U1 snRNA. FIG. 4 illustrates such lack of base pairing between the engineered polynucleotide and the RBD of the spliceosomal moiety, where the RBD site of U1 snRNA has the following sequence: 3′-GUCCAUUCAUA-5′ and forms base pairing with the target sequence. In some instances, when the engineered polynucleotide and the spliceosomal moiety are bound, the engineered polynucleotide exhibits substantially no base-specific interaction with U1-C spliceosomal moiety. In some embodiments, the engineered polynucleotide is configured to specifically interact with zinc-finger of U1-C protein or U1-1 spliceosomal moiety. FIG. 10C illustrates a representative sequence of U1-C snRNP containing 145 amino acids, with the highlighted 36 amino acids (YYCDYCDTYLTHDSPSVRKTHCTGRKHRDNVKF (SEQ ID NO: 5)) comprising the zinc-finger domain.

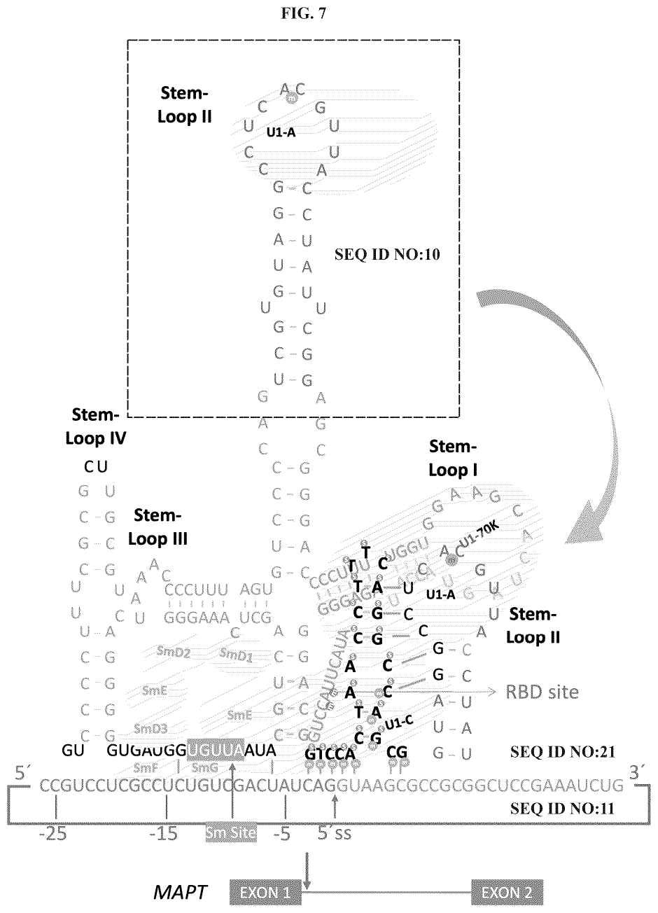

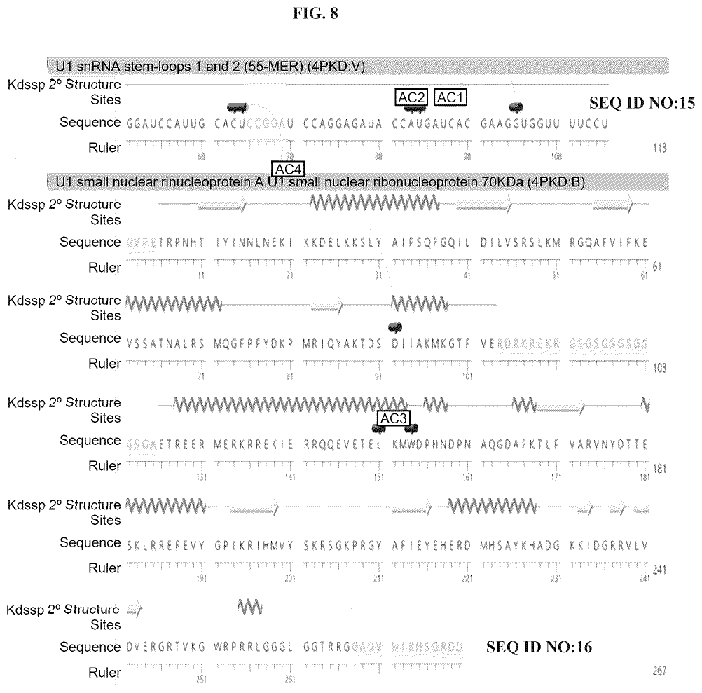

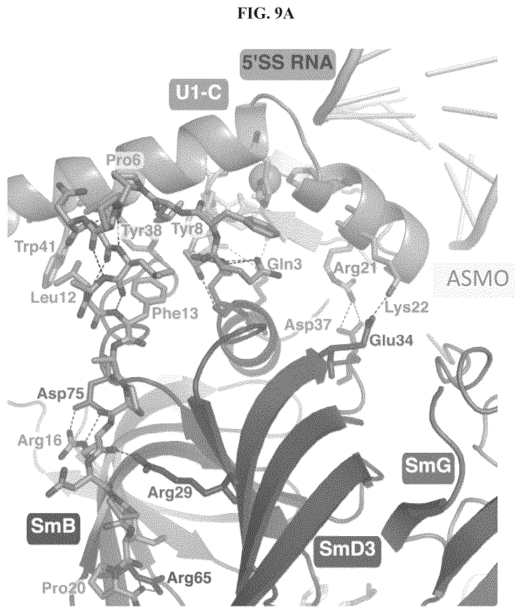

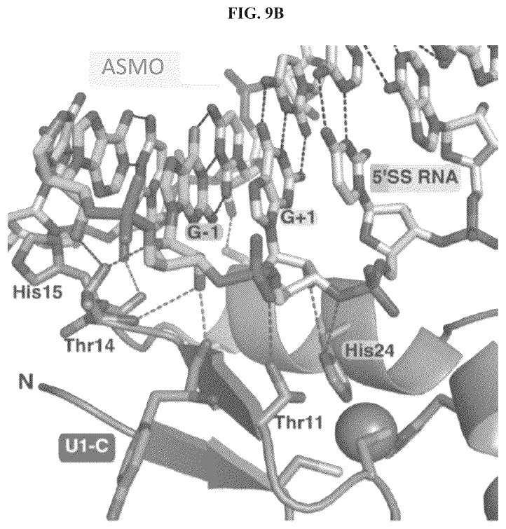

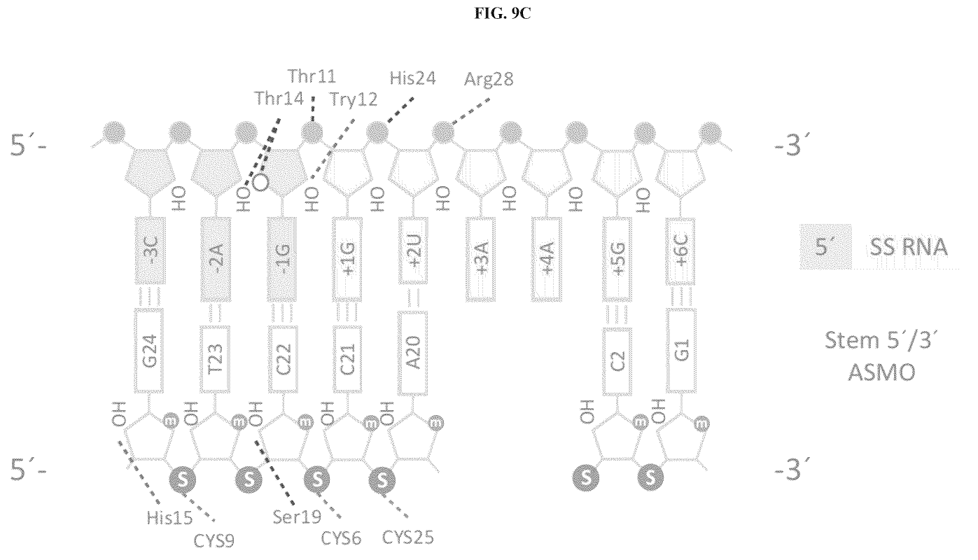

In some embodiments, the engineered polynucleotide is configured to covalently interact (e.g., via disulfide bonding) with zinc-finger of U1-C protein or U1-1 spliceosomal moiety. In some embodiments, the engineered polynucleotide is configured to non-covalently interact (e.g., via hydrogen bonding) with zinc-finger of U1-C protein or U1-1 spliceosomal moiety. In some embodiments, the engineered polynucleotide (e.g., ASMO1 described herein) comprises a nucleotide sequence complementary to U1 snRNA. In some embodiments, the engineered polynucleotide comprises a nucleotide sequence complementary to a partial sequence of Stem-Loop II (SL2) of U1 snRNA. In some embodiments, a side of a stem-loop secondary structure of the engineered polynucleotide comprises a nucleotide sequence complementary to a partial sequence of Stem-Loop II (SL2) of U1 snRNA. In some embodiments, the partial sequence comprises the sequence corresponding to 5′-GGCCU-3′ of SL2 of U1 snRNA. In some embodiments, the partial sequence does not comprise the sequence corresponding to 5′-CACGUUA-3′ of SL2 of U1 snRNA. In some embodiments, the engineered polynucleotide exhibits substantially no base pairing with an anchoring sequence of SL2 of U1 snRNA. In some cases, the internal loop of the engineered polynucleotide exhibits substantially no base pairing with the anchoring sequence of the SL2 of U1 snRNA. In some aspects, the lower stem of the engineered polynucleotide exhibits substantially no base pairing with the anchoring sequence of the SL2 of U1 snRNA. In some aspects, the anchoring sequence comprises the sequence corresponding to 5′-CACGUUA-3′. In some cases, the engineered polynucleotide exhibits substantially no base pairing with H helix of U1 snRNA, where the engineered polynucleotide does not comprise any intramolecular disulfide bond. For example, FIG. 2A illustrates the lack of intramolecular disulfide bond due to the presence of chemical modification with phosphorothioate-type internucleotide bonds. Stabilization of the U1 snRNP complex can be observed through the strong ionic attraction of the Zinc Finger of U1-C, induced by disulfide bridges with thiol of the engineered polynucleotide (ASMO) targeting moieties at 5′- or/and 3-end. The pre-mRNA/engineered polynucleotide (ASMO) duplex bond can be stabilized by hydrogen bonds and electrostatic interactions between U1-C and the backbone of the pre-mRNA around the seam joint, but U1-C does not make base-specific contacts with pre-mRNA. The structure demonstrates that the selection of nucleotides of 5′-splices by U1 snRNP is achieved predominantly through the interaction between Stem 5′/3′ with pre-mRNA. Meanwhile. U1-C adjusts relative affinities of incompatible sites of 5′-splices and stabilizes the central core of spliceosome machinery by the interaction bridge between U1-70 KDa and the Sm ring (see FIGS. 7-9). Of the U1 snRNP specific proteins, U1-70k and U1-C have important roles in aiding recognition of the pre-mRNA transcript. U1-70k has an N-terminus that while highly conserved is predicted to be unstructured (residues ˜2-60), an RNA binding domain (or RBD) that mediates its interaction with a stem-loop of U1 snRNA (residues 92-202), and a C-terminus rich in repeats of arginine and serine residues (an RS domain) as well as R-(D/E) residues. Although this C-terminal domain is not conserved the RS ‘domain’ is important for interaction with non-snRNP splicing factors such as ASF/SF2. Serines in this region are subjected to post-translational modification (phosphorylation) and are important to splicing activity. U1-C consists of an N-terminal zinc-finger domain and a C-terminal region rich in repeats of RG residues. Arginines in this region of U1-C are subjected to post-translational modification (methylation). In contrast to U1-70k, U1-C does not bind to free U1 snRNA but requires the prior binding of the Sm proteins and U1-70k. Mutations in the zinc-finger region of U1-C have a significant effect on recognition of the 5′ splice site by the U1 snRNP, indicating that this protein has a direct role to play in this activity. The assembly and function of U1 snRNP has been greatly enhanced initially by cryo electron microscopy studies and more recently by elucidation of its three-dimensional structure by X-ray crystallography. Previously, crystal structures of four of seven Sm proteins led to the modeling of the remaining three (Sm-F, Sm-E and Sm-G) and the proposal that together they would interact to form a seven-membered ring. The crystal structure of a completely recombinant human U1 snRNP reveals that Sm proteins do form a heptameric ring, composed of a single copy of each Sm protein, and passing through its center is the Sm site of U1 snRNA. In the crystal structure, U1-C is in a position to recognize the duplex formed when the 5′ end of U1 snRNA base-pairs to the 5′ splice site. The finding that the N-terminus of U1-70k extends 180 Å from the RBD and wraps around one face of the Sm ring, crossing Sm-D2 and Sm-D3/B, could therefore ensure the correct structure and positioning of U1-C for interaction with the U1 snRNA:5′ splice site duplex (FIG. 9).

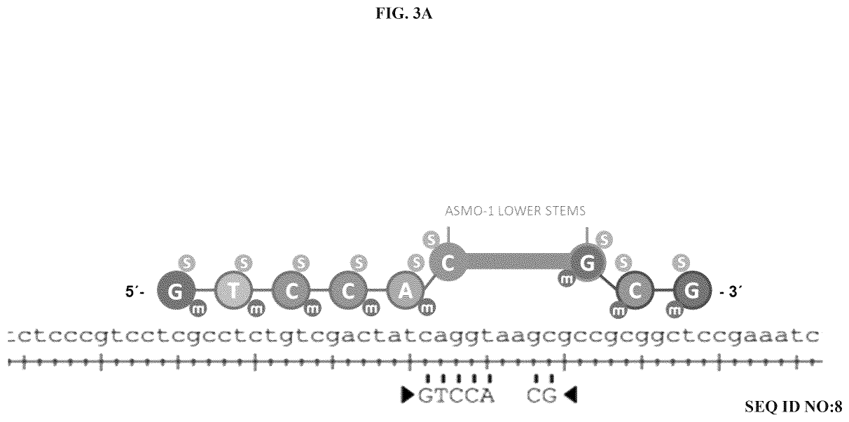

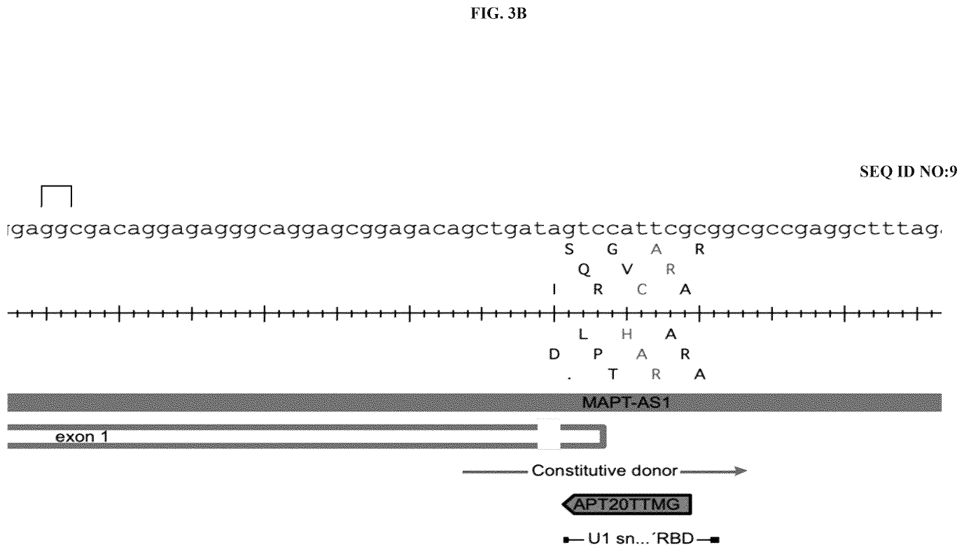

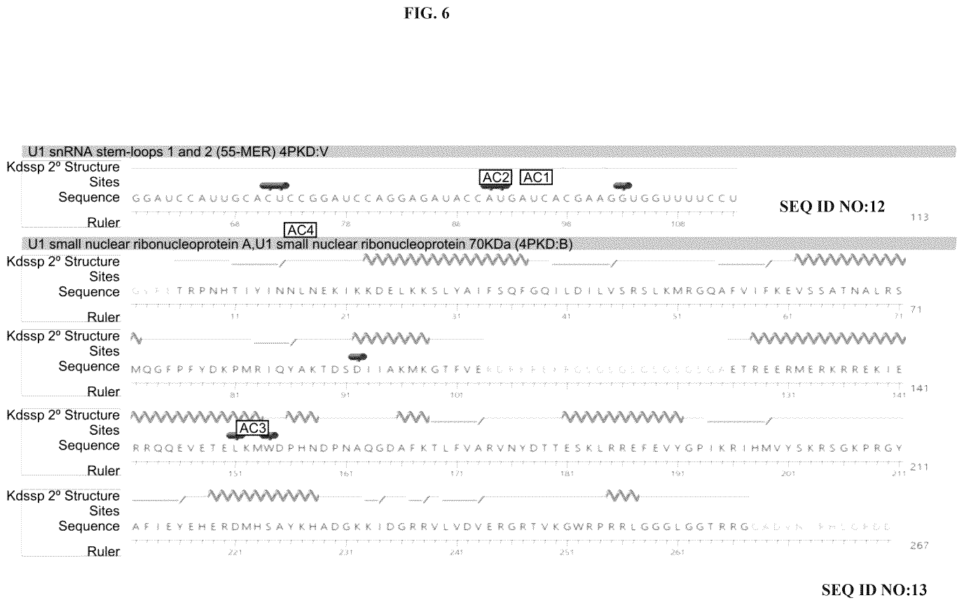

In some embodiments, the engineered polynucleotide, when associated with the spliceosomal moiety described herein, the pre-mRNA exhibits substantially no base pairing with an RNA binding domain (RBD) of U1 snRNA. In some cases, the engineered polynucleotide, when associated with the spliceosomal moiety, the pre-mRNA exhibits substantially no base-specific interaction with U1-C protein. FIG. 4 illustrates that in the absence of the engineered polynucleotide, the RBD of the U1 snRNA binds in the conserved regions of constitutive donor. Meanwhile, in the presence of the engineered polynucleotide the stem 5′/3′ blocked the RBD interaction of the U1 snRNA with the pre-mRNA (FIG. 3 and FIG. 6).

In some embodiments, the engineered polynucleotide is configured to specifically interact with zinc-finger of U1-C protein. In some embodiments, a 5′-targeting moiety of the engineered polynucleotide is configured to specifically interact with zinc-finger of U1-C protein. In some embodiments, the engineered polynucleotide is configured to covalently interact (e.g., via disulfide bonding) with zinc-finger of U1-C protein. In some embodiments, the engineered polynucleotide is configured to non-covalently interact (e.g., via hydrogen bonding) with zinc-finger of U1-C protein. The formation of the pre-mRNA/engineered polynucleotide (ASMO) duplex that interacts with amino acid residues from the U1-C zinc finger stabilizes the 5; region (FIG. 9). Then, favorable molecular dynamics for formation by disulfide bonds formed by atoms in the main and side chain of the U1-C zinc finger with Stem 5′ of the engineered polynucleotide can be observed. A strong ionic bond can also form as ASMO presents interaction with all the cysteines present in the U1-C zinc finger (FIG. 9 and FIG. 10). Additional example interaction between U1-C and pre-mRNA in the presence or in the absence of the engineered polynucleotide described herein is shown in Table 5.

TABLE 5
Example interaction between U1-C and pre-mRNA mediated
by the presence of the engineered polynucleotide

| | In presence of engineered | In absence of engineered |
|---|---|---|
| Domain | polynucleotide | polynucleotide |
| Constitutive | The binding of the 5′/3′ Stem allows | The presence of mutations in the |
| Donor 5′ | the interaction with the conserved site | RBD domain of U1-snRNA can |
| | of the constitutive donor, by silencing | induce a crypto splice or lack of |
| | the U1 snRNA RNA-binding domain | recognition of the 5′ region |
| | (RBD) | |
| Duplex | The pre-mRNA/engineered | Unfavorable molecular dynamics |
| recognition 5′ | polynucleotide duplex bond is | due to the low ionic attraction of the |
| by U1-C | stabilized by hydrogen bonds and | U1-C Zinc finger. It may induce a |
| | electrostatic interactions between U1- | crypto splice or instability in the |
| | C and the pre-mRNA backbone around | formation of the U1 complex in the |
| | the junction region, but U1-C does not | 5′ region |
| | make specific base contacts with pre- | |
| | mRNA. It is important to note that the | |
| | 2′-OME group favors the interaction | |
| | of hydrogen bonds with U1-C | |
| Stabilization | The pre-mRNA/engineered | Unfavorable molecular dynamics |
| of the | polynucleotide duplex bond is | due to the |
| 5′duplex by | stabilized by hydrogen bonds and | low ionic attraction of the U1-C |
| U1-C | electrostatic interactions between U1- | Zinc finger. It may induce a crypto |
| | C and the pre-mRNA backbone around | splice or instability in the formation |
| | the junction region, but U1-C does not | of the U1 complex in the 5′ region |
| | make specific base contacts with pre- | |
| | mRNA | |
| | The 2′-OME group favors the | |
| | interaction of hydrogen bonds with | |
| | U1-C | |
| Stabilization | U1-C adjusts high-precision relative | U1-C sits on SmD3 and its binding |
| of the | affinities of incompatible 5′-splices | is stabilized by the N-terminus of |
| spliceosome | and stabilizes the central core of | U1-70k |
| central | spliceosome machinery through the | |
| nucleus | interaction bridge between U1-70 KDa | |
| | and the Sm ring | |
| Stem-loop | Hydrogen bridges of Stem-Loop II | SNRPA binds stem loop II of U1 |
| | with specific Hairpin-2 (a.k.a., upper | snRNA. In a snRNP-free form (SF- |
| | stem, see FIG. 2B) base and internal | A) may be involved in coupled pre- |
| | loop, associated with the modulation | mRNA splicing and |
| | of the polyadenylation and acetylation | polyadenylation process. May bind |
| | signal by U1-A. It is noted that the | preferentially to the 5′-UGCAC-3′ |
| | anchoring domain of U1-A in Stem- | motif on RNAs. The loss of U1-A |
| | Loop II, is not silenced by Hairpin-2, | self-regulation can induce |
| | inducing the modulation of levels of | premature poly (A) signal, |
| | gene expression and acetylation. The | dysregulation of gene expression |
| | presence of 2′-OME groups induces a | and inadequate acetylation. |
| | change in the molecular dynamics of | |
| | the medium facilitating the | |
| | conformational alteration of U1- | |
| | snRNA and approximation of Stem- | |
| | loop II to engineered polynucleotide. | |

In some embodiments, the engineered polynucleotide (e.g., ASMO1 described herein) comprises a nucleotide sequence complementary to U1 snRNA. In some aspects, the engineered polynucleotide comprises a nucleotide sequence complementary to a partial sequence of Stem-Loop II (SL2) of U1 snRNA. In other aspects, described herein is a side of a stem-loop structure of the engineered polynucleotide comprises a nucleotide sequence complementary to a partial sequence of Stem-Loop II (SL2) of U1 snRNA. In some instances, the partial sequence comprises the sequence corresponding to 5′-GGCCU-3′ of SL2 of U1 snRNA, where the partial sequence does not comprise the sequence corresponding to 5′-CACGUUA-3′ of SL2 of U1 snRNA and where the engineered polynucleotide exhibits substantially no base pairing with an anchoring sequence of SL2 of U1 snRNA. In some aspects, the engineered polynucleotide comprises an internal loop of the engineered polynucleotide exhibits substantially no base pairing with the anchoring sequence of the SL2 of U1 snRNA. In some embodiments, the engineered polynucleotide comprises a lower stem of the engineered polynucleotide exhibiting substantially no base pairing with the anchoring sequence of the SL2 of U1 snRNA. In some embodiments, the anchoring sequence comprises the sequence corresponding to 5′-CACGUUA-3′, where then engineered polynucleotide exhibits substantially no base pairing with H helix of U1 snRNA.

### Set(s) of Engineered Polynucleotides

Described herein, in some embodiments, include a set of engineered polynucleotides each independently described herein. For example, the polynucleotides of the set may independently comprise: (i) one or more targeting moiety (such as described herein) configured to bind a ribonucleic acid (RNA) (such as described herein) (e.g., a messenger ribonucleic acid (mRNA), such as a pre-messenger ribonucleic acid (pre-mRNA)) at a target sequence (such as described herein), and (ii) a recruiting moiety (such as described herein) configured to recruit a post-transcriptional regulating moiety (e.g., a spliceosomal moiety) (such as described herein), wherein the set of engineered polynucleotides are configured to specifically bind the RNA (e.g., the mRNA, such as the pre-mRNA) at a plurality of target sequences comprising the target sequence (such as described herein).

### Vectors

In some embodiments described herein include a vector or a plasmid comprising a nucleic acid sequence encoding an engineered polynucleotide as described herein.

In some embodiments described herein include a plurality of vectors or a plurality of plasmids comprising a plurality of nucleic acid sequences each encoding an engineered polynucleotide as described herein. In some embodiments, the plurality of vectors or the plurality of plasmids comprise the plurality of nucleic acid sequences encoding more than one engineered polynucleotide as described herein. In some embodiments, the plurality of vectors or the plurality of plasmids comprise the plurality of nucleic acid sequences encoding a plurality of engineered polynucleotides (each independently described herein).

### Pharmaceutical Composition(s)

Described herein, in some embodiments, is a pharmaceutical composition comprising the engineered polynucleotide described herein, or a plasmid, a vector, or an isolated DNA encoding the sequence thereof. Pharmaceutical composition, as used herein, refers to a mixture of at least one engineered polynucleotide or a vector encoding the at least one engineered polynucleotide, with other chemical components (i.e. pharmaceutically acceptable inactive ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. Optionally, the compositions include two or more pharmaceutical composition as discussed herein. In practicing the methods of treatment or use provided herein, therapeutically effective amounts of pharmaceutical compositions described herein are administered in a pharmaceutical composition to a mammal having a disease, disorder, or condition to be treated. In some embodiments, the mammal is a human. A therapeutically effective amount can vary widely depending on the severity of the disease, the age and relative health of the subject, the potency of the pharmaceutical composition used and other factors. The pharmaceutical compositions can be used singly or in combination with one or more pharmaceutical compositions as components of mixtures. The pharmaceutical commotions described herein comprise the engineered polynucleotide, the compositions, the cells contacted with the engineered polynucleotide or contacted with the composition comprising the engineered polynucleotide, or a combination thereof.

The pharmaceutical formulations described herein are administered to a subject by appropriate administration routes, including but not limited to, intravenous, intrathecal, intracerebroventricular, intraarterial, oral, parenteral, buccal, topical, transdermal, rectal, intramuscular, subcutaneous, intranasal, intravitreal, intraosseous, transmucosal, inhalation, or intraperitoneal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

Pharmaceutical compositions including a pharmaceutical composition are manufactured in a conventional manner, such as, by way of example only, by means of conventional mixing, dissolving, granulating, dragee-making, levigating, emulsifying, encapsulating, entrapping or compression processes.

### Kit(s)

Described herein, in some embodiments, are kits for using the engineered polynucleotide, the compositions, or the pharmaceutical compositions described herein. In some embodiments, the kits disclosed herein may be used to treat a disease or condition in a subject. In some embodiments, the kit comprises an assemblage of materials or components apart from the engineered polynucleotide, the composition, or the pharmaceutical composition. In some embodiments, the kit comprises the components for assaying and selecting for suitable oligonucleotide for treating a disease or a condition. In some embodiments, the kit comprises components for performing assays such as enzyme-linked immunosorbent assay (ELISA), single-molecular array (Simoa), PCR, or qPCR. The exact nature of the components configured in the kit depends on its intended purpose. For example, some embodiments are configured for the purpose of treating a disease or condition disclosed herein in a subject. In some embodiments, the kit is configured particularly for the purpose of treating mammalian subjects. In some embodiments, the kit is configured particularly for the purpose of treating human subjects.

Instructions for use may be included in the kit. In some embodiments, the kit comprises instructions for administering the composition to a subject in need thereof. In some embodiments, the kit comprises instructions for further engineering the engineered polynucleotide. In some embodiments, the kit comprises instructions thawing or otherwise restoring biological activity of the engineered polynucleotide, which may have been cryopreserved or lyophilized during storage or transportation. In some embodiments, the kit comprises instructions for measuring efficacy for its intended purpose (e.g., therapeutic efficacy if used for treating a subject).

Optionally, the kit also contains other useful components, such as, diluents, buffers, pharmaceutically acceptable carriers, syringes, catheters, applicators, pipetting or measuring tools, bandaging materials or other useful paraphernalia. The materials or components assembled in the kit may be provided to the practitioner stored in any convenient and suitable ways that preserve their operability and utility. For example, the engineered polynucleotide, the composition, or the pharmaceutical composition may be in dissolved, dehydrated, or lyophilized form. The components are typically contained in suitable packaging material(s).

### Methods

Described herein are methods for utilizing an engineered polynucleotide (such as described herein), such as a method for altering a ribonucleic acid (RNA) (e.g., a messenger ribonucleic acid (mRNA), such as a pre-messenger ribonucleic acid (pre-mRNA)) in a cell. The method may comprise contacting the cell with the engineered polynucleotide (such as described herein) that comprises one or more targeting moiety and a recruiting moiety. The one or more targeting moiety may bind to the RNA (e.g., the mRNA, such as the pre-mRNA) (such as described herein) at a target sequence (such as described herein) therein, and the recruiting moiety recruits a post-transcriptional regulating moiety (e.g., a spliceosomal moiety) (such as described herein) within proximity of the target sequence of the RNA (e.g., the mRNA, such as the pre-mRNA) to alter the RNA (e.g., the mRNA, such as the pre-mRNA) in the cell, thereby yielding one or more altered RNA (e.g., one or more altered mRNA, such as one or more altered pre-mRNA). In some embodiments, the method alters an expression or activity of the target gene. In some embodiments, prior to the contacting, the cell exhibits an aberrant messenger ribonucleic acid (mRNA) or protein corresponding to the target gene. In some embodiments, a targeting moiety of the one or more targeting moiety is sufficiently identical or complementary to a consensus sequence in the target sequence of a target gene (e.g., microtubule associated protein tau (MAPT)).

Described herein include a method for altering a ribonucleic acid (RNA) (e.g., a messenger ribonucleic acid (mRNA), such as a pre-messenger ribonucleic acid (pre-mRNA)) at a plurality of locations in a cell. The method may comprise contacting the cell with a set of engineered polynucleotides (such as each independently described herein). An engineered polynucleotide may comprise one or more targeting moiety and a recruiting moiety. The one or more targeting moiety may bind to the RNA (e.g., the mRNA, such as the pre-mRNA) (such as described herein) at a plurality of target sequences (such as described herein) therein. Each recruiting moiety may recruit a post-transcriptional regulating moiety (e.g., a spliceosomal moiety) (such as described herein) within proximity of a target sequence of the RNA (e.g., the mRNA, such as the pre-mRNA) to alter the RNA (e.g., the mRNA, such as the pre-mRNA) in the cell, thereby yielding one or more altered RNA (e.g., one or more altered mRNA, such as one or more altered pre-mRNA). In some embodiments, the method alters an expression or activity of the target gene by altering (e.g., cleaving or/and chemically modifying) the RNA (e.g., the mRNA, such as the pre-mRNA) at the plurality of locations. In some embodiments, prior to the contacting, the cell exhibits an aberrant messenger ribonucleic acid (mRNA) or protein corresponding to the target gene. In some embodiments, one or each targeting moiety of the one or more targeting moiety is sufficiently identical or complementary to a consensus sequence in the target sequence of a target gene (e.g., microtubule associated protein tau (MAPT)).

In some embodiments, the method comprises delivering the engineered polynucleotide into a cell. In some embodiments, the method comprises delivering a polynucleotide encoding the engineered polynucleotide into a cell and subsequently expressing the engineered polynucleotide for modulating the expression or the activity of a gene encoded by the target sequence described herein. In some embodiments, the method comprises using the engineered polynucleotide for treating a disease or condition in a subject in need thereof. The disease or condition may be associated with an aberrant expression or activity of a target gene encoded by the RNA (e.g., the mRNA, such as the pre-mRNA). In some embodiments, the RNA (e.g., the mRNA, such as the pre-mRNA) corresponds to a target gene (e.g., microtubule associated protein tau (MAPT)).

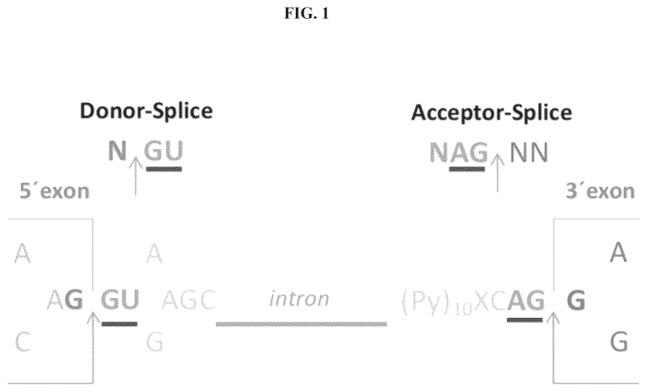

FIG. 1 illustrates a schematic diagram for identifying splice donor and acceptor for designing the nucleotide sequence of the engineered polynucleotide, where the engineered polynucleotides or the methods described herein present an improvement over the currently available approaches for modulating expression or activity of a gene for treating a disease or condition. In some embodiments, the methods described herein modulate the expression or activity of a gene by the engineered polynucleotide targeting the transcript of the gene that causes a disease or condition. In some embodiments, the methods described herein comprise administering the engineered polynucleotide described herein to a subject in need thereof. In some cases, the methods described herein comprise utilizing the engineered polynucleotide to recruit a regulating moiety to modulate the expression or activity of the gene that causes the disease or condition, thereby treating the disease or condition. In some aspects, the methods described herein comprise utilizing the engineered polynucleotide to stabilize the assembly of the regulating moiety to modulate the expression or activity of the gene that causes the disease or condition, thereby treating the disease or condition.

Described herein, in some embodiments, are methods of delivering the engineered polynucleotides described herein to a cell. In some embodiments, the method comprises delivering directly or indirectly engineered polynucleotides to the cell. In some embodiments, the method comprises contacting the cell with a composition comprising the engineered polynucleotide described herein. In some embodiments, the method comprises expressing the engineered polynucleotide described herein in the cell. In some embodiments, the engineered polynucleotide or vector encoding the engineered polynucleotide can be delivered into the cell via any of the transfection methods described herein. In some embodiments, the engineered polynucleotide can be delivered into the cell via the use of expression vectors. In the context of an expression vector, the vector can be readily introduced into the cell described herein by any method in the art. For example, the expression vector can be transferred into the cell by physical, chemical, or biological means.

Physical methods for introducing the engineered polynucleotide or vector encoding the engineered polynucleotide into the cell can include calcium phosphate precipitation, lipofection, particle bombardment, microinjection, gene gun, electroporation, and the like. Methods for producing cells comprising vectors and/or exogenous nucleic acids are suitable for methods herein. One method for the introduction of engineered polynucleotide or vector encoding the engineered polynucleotide into a host cell is calcium phosphate transfection.

Chemical means for introducing the engineered polynucleotide or vector encoding the engineered polynucleotide into the cell can include colloidal dispersion systems, such as macromolecule complexes, nanocapsules, microspheres, beads, and lipid-based systems including oil-in-water emulsions, micelles, mixed micelles, spherical nucleic acid (SNA), liposomes, or lipid nanoparticles. An example colloidal system for use as a delivery vehicle in vitro and in vivo is a liposome (e.g., an artificial membrane vesicle). Other methods of state-of-the-art targeted delivery of nucleic acids are available, such as delivery of engineered polynucleotide or vector encoding the engineered polynucleotide with targeted nanoparticles.

In the case where a non-viral delivery system is utilized, an example delivery vehicle is a liposome. The use of lipid formulations is contemplated for the introduction of the engineered polynucleotide or vector encoding the engineered polynucleotide into a cell (in vitro, ex vivo, or in vivo). In another aspect, the engineered polynucleotide or vector encoding the engineered polynucleotide can be associated with a lipid. The engineered polynucleotide or vector encoding the engineered polynucleotide associated with a lipid can be encapsulated in the aqueous interior of a liposome, interspersed within the lipid bilayer of a liposome, attached to a liposome via a linking molecule that is associated with both the liposome and the engineered polynucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, in some embodiments, they are present in a bilayer structure, as micelles, or with a “collapsed” structure. Alternately, they are simply be interspersed in a solution, possibly forming aggregates that are not uniform in size or shape. Lipids are fatty substances which are, in some embodiments, naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use are obtained from commercial sources. Stock solutions of lipids in chloroform or chloroform/methanol are often stored at about −20° C. Chloroform is used as the only solvent since it is more readily evaporated than methanol, “Liposome” is a generic term encompassing a variety of single and multilamellar lipid vehicles formed by the generation of enclosed lipid bilayers or aggregates. Liposomes are often characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers. However, compositions that have different structures in solution than the normal vesicular structure are also encompassed. For example, the lipids, in some embodiments, assume a micellar structure or merely exist as nonuniform aggregates of lipid molecules. Also contemplated are lipofectamine-nucleic acid complexes.

In some cases, non-viral delivery method comprises lipofection, nucleofection, microinjection, biolistics, virosomes, liposomes, immunoliposomes, exosomes, polycation or lipid:cargo conjugates (or aggregates), naked polypeptide (e.g., recombinant polypeptides), naked DNA, artificial virions, and agent-enhanced uptake of polypeptide or DNA. In some embodiments, the delivery method comprises conjugating or encapsulating the compositions or the engineered polynucleotides described herein with at least one polymer such as natural polymer or synthetic materials. The polymer can be biocompatible or biodegradable. Non-limiting examples of suitable biocompatible, biodegradable synthetic polymers can include aliphatic polyesters, poly(amino acids), copoly(ether-esters), polyalkylenes oxalates, polyamides, poly(iminocarbonates), polyorthoesters, polyoxaesters, polyamidoesters, polyoxaesters containing amine groups, and poly(anhydrides). Such synthetic polymers can be homopolymers or copolymers (e.g., random, block, segmented, graft) of a plurality of different monomers, e.g., two or more of lactic acid, lactide, glycolic acid, glycolide, epsilon-caprolactone, trimethylene carbonate, p-dioxanone, etc. In an example, the scaffold can be comprised of a polymer comprising glycolic acid and lactic acid, such as those with a ratio of glycolic acid to lactic acid of 90/10 or 5/95. Non-limiting examples of naturally occurring biocompatible, biodegradable polymers can include glycoproteins, proteoglycans, polysaccharides, glycosamineoglycan (GAG) and fragment(s) derived from these components, elastin, laminins, decrorin, fibrinogen/fibrin, fibronectins, osteopontin, tenascins, hyaluronic acid, collagen, chondroitin sulfate, heparin, heparan sulfate, ORC, carboxymethyl cellulose, and chitin.

In some cases, the engineered polynucleotide or vector encoding the engineered polynucleotide described herein can be packaged and delivered to the cell via extracellular vesicles. The extracellular vesicles can be any membrane-bound particles. In some embodiments, the extracellular vesicles can be any membrane-bound particles secreted by at least one cell. In some instances, the extracellular vesicles can be any membrane-bound particles synthesized in vitro. In some instances, the extracellular vesicles can be any membrane-bound particles synthesized without a cell. In some cases, the extracellular vesicles can be exosomes, microvesicles, retrovirus-like particles, apoptotic bodies, apoptosomes, oncosomes, exophers, enveloped viruses, exomeres, or other very large extracellular vesicles.

Described herein, in some aspects, are methods for modulating or altering expression or activity of a gene encoded by a target sequence in a cell. In some embodiments, the target sequence is a pre-messenger ribonucleic acid (pre-mRNA) in a cell. In some embodiments, the method comprises contacting the cell with an engineered polynucleotide comprising one or more targeting moieties and a recruiting moiety. In some embodiments, the one or more targeting moieties bind to the pre-mRNA at a target sequence therein. In some embodiments, the recruiting moiety recruits a post-transcriptional regulating moiety (e.g., a spliceosomal moiety) within proximity of the target sequence of the pre-mRNA to alter the pre-mRNA in the cell, thereby yielding one or more altered pre-mRNA. In some embodiments, the pre-mRNA corresponds to a target gene such as microtubule associated protein tau (MAPT). In some embodiments, the method, when the engineered polynucleotide binds and recruits the spliceosomal moiety to the target sequence, increases an expression or activity of the target gene. In some embodiments, the method, when the engineered polynucleotide binds and recruits the spliceosomal moiety to the target sequence, decreases an expression or activity of the target gene. In some embodiments, the method, when the engineered polynucleotide binds and recruits the spliceosomal moiety to the target sequence, corrects aberrant messenger ribonucleic acid (mRNA) or protein corresponding to the target gene.

In some embodiments, the method comprises contacting or delivering two or more engineered polynucleotides into a single cell, where the engineered polynucleotides each comprise one or more targeting moieties configured to bind to two or more target sequences. The two or more target sequences can be located on the same strand of the pre-mRNA encoding a target gene. The two or more target sequences can be located on different strands of pre-mRNA encoding the same target gene. The two or more target sequences can be located on different strands of pre-mRNA, where each strand of the pre-mRNA can encode different target gene. In some embodiments, the method comprises two or more engineered polynucleotides configured to specifically bind the pre-mRNA at a plurality of target sequences comprising the target sequence.

Disclosed herein, in some embodiments, are methods of treating a disease or condition by modulating expression or activity of a target gene in a cell, thereby treating the disease or condition. In some embodiments, the method comprises treating a disease or condition by correcting aberrant messenger ribonucleic acid (mRNA) or protein corresponding to the target gene. In some embodiments, the disease or condition is associated with increased expression or activity of any one of the target gene described herein. In some embodiments, the disease or condition is associated with decreased expression or activity of any one of the target gene described herein. In some embodiments, the disease or condition is associated with splicing of aberrant messenger ribonucleic acid (mRNA) or protein corresponding to of any one of the target gene described herein.

In some embodiments, the method of treating a disease or condition may comprise modulation modulating expression or activity of genes in a cell. The genes may comprise genes that are not the target gene and/or do not directly interact with the engineered polynucleotide. Upon administration of the engineered polynucleotide or pharmaceutical composition comprising the engineered polynucleotide, the expression of genes in a cell or subject may be altered and a different gene expression profile may be generated. For example, genes of the cell or subject may be down regulated or upregulated as a result of the administration of the engineered polynucleotide. The alteration of genes in the cell may provide a benefit that alleviates the symptoms or the causes of the disease or condition.

In some embodiments, the engineered polynucleotide or pharmaceutical composition comprising the engineered polynucleotide can be administered to the subject alone (e.g., standalone treatment). In some embodiments, the engineered polynucleotide or pharmaceutical composition comprising the engineered polynucleotide is administered in combination with an additional agent. In some cases, the additional agent as used herein is administered alone. The engineered polynucleotide or pharmaceutical composition comprising the engineered polynucleotide and the additional agent can be administered together or sequentially. The combination therapies can be administered within the same day, or can be administered one or more days, weeks, months, or years apart.

In some embodiments, the engineered polynucleotide or pharmaceutical composition comprising the engineered polynucleotide is administered at a concentration of at least 5 nM. In some embodiments, the engineered polynucleotide or pharmaceutical composition comprising the engineered polynucleotide is administered at a concentration of at least 10 nM. In some embodiments, the engineered polynucleotide or pharmaceutical composition comprising the engineered polynucleotide is administered at a concentration of at least 15 nM. In some embodiments, the engineered polynucleotide or pharmaceutical composition comprising the engineered polynucleotide is administered at a concentration of at least 20 nM. In some embodiments, the engineered polynucleotide or pharmaceutical composition comprising the engineered polynucleotide is administered at a concentration of at least 1 nM, 2 nM, 3 nM, 4 nM, 5 nM, 6 nM, 7 nM, 8 nM, 9 nM, 10 nM, 11 nM, 12 nM, 13 nM, 14 nM, 15 nM, 16 nM, 17 nM, 18 nM, 19 nM, 20 nM, 21 nM, 22 nM, 23 nM, 24 nM, 25 nM, 26 nM, 27 nM, 28 nM, 29 nM, 30 nM, or more. In some embodiments, the engineered polynucleotide or pharmaceutical composition comprising the engineered polynucleotide is administered at a concentration of no more than 1 nM, 2 nM, 3 nM, 4 nM, 5 nM, 6 nM, 7 nM, 8 nM, 9 nM, 10 nM, 11 nM, 12 nM, 13 nM, 14 nM, 15 nM, 16 nM, 17 nM, 18 nM, 19 nM, 20 nM, 21 nM, 22 nM, 23 nM, 24 nM, 25 nM, 26 nM, 27 nM, 28 nM, 29 nM, 30 nM, or less. In some embodiments, the engineered polynucleotide or pharmaceutical composition comprising the engineered polynucleotide is administered at a concentration of about 5 nM to 20 nM.

In some embodiments, the engineered polynucleotide or pharmaceutical composition comprising the engineered polynucleotide is a first-line treatment for the disease or condition. In some embodiments, the engineered polynucleotide or pharmaceutical composition comprising the engineered polynucleotide is a second-line, third-line, or fourth-line treatment. In some embodiments, the engineered polynucleotide or pharmaceutical composition comprising the engineered polynucleotide comprises at least one, two, three, four, five, six, seven, eight, nine, 10, 20, 30 or more oligonucleotide. In general, method disclosed herein comprises administering the engineered polynucleotide or pharmaceutical composition comprising the engineered polynucleotide by intrathecal or intravenous (“i.v.”) administration. However, in some instances, method comprises administering the engineered polynucleotide or pharmaceutical composition comprising the engineered polynucleotide by intraperitoneal injection. In some instances, the method comprises administering the engineered polynucleotide or pharmaceutical composition comprising the engineered polynucleotide by oral or intranasal administration. It is conceivable that one can also administer the engineered polynucleotide or pharmaceutical composition comprising the engineered polynucleotide disclosed herein by other routes such as subcutaneous injection, intramuscular injection. Intracerebroventricular injection, intravitreal injection, intradermal injection, transdermal injection percutaneous administration, intranasal administration, intralymphatic injection, rectal administration intragastric administration, or any other suitable parenteral administration. In some embodiments, routes for local delivery closer to site of injury or inflammation are preferred over systemic routes. Routes, dosage, time points, and duration of administrating therapeutics can be adjusted. In some embodiments, administration of therapeutics is prior to, or after, onset of either, or both, acute and chronic symptoms of the disease or condition.

Suitable dose and dosage administrated to a subject is determined by factors including, but no limited to, the particular the engineered polynucleotide, composition, or pharmaceutical composition, disease condition and its severity, the identity (e.g., weight, sex, age) of the subject in need of treatment, and can be determined according to the particular circumstances surrounding the case, including, e.g., the specific agent being administered, the route of administration, the condition being treated, and the subject being treated.

Described herein are methods of treating neurodegenerative diseases using the engineered polynucleotide or vector encoding the engineered polynucleotide. In some aspects, the neurodegenerative disease is Alzheimer's Disease. The engineered polynucleotide can be a engineered polynucleotide as described in PCT/US2022/037391, of which is incorporated by reference in its entirety.

Described herein is a method for treating neurodegenerative disease in a subject in need thereof, comprising: administering to a subject comprising administering to the subject a pharmaceutical composition comprising an engineered polynucleotide or vector encoding the engineered polynucleotide. In some embodiments, the engineered polynucleotide comprises: (i) one or more targeting moieties configured to specifically bind a pre-messenger ribonucleic acid (pre-mRNA) at a target sequence therein; and (ii) a recruiting moiety configured to recruit a spliceosomal moiety, wherein, when associated with the pre-mRNA and the engineered polynucleotide, the spliceosomal moiety alters the pre-mRNA in or in proximity to the target sequence.

In some embodiment, the method comprises contacting a cell of the subject with the engineered polynucleotide or vector encoding the engineered polynucleotide. In some embodiments, the cell is microglia. In some embodiments, the cell is an astrocyte. In some embodiments, the cell is a neuron. In some embodiments, the cell is an excitatory neuron. In some embodiments, the cell is from a patient diagnosed with Alzheimer's Disease. In some embodiments, the cell is a neuron from a healthy patient. In some embodiments, the cell is a neuron from a patient diagnosed with Alzheimer's disease. In some embodiments, the cell is an excitatory neuron from a patient diagnosed with Alzheimer's disease. In some embodiments, the cell is from a hippocampal or cortical region.

In some embodiments, the neurodegenerative disease is a neurodegenerative disease associated with presence of Tau in a subject's brain. For example, the neurodegenerative disease can be a tau-opathy or other disease in which the presence of tau is indicative of a pathology. As described elsewhere in this disclosure, engineered polynucleotides when administered to subject or a cell, can reduce the amount of tau, alter the expression of tau, or reduce the expression of tau. As the presence of tau is altered, the administration of the engineered polypeptide can alleviate or ameliorate the symptoms of neurogenerative disease, or otherwise treat the neurodegenerative disease. In some embodiment, the neurodegenerative disease is selected from the group consisting of: Alzheimer's disease. Pick's disease, chronic traumatic encephalopathy, progressive supranuclear palsy, argyrophilic grain disease. Amyotrophic Lateral Sclerosis, and Frontotemporal Dementia.

In some embodiments, contacting a cell with the engineered polynucleotide or vector encoding the engineered polynucleotide results in an increase in fiber breadth, branching number, branching junctions, neurite straightness, and total axonal material amount in a cell. In some embodiments, contacting a cell with the engineered polynucleotide or vector encoding the engineered polynucleotide results in improved axonal functions related to synapse and cognitive functions. In some embodiments, contacting a cell with the engineered polynucleotide or vector encoding the engineered polynucleotide results in reduced levels of Tau protein in the cell. In some embodiments, contacting a cell with the engineered polynucleotide or vector encoding the engineered polynucleotide results in the differential expression of genes associated with the generation of neurons, neurogenesis, neuron differentiation, and regulation of synapsis assembly. In some embodiments, contacting a cell with the engineered polynucleotide or vector encoding the engineered polynucleotide results in the enrichment of genes related to steroid biosynthesis pathways. MAPK signaling, cell cycle. PI3K-Akt signaling, cellular senescence pathways; fatty acid metabolism and biosynthesis, cholesterol metabolism. AMPK signaling pathway, and Ras signaling pathways. In some embodiments, contacting a cell with the engineered polynucleotide or vector encoding the engineered polynucleotide results in the enrichment of genes related to increased mRNA transport. In some embodiments, contacting a cell with the engineered polynucleotide or vector encoding the engineered polynucleotide results in the prevention of Alzheimer's disease-related phenotypes in the cell. In some embodiments, contacting a cell with the engineered polynucleotide or vector encoding the engineered polynucleotide results in increased synapse formation. In some embodiments, contacting a cell with the engineered polynucleotide or vector encoding the engineered polynucleotide results in improved synaptic integrity and synaptic plasticity. In some embodiments, contacting a cell with the engineered polynucleotide decreases premature polyadenylation of one or more transcripts of the cell. In some embodiments, contacting a cell with the engineered polynucleotide or vector encoding the engineered polynucleotide results in decreased excitotoxicity. In some embodiments, contacting a cell with the engineered polynucleotide or vector encoding the engineered polynucleotide results in improvements in neuronal pathways and morphology associated with the synapse in the cell. In some embodiments, contacting a cell with the engineered polynucleotide or vector encoding the engineered polynucleotide results in an increase in neuronal activity. In some embodiments, contacting a cell with the engineered polynucleotide or vector encoding the engineered polynucleotide results in reducing U1-70K aggregation or mislocalization in a cell. In some embodiments, contacting a cell with the engineered polynucleotide or vector encoding the engineered polynucleotide results in reducing amyloid β expression or aggregation in a cell. In some embodiments, contacting a cell with the engineered polynucleotide or vector encoding the engineered polynucleotide results in modulating astrogliosis.

In some embodiments, the cell is a neuron obtained from induced pluripotent stem cells. In some embodiments, the cell is an excitatory neuron obtained from induced pluripotent stem cells. In some embodiments, the cell is from a healthy patient. In some embodiments, the cell is an excitatory neuron from a healthy patient. In some embodiments, the cell is from a hippocampal or cortical region.

In some embodiments, the engineered polynucleotide is ASMO1. In some embodiments, ASMO1 interacts directly or indirectly with U1-C and U1-70K. In some embodiments, ASMO1 interacts directly or indirectly with the U1 complex and with MAPT pre-mRNA.

Use of absolute or sequential terms, for example, “will,” “will not,” “shall,” “shall not,” “must,” “must not,” “first,” “initially,” “next.” “subsequently,” “before.” “after,” “lastly,” and “finally,” are not meant to limit scope of the present embodiments disclosed herein but as example(s).

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

Any systems, methods, software, and platforms described herein are modular. Accordingly, terms such as “first” and “second” do not necessarily imply priority, order of importance, or order of acts.

The term “about” when referring to a number or a numerical range means that the number or numerical range referred to is an approximation within experimental variability (or within statistical experimental error), and the number or numerical range may vary from, for example, from 1% to 15% of the stated number or numerical range. In examples, the term “about” refers to ±10% of a stated number or value.

The terms “increased”, “increasing”, or “increase” are used herein to generally mean an increase by a statically significant amount. In some aspects, the terms “increased,” or “increase.” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level.

The terms “decreased”, “decreasing”, or “decrease” are used herein generally to mean a decrease by a statistically significant amount. In some aspects, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### LIST OF EMBODIMENTS

Embodiment 1. An engineered polynucleotide comprising: one or more targeting moieties configured to specifically bind a pre-messenger ribonucleic acid (pre-mRNA) at a target sequence therein; and a recruiting moiety configured to recruit a post-transcriptional regulating moiety (e.g., a spliceosomal moiety), wherein, when associated with said pre-mRNA and said engineered polynucleotide, said post-transcriptional regulating moiety alters said pre-mRNA in or in proximity to said target sequence.

Embodiment 2. The engineered polynucleotide of embodiment 1, wherein a targeting moiety of said one or more targeting moieties is sufficiently identical or complementary to a consensus sequence in said target sequence of a target gene.

Embodiment 3. The engineered polynucleotide of embodiment 1 or 2, wherein said one or more targeting moieties comprise (1) a first targeting moiety configured to specifically bind a first targeted sequence in said target sequence of said pre-mRNA, and (2) a second targeting moiety configured to specifically bind a second targeted sequence in said target sequence of said pre-mRNA.

Embodiment 4. The engineered polynucleotide of embodiment 3, wherein said first targeted sequence comprises a consensus sequence in said target sequence.

Embodiment 5. The engineered polynucleotide of embodiment 3 or 4, wherein said second targeted sequence comprises a consensus sequence in said target sequence.

Embodiment 6. The engineered polynucleotide of any one of embodiments 3 to 5, wherein said first and second targeted sequences are apart in said target sequence by a spacing sequence of no more than five nucleotides (e.g., one or two nucleotides).

Embodiment 7. The engineered polynucleotide of any one of embodiments 1 to 6, wherein said target sequence comprises an exon-intron boundary in said pre-mRNA.

Embodiment 8. The engineered polynucleotide of embodiment 7, wherein said first and second targeted sequences are both 5′ or 3′ with respect to said exon-intron boundary.

Embodiment 9. The engineered polynucleotide of embodiment 7, wherein one of said first and second targeted sequences is 5′ with respect to said exon-intron boundary; and wherein the other of said first and second targeted sequences is 3′ with respect to said exon-intron boundary.

Embodiment 10. The engineered polynucleotide of any one of embodiments 1 to 9, wherein said target sequence comprises a splice site in said pre-mRNA.

Embodiment 11. The engineered polynucleotide of embodiment 10, wherein said first or second targeted sequence comprises a splice site (e.g., 5′ ss) in said pre-mRNA.

Embodiment 12. The engineered polynucleotide of any one of embodiments 1 to 11, wherein one of said first and second targeting moieties is 5′ with respect to said recruiting moiety, and the other of said first and second targeting moieties is 3′ with respect to said recruiting moiety

Embodiment 13. The engineered polynucleotide of embodiment 12, wherein said first targeting moiety or said second targeting moiety comprises a sequence identical or complementary to a sequence set forth in Table 1.

Embodiment 14. The engineered polynucleotide of embodiment 12, wherein said first targeting moiety comprises a sequence identical or complementary to a sequence selected from the exon sequence column of Table 1; and wherein said second targeting moiety comprises a sequence identical or complementary to a sequence set forth in the intron sequence column of Table 1.

Embodiment 15. The engineered polynucleotide of embodiment 12, wherein said first targeting moiety comprises a sequence identical or complementary to a sequence set forth in the intron sequence column of Table 1; and wherein said second targeting moiety comprises a sequence identical or complementary to a sequence set forth in the exon sequence column of Table 1.

Embodiment 16. The engineered polynucleotide of embodiment 12, wherein said first targeting moiety or said second targeting moiety comprises a sequence identical or complementary to a consensus sequence of an intron donor site (e.g., selected from GU, GT, GC, and CA).

Embodiment 17. The engineered polynucleotide of embodiment 12, wherein said first targeting moiety or said second targeting moiety comprises a sequence identical or complementary to a consensus sequence of an exon donor site (e.g., G).

Embodiment 18. The engineered polynucleotide of embodiment 12, wherein said first targeting moiety or said second targeting moiety comprises a sequence identical or complementary to a consensus sequence selected from GU, GC, G, and CA.

Embodiment 19. The engineered polynucleotide of any one of embodiments 1 to 18, wherein said spliceosomal moiety is selected from a spliceosomal ribonucleoprotein complex, a spliceosomal small nuclear ribonucleic acid (snRNA), a spliceosomal protein, a functional variant thereof, or a functional fragment thereof.

Embodiment 20. The engineered polynucleotide of embodiment 19, wherein said spliceosomal moiety comprises U1 snRNA and a spliceosomal protein.

Embodiment 21. The engineered polynucleotide of any one of embodiments 19 to 20, wherein said spliceosomal snRNA is selected from U1, U2, U4, U5, U6, U11, U12, U14atac, U6atac, and combinations thereof.

Embodiment 22. The engineered polynucleotide of any one of embodiments 19 to 21, wherein said spliceosomal protein is selected from Sm, U1-70k, U1A, U1C, and combinations thereof.

Embodiment 23. The engineered polynucleotide of any one of embodiments 1 to 22, wherein said recruiting moiety comprises a nucleotide sequence that is at least 70%, 80%, 85%, or 90% identical or complementary to any one of SEQ ID NOs: 1-2.

Embodiment 24. The engineered polynucleotide of embodiment 23, wherein said recruiting moiety comprises a nucleotide sequence that is identical or complementary to any one of SEQ ID NOs: 1-2.

Embodiment 25. The engineered polynucleotide of any one of embodiments 1 to 24, wherein said engineered polynucleotide comprises a (e.g., secondary) structural feature.

Embodiment 26. The engineered polynucleotide of embodiment 25, wherein said engineered polynucleotide comprises an apical loop, an upper stem, an internal loop, a lower stem, or a combination thereof.

Embodiment 27. The engineered polynucleotide of embodiment 25, wherein said engineered polynucleotide comprises a loop (e.g., an internal loop) adjacent to a stem (e.g., a lower stem or an upper stem) comprising two complementary stem sequences.

Embodiment 28. The engineered polynucleotide of embodiment 27, wherein a stem sequence of said stem (e.g., said lower stem or said upper stem) comprises no more than about five, four, or three nucleotides.

Embodiment 29. The engineered polynucleotide of embodiment 27 or 28, wherein said loop is an internal loop adjacent to said stem (e.g., said lower stem) and a further stem (e.g., an upper stem) comprising two complementary stem sequences.

Embodiment 30. The engineered polynucleotide of embodiment 29, wherein said internal loop comprises a nucleic acid sequence of no more than 10, 9, or 8 nucleotides.

Embodiment 31. The engineered polynucleotide of embodiment 29 or 30, wherein a stem sequence of said further stem (e.g., said upper stem) comprises no more than about five, four, or three nucleotides.

Embodiment 32. The engineered polynucleotide of any one of embodiments 29 to 31, wherein said engineered polynucleotide further comprises an apical loop.

Embodiment 33. The engineered polynucleotide of embodiment 32, wherein said apical loop comprises a nucleic acid sequence of no more than 10, 9, 8, 7, 6, or 5 nucleotides.

Embodiment 34. The engineered polynucleotide of any one of embodiments 1 to 33, wherein said engineered polynucleotide does not comprise any intramolecular disulfide bond.

Embodiment 35. The engineered polynucleotide of any one of embodiments 1 to 34, wherein, when associated with said engineered polynucleotide and said spliceosomal moiety, said pre-mRNA exhibits substantially no base pairing with an RNA binding domain (RBD) of U1 snRNA.

Embodiment 36. The engineered polynucleotide of any one of embodiments 1 to 35, wherein, when associated with said engineered polynucleotide and said spliceosomal moiety, said pre-mRNA exhibits substantially no base-specific interaction with U1-C protein.

Embodiment 37. The engineered polynucleotide of any one of embodiments 1 to 36, wherein said engineered polynucleotide is configured to specifically interact with zinc-finger of U1-C protein.

Embodiment 38. The engineered polynucleotide of embodiment 37, wherein a 5′-targeting moiety of said engineered polynucleotide is configured to specifically interact with zinc-finger of U1-C protein.

Embodiment 39. The engineered polynucleotide of embodiment 37 or 38, wherein (e.g., said 5′-targeting moiety of) said engineered polynucleotide is configured to covalently interact (e.g., via disulfide bonding) with zinc-finger of U1-C protein.

Embodiment 40. The engineered polynucleotide of any one of embodiments 37 to 39, wherein (e.g., said 5′-targeting moiety of) said engineered polynucleotide is configured to non-covalently interact (e.g., via hydrogen bonding) with zinc-finger of U1-C protein.

Embodiment 41. The engineered polynucleotide of any one of embodiments 1 to 40, wherein said engineered polynucleotide comprises a nucleotide sequence complementary to a partial sequence of Stem-Loop II (SL2) of U1 snRNA.

Embodiment 42. The engineered polynucleotide of embodiment 41, wherein a side of a stem-loop structure of said engineered polynucleotide comprises a nucleotide sequence complementary to a partial sequence of Stem-Loop II (SL2) of U1 snRNA.

Embodiment 43. The engineered polynucleotide of embodiment 41 or 42, wherein said partial sequence comprises the sequence corresponding to 5′-GGCCU-3′ of SL2 of U1 snRNA.

Embodiment 44. The engineered polynucleotide of any one of embodiments 41 to 43, wherein said partial sequence does not comprise the sequence corresponding to 5′-CACGUUA-3′ of SL2 of U1 snRNA.

Embodiment 45. The engineered polynucleotide of any one of embodiments 1 to 44, wherein said engineered polynucleotide exhibits substantially no base pairing with an anchoring sequence of SL2 of U1 snRNA.

Embodiment 46. The engineered polynucleotide of embodiment 45, wherein an internal loop of said engineered polynucleotide exhibits substantially no base pairing with said anchoring sequence of said SL2 of U1 snRNA.

Embodiment 47. The engineered polynucleotide of embodiment 45 or 46, wherein a lower stem of said engineered polynucleotide exhibits substantially no base pairing with said anchoring sequence of said SL2 of U1 snRNA.

Embodiment 48. The engineered polynucleotide of any one of embodiments 45 to 47, wherein said anchoring sequence comprises the sequence corresponding to 5′-CACGUUA-3′.

Embodiment 49. The engineered polynucleotide of any one of embodiments 1 to 48, wherein said engineered polynucleotide exhibits substantially no base pairing with H helix of U1 snRNA.

Embodiment 50. The engineered polynucleotide of any one of embodiments 1 to 49, wherein said engineered polynucleotide comprises at least one chemical modification.

Embodiment 51. The engineered polynucleotide of embodiment 50, wherein said engineered polynucleotide comprises at least one 2′-modified (e.g., 2′-methoxy, 2′-methoxymethyl, or 2′-methoxyethyl) nucleotides.

Embodiment 52. The engineered polynucleotide of embodiment 50 or 51, wherein at least about 50%, 60%, 70%, 80%, or 90% nucleotides of said engineered polynucleotide are chemically modified nucleotides.

Embodiment 53. The engineered polynucleotide of embodiment 50 or 51, wherein at least about 50%, 60%, 70%, 80%, or 90% nucleotides of said engineered polynucleotide are 2′-modified (e.g., 2′-methoxy, 2′-methoxymethyl, or 2′-methoxyethyl) nucleotides.

Embodiment 54. The engineered polynucleotide of any one of embodiments 50 to 53, wherein said engineered polynucleotide comprises at least one phosphorothioate internucleotide bond.

Embodiment 55. The engineered polynucleotide of any one of embodiments 50 to 54, wherein at least about 50%, 60%, 70%, 80%, or 90% internucleotide linkages of said engineered polynucleotide are chemically modified.

Embodiment 56. The engineered polynucleotide of any one of embodiments 50 to 54, wherein at least about 50%, 60%, 70%, 80%, or 90% internucleotide linkages are phosphorathioate.

Embodiment 57. The engineered polynucleotide of any one of embodiments 1 to 56, wherein said engineered polynucleotide comprises about 10 to about 40 nucleotides, about 10 to about 35 nucleotides, about 10 to about 30 nucleotides, or about 10 to about 25 nucleotides.

Embodiment 58. The engineered polynucleotide of any one of embodiments 1 to 57, wherein said recruiting moiety comprises about 10 to about 30 nucleotides, or about 10 to about 20 nucleotides.

Embodiment 59. The engineered polynucleotide of any one of embodiments 1 to 58, wherein said one or more targeting moieties each independently comprises about 2 to about 15 nucleotides, about 2 nucleotides to about 10 nucleotides, or about 2 nucleotides to about 8 nucleotides.

Embodiment 60. The engineered polynucleotide of any one of embodiments 1 to 59, wherein one of said first and second targeting moieties comprises about 2 nucleotides, and the other of said first and second targeting moieties comprises about 5 or 6 nucleotides.

Embodiment 61. The engineered polynucleotide of any one of embodiments 1 to 60, wherein, when associated with said engineered polynucleotide and said pre-mRNA, said spliceosomal moiety cleaves or splices said pre-mRNA in said target sequence.

Embodiment 62. The engineered polynucleotide of any one of embodiments 1 to 61, wherein said spliceosomal moiety further facilitates modification of a cleaved pre-mRNA.

Embodiment 63. An engineered polynucleotide comprising a nucleotide sequence that is at least 70%, 80%, 85%, or 90% identical or complementary to any one of SEQ ID NOs: 1-4, which engineered polynucleotide is characterized by a (e.g., secondary) structural feature.

Embodiment 64. The engineered polynucleotide of embodiment 63, wherein said nucleotide sequence is identical or complementary to any one of SEQ ID NOs: 1-4.

Embodiment 65. The engineered polynucleotide of embodiment 63 or 64, wherein said structural feature comprises one or more stem-loop structures.

Embodiment 66. The engineered polynucleotide of embodiment 63 or 64, wherein said structural feature comprises an apical loop, an upper stem, an internal loop, a lower stem, or a combination thereof.

Embodiment 67. The engineered polynucleotide of any one of embodiments 63 to 66, wherein said engineered polynucleotide comprises a loop (e.g., an internal loop) adjacent to a stem (e.g., a lower stem or an upper stem) comprising two complementary stem sequences.

Embodiment 68. The engineered polynucleotide of embodiment 67, wherein a stem sequence of said stem (e.g., said lower stem or said upper stem) comprises no more than about five, four, or three nucleotides.

Embodiment 69. The engineered polynucleotide of any one of embodiments 63 to 68, wherein said loop is an internal loop adjacent to said stem (e.g., said lower stem) and a further stem (e.g., an upper stem) comprising two complementary stem sequences.

Embodiment 70. The engineered polynucleotide of embodiment 69, wherein said internal loop comprises a nucleic acid sequence of no more than 10, 9, or 8 nucleotides.

Embodiment 71. The engineered polynucleotide of embodiment 69 or 70, wherein a stem sequence of said further stem (e.g., said upper stem) comprises no more than about five, four, or three nucleotides.

Embodiment 72. The engineered polynucleotide of any one of embodiments 69 to 71, wherein said engineered polynucleotide further comprises an apical loop.

Embodiment 73. The engineered polynucleotide of embodiment 72, wherein said apical loop comprises a nucleic acid sequence of no more than 10, 9, 8, 7, 6, or 5 nucleotides.

Embodiment 74. The engineered polynucleotide of any one of embodiments 63 to 73, wherein said engineered polynucleotide further comprises one or more targeting moieties sufficiently identical or complementary to a target sequence of a target gene.

Embodiment 75. The engineered polynucleotide of embodiment 74, wherein a targeting moiety of said one or more targeting moieties is sufficiently identical or complementary to a consensus sequence in said target sequence of said target gene.

Embodiment 76. The engineered polynucleotide of embodiment 74, wherein said target gene is microtubule associated protein tau (MAPT).

Embodiment 77. The engineered polynucleotide of any one of embodiments 63 to 75, wherein said engineered polynucleotide comprises at least one chemical modification.

Embodiment 78. The engineered polynucleotide of embodiment 77, wherein said engineered polynucleotide comprises at least one phosphorothioate internucleotide bond.

Embodiment 79. The engineered polynucleotide of embodiment 77, wherein at least about 50%, 60%, 70%, 80%, or 90% internucleotide linkages of said engineered polynucleotide are chemically modified.

Embodiment 80. The engineered polynucleotide of embodiment 77, wherein at least about 50%, 60%, 70%, 80%, or 90% internucleotide linkages are phosphorathioate.

Embodiment 81. The engineered polynucleotide of any one of embodiments 77 to 80, wherein said engineered polynucleotide comprises at least one 2′-modified (e.g., 2′-methoxy, 2′-methoxymethyl, or 2′-methoxyethyl) nucleotides.

Embodiment 82. The engineered polynucleotide of any one of embodiments 77 to 80, wherein at least about 50%, 60%, 70%, 80%, or 90% nucleotides of said engineered polynucleotide are chemically modified nucleotides.

Embodiment 83. The engineered polynucleotide of any one of embodiments 77 to 80, wherein at least about 50%, 60%, 70%, 80%, or 90% nucleotides of said engineered polynucleotide are 2′-modified (e.g., 2′-methoxy, 2′-methoxymethyl, or 2′-methoxyethyl) nucleotides.

Embodiment 84. The engineered polynucleotide of any one of embodiments 64 to 84, wherein said engineered polynucleotide comprises about 10 to about 40 nucleotides, about 10 to about 35 nucleotides, about 10 to about 30 nucleotides, or about 10 to about 25 nucleotides.

Embodiment 85. A method for altering a pre-messenger ribonucleic acid (pre-mRNA) in a cell, the method comprising contacting said cell with an engineered polynucleotide that comprises one or more targeting moieties and a recruiting moiety, wherein said one or more targeting moieties bind to said pre-mRNA at a target sequence therein, and said recruiting moiety recruits a post-transcriptional regulating moiety (e.g., a spliceosomal moiety) within proximity of said target sequence of said pre-mRNA to alter said pre-mRNA in said cell, thereby yielding one or more altered pre-mRNA.

Embodiment 86. The method of embodiment 85, wherein a targeting moiety of said one or more targeting moieties is sufficiently identical or complementary to a consensus sequence in said target sequence of a target gene.

Embodiment 87. The method of embodiment 85 or 86, wherein said pre-mRNA corresponds to a target gene.

Embodiment 88. The method of embodiment 87, wherein said target gene is microtubule associated protein tau (MAPT).

Embodiment 89. The method of any one of embodiments 85 to 88, wherein the method alters an expression or activity of said target gene.

Embodiment 90. The method of embodiment any one of embodiments 85 to 89, wherein, prior to said contacting, said cell exhibits an aberrant messenger ribonucleic acid (mRNA) or protein corresponding to said target gene.

Embodiment 91. A set of engineered polynucleotides each independently comprise: (i) one or more targeting moieties configured to bind a pre-messenger ribonucleic acid (pre-mRNA) at a target sequence, and (ii) a recruiting moiety configured to recruit a post-transcriptional regulating moiety (e.g., a spliceosomal moiety), wherein said set of engineered polynucleotides are configured to specifically bind said pre-mRNA at a plurality of target sequences comprising said target sequence.

### EXAMPLES

The following illustrative examples are representative of embodiments of the stimulation, systems, and methods described herein and are not meant to be limiting in any way.

### Example 1. Modulating Expression of Target Gene with Engineered Polynucleotide

Cells obtained from a cell like (e.g., HEK293 cell line) are cultured and maintained in cell culture medium. The cells can then be contacted with the engineered polynucleotide or a vector encoding the engineered polynucleotide for delivery of the engineered polynucleotide or a vector encoding the engineered polynucleotide into the cells by any one of the delivery method described herein. After the engineered polynucleotide is delivered into the cells, the cells can be cultured for a period of time to allow the engineered polynucleotide to modulate the expression or activity of the target gene. Cells can then be harvested and lysed for measurement of the expression or activity of the target gene. For example, the cells can be harvested and lysed and examined for the abundance of pre-mRNA. mRNA, or protein of the target gene modulated by the engineered polynucleotide. In other cases, the cells can be fixed and prepared for microscopic examination. For example, the cells can be examined under microscope for the presence or changes of the abundance of the inclusion body or amyloid plaque associated with any one of the target gene described herein (e.g., tau neurofibrillary tangles encoded by MAPT target gene or amyloid plaques encoded by APP target gene).

### Example 2. Treating a Neurological Disease by Editing RNA

A subject is diagnosed with Alzheimer's disease stemmed from an aberrant splicing or polyadenylation activity of pre-mRNA the subject's brain. The subject is prescribed a dosing regimen of a pharmaceutical composition comprising a chemically modified engineered polynucleotide disclosed herein for recruiting and stabilizing least one regulating moiety to decrease the aberrant splicing or polyadenylation activity of multiple different pre-mRNA, thereby decreasing symptoms of Alzheimer's. Some of the pre-mRNA (among other pre-mRNA) may comprise MAPT pre-mRNA. The chemically modified engineered polynucleotide, upon binding to the MAPT pre-mRNA recruits and stabilizes at least one regulating moiety for correctly splicing the MAPT pre-mRNA. In some embodiments, the chemically modified engineered polynucleotide increases the specificity or efficiency of recruiting and stabilizing the at least one regulating moiety RNA editing entity. The modulation of the MAPT pre-mRNA by the engineered polynucleotide decreases the amount of tau plaques in the subject, thereby treating or decreasing the symptoms of Alzheimer's in the subject.

### Example 3. Assessment of Mitochondrial Activity after Incubation with ASMO1 in Neurons from Induced Pluripotent Stem Cells (IPSCs)

Mitochondria are central for various cellular processes that include ATP production, intracellular calcium signaling, and generation of reactive oxygen species. Neurons critically depend on mitochondrial function to establish membrane excitability and to execute the complex processes of neurotransmission and plasticity. In addition to evaluating the metabolism of a cell, feasibility studies are used to determine whether a particular molecule is toxic to a test system, being of fundamental importance for the determination of working concentrations and safety profile. Cell viability is defined as the number of healthy cells in a sample and can be used as a marker for death after exposure to toxic agents.

The CyQUANT MTT Cell Viability Assay utilizes the MTT reagent to determine mammalian cell viability. Measuring changes to cell viability is a method for assessing cell health and determining genotoxicity. The redox potential in viable mammalian cells causes the water soluble MTT reagent (3-(4,5-dimethylthiazol-2-yl)-2,5-diphenyltetrazolium bromide) to convert to an insoluble formazan product. After solubilization of the formazan with SDS (sodium dodecyl sulfate) reagent, the concentration of the colorimetric probe is determined by an optical density measurement at 540-570 nm (CyQUANT™ MTT Cell Viability Assay).

Cells from healthy patient (HDC) and diagnosed with Alzheimer's disease (ADC) were cultured for 6 days, followed by addition of ASMO1 (6.17, 18.52, 55.56, 166.67, 500 nM) or medium only for 7 days (day 13). The culture medium was replaced by fresh medium containing MTT solution. The cells were kept in a CO₂ incubator (37° C., 5% CO₂) for 4 hours, followed by the addition of SDS-HCl. Contents of the different wells were homogenized, and the absorbance reading was performed in a microplate reader at 540 nm. Medians were obtained for each biological replicate. 3 independent experiments were performed in 3 technical replicates. Values were expressed as mean±standard error of the mean (SEM). To assess the statistical difference between the groups, the one-way ANOVA test was performed followed by the Tukey's post-test. P values less than 0.05 were considered as indicative of significance. GraphPad Prism 9 software (GraphPad Software Inc., San Diego, CA, USA) was used for statistical analyses.

As shown in FIG. 11A-B, excitatory neurons from (A) HDC or (B) ADC were cultured for 6 days before treatment incubation with APT20TTMG. After 7 days of treatment, neurons were evaluated through commercial kit for mitochondrial activity.

No concentration of ASMO1 alters mitochondrial activity, suggesting that it does not alter the viability of neurons obtained from iPSCs from healthy patient and patient diagnosed with Alzheimer's Disease.

### Example 4. Assessment of Mitochondrial Activity after Incubation with ASMO1 in Central Nervous System Cells

Mitochondria are increasingly recognized as key hubs in immune responses mediated by astrocytes and microglia. A pathologic feature of AD is reduced mitophagy, the cellular process in which damaged mitochondria are eliminated from the cell by autophagy. Disruption of cell energetics is an important factor underlying the pathogenesis of AD, supporting the idea that alteration of mitochondrial functions may be the cause or the result of the pathological hallmarks of the disease.

Similar to Example 3. The CyQUANT MTT Cell Viability Assay is used to determine mammalian cell viability. Neurons and microglia from healthy patient (HDC) and microglia from patient diagnosed with Alzheimer's Disease (ADC) were plated and cultured for 6 days, followed by addition of ASMO1 (18.52, and 500 nM) or medium only for 7 days (day 13). Astrocytes were plated and cultured for 1 day, followed by addition of ASMO1 (18.52 and 500 nM) or medium only for 24 hours. After, culture medium was replaced by fresh medium containing MTT solution. The cells are kept in a CO₂ incubator (37° C., 5% CO₂) for 4 hours, followed by the addition of SDS-HCl. Contents of the different wells were homogenized, and the absorbance reading was performed in a microplate reader at 540 nm. 2-3 independent experiments were performed in 3 technical replicates, and statistical analysis is performed as described in Example 3.

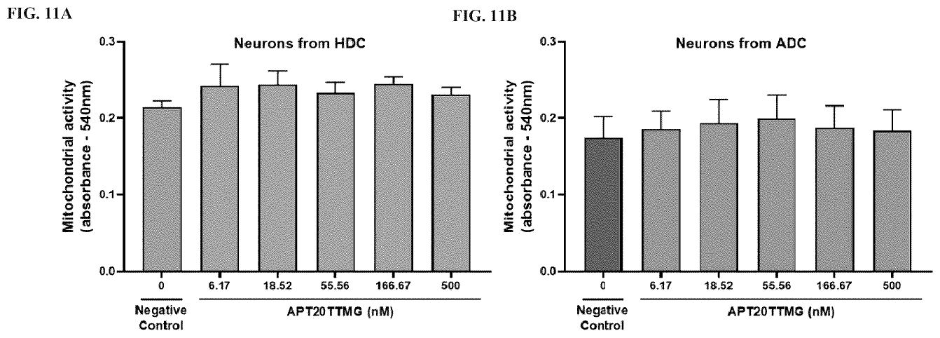

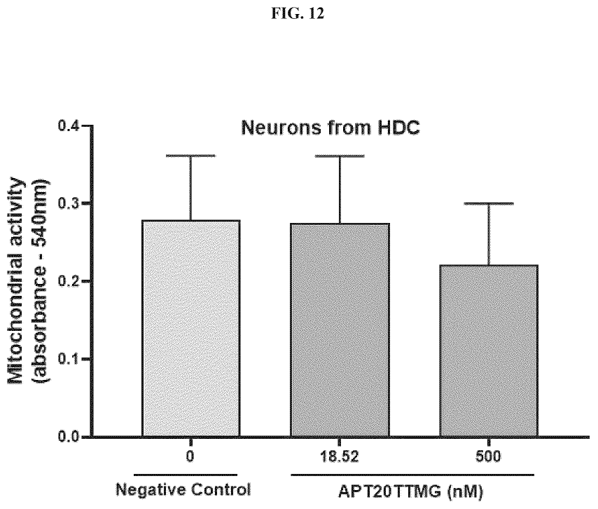

As shown in FIG. 12, excitatory neurons from HDC were cultured for 6 days before treatment incubation with APT20TTMG. After 7 days of treatment, neurons were evaluated through a commercial kit for mitochondrial activity. Analysis of variance (ANOVA) followed by Tukey post hoc. Data of 3 independent experiments performed in three technical replicates. P values <0.05 were considered statistically significant, compared to the control group (no treated cells).

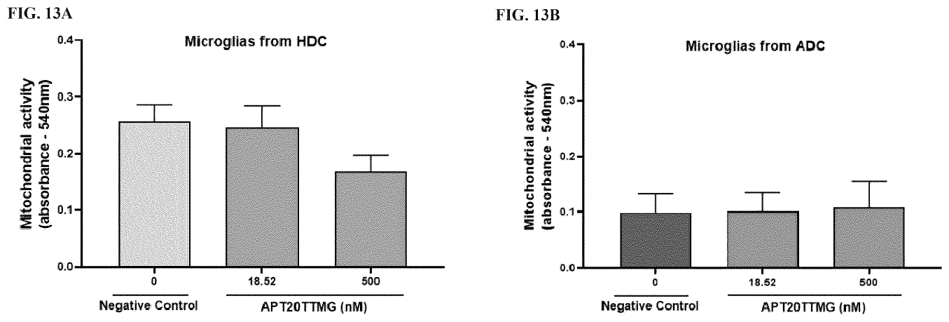

As shown in FIG. 13A-B, microglia from (A) HDC or (B) ADC were cultured for 6 days before treatment incubation with APT20TTMG. After 7 days of treatment, microglias were evaluated through a commercial kit for measuring mitochondrial activity. Analysis of variance (ANOVA) followed by Tukey post hoc. Data of 2 independent experiments performed in three technical replicates. P values <0.05 were considered statistically significant, compared to control group (no treated cells).

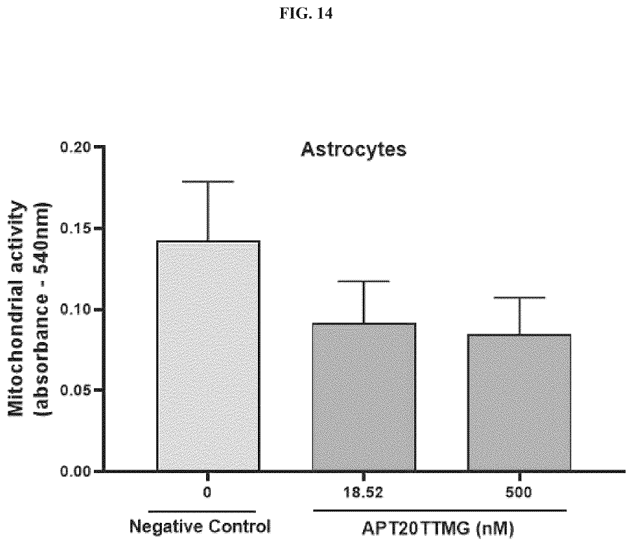

As shown in FIG. 14, astrocytes were cultured for 1 day before treatment incubation with APT20TTMG. After 24 hours of treatment, astrocytes were evaluated through a commercial kit for mitochondrial activity. Analysis of variance (ANOVA) followed by Tukey post hoc. Data of 3 independent experiments performed in three technical replicates. P values<0.05 were considered statistically significant, compared to the control group (no treated cells).

After incubation with ASMO1, no change in mitochondrial activity is observed in neurons, microglia, and astrocytes. However, in neurons and microglia from healthy patients, there minor reduction in this parameter at the highest concentration (500 nM) and, in astrocytes, there is a minor reduction in in the two concentrations evaluated, which may be related to the reduction of reactivity in astrocytes and, as a consequence, of cellular stress.

### Example 5. Assessment of Glutamate Levels after Incubation with ASMO1 in Neurons from Induced Pluripotent Stem Cells (iPSCs)

Glutamate is the most abundant excitatory neurotransmitter in the mammalian Central Nervous System (CNS). It is extensively distributed in the CNS whereas it is almost exclusively located intracellularly. The amount of available extracellular glutamate is subject to strict regulation to allow an appropriate level of signaling. Most of the excitatory neurotransmission in the mammalian CNS is mediated by glutamate and its receptors, mainly ligand-gated ionotropic glutamate receptors (iGluRs). These receptors also play fundamental roles in synaptic plasticity, the underlying molecular mechanism of learning and memory. One subgroup of iGluRs is selectively gated by the specific agonist N-methyl-d-aspartate (NMDA), thus named NMDA receptors (NMDARs), which are essential for their crucial role in synaptic function and plasticity. Insufficient synaptic NMDAR signaling compromises neuronal cell survival. Excessive stimulation of glutamatergic signaling, however, results in excitotoxicity, in which nerve cells are damaged or killed. Besides acute effects, many studies indicate a role for glutamate excitotoxicity in delayed slowly evolving neurodegeneration.

The Glutamate-Glo™ Assay is a bioluminescent assay for the detection of glutamate in biological samples. The assay is based on the conversion of glutamine to glutamate by glutaminase enzyme. Next, glutamate oxidation and NADH production are coupled with a bioluminescent NADH detection system. Glutamate dehydrogenase uses glutamate and NAD+ to produce α-ketoglutarate and NADH. In the presence of NADH, a pro-luciferin Reductase Substrate is converted by Reductase to luciferin that is then used by Ultra-Glo™ Recombinant Luciferase to produce light.

Cells from healthy patient (HDC) and patient diagnosed with Alzheimer's Disease (ADC) were cultured for 6 days, followed by addition of ASMO1 (6.17, 18.52, 55.56, 166.67 nM, and 500 nM) or medium only for 7 days (day 13) and extracellular medium collection for further analysis. Samples were prepared for relative quantification of glutamate, using the Glutamate-Glo™ Assay, and glutamate levels are reported in Relative Luminescence Units (RLUs). 3 independent experiments were performed in 6 technical replicates, and statistical analysis was performed as described in Example 3.

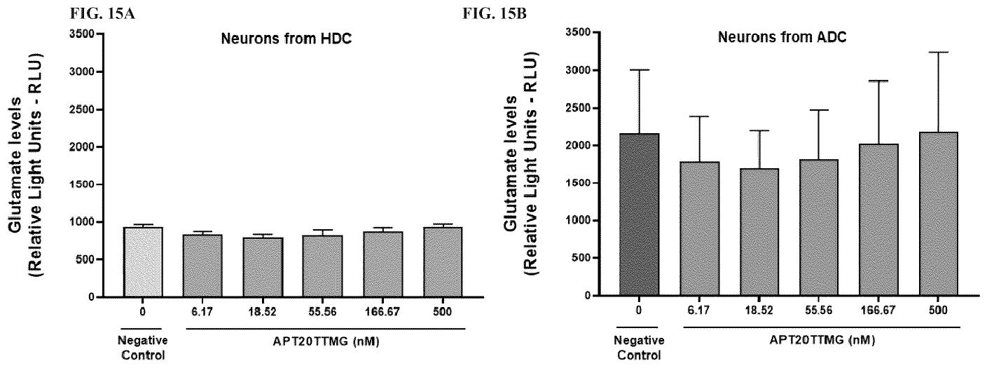

As shown in FIG. 15A-B, excitatory neurons from (A) HDC or (B) ADC were cultured for 6 days before treatment incubation with APT20TTMG. After 7 days of treatment, neurons were evaluated through commercial kit for glutamate release.

No significant change in extracellular glutamate levels was observed after incubation with ASMO1 in neurons obtained from iPSCs from healthy patient and patient diagnosed with Alzheimer's disease, corroborating previous data in Examples 3-4 that shows that at these concentrations and conditions. ASMO1 appears to be safe.

### Example 6. Evaluation of Morphological Changes of Neurons from Induced Pluripotent Stem Cells (iPSCs) after Incubation with ASMO1

Neuronal populations affected in Alzheimer's disease (AD) feature abnormalities in synapse morphology and marked reductions in the total number of synapses, which is thought to underlie the memory and cognitive deficits that are characteristic of this disease. Proper function of axons and axon terminals relies on the transport of proteins, organelles, vesicles, and other elements from the site of synthesis in the cell body. So, axonal transport is a critical cellular process underlying axonal and synaptic function. Several studies demonstrate that dystrophic axons are observed before detectable deposition of classical tau and amyloid pathologies. Understanding the temporal course of the axonal pathology is of high relevance to comprehend the progression of the disease over time and define possible therapeutic targets and the window time where a treatment might be effective.

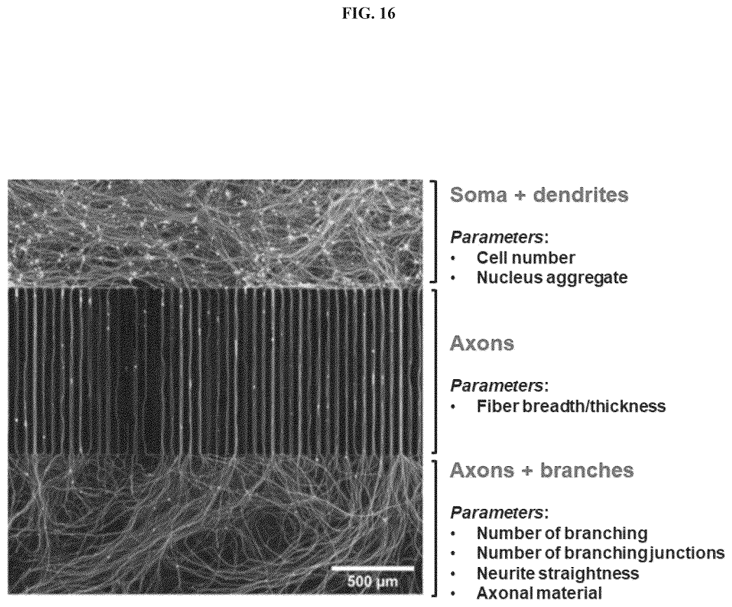

Cells from healthy patient (HDC) and patient diagnosed with Alzheimer's disease (ADC) were cultured for 6 days in plates from NeuroHTS™ technology, followed by addition of ASMO1 (6.17, 18.52, 55.56, 166.67 nM, and 500 nM) or medium only for 7 days (day 13). After, all neurons were evaluated in 7-factor neuronal morphological profile through the software MetaXpress. As shown in FIG. 16, neurons were evaluated in three distinct regions and parameters. In the upper compartment of the well, cell number and nuclear aggregate were evaluated in the soma and dendrites, in the middle compartment of the channel (axons) the thickness of the axonal fiber was verified and, in the bottom compartment, where the axons and dendrites are located, 4 parameters were evaluated: number of branching, number of branching junctions, neurite straightness, and axonal material, 3 independent experiments were performed in up to 3 technical replicates.

Values were expressed as mean±standard error of the mean. To assess the statistical difference between the groups, the one-way ANOVA test was performed followed by the Tukey's (parametric) to cell aggregate, fiber breadth, and neurite straightness, or Kruskal-Wallis (nonparametric) with post-test to cell number, number of branching, number of branching per cell, branching junction, branching junction per cell, total axonal material and, axonal material per cell. P values less than 0.05 were considered as indicative of significance. GraphPad Prism 9 software (GraphPad Software Inc., San Diego, CA, USA) was used for statistical analyses.

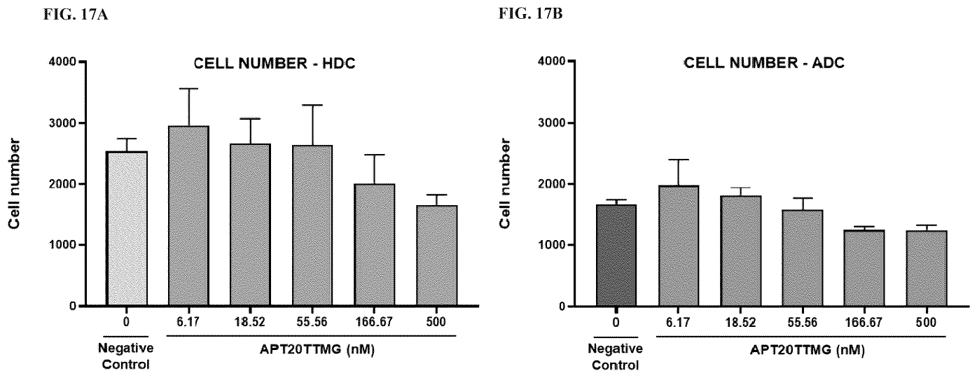

As shown in FIG. 17A-B, excitatory neurons were cultured for 6 days before incubation with ASMO1. After 7 days of treatment, number of neurons were evaluated through the software MetaXpress. (A) Cells number from healthy patient cells (HDC); (B) Cells number from AD patient cells (ADC). Number of cells is an indirect indication of cell death. This parameter is extremely important to understand the safety profile of ASMO1 in these cells, helping to choose the concentrations that will be used in later assays. In addition, some parameters analyzed will need to be normalized in relation to the total number of cells at the end of the experiment, to avoid masking the results obtained. Although no concentration reduced the number of cells (in a statistically significant manner), there appears to be a reduction in the two highest concentrations (166.67 and 500 nM) in HDC and ADC.

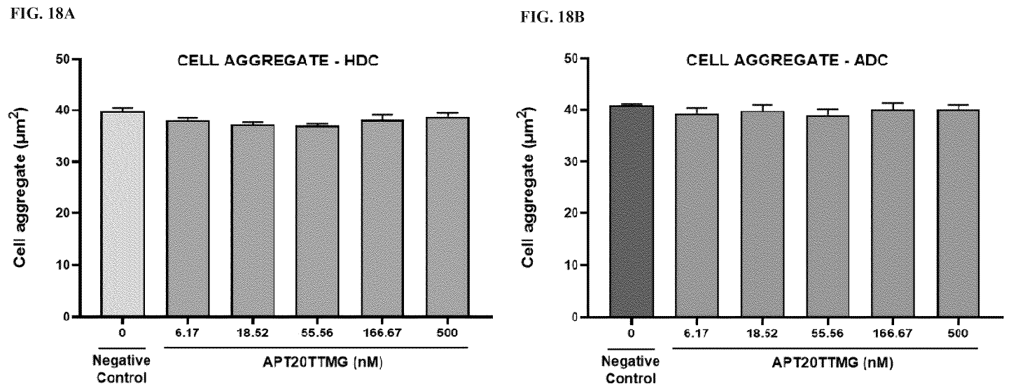

As shown in FIG. 18A-B, excitatory neurons were cultured for 6 days before incubation with ASMO1. After 7 days of treatment, cell aggregates were evaluated through the software MetaXpress. (A) Cells aggregate from healthy patient cells (HDC); (B) Cells aggregate from AD patient cells (ADC).

This parameter may be related to the clumping of neurons in culture, that is, due to the presence of cellular debris and even the presence of free DNA. These two may appear as indicative of toxicity after addition of molecules. Incubation with ASMO1 at different concentrations did not change the clumping of the cells, a new safety indicator in the presence of ASMO1.

As shown in FIG. 19A-B, excitatory neurons were cultured for 6 days before incubation with ASMO1. After 7 days of treatment, fiber breadth was evaluated through the software MetaXpress. (A) Fiber breadth from healthy patient cells (HDC); (B) Fiber breadth from AD patient cells (ADC).

Fiber breadth/thickness is an indication for axonal bundling, which is comprised of an intertwined group of axons. Any impairment of fiber thickness could result in alterations in axonal transport, which plays a key role in the pathogenesis of AD. After treatment with all concentrations of ASMO1, both in HDC and ADC, there was a trend of increasing fiber thickness. In addition. ADC neurons treated with the ASMO1 in all evaluated concentrations showed a similar profile to the negative HDC control. Even in HDC incubation with the ASMO1 appeared to increase fiber thickness. Although the results did not show statistical difference, this parameter was considered when choosing concentrations in later studies. These combined results support a hypothesis that the ASMO1 can improve axonal transport.

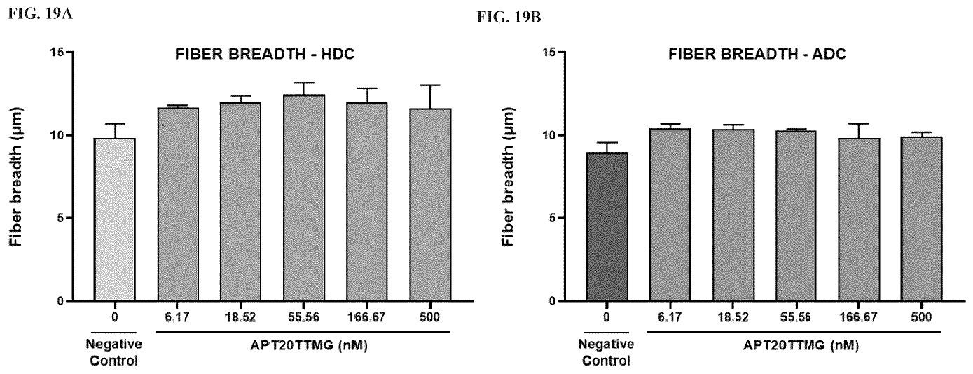

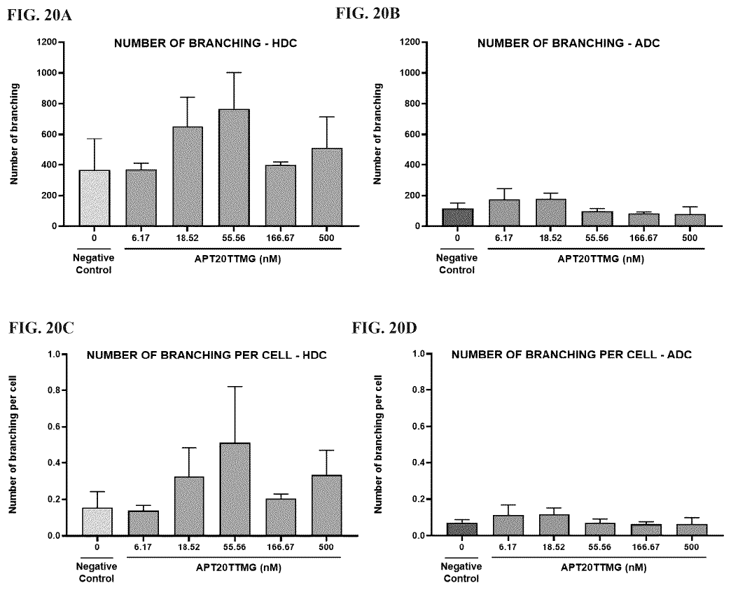

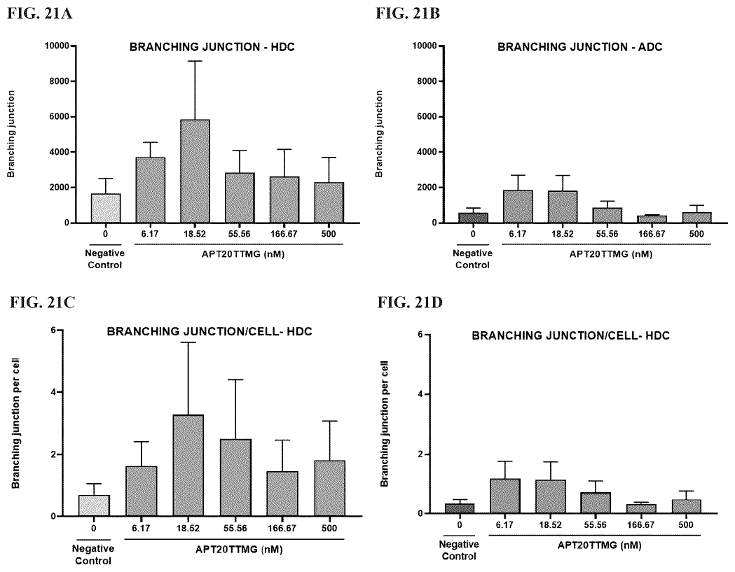

As shown in FIG. 20A-D, the number of branching is an indication for neuronal growth regeneration and/or growth rate. Neurons output information through an astonishing variety of axon morphologies. One of the processes that generates this diversity is axon branching. From reaching multiple targets to defining innervation fields within specific targets, axon branching allows neurons to establish unique patterns of connectivity. Changes in axon branching patterns are achieved through regulation of a variety of morphological processes, including branch initiation, branch elongation, the development of arbor complexity, branch retraction/pruning, and terminal arborization. One of the classes of proteins related to the regulation of axonal branching is cytoskeleton-associated proteins. Axonal degeneration, that is, the reduction in the level of axonal branches, is directly related to neurodegeneration. A molecule capable of preventing degeneration or even regenerating neurons susceptible to degeneration is very relevant for AD. Indirectly, this parameter may be related to the increase in synapses at a later stage. For the two analyses (number of branches and branches per cell), HDC neurons the concentration of 18.52 nM showed an interesting profile, with an increase in the number of branching around 80% and twice the number of branching per cell. In ADC neurons, this same concentration increases both the number of branching and the number of branching per cell, by approximately 50% and 60%, respectively, suggesting a protection of neurons from degeneration or increased axonal growth. (A) Number of branching from healthy patient cells (HDC); (B) Number of branching from AD patient cells (ADC); (C) Number of branching per cell from healthy patient cells (HDC); (D) Number of branching per cell from AD patient cells (ADC). As shown in FIG. 21A-D, branching junction is the indication for development of arbor complexity and potential synapses of neurons. It is a complementary parameter to branching number; thus, it refers to the potential of the axon of one neuron to communicate with the dendrite of another neuron. Thus, the greater the number of branching junctions, the greater the chance of communication and synapses. The most interesting profiles were for the treatments with 6.17 and 18.52 nM, for both test systems (HDC and ADC). Regarding the total branching junction. HDC treated with a concentration of 6.17 nM doubled this parameter. For ADC, it increased by approximately 3.5 times. (A) Branching junction from healthy patient cells (HDC); (B) Branching junction from AD patient cells (ADC); (C) Branching junction per cell from healthy patient cells (HDC); (D) Branching junction per cell from AD patient cells (ADC).

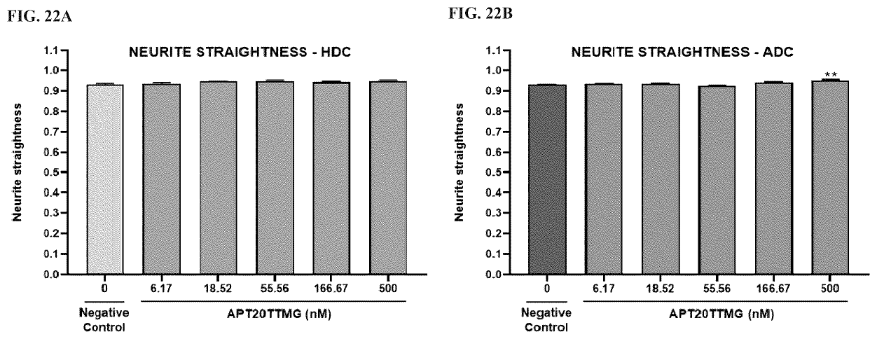

A neurite is any projection from the cell body of a neuron, a process of differentiation of neurons into dendrites and axons, and may be critical for the communication between different neurons. Thus, neurite straightness is a parameter for evaluating the quality of the neuron regarding the transport of molecules and the action potential within it. The straighter this region, the greater the chance of information passing along the neuron. FIG. 22A-B shows the neurite straightness for a different cells. There was a statistically significant increase (**p=0.0028) in ADC treated with the highest concentration (500 nM), when compared to the negative control (no treatment) in the same system. (A) Neurite straightness from healthy patient cells (HDC); (B) Neurite straightness from AD patient cells (ADC).

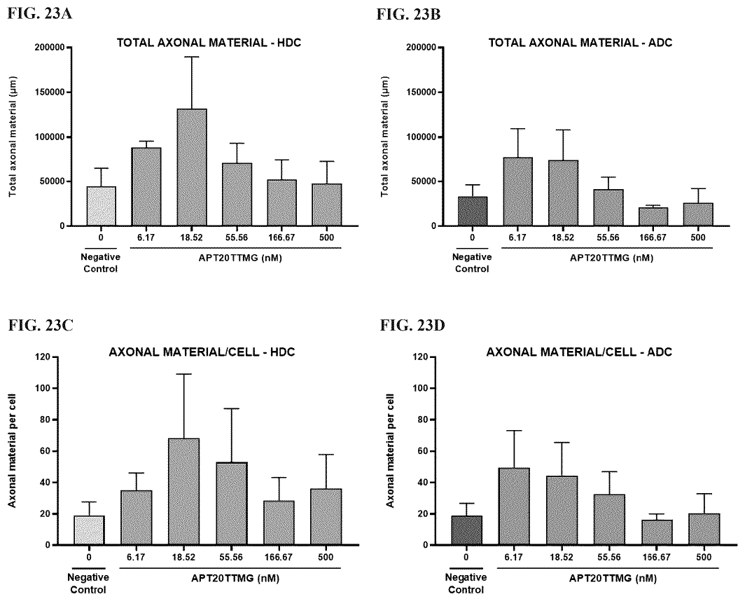

As shown in FIG. 23A-D, at concentrations of 6.17 and 18.52 nM, both total axonal material and axonal material per cell present an increased yield of global neuronal content. In the case of axonal material per cell (cells normalized with total number of cells). ADC treated with the lowest ASMO1 concentration increased this parameter by approximately 160% and with the concentration of 18.52 nM increased by 130%. In HDC cells, the treatment with the same concentrations increased by around 80% and 3.5 times the content of axonal material per cell, respectively. In addition to some concentrations having the potential to increase the axonal material, it is worth mentioning that there is a tendency for the parameter to improve in ADC treated with the first 3 concentrations. (A) Total axonal material from healthy patient cells (HDC); (B) Total axonal material from AD patient cells (ADC); (C) Axonal material per cell from healthy patient cells (HDC); (D) Axonal material per cell from AD patient cells (ADC).

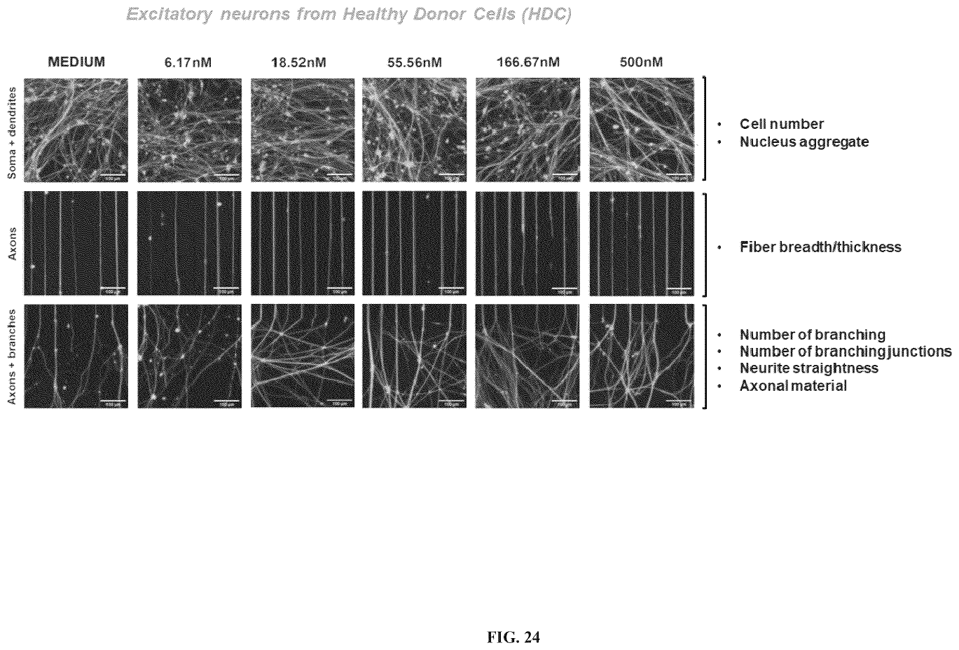

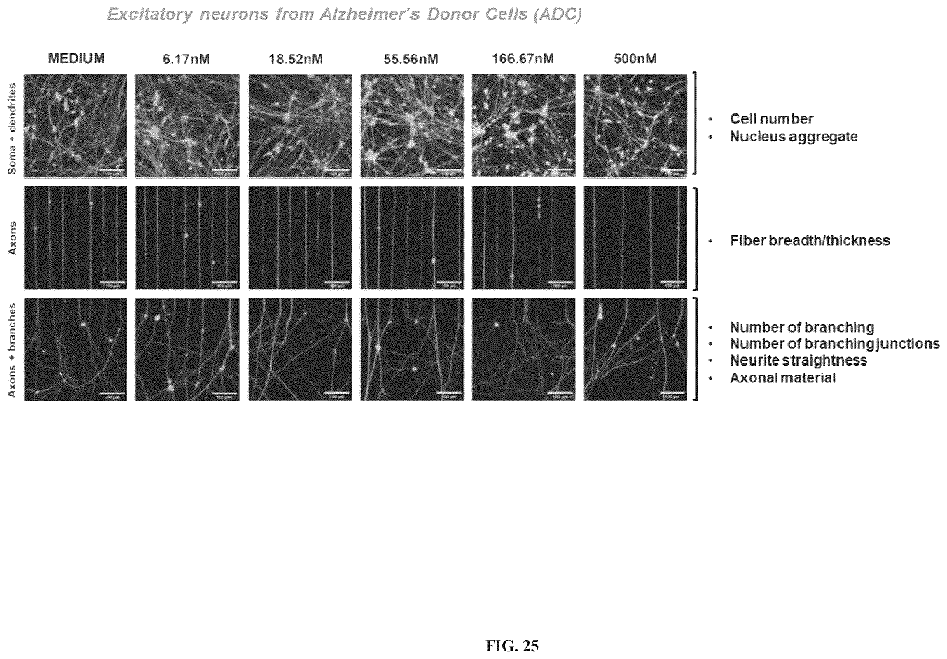

As shown in FIGS. 24-25, excitatory neurons were cultured for 6 days before incubation with ASMO1. After 7 days of treatment, all morphological changes were evaluated. Parameters evaluated: soma and dendrites (cell number and nuclear aggregate), axons (fiber breadth) and axons and dendrites (number of branching, number of branching junctions, neurite straightness, and axonal material).

Incubation with ASMO1 does not cause toxicity in HDC and ADC neurons, as demonstrated by the cell number and cell aggregate parameter. Some analyzed parameters show a tendency to increase after incubation with ASMO1, which seems to be favorable for preventing neurodegeneration and improving axonal functions, directly related to synapse and cognitive functions, such as fiber breadth, branching number, branching junctions, neurite straightness and total axon material. Thus, it is possible to affirm that ASMO1 has a beneficial potential for both exposure in healthy neurons and neurons with AD-related phenotypes, helping to prevent degeneration of vulnerable neurons in AD.

### Example 7. Evaluation of Complex U1 Proteins Binding by Immunoprecipitation in Neuroblastoma Cell Line after Incubation with ASMO-1

For the protein immunoprecipitation assay, cell lysate (300 μg per IP) from SK-H-SH was added to 0.5 mg streptavidin beads pre-conjugated with either 90 pmol biotinylated APT20TTMG or beads only (negative control) and incubated overnight at 4° C. Flow-through was collected and beads were washed ten times with ice-cold IP lysis buffer. Proteins were eluted from beads in 30 μl of 4× Laemmli Sample Buffer/2-mercaptoethanol for 5 minutes at 90° C. Samples (5 μg of input, 2.5% of flow-through and 50% of the eluate fraction) were separated on NuPAGE and electrophoresed. Resolved proteins were transferred to polyvinylidene difluoride (PVDF) membranes, blocked, and then incubated overnight at 4° C. with the following primary antibodies: U1-70K (Abcam. Cat. #ab83306), U1-A (Abcam. Cat. #ab166890), U1-C (Abcam, Cat. #ab192028), GAPDH (Abcam. Cat. #ab8245), followed by incubation with HRP-conjugated secondary antibody. Proteins were visualized with enhanced chemiluminescence (ECL) reagent (Thermo Fisher Scientific, USA) and images were captured using a Jess Simple Western Imaging suite. Evaluations were performed with three biological replicates (N=3).

For the RNA Immunoprecipitation using biotinylated APT20TTMG, cells were rinsed once with ice-cold PBS, collected and pelleted by centrifugation at 500 g for 5 minutes. Each sample was re-suspended and lysed in 150 μl ice-cold RSB-100 buffer (10 mM Tris-HCl PH 7.4, 100 mM NaCl, 2.5 mM MgCl2, 0.5% Triton X-100) containing complete Ultra EDTA-free protease inhibitor cocktail mini tablet (Roche) and 100 U/ml Super-ASE RNase inhibitor (Thermo Fisher Scientific) for 20 min at 4° C. To increase protein yields, each sample was homogenized using a 29 G×13 mm needle and syringe. Cell lysates were clarified by centrifugation at 17.000 g for 20 minutes at 4° C. Supernatants were collected and total protein concentration was determined using the BCA protein assay kit (Thermo Fisher Scientific) according to the manufacturer's instructions. Each sample was adjusted to 1.00 mg/ml using RSB-100 lysis buffer and a 100 μl aliquot was taken from each sample (input) and stored at −20° C. until required. For RNA manipulation, 0.5 mg Dynabeads M-270 streptavidin magnetic beads (Thermo Fisher Scientific) were washed and prepared, according to the manufacturer's instructions. Beads were conjugated with 90 pmol of biotinylated APT20TTDC or APT20TTMG (beads without oligo used as negative control) for 30 minutes at room temperature. After washing, 100 μl of SK-N-SH cell lysate (100 μg) was added to the beads immobilized with biotinylated oligo (and control) and incubated overnight at 4° C. with gentle rotation. RNA was isolated from input, flow-through and eluate samples using Direct-zol RNA Microprep (Cambridge Bioscience) according to the manufacturer's instructions. RNA (50% of input and flow-through and 100% of the eluate fraction) was converted to cDNA using the SuperScript IV First-Strand Synthesis System (Thermo Fisher) according to the manufacturer's instructions. After RNA removal, 1 μl of cDNA was amplified by qPCR using the following Taqman probes (Thermo Fisher); U1 (RNVU1-18, Hs04940459_gH). Total tau (MAPT. Assay ID: Hs00902193_ml). GAPDH (GAPDH. Assay ID: Hs99999905_ml). Gene expression was analyzed using the Applied Biosystems QuantStudio™ 12K Flex Real-Time PCR System.

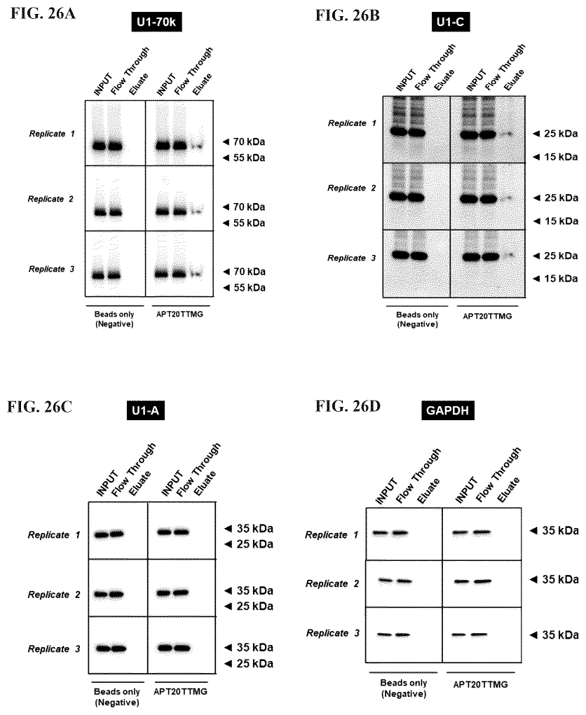

FIG. 26A-D shows a various western blots to detect the present of specific proteins in the samples. Cell lysate (100 μg per IP) from SK-H-SH was added to 0.5 mg streptavidin beads pre-conjugated with either 90 pmol biotinylated APT20TTMG, or beads only (negative control) and incubated overnight at 4° C. Samples (5 μg of input, 2.5% of flow-through and 50% of the eluate fraction) were evaluated and electrophoresed. Immunoprecipitation of proteins was assessed by Western Blotting using specific antibodies. GAPDH was used as a negative control (n=3 technical replicates).

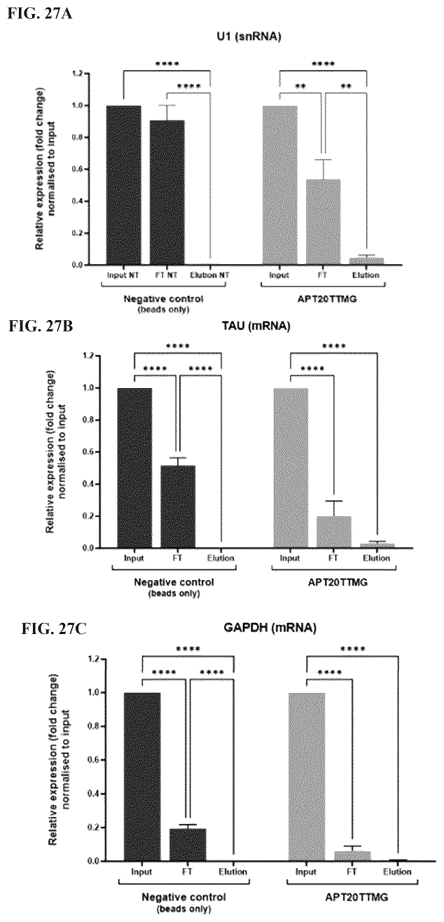

FIG. 27A-C shows relative expression of specific proteins in samples. Human neuroblastoma SK-N-SH cells were lysed and prepared for qRT-PCR immunoprecipitation assays, performed with RNA isolated from input (50%), flow-through-FT (50%), and eluate (100%) samples. Relative expression of (A) U1 snRNA, (B) Tau pre-mRNA, and (C) GAPDH. Data are represented as mean #standard error of the mean. Graphs show data from three independent experiments, analyzed using ANOVA followed by Tukey's post hoc test or t-test. **p<0.01, ****p<0.0001.

Immunoprecipitation assays were carried out using SK-N-SH neuroblastoma cells aiming to validate the APT20TTMG/U1-pre-mRNA complex assembly. From these assays, it was possible to confirm that APT20TTMG binds to key proteins of the U1 complex, such as U1-70K and U1-C proteins, but not to U1-A snRNP. The absence of binding to GAPDH protein was expected as this protein was used as a negative control of the experiment. The qRT-PCR confirmed the binding of APT20TTMG to U1 snRNA and to tau and glyceraldehyde 3-phosphate dehydrogenase (GAPDH) pre-mRNAs. This binding pattern indicates an interaction with a functional U1 snRNP complex, which also contains a set of proteins, named Sm core, not evaluated in this study. Furthermore, the binding of APT20TTMG to U1-70K also provides evidence for the probable Sm core assembly within the immunoprecipitated pool, as U1-70K is responsible for bridging pre-U1 (U1 snRNA precursor) and U1 snRNA to the SMN complex, which mediates Sm core assembly. The assembled Sm core plays an important role in the stable binding of the U1-70K to the U1 snRNA. Consequently, the binding of APT20TTMG to these proteins, as well as to pre-mRNAs and U1 snRNA, strongly suggests a correct assembly of the initial machinery of splicing, indicating a subsequent spliceosome recruitment. This is noteworthy as the U1 complex needs to be properly assembled for correct splicing and this process could be impaired by the typical U1 snRNP splicing dysfunction present in AD.

The evidence of direct or indirect interaction of ASMO1 with U1-C and U1-70K indicates that the expected target for ASMO is correct, that is. ASMO interact with the U1 complex and with pre-mRNAs in general, represented in these assays by the MAPT and GAPDH pre-mRNAs, enabling the correct U1-pre-mRNA complex assembly.

### Example 8. Assessment of Tau Protein Modulation after Incubation with ASMO1 in Neurons from Induced Pluripotent Stem Cells (iPSCs)

Along with the extracellular deposition of insoluble Amyloid-β that initiates before early-onset Alzheimer's disease (AD), early AD is characterized by an intracellular accumulation of Neurofibrillary Tangles (NFTs) composed of aggregated hyperphosphorylated Tau. Despite this pathological role of tau in AD, in physiological conditions, this protein is a phosphoprotein that promotes microtubule assembly and its stabilization in neurons. Tau also has roles in chromatin structure, signal transduction, synaptic plasticity, and nucleic acid protection. In AD, abnormal tau hyperphosphorylation hampers its ability to bind to microtubules, leading to aberrant tau self-assembly, accumulation, aggregation, and NFT formation in neurons. Pathogenic forms of tau are suggested yet to be involved in the disruption of axonal maintenance processes in AD, triggering the loss of neuronal connectivity, inhibiting axonal transport, and accumulating in neuropil threads.

Cells from healthy patient (HDC) and patient diagnosed with Alzheimer's disease (ADC) were cultured for 6 days, followed by addition of ASMO1 (6.17 nM, 18.52 nM, and 500 nM) or medium only for 7 days (day 13). After, cells were lysed and samples from each experimental group are evaluated using the commercial ab273617 Human Tau SimpleStep ELISA® Kit. For each biological replicate, quantification curves are generated, 3 independent experiments were performed in 3 technical replicates. Values were expressed as mean±standard error of the mean. To assess the statistical difference between the groups, the one-way ANOVA test is performed followed by Dunnett's multiple comparisons test. P values less than 0.05 are considered as indicative of significance. GraphPad Prism 9 software (GraphPad Software Inc., San Diego, CA, USA) was used for statistical analyses.

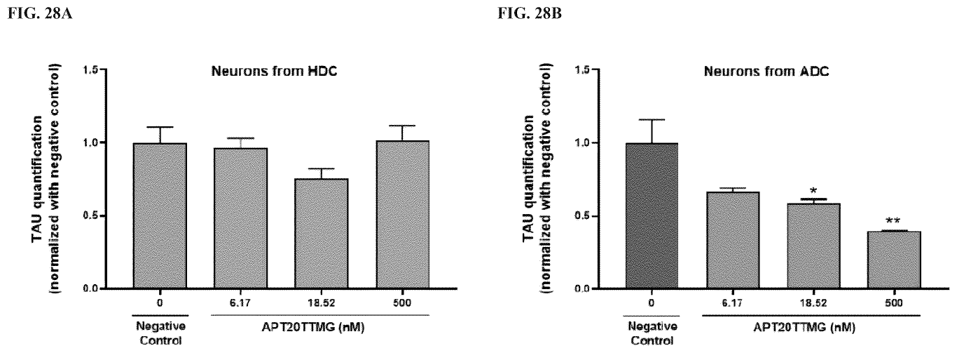

As shown in FIG. 28A-B, excitatory neurons from HDC (A) or ADC (B) were cultured for 6 days before treatment incubation with medium (negative control). After 7 days of treatment, tau levels were evaluated through commercial kit.

In neurons from healthy patient (HDC), there was no change in tau protein levels after incubation with the three concentrations of APT20TTMG. However, the same profile was not observed in neurons obtained from a patient diagnosed with AD (ADC). After incubation with the concentration of 18.52 nM and 500 nM of APT20TTMG, protein levels reduced by 41.4±2.9 (*p=0.0196) and 60.4±0.8% **p=0.0023), respectively, suggesting a concentration-dependent effect. Although the concentration of 6.17 nM also showed a reduction (33.7±2.7%), this result was not statistically different. It is noteworthy that the proposed mechanism of action of the APT20TTMG acts on pathways that precede the formation of neurofibrillary tangles, composed of tau protein aggregation, in addition to acting on mechanisms that precede the formation of amyloid-beta plaques, two of the main hallmarks of AD. Thus, through this study, it was quite clear that ASMO1 reduces the amount of tau protein in stages and times that precede its increase in cultured neurons. Since the levels are already lower in cells that mimic some of the pathophysiological characteristics of the disease, it can be concluded that APT20TTMG has the potential of acting in earlier stages of the disease, preventing the excessive formation of the tau protein.

ASMO1 reduces tau protein in a concentration-dependent manner only in neurons from patient diagnosed with AD and not in neurons from healthy patient, suggesting its potential to act in earlier stages of the disease and reduce its progression.

### Example 9. RNA-Seq Differential Gene Expression Analysis of Neurons from Induced Pluripotent Stem Cells (iPSCs) after Incubation with ASMO1

This study involved RNA Sequencing (RNA-seq) differential gene expression analysis of neurons from induced pluripotent stem cells (IPSCs) after incubation with APT20TTMG. Gene expression occurs when DNA is transcribed into RNA, mainly mRNA transcripts. The levels of mRNA transcripts are often used to infer protein abundance. This set of genes expressed by the genome is called the transcriptome and the measurement of RNA transcript levels could be accessed by RNA-Seq, which is an efficient methodology for sequencing the transcriptome using Next-Generation Sequencing technologies (NGS). Once transcript levels are quantified and normalized, it is possible the investigation of up or downregulated genes (differential expression is therefore measured) of a transcriptome in response to treatment with ASMO1. This differential expression analysis enables insights about the molecular mechanisms of action, theoretical safety assessment, and potential side effects underlying therapeutic intervention.

In this context, identifying genes that are influenced directly or indirectly by ASMO1 (APT20TTMG) could be vital to understanding its potential effect in target cells like neurons. To this end. RNA-seq of APT20TTMG treated and non-treated neurons derived from iPSCs from healthy patient and patient diagnosed with Alzheimer's disease followed by differential gene expression analysis was the chosen methodology to answer the desired biological questions. The purpose of this methodology was to identify the key differences in gene expression and associated pathways between neurons derived from iPSCs from healthy patient (HDC) and patient diagnosed with Alzheimer's disease (ADC) incubated with two concentrations of the ASMO1 (APT20TTMG) in relation to their non-treated controls. Neurons derived from IPSCs from healthy patient (HDC) and patient with Alzheimer's disease (ADC) were cultured for 6 days, followed by the addition of treatments (APT20TTMG-18.52 nM. APT20TTMG-500 nM, or medium only) for 7 days (day 13). It is important to note that this time point of treatment was chosen to allow the study of upstream events (before the APP and MAPT increase in ADC in relation HDC) that would lead to the development of the disease and, therefore, to evaluate the effects of APT20TTMG on the ADC cells prior to the disease phenotype establishment.

After RNA extraction, libraries were generated from 250 ng of total RNA as follows: mRNA enrichment was performed using the NEBNext Poly(A) Magnetic Isolation Module. cDNA synthesis was achieved with the NEBNext RNA First Strand Synthesis and NEBNext Ultra Directional RNA Second Strand Synthesis Modules. The remaining steps of library preparation were done using the NEBNext Ultra II DNA Library Prep Kit for Illumina. The libraries were normalized and pooled and then denatured in 0.02N NaOH and neutralized using HT1 buffer. The pool was loaded at 175 pM on a NovaSeq S4 lane using Xp protocol and a NovaSeq 6000 (Illumina) was then used to sequence all sample libraries. The run was performed for 2×100 cycles (paired-end mode). A phiX library was used as a control and mixed with libraries at 1% level. Base calling was performed with RTA v3. Program bcl2fastq2 v2.20 was then used to demultiplex samples and generate FASTQ reads. Reads were paired end with a read length of 101, aiming to achieve 100 million reads per sample. This experiment was performed with three replicates and only one repeat.

The quality of the raw reads (FASTQ files) was assessed with FASTQC v0.11.8 and combined with MultiQC. After examining the quality of the raw reads, trimming was not considered necessary. The reads were aligned to the human reference genome with STAR v2.7.6a. The GTF annotation file of the human reference genome used was downloaded from Ensembl (release 102). The raw counts were calculated with FeatureCounts v1.6.0. Differential expression analysis to determine differentially expressed genes (DEGs) was performed using the DESeq2 R package. In total, five main comparisons were performed each one having its own set of DEGs: (i) ADC treated with 18.52 nM of APT20TTMG in relation to non-treated ADC (medium only); (ii) ADC treated with 500 nM of APT20TTMG in relation to non-treated ADC (medium only); (iii) HDC treated with 18.52 nM of APT20TTMG in relation to non-treated HDC (medium only); (iv) HDC treated with 500 nM of APT20TTMG in relation to non-treated HDC (medium only); (v) Non-treated HDC (medium only) in relation to non-treated ADC (medium only). For graphical representation purposes, it was used the first threshold yielding less than 1000 DEGs in each comparison in order to generate both heatmaps and volcano plots (used thresholds are specified in the title or legend of these figures). DEGs heatmap was drawn based on the z-score of the normalized count. The volcano plot was based on the adjusted p-values (FDR) and shrunken log 2 fold change (Graph generated from DESeq2 results). Functional enrichment analysis of DEGs (gene ontology and pathway enrichment) was performed with the gprofiler2 R package, using a cutoff for term size of equal to or greater than 16 and equal to or lower than 2000.

For the network and enrichment analysis of metabolic pathways of DEGs in neurons from ADC treated with 18.52 nM APT20TTMG compared to non-treated neurons from ADC, the STRING app (v2.0.1) of Cytoscape (v3.11.1: The Cytoscape Consortium, USA) was applied using the functional enrichment option and focusing on the KEGG classification system. Further visual annotation with labels of the enrichment terms names and clustering of the enrichment network was performed with the Cytoscape AutoAnnotate app (v1.4.1). Clusters comprising more than six genes, and enriched pathways with the most significant (lowest) false discovery rates were prioritized for detailed evaluation. Maps of KEGG pathways with up and downregulated genes from the differential gene expression analyses were created using KEGG mapper website directly.

For the premature polyadenylation profile and size of DEGs associated with aging (U1-70K, SNRP200, MAPT, and APP), the FASTQ reads obtained from the RNA-Seq were further processed and analyzed using the Lasergene Genomics Suite (v. 17.5.0.48; DNASTAR, USA). The reads were imported into SeqMan NGen (v. 17.5.0.48; DNASTAR, USA) and aligned to the human reference genome (GRCh38 from NCBI) using the default parameters of the RNA-seq workflow. From the RNA-seq assemblies, sashimi plots were generated using the Gen Vision Pro (v. 17.5.0.48; DNASTAR, USA), enabling the quantification of splicing junctions. The read counts of these junctions were normalized and used to calculate the 3′ UTR/CDS ratios, providing insights into the premature polyadenylation profile of the selected genes. The CDS expression was calculated based on the average read densities at the last three junctions of the constitutive exons, while the 3′ UTR value was calculated based on the read density of the last junction.

For the assessment of transcripts associated with aging. DEGs of neurons from ADC treated with 18.52 nM APT20TTMG compared to non-treated neurons from ADC, obtained using DESeq2 in Array Star software (v. 17.5.0.48; DNASTAR, USA), were compared to genes associated with aging in the Human Aging Genomic Resources database. The intersected genes were evaluated regarding their expression level and sizes. For the statistical analysis, genes were stratified into four categories: very short (<10 kb), short (10-22 kb), long (22-110 kb), and very long (>110 kb), with only short and long categories being considered in the analysis.

Statistical analysis to determine DEGs was performed using the DEseq2 software. In this case, the null hypothesis for each gene is that there is no differential expression between the two compared groups (LFC==0). DESeq2 uses the Wald test as a statistical test to determine along with the p-value of the test if the null hypothesis is true based on the observed data. The Wald test P values from the subset of genes that pass an independent filtering step (significance cutoff by default of 0.1) are adjusted for multiple testing using the Benjamini-Hochberg procedure, generating the adjusted p-values.

In the case of the enrichment analysis, the statistical analysis was performed by the gprofiler2 software itself. This software performs a functional enrichment analysis on the input DEG list for each comparison and maps the genes to known functional information sources and detects statistically significantly enriched biological processes, pathways, regulatory motifs, and protein complexes. The statistical test used for gprofiler2 to determine over-representation terms is the hypergeometric test to measure the significance of functional terms in the input gene list. To reduce the amount of false positive findings, gprofiler2 and STRING app perform multiple testing correction, the False Discovery Rate in this study case, and report only the adjusted enrichment P-values. For the statistical analysis premature polyadenylation profile (3′ UTR/CDS ratios) of the genes (U1-70K, SNRP200, MAPT, and APP of neurons from ADC treated with 18.52 nM APT20TTMG compared to non-treated neurons, a t-test was performed.

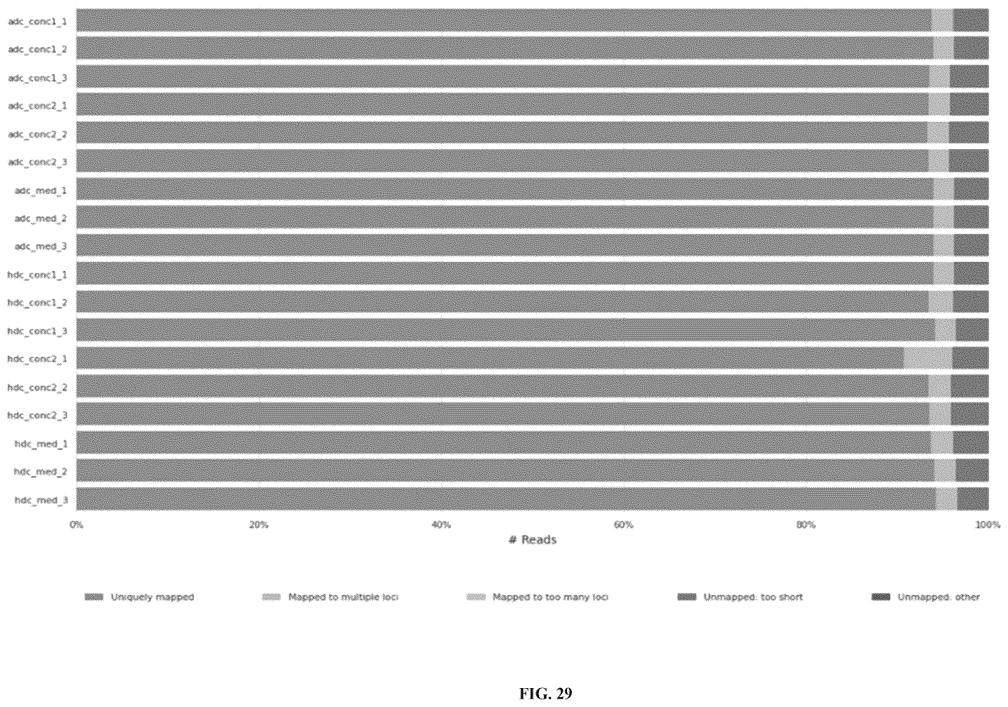

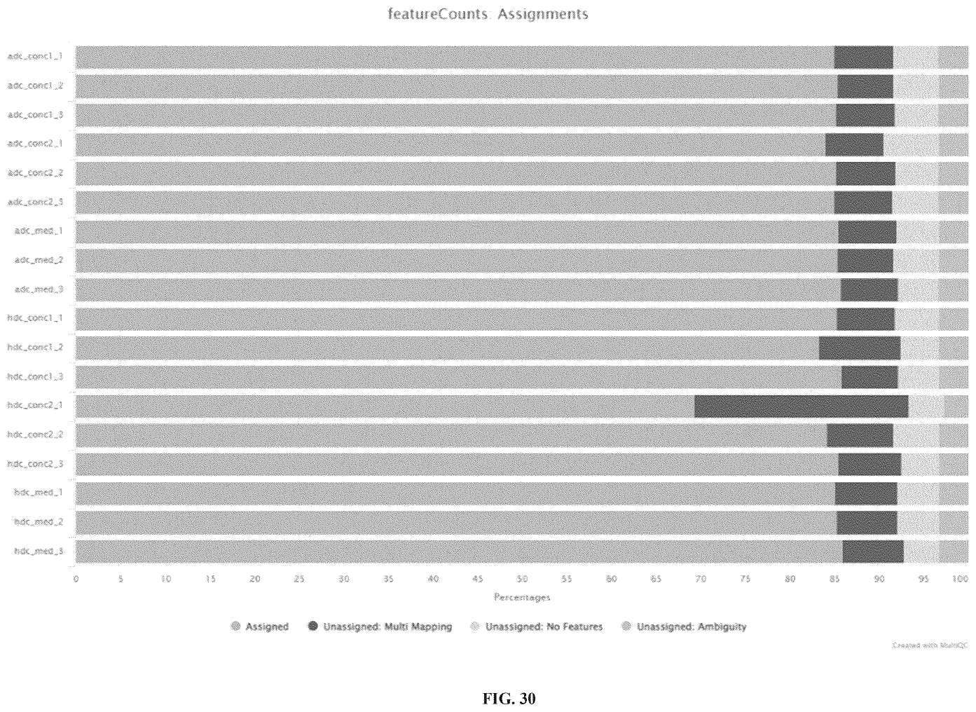

As shown in FIG. 29, the first step for a differential expression analysis is the mapping of reads on a reference genome and enables the identification of what genes are being expressed. As a result of mapping, less than 10% of unmapped reads were observed and an average of 94% of reads uniquely map to a single location in the human reference genome. As the uniquely mapped mean value should be higher than 80%, the mapping metrics indicate a high-quality result. As shown in FIG. 30, all samples, except for HDC cells treated with 500 nM ASMO1 (replicate 1—hdc_conc2_1), have more than 80% of reads successfully assigned to a genomic feature and less than 10% of reads not assigned to any genomic features.

To identify quantitative changes in gene expression levels between groups and the statistical significance of the observed changes, differential expression analysis with DESEQ2 was performed from the normalized read count data. Table 6 shows the number of genes differentially expressed in each possible pairwise comparison considering the adjusted p-value (cutoff of 0.05) and log 2 fold change. As can be observed, the comparison of ADC and HDC non-treated controls has one of the highest numbers of DE genes representing 10251, 1107 with log 2FoldChange of 2, and 179 with log 2FoldChange of 4. In both comparisons of ADC treated with 18.52 nM and 500 nM ASMO1 in relation to non-treated ADC (medium only) around 600 genes are differentially expressed, and a slightly lower number is observed in HDC treated-controls comparisons. All comparisons have a log 2FoldChange of less than 2. Comparisons of ADC treated conditions in relation to HDC controls are not explored since the obtained DE genes are influenced both by treatment and by the intrinsic differences in ADC and HDC cells themselves, as could be observed in the controls. Therefore, the focus is to determine what is the differential expression caused by treatment with ASMO1. Table 6 shows highlighted DE thresholds and the amount of data used for the generation of plotted data and enrichment analysis.

TABLE 6
Number of differentially expressed genes in each pairwise comparison
considering the adjusted p-value (cutoff of 0.05) and log2 fold change.
In bold and italics are the DE thresholds used for analysis.

| ADC concentration 1 vs | | | HDC concentration 1 vs | | | HDC medium vs ADC | | |
|---|---|---|---|---|---|---|---|---|
| ADC medium | | | HDC medium | | | medium | | |
| | padj < | padj < | | padj < | padj < | | padj < | padj < |
| LFC | 0.05 | 0.01 | LFC | 0.05 | 0.01 | LFC | 0.05 | 0.01 |
| all | 602 | 332 | all | 344 | 160 | all | 10251 | 8424 |
| FC > 2 | 0 | 0 | FC > 2 | 0 | 0 | FC > 2 | 1107 | 1107 |
| FC > 4 | 0 | 0 | FC > 4 | 0 | 0 | FC > 4 | 179 | 179 |
| FC > 8 | 0 | 0 | FC > 8 | 0 | 0 | FC > 8 | 53 | 53 |

| ADC concentration 2 vs | | | HDC concentration 2 vs | | |
|---|---|---|---|---|---|
| ADC medium | | | HDC medium | | |
| | padj < | padj < | | padj < | padj < |
| LFC | 0.05 | 0.01 | LFC | 0.05 | 0.01 |
| all | 600 | 349 | all | 544 | 307 |
| FC > 2 | 0 | 0 | FC > 2 | 0 | 0 |
| FC > 4 | 0 | 0 | FC > 4 | 0 | 0 |
| FC > 8 | 0 | 0 | FC > 8 | 0 | 0 |

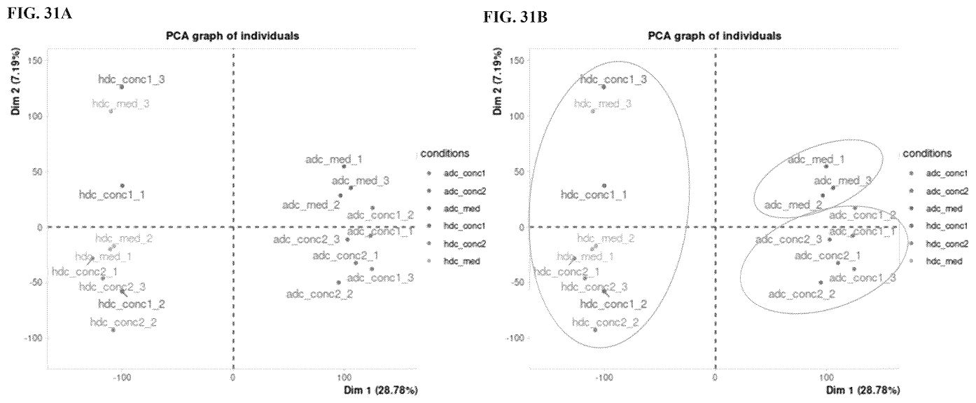

To visualize the relatedness of the individual samples, principal components analysis (PCA) plots were generated, enabling the reduction and visualization of the highly dimensional generated gene expression data (Log 2-transformed normalized counts). As shown in FIG. 31A, the two first components of PCA are plotted and explain almost 35% of the variation in the DE gene data and appear to have three main clusters (FIG. 31B). The first cluster involves the HDC non-treated controls and the HDC treated conditions, which are similar despite the ASMO1 treatment. The second cluster represents just the ADC non-treated control samples and the third cluster comprises only the ADC conditions treated with ASMO1. These results could indicate that the treatment with ASMO1 does not change HDC cells enough to distinguish the treated conditions from their controls, which could be a good sign for the safety profile of ASMO1. There is observed however a distinct separation of treated ADC cells from their ADC controls. In addition, it is also noticeable that HDC and ADC conditions occupy distinct positions in the plot, which could be due to intrinsic variation in HDC and ADC non-treated controls themselves as is shown in Table 6.

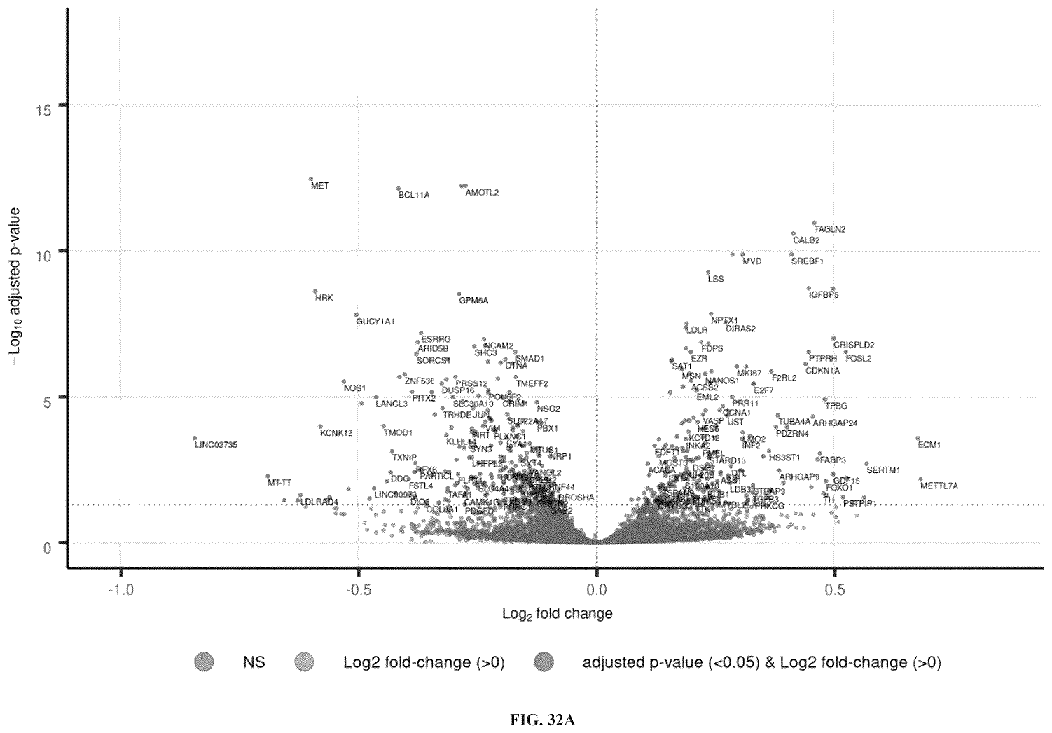

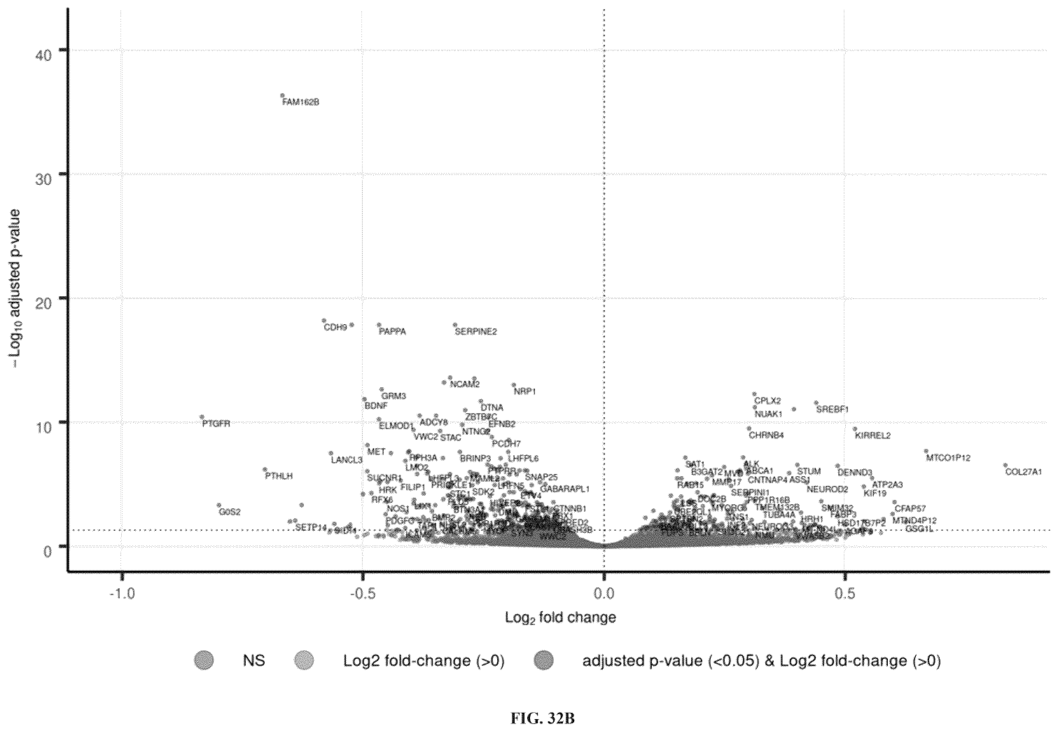

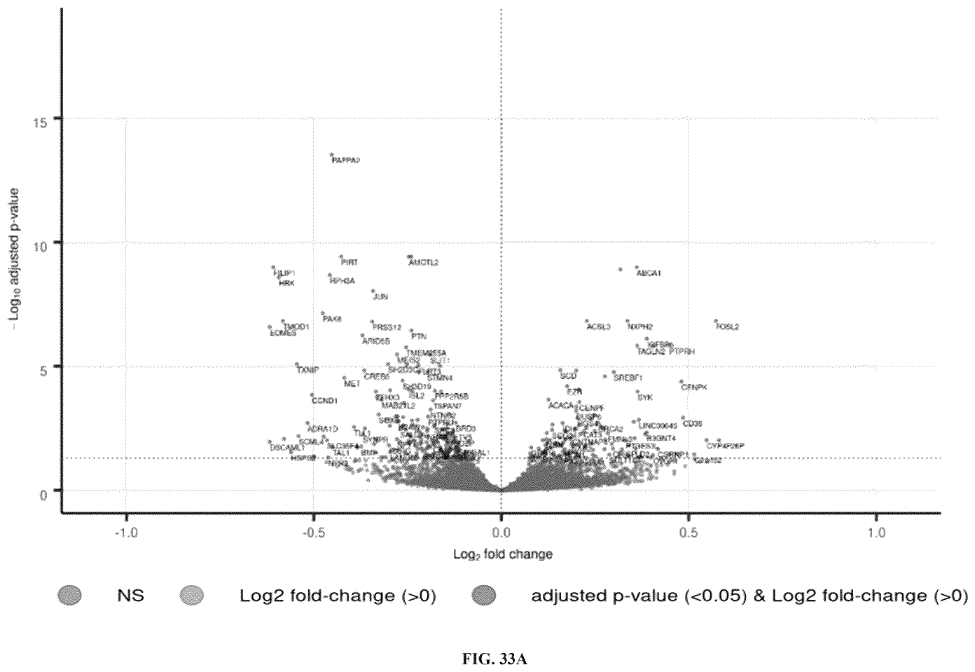

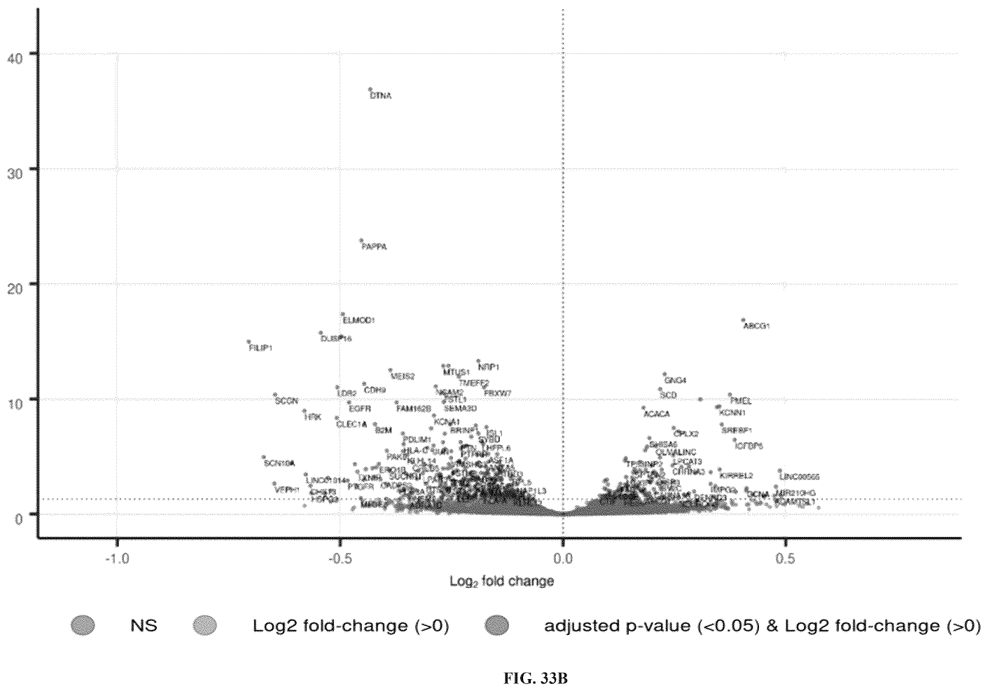

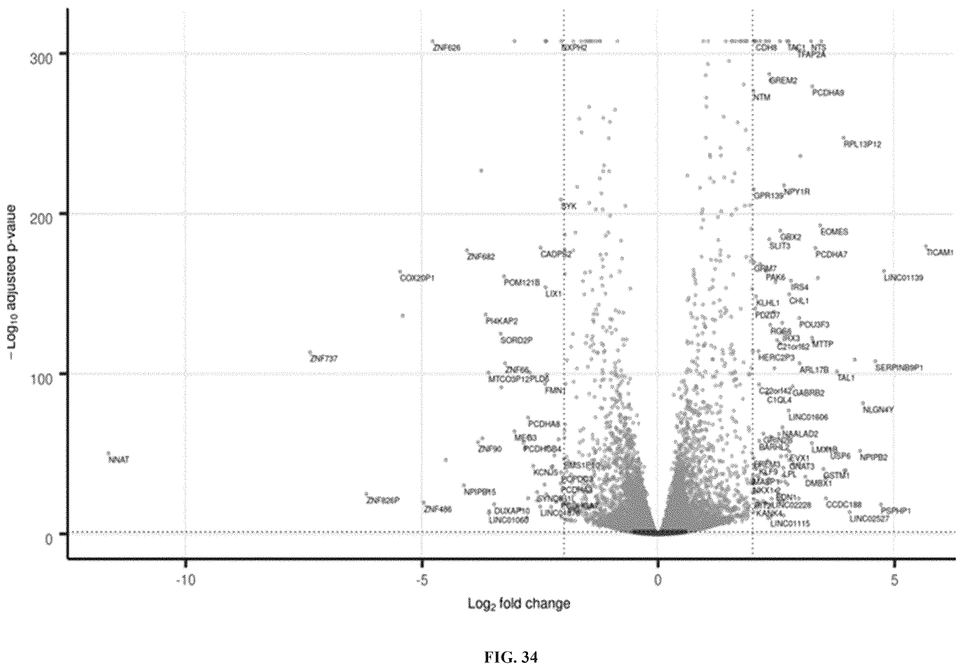

To determine what the main DE genes of each comparison are, volcano plots were generated depicting fold change (X axis) versus adj p-value (Y axis) as shown in FIGS. 32-34. Genes present in the upper left and upper right sectors have particularly low adjusted p-values and large fold changes, but the genes shown in red exhibit the greatest fold changes. The gray and blue dots indicate genes that have non-significant p-values.

Regarding the hallmark genes of Alzheimer's Disease (APP and MAPT) and the main ASMO1 targets (U1-70K and U1-C), none of these are differentially expressed in the ASMO1 treated conditions in relation to their non-treated controls as shown in Table 7. Regarding the non-treated controls comparison, MAPT and APP were upregulated in HDC. These results were partially expected since APP and MAPT proteins are slightly increased in HDC at this chosen time point (less than two weeks from differentiation-data reported by the ANANDA CRO executing the study), although no statistical difference was observed in relation to ADC. Therefore, as expected for a potentially homeostatic drug, no alteration caused by APT20TTMG was observed in APP, MAPT and target snRNPs at this stage and at mRNA level in treated neurons.

TABLE 7
Differential expression results of hallmark genes of Alzheimer's Disease
(APP and MAPT) and the main ASMO1 targets (U1-70K, U1-C, and U1-A).

| | HDC medium × | | | | ADC concentration 1 vs | | | |
|---|---|---|---|---|---|---|---|---|
| Gene | ADC medium | | | | ADC medium | | | |
| name | LFC | pvalue | padj | Sig. | LFC | pvalue | padj | Sig. |
| MAPT | 0.222 | 0.000 | 0.000 | *** | −0.048 | 0.164 | 0.615 | ns |
| SNRNP70 | −0.075 | 0.364 | 0.552 | ns | 0.073 | 0.380 | 0.810 | ns |
| SNRPC | 0.038 | 0.436 | 0.621 | ns | 0.027 | 0.577 | 0.899 | ns |
| SNRPA | −0.108 | 0.044 | 0.109 | ns | 0.000 | 0.999 | 1.000 | ns |
| APP | 0.133 | 0.000 | 0.000 | *** | −0.046 | 0.069 | 0.436 | ns |
| | HDC concentration 1 vs | | | | HDC concentration 2 vs | | | |
| Gene | HDC medium | | | | HDC medium | | | |
| name | LFC | pvalue | padj | Sig. | LFC | pvalue | padj | Sig. |
| MAPT | −0.045 | 0.194 | 0.605 | ns | 0.018 | 0.611 | 0.875 | ns |
| SNRNP70 | 0.065 | 0.435 | 0.793 | ns | 0.058 | 0.482 | 0.813 | ns |
| SNRPC | −0.014 | 0.767 | 0.934 | ns | −0.021 | 0.668 | 0.900 | ns |
| SNRPA | −0.053 | 0.324 | 0.724 | ns | −0.033 | 0.529 | 0.838 | ns |
| APP | −0.038 | 0.132 | 0.527 | ns | −0.044 | 0.079 | 0.413 | ns |

| | ADC concentration 2 vs | | | |
|---|---|---|---|---|
| Gene | ADC medium | | | |
| name | LFC | pvalue | padj | Sig. |
| MAPT | −0.037 | 0.281 | 0.680 | ns |
| SNRNP70 | 0.129 | 0.120 | 0.497 | ns |
| SNRPC | 0.041 | 0.397 | 0.767 | ns |
| SNRPA | −0.079 | 0.143 | 0.532 | ns |
| APP | −0.039 | 0.121 | 0.499 | ns |

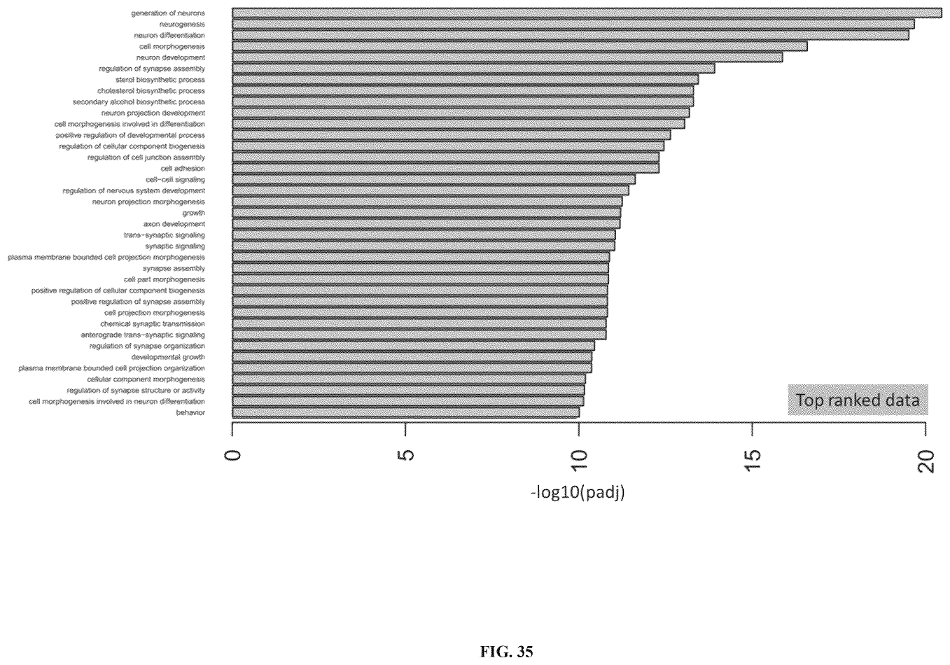

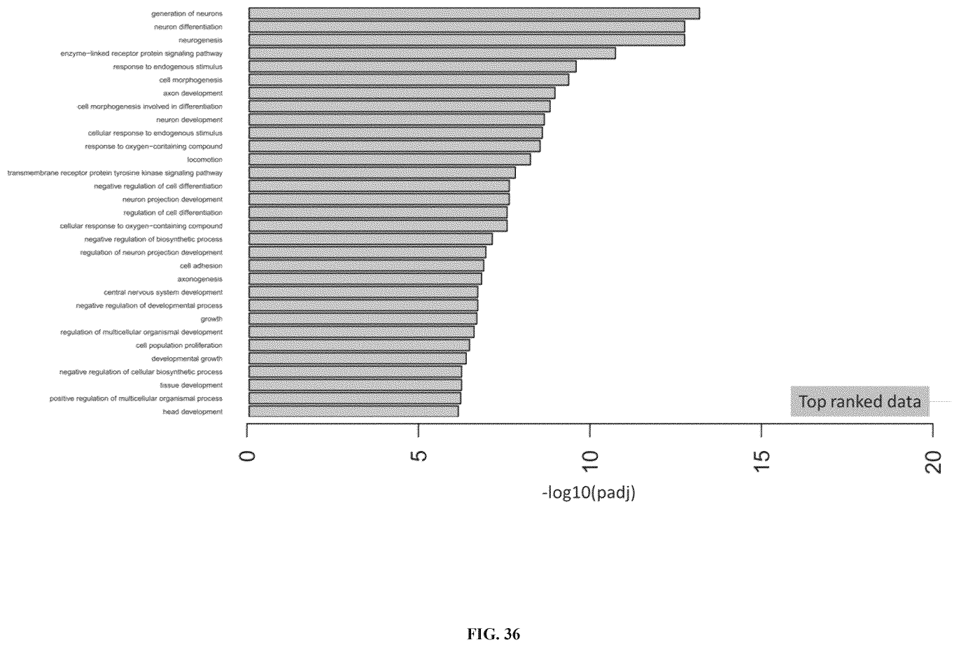

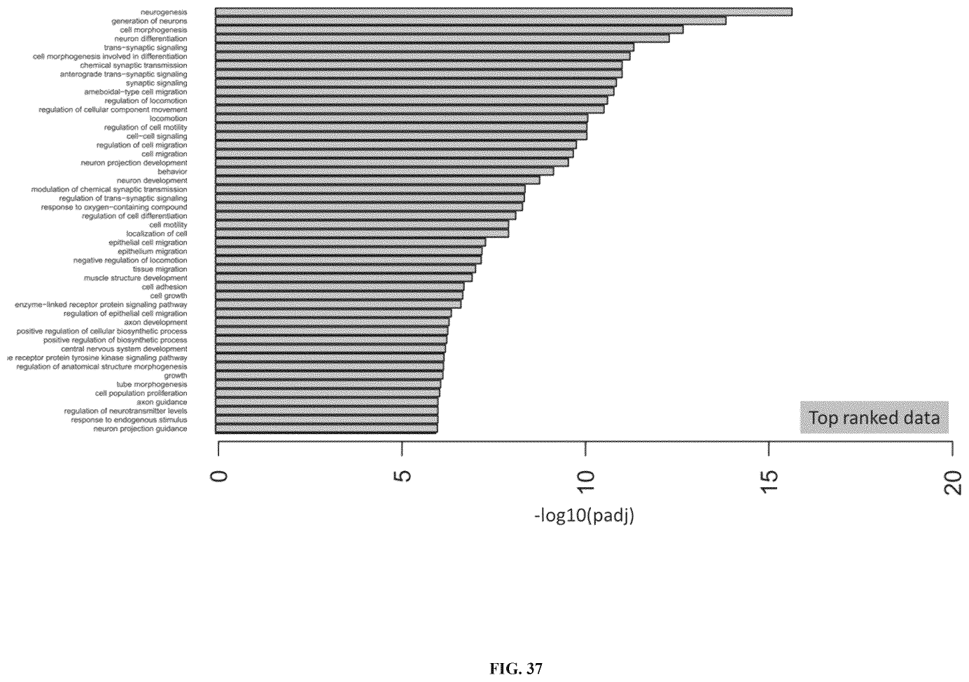

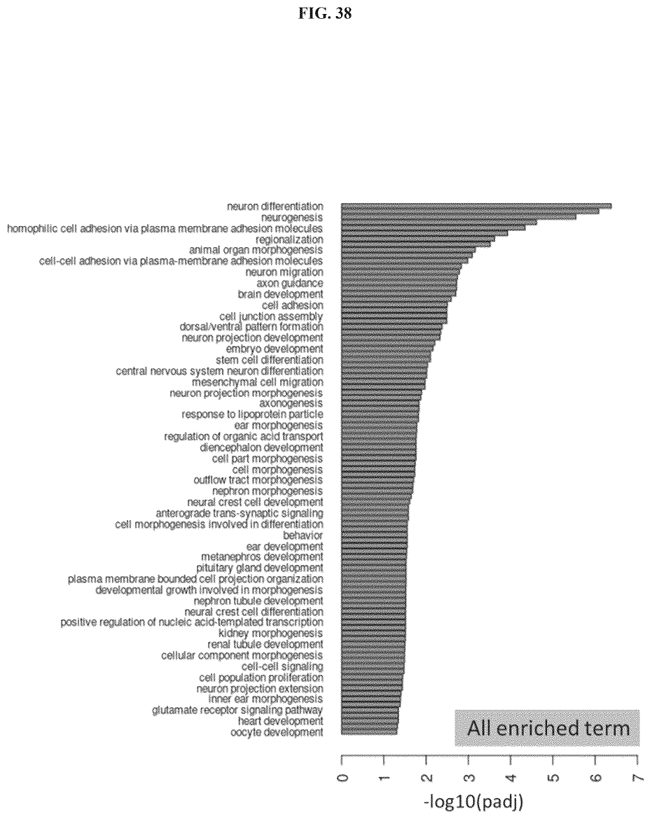

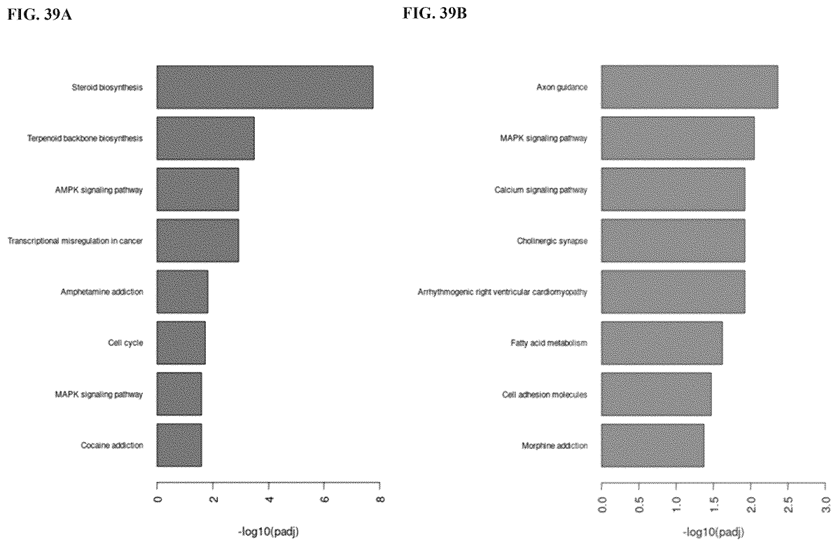

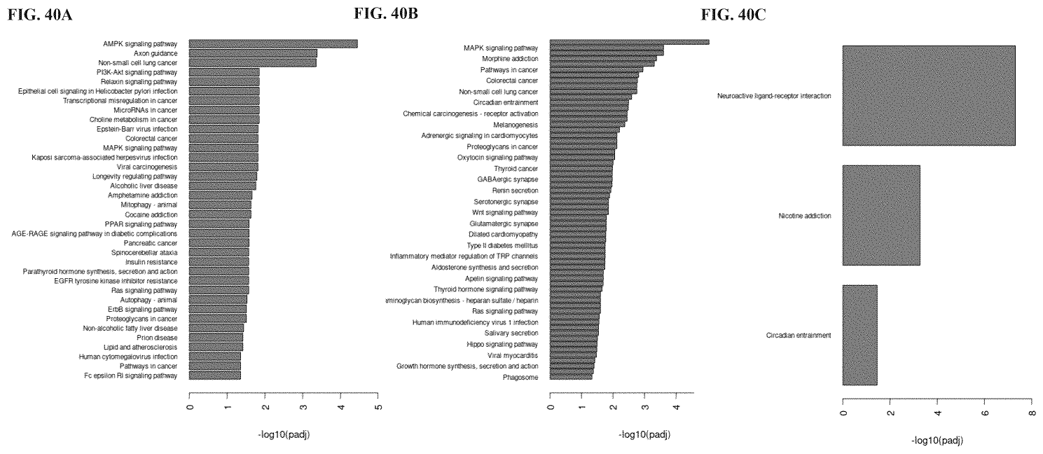

Enrichment analysis is a robust approach to interpreting differential gene expression data based on the functional annotation of the observed DEG. More than that, enrichment analysis allows discovering if the identified DEGs are associated with a certain biological process or molecular function and if these processes/functions are statistically over-represented in our DEGs data set. FIGS. 35-38 show the top-ranked terms regarding biological processes (GO: BP), molecular functions from Gene Ontology (GO: MF), and FIG. 39 and FIG. 40 the top-ranked terms related to KEGG (Kyoto Encyclopedia of Genes and Genomes) terms for each comparison. Regarding biological processes, all comparisons of ADC and HDC treated with APT20TTMG in relation to their respective controls were associated with terms like generation of neurons, neurogenesis, neuron differentiation, cell morphogenesis, and regulation of synapsis assembly. Although this result represents all up and downregulated genes together, it could be considered a good indicator of the APT20TTMG benefits as it agrees with the observed tendency of increasement of the 7 factors metrics under treatment with APT20TTMG. Moreover, molecular functions, such as cell adhesion molecule binding and calcium ion binding, were significantly enriched in more than one pairwise comparison.

Among the comparisons of the treated conditions in relation to their controls, pathways like steroid biosynthesis. AMPK signaling. MAPK signaling are worth noting, relating to lower concentration treatment of APT20TTMG. For the comparison involving the HDC and ADC controls, the neuroactive ligand-receptor interaction pathway (that consists of a group of neuroreceptor genes, such as dopamine receptor and acetylcholine receptor, and is involved in environmental information processing and signaling molecules and interaction) was the one of the only enriched pathway.

Overall, these results may suggest that APT20TTMG treatment triggers in both ADC and HDC key signaling pathways related to neuron generation and calcium binding-calcium is associated with an early signal that initiates the formation of the nervous system metabolism. In addition, the enrichment of DEGs related to steroid biosynthesis is of interest since recent studies show that dysregulation of steroid endogenous concentrations and their biosynthetic enzymes play a role in neurological diseases, including AD, with a general trend of lower levels of neuroactive steroids in different brain regions of AD patient than non-demented controls. In the same way, enrichment related to MAPK is also interesting since the dysregulation of the MAPK signaling pathway has been largely associated with Alzheimer's disease (AD), and similarly there is also literature that associates AMPK signaling.

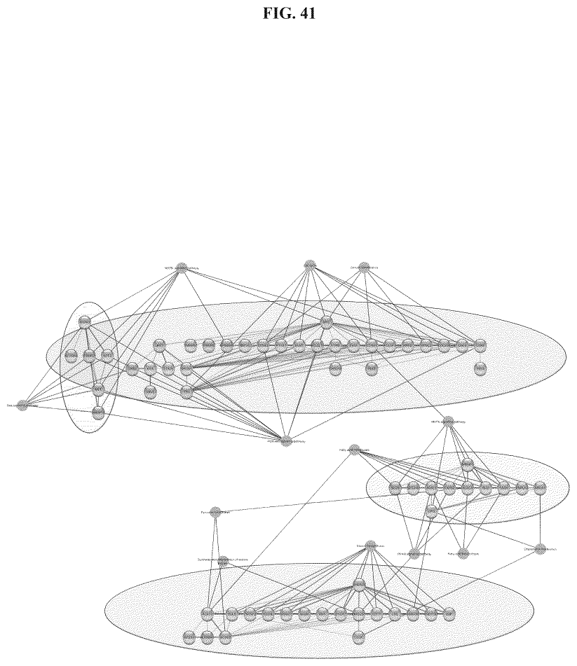

Metabolic pathway enrichment and networks analysis were carried out from the RNA-seq DEGs of neurons from ADC, treated with 18.52 nM APT20TTMG, using the Cytoscape software (v3.11.1; The Cytoscape Consortium, USA) and StringAPP (v2.0.1), based on Kyoto Encyclopedia of Genes and Genomes (KEGG) terms. The AutoAnnotate App (v1.4.1) further assisted in the annotation and clustering of these networks. This analysis identified four prominent clusters, each containing more than six DEGs. (FIG. 41).

FIG. 41 shows enrichment analysis of metabolic pathways of DEGs in neurons from ADC treated with 18.52 nM APT20TTMG using STRING and Auto Annotate apps from Cytoscape. Inside each cluster, the nodes represent protein products of the investigated DEGs and the edges denote protein-protein interactions. The blue nodes represent the main enriched metabolic pathways related to the protein clusters. Blue genes were upregulated, while red genes were downregulated a Cluster 1: cell cycle (FDR value 2.95E-7, 12 DEGs), PI3K-Akt signaling (FDR value 3.43E-6, 17 DEGs), and cellular senescence (FDR value 5.65E-5, 10 DEGs) pathways; b Cluster 2: steroid biosynthesis (FDR value 3.64E-11, 10 DEGs), synthesis and degradation of ketone bodies (FDR value 0.0171, 2 DEGs), and pyruvate metabolism (FDR value 0.018, 3 DEGs) pathways; c Cluster 3: fatty acid metabolism (FDR value 3.7E-5, 7 DEGs) and biosynthesis (FDR value 0.004, 3 DEGs), cholesterol metabolism (FDR value 0.0063, 4 DEGs), and AMPK signaling pathway (FDR value 3.71E-9, 14 DEGs); d Cluster 4: MAPK (FDR value 7.45E-9, 19 DEGs) and Ras (FDR value 5.54E-5, 12 DEGs) signaling pathways.

The first cluster containing more genes was associated with the cell cycle (FDR value 2.95E-7, 12 DEGs). PI3K-Akt signaling (FDR value 3.43E-6, 17 DEGs), and cellular senescence (FDR value 5.65E-5, 10 DEGs) pathways. The PI3K-Akt signaling pathway, pivotal in modulating several pathways such as apoptosis, protein synthesis, and cell cycle through phosphorylation, gains importance in AD due to its role in neuronal survival and its interplay with Aβ toxicity. In this pathway, upon treatment with APT20TTMG, although PI3K and AKT alterations are not detected, an upregulation of BRCA1 is observed, indicating an increase in DNA repair mechanism along with a reduction in apoptosis through the downregulation of pro-apoptotic genes (BCL2L11, FOXO3), potentially benefiting neurons. Furthermore, the downregulation of FOXO3 could have an impact on reducing Aβ(1-42) levels, as active FOXO3 was shown to increase the activity of APP promoter and neurotoxic Aβ processing. The upregulation of SYK, a critical regulator of microglial responses in neurodegenerative diseases, could also have a positive impact on Aβ clearance, as its deletion leads to elevated levels of Aβ deposition, aggravated neuropathology, and cognitive impairment in a mouse model of AD. While decreased MET and PDGFD levels might limit aberrant neuronal growth signals and, in the case of PDGFD, cause a minor promotion of inflammation under pathological conditions, MET's reduction could exacerbate synaptic pathology in AD. In contrast, the upregulation of NTF3 and NTRK1, downregulated in AD, could help in promoting neuronal survival and differentiation, while the downregulation of JAKI might reduce neuroinflammatory responses. The regulation of PI3K-Akt signaling key genes seems favorable in reducing AD pathophysiology, potentially reducing apoptosis, and neuroinflammation and promoting neuronal health. Similarly, the cell cycle was characterized by alteration of key genes, such as the downregulation of MYC (involved in the cell cycle progression) and upregulation of CDKN1A/p21 (a potent cyclin-dependent kinase inhibitor) and GADD45A (a mediator of growth arrest). It could induce cell cycle arrest, a protective response to prevent neurons from cell cycle reentry and the consequent neuron death. While upregulated genes such as CCNA1/CycA, CCNE2/CycE, and CDK6 indicate a complex cell cycle interplay, they are known to be inhibited by the upregulated CDK inhibitor CDKN1A/p21 in the cell cycle pathway. These observations are particularly important as the cell cycle, altered in early AD, is suggested to be associated with dysfunctional neuronal cell cycle reentry and resulting apoptotic cell death as neurons are not able to complete cell division. Additionally, to the expected cell cycle arrest, the MYC downregulation due to APT20TTMG treatment could have a positive impact on neurons from ADC, as studies have seen an increased immunoreactivity to c-MYC in dystrophic neurites and neurons with NFTs in AD, and neurons and glial cells bearing abnormal tau deposits. The activation of the cellular senescence pathway is intrinsically related to the cell cycle arrest, as the downregulation of FOXO3 and MYC, combined with the upregulation of CDKN1A and GADD45A that are also involved in this pathway, might drive cells towards a non-proliferative state, reversing cell cycle reentry and safeguarding neurons against apoptosis.

The steroid biosynthesis (FDR value 3.64E-11, 10 DEGs), synthesis and degradation of ketone bodies (FDR value 0.0171, 2 DEGs), and pyruvate metabolism (FDR value 0.018, 3 DEGs) pathways were enriched in the second cluster. One of the key genes involved in the steroid biosynthesis pathway, DHCR24, which encodes an enzyme that catalyzes the reduction of the delta-24 double bond of sterol intermediates, was upregulated. This upregulation also affected other genes involved in this pathway and that are typically downregulated in AD, such as LSS and TM7SF2. These three genes are not only related to AD pathology but could also mitigate some of its hallmarks. Both genes related to ketone bodies. HMGCS1 and ACAT2, were upregulated. Ketone bodies, produced by the liver from fatty acids during periods of low food intake, carbohydrate restriction, or prolonged exercise, play a crucial role in resistance to oxidative stress and inflammation. HMGCS1 is involved in the synthesis of a key intermediate in the production of ketone bodies, called 3-hydroxy-3-methylglutaryl-CoA, through the condensation of acetyl-CoA and acetoacetyl-CoA. ACAT2 converts these two previous molecules and is the first enzyme in the cholesterol synthesis pathway. Their upregulation is a great finding, as ketogenic diets have shown beneficial effects for AD patients by restoring metabolic dysfunction. While some studies suggest that inhibition of ACAT1 decreases AD-related damage, blocking ACAT2 does not change AD. Besides this inconclusive data for ACAT2, increased HMGCS1 levels restore the major component of ketone bodies (β-hydroxy butyrate). Along with ACAT2, the ACSS2 gene was also upregulated in the pyruvate metabolism pathway. ACSS2 is involved in the synthesis of acetyl COA and is required for brain histone acetylation. Its expression is reduced in both AD animals and patients, however, its upregulation improved the cognitive function of animals. The third cluster encompasses DEGs predominantly associated with fatty acid metabolism (FDR value 3.7E-5, 7 DEGs) and biosynthesis (FDR value 0.004, 3 DEGs), cholesterol metabolism (FDR value 0.0063, 4 DEGs), and AMPK signaling pathway (FDR value 3.71E-9, 14 DEGs). Within this cluster, genes such as ACSL3, FASN, ACACA, SCD, FADS2, SREBF1, SEC24D, PNPLA3, ABCA1, and LIPG, were upregulated, while SCD5 was downregulated. This suggests an increase in fatty acid synthesis, particularly of polyunsaturated fatty acids, leading to a lipidogenic environment. The AMPK pathway is upstream of the fatty acid biosynthesis pathway and holds a central role in regulating glucose and lipid metabolism, having implications for AD. While no alterations were observed in the AMPK gene itself, the activation of fatty acid synthesis within this pathway is evident, marked by the upregulation of FASN, SREBF1, ACACA, and SCD, with an important CD36 upregulation. CD36 encodes a scavenger receptor that enables tissue uptake of long-chain fatty acids and contributes to Aβ clearance by phagocytosis in both astrocytes and microglia. This gene downregulates as AD progresses, leading microglial cells to adopt a pro-inflammatory phenotype. The peroxisome proliferator-activated receptor (PPAR) pathway, also enriched (FDR value 9.2E-4, 6 DEGs), is intrinsically linked to the regulation of fatty acids metabolism, particularly their catabolismowever, none of the key PPARs (PPARalpha, PPARbeta/delta, PPARgamma) exhibited alterations, indicating that APT20TTMG treatment just triggers the activation of fatty acid synthesis. This is remarkable as the CNS holds the second-highest lipid concentration in the body and evidence suggests that adjusting membrane lipid levels may reverse AD-related pathogenic events. Furthermore, a systematic review has highlighted a significant reduction of 27.2% in total fatty acids in individuals with AD compared to controls, with most randomized trials involving fatty acid supplementation reporting positive outcomes. Regarding cholesterol metabolism, the upregulation of ABCA1 and LDLR seems to have a positive impact on AD. Recent studies indicate that induction of ABCA1 activity, using the agonist CS-6253, potentially promotes Aβ clearance and ApoE lipidation in cynomolgus monkeys' brains. Furthermore, upregulating LDLR in tauopathy mice has been shown to decrease brain ApoE, tau pathology, and neurodegeneration. It also modulates the glial response in AD mouse models, altering Aβ deposition. The upregulation of LIPG, an endothelial lipase involved in lipid hydrolysis, has not been directly correlated with AD yet. However, it is noteworthy that individuals with elevated plasma LIPG levels have a higher risk of cognitive impairment. Nonetheless, in general, the potential enhancement of fatty acid biosynthesis combined with the regulation of cholesterol metabolism, which might aid in Aβ clearance and ApoE lipidation, suggests a modulation of key AD hallmarks upon treatment with APT20TTMG.

The fourth cluster consisted of DEGs associated with the MAPK (FDR value 7.45E-9, 19 DEGs) and Ras (FDR value 5.54E-5, 12 DEGs) signaling pathways. The MAPK signaling pathway, involved in apoptosis, cell differentiation, growth, migration, and proliferation, is a highly conserved pathway activated by various extracellular signals. During AD development, this pathway becomes overactive, leading to increased Aβ deposition, neuroinflammation, and neuronal degeneration. Several MAPK-related genes, such as CASP3 and MYC, were downregulated due to APT20TTMG treatment. The proteins encoded by these genes are elevated in AD and are associated with Aβ burden and increased neuronal death. In contrast, some of the upregulated genes, such as GADD45A, NTF3, PRKCG, and TGFB3, are linked to the expression of the anti-apoptotic protein BCL-2, reducing apoptosis induced by DNA damage, Aβ production, and neuroinflammation. The decreased activation of the MAPK signaling would be related to the observed normalized mitochondrial activity, glutamate levels, and electrical activity of iPSC-derived neurons treated with APT20TTMG. This hypothesis is supported by DEGs related to the Ras signaling pathway. Ras proteins play a crucial role in the MAPK pathway as they activate several protein kinases to propagate signal transduction, and its overactivation also contributes to neurodegeneration. In addition to the NTF3 and PRKCG genes of the MAPK pathway, the HTR7 gene, which is decreased in AD, was upregulated, while AD-associated genes, such as ANGPT2, GAB2, INSR, MET, PDGFD, and SHC3, which are upregulated in AD and increase the risk of developing the disease, were downregulated. The involvement of metabolic pathways in AD has been the focus of several studies, with metabolic components being highly dysregulated in the disease. Our data clearly show that APT20TTMG specifically modulates several AD-related genes.

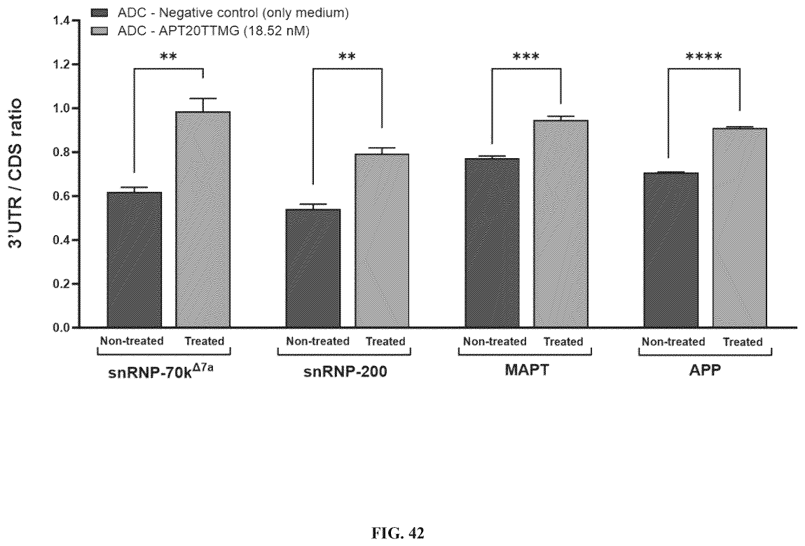

The analysis of 3′ UTR/CDS ratios for U1-70K, SNRP200, MAPT, and APP genes suggests that APT20TTMG treatment may also prevent premature polyadenylation events in neurons from ADC (FIG. 42). We observed a significant increase in the 3′ UTR/CDS ratio of these genes to levels resembling healthy neurons in most cases. These findings imply higher 3″ UTR expression in treated neurons from both HDC and ADC in comparison to non-treated neurons from ADC. It means that these neurons present less premature polyadenylation, which reduces shorter mRNAs with truncated 3′ UTRs. This effect is probably associated with the restoration of U1 snRNP function and the presence of U1-A-free molecules that are capable of suppressing premature cleavage and polyadenylation on cryptic poly(A) sites. It is particularly relevant as RNA-seq studies of AD brains have shown an increased prevalence of poly(A)-containing reads in the 5′ end of transcripts compared to healthy controls, indicating enhanced premature cleavage and polyadenylation in AD. Overall. APT20TTMG appears to mitigate this premature polyadenylation process associated with U1 snRNP dysfunction observed in AD.

As shown in FIG. 42, RNA was extracted from iPSC-derived neurons cultured for 6 days and incubated with 1 concentration of APT20TTMG (18.52 nM), for 7 days, using the RNeasy Micro Kit. Libraries were generated from 250 ng of total RNA and sequenced. The FASTQ reads, paired with a read length of 101 to achieve 100 million reads per sample, were aligned to the human reference genome. Reads counts of splicing junctions, quantified from sashimi plots generated from the RNA-seq assemblies, were normalized and used to calculate the 3′ UTR/CDS ratios of the U1-70K, SNRP200, MAPT, and APP genes. The CDS expression was calculated based on the average read densities at the last three junctions of the constitutive exons, while the 3′ UTR value was calculated based on the read density of the last junction.

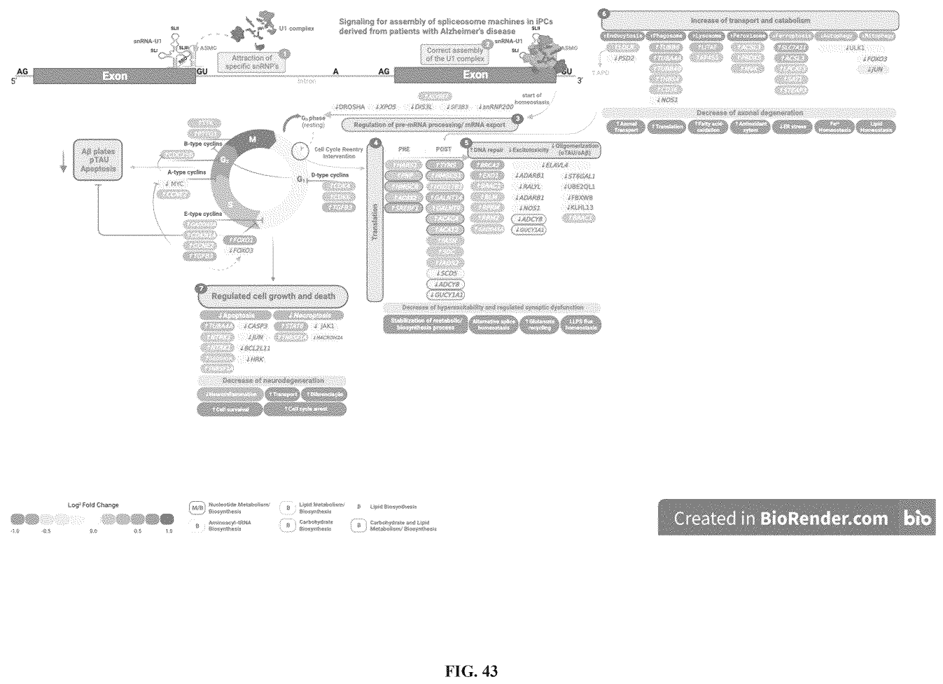

FIG. 43 shows the DEGs observed in ADC treated with 18.52 nM APT20TTMG and their relationship with AD and proposed main roles in the proposed mechanism of action of APT20TTMG. The first step (item 1 of FIG. 43) of the APT20TTMG mechanism of action is the attraction of specific U1 snRNPs and the recognition of the splicing donor site of pre-mRNA that is where APT20TTMG binds to start the correct assembly of the U1 complex (item 2). In this way. APT20TTMG is proposed to allow for a proper distancing of the U1 protein complex leading to a homeostasis of splicing and gene expression at a proper level of up or downregulation. Consequently, a favorable regulation of genes related to pre-mRNA processing and mature mRNA exportation (item 3) was observed like XPO5, SF3B3, and snRNP200 genes, which probably helps in controlling all the LLPS process (Liquid-liquid phase separation process, involved in the nucleus-cytoplasmic flux). It is important to note that deregulation of LLPS, common in AD, with leakage of snRNPs into the cytoplasm, can trigger a cell cycle reentry in neurons, which are post-mitotic and generally non-divisible. That CCR in neurons causes mitotic catastrophe and neuron death. Therefore, the regulation of these genes by APT20TTMG leads to cell cycle arrest in neurons, decreasing apoptotic and neurodegeneration processes. Besides, the observed upregulation of genes related to cyclins inhibition also prevents the cell cycle progression. APT20TTMG also interfered with genes related to pre- and post-translational events (item 4), some of them also involved in nucleotide metabolism, lipid metabolism, and carbohydrate biosynthesis. Upregulation of genes related to DNA repair (as BRCA 1 and 2) and decreased expression of genes related to stress granules aggregation and formation are also observed (item 5). As an outcome, this will likely avoid the formation of Aβ and tau oligomers. In general, AD neurons are hyperexcited neurons with an increase in synapsis due to alternative splicing errors causing the overproduction of glutamate receptors. ELAVL4 and ADARB1 are downregulated and potentially involved with the reduction of excitotoxicity. ELAVL4 regulates RNA splicing, glutamate levels, and neuronal excitability and binds to the glutamatergic receptor GRM5 and other genes within the long-term potentiation (LTP) pathway. Besides co-localized with tau protein and the stress granule markers TIA1 and G3BP1. ADARB1 is also involved with glutamate receptor subunit B being responsible for pre-mRNA editing of the site-specific deamination of adenosines. APT20TTMG also increases the transport and catabolism of proteins (item 6), therefore, there is a regulation of genes related to the activation of clearance pathways, such as endocytosis, phagocytosis, and formation of lysosome and peroxisomes. No activation of autophagy related genes was observed, but instead an increase of genes related to the pre-autophagic clearance process, with a decrease of stress avoiding the need for the autophagy process. For example, the previously described upregulated LDLR gene is an APOE endocytic receptor, helping in the Aβ clearance. Of note APOE-Aβ interaction controls Aβ aggregation and clearance and therefore directly affects the development of amyloid plaques, playing a key role in AD pathogenesis. Ferroptosis, an iron-dependent type of cell death related to lipid peroxidation, has been proposed to happen in AD. APT20TTMG regulates iron and lipid homeostasis genes, which can lead to Ferroptosis homeostasis avoiding oxidative stress. An expected consequence of this overall regulation is a decrease in axonal degeneration. Finally, cell growth and cell death regulation are expected since genes related to a decrease in apoptosis and necroptosis were observed. Furthermore, it is anticipated upon APT20TTMG treatment an increase in intracellular transport, arrest of the cell cycle, an increase of differentiation, neuron survival, and a decrease in neuroinflammation.

The present study involved a high-quality RNA-seq of neurons (ADC and HDC) from IPSCs after incubation with the APT20TTMG that allowed a robust differential gene expression analysis to investigate the expected therapeutic effects and safety profile of APT20TTMG. The results corroborate our previous studies that indicate the potential safety of the APT20TTMG for healthy cells, with fewer genes being differentially expressed HDC in relation to ADC, while causing a sufficient effect in ADC conditions to enable their distinction in relation to their ADC non-treated controls. Besides that the DEGs genes are associated with the generation of neurons, neurogenesis, neuron differentiation, and regulation of synapsis assembly in agreement with the observed tendency of increasement of the 7 morphological factors in neurons under APT20TTMG treatment. Furthermore, a favorable enrichment of DEGs related to pathways like fatty acids and steroid biosynthesis and MAPK signaling indicates a favorable potential of APT20TTMG in preventing AD-related phenotypes in neurons. In general, as far as we analyzed the RNA-seq results, we observed a DE pattern in response to APT20TTMG treatment that indicates a potential protective outcome in AD treated cells.

### Example 11. Evaluation of Firing Rate of Neurons Through Electrophysiology after Incubation with ASMO1 in Neurons from Induced Pluripotent Stem Cells (iPSCs)

As demonstrated herein, morphological improvement was associated with incubation with ASMO1 in HDC and ADC neurons, and that these observed changes (increase in axonal material, axonal fiber thickness, number of branches, and branching junctions) are directly related to the improvement of passage of signals between neurons and increase in synapses. Additional studies were performed to verify whether the changes caused by APT20TTMG would reflect in the increase in signals evaluated by the electrophysiology technique in neurons from healthy patient diagnosed with AD.

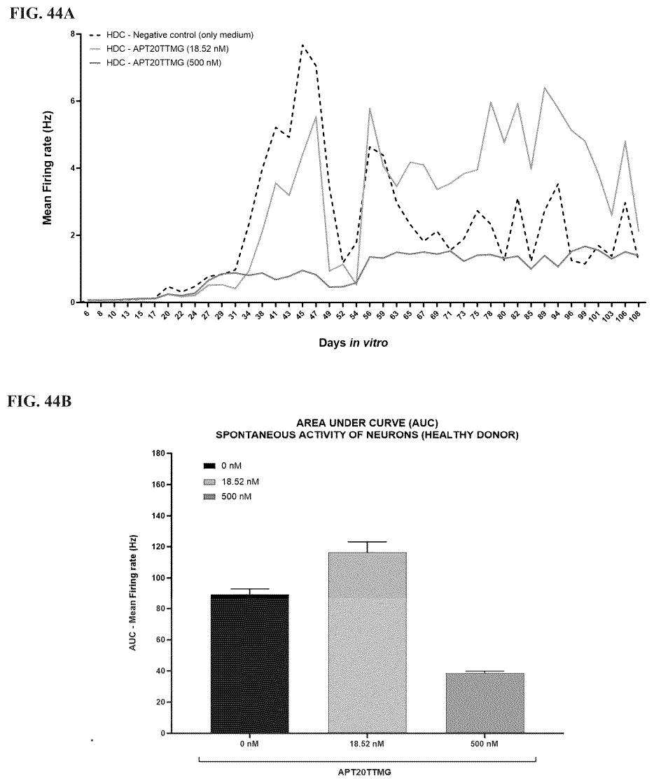

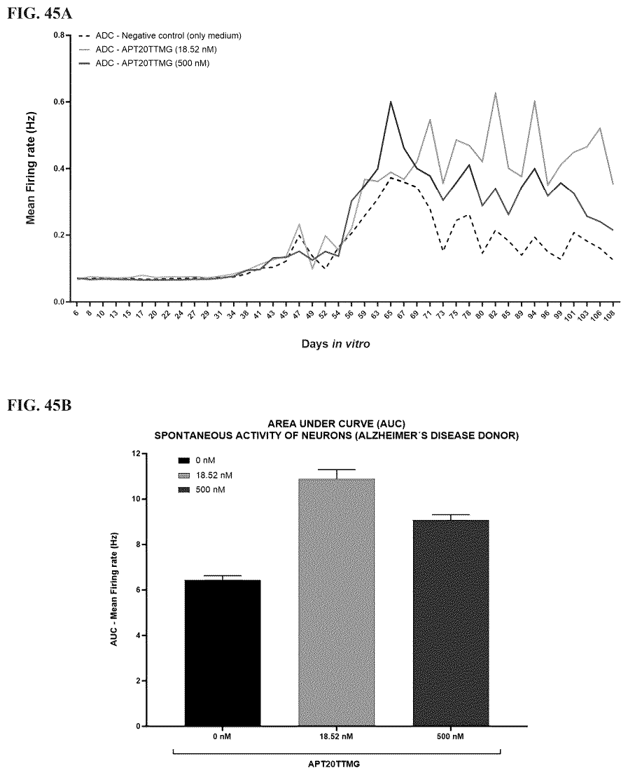

Excitatory neurons were cultured for 6 days before incubation with APT20TTMG (18.52 and 500 nM) or medium (negative control) in CytoView MEA 48 (Axion Biosystems) plate. Cells were treated at different time points (3 times per week), 4 hours before measurements. The total recording time was 5 min, after 4 h of treatment. Spontaneous firing measurements were performed 2 min before and 2 min after stimulation. The parameters were adopted for stimulation: Total time: 1 min; breakdown of stimulation: 12 times a 5-second stimulation repeat (12×5 s=1 min); each repeat is 36400 us of 0.5 V stimulation followed by 4.9636 s interval (0.036400 s+4.9636 s=5 s). Evaluated days: 6, 8, 10, 13, 15, 17, 20, 22, 24, 27, 29, 31, 34, 38, 41, 43, 45, 47, 49, 52, 54, 56, 59, 63, 65, 67, 69, 71, 73, 75, 78, 80, 82, 85, 89, 94, 96, 99, 101, 103, 106, and 108. The mean firing rate was computed as the number of spikes observed during the 5-minute period and data are plotted as mean firing rate (Hz)—average of 6 technical replicates. The electrical activity of neurons from HDC was detected after a few days, while for neurons from ADC was detected after 40 days and was lower than the mean firing rate observed in HDC (FIG. 44A and FIG. 45A). Following treatment with APT20TTMG in a low concentration (18.52 nM), the electrical activity of neurons from both HDC and ADC tended to improve, with an increase in approximately 23.9% and 69.1%, respectively, as could be observed in the AUC values (FIG. 44B and FIG. 45B). Nonetheless, the highest concentration (500 nM) reduced AUC values by 56.8% in neurons from HDC, while it seems to improve electrical activity in neurons from ADC by 40.8%. These results indicate that HDC and ADC neurons respond differently to different concentrations of APT20TTMG, but, overall, concentrations lower than 500 nM are not toxic to long-term neuronal cultures (over 100 days in vitro) of iPSC-derived healthy and diseased neurons, potentially improving the neuronal effects related to the increase/improvement synapses.

As shown in FIG. 44A-B, excitatory iPSC-derived neurons from HDC cultured for 6 days were treated with two concentrations of APT20TTMG, at different time points (3 times per week), with measurements 4 hours before treatment and a total recording time of 5 minutes. The spontaneous firing was performed 2 minutes before and after stimulation, and the spontaneous activity was recorded from days 6-108. (A) Mean firing rate; and (B) Area Under Curve. Data are expressed as mean firing rate (Hz). Graphs show data from one biological replicate in six technical replicates.

As shown in FIG. 45A-B, excitatory iPSC-derived neurons from ADC cultured for 6 days were treated with two concentrations of APT20TTMG, at different time points (3 times per week), with measurements 4 hours before treatment and a total recording time of 5 minutes. The spontaneous firing was performed 2 minutes before and after stimulation, and the spontaneous activity was recorded from days 6-108. (A) Mean firing rate; and (B) Area Under Curve. Data are expressed as mean firing rate (Hz). Graphs show data from one biological replicate in six technical replicates.

In conclusion, ASMO1 concentration of 18.52 nM tended to improve neuronal activity, both in HDC and in ADC cells, which mimic pathophysiological features of AD.

### Example 12. Evaluation of Biochemical and Molecular Effects after ASMO1 Treatment in an Animal Model of Senescence (SAMP-8)

The Senescence Accelerated Mouse-Prone 8 (SAMP8) model is a naturally occurring mouse line that reveals a phenotype for accelerated aging. The SAMP mice, including SAMP8, have been used in aging research to study phenotypes such as immune dysfunction, osteoporosis, and brain atrophy. The mice have normal early development with no evidence of growth retardation but develop a progressive, age-associated decline in brain function. Age-associated behavioral impairments including learning and memory difficulties, emotional disorders, such as reduced anxiety-like behavior and depressive behavior and altered circadian rhythms of spontaneous motor activity and water consumption. The neuropathology includes neuroinflammation. Aβ accumulation, and tau hyperphosphorylation.

Female SAMP8/TaHsd mice at an age of 7-8 weeks were purchased from Envigo and were allocated to two groups with n=11-13 animals each. At an age of ˜20 weeks, animals received either APT20TTMG, at 1 dose (0.3 μg/day), or vehicle (artificial cerebrospinal fluid, aCSF) injected via intracerebroventricular (icv) using Alzet® Mini-Osmotic Pump. Additionally, to standard analgesia (Buprenorphine). Carprofen was administered s.c. before surgery, as well as after surgery for 2 days. For the continuous infusion, the Alzet® Mini-Osmotic Pumpmodel 2006 (volume 200 μl, flowrate 0.15 μl/hour) was used, allowing an infusion for 42 days. Health status was monitored three times per day for three days, followed by health monitoring once per day for additional 11 days. Body weight of all animals was assessed on a weekly basis (7 times: baseline, week 1-6). In vivo mandibular blood sampling was performed two times, once at baseline before treatment start and once 3 weeks after treatment start. In vivo blood samples were processed to plasma (2 aliquots) and stored at −80° C. until further analysis. All mice were terminally anesthetized by i.p. injection of Pentobarbital (600 mg/kg, dosing 10 μl/g body weight). After, CSF sampling, terminal blood sampling and plasma preparation were performed.

Brains were transcardially perfused with ice-cold phosphate buffered saline (PBS) and the skull was opened and afterwards the brain was removed carefully and hemisected on a cooled surface. The left hemibrain was further dissected on a cooled surface into cortex, hippocampus, and remaining brain. All brain parts were weighed, snapped frozen on dry ice and stored at −80° C. The right hemibrain was fixed by immersion in freshly prepared 4% paraformaldehyde/PB (pH=7.4) for 48 hours at 4° C. Right hemibrains of all animals were processed and embedded in cryomolds for further immunohistochemical analysis.

Left cortex and hippocampus samples were further analyzed for levels of total tau, p-tau (T231), Aβ40, Aβ42, and GFAP. Furthermore, plasma samples were analyzed for cytokines. Soluble and insoluble brain fractions of cortex and hippocampus samples of n=8 animals per treatment groups were analyzed. Total tau as well as phosphorylated tau at residue Thr231 were measured in the soluble and insoluble fractions from all hippocampal and cortical samples by an immunosorbent assay (Meso Scale Discovery) according to the instructions of the manufacturer and assessed on the MESO QuickPlex SQ 120. Data were evaluated in comparison to calibration curves provided in the kit and were expressed as pg per μg total protein for total tau or arbitrary units (AU) for ptau231. Both soluble and insoluble fractions of cortex and hippocampus (of n=8 animals per group) were analyzed by immunosorbent assays from Mesoscale Discovery (V-PLEX Aβ Peptide Panel (4G8) Kit) according to the instructions of the manufacturer. Aβ levels in study samples were evaluated in comparison to calibration curves provided in the kit and are expressed as pg per μg total protein. GFAP was measured in the soluble fractions from all hippocampus and cortex samples by an immunosorbent assay (Meso Scale Discovery, MSD, Cat #K1511MR) according to the instructions of the manufacturer and assessed on the MESO QuickPlex SQ 120 (SOP NEQU162). GFAP levels were evaluated in comparison to calibration curves provided in the kit and are expressed as pg per μg total protein. Four cytokines (TNF-α, IFN-γ, IL-1β, IL-6) were measured in both in vivo and terminal plasma samples of n=8 animals per treatment group by a customized 4-plex inflammation marker panel kit from Meso Scale Discovery (MSD) according to the instructions of the manufacturer and assessed on the MESO QuickPlex SQ 120 (SOP NEQU162). Cytokine levels were evaluated in comparison to calibration curves provided in the kit and are expressed as pg per ml plasma.

Following fixation by immersion in freshly prepared ice-cold 4% paraformaldehyde in phosphate buffer (PB; pH 7.4) for 48 hours at 4° C. right hemibrains of all animals (n=37 hemibrains in total) were transferred to 15% sucrose/PBS for 24 hours at 4° C. to ensure cryoprotection. Tissue blocks were then trimmed as needed, transferred to cryomolds, embedded in OCT medium, frozen in dry ice-cooled isopentane and stored in an ultra deep freezer (set at −80° C.).

For histology/immunohistochemistry analysis, right hemibrain tissues of all animals were prepared, sectioned and immunofluorescence was evaluated with beta-Amyloid (4G8). GFAP, pSer202/Thr205-tau, and U1-70K. For each incubation a uniform systematic random set of five sections per mouse was selected (one section each from levels 2, 4, 6, 8, 10). All sections were counterstained with the nuclear dye DAPI. Binding of primary antibodies was visualized using highly cross-absorbed secondary antibodies. Sections were incubated with the following primary antibodies: (i) Mouse anti-beta-Amyloid (17-24) antibody (clone 4G8; Biozym Scientific GmbH), 1:5000; (ii) Rabbit anti-glial fibrillary acidic protein (GFAP) polyclonal antibody (DAKO. Z0334), 1:2000; (iii) Mouse anti-phosphorylated tau (pSer202/Thr205), PHF-tau, [AT8], Thermo Fisher Scientific. MN1020, 1:300; (iv) Rabbit anti-U1-70K polyclonal antibody (Sigma-Aldrich, cat #06-1297), 1:100. Following that the secondary antibodies used on the sections were: (i) donkey anti-mouse IgG H+L DyLight 650-conjugated (Thermo Scientific, SA5-10169), 1:500; (ii) donkey anti-rabbit IgG H+L Alexa 750-conjugated (abcam, ab175728), 1:500; (iii) donkey anti-mouse IgG H+L DyLight 550-conjugated (Thermo Scientific, SA5-10167), 1:500; (iv) donkey anti-rabbit IgG H+L DyLight 650-conjugated (abcam, ab96922), 1:500. Whole slide scans of the stained sections were recorded on a Zeiss automatic microscope AxioScan Z1 with high aperture lenses, equipped with a Zeiss Axiocam 506 mono and a Hitachi 3CCD HV-F202SCL camera and Zeiss ZEN 3.3 software. Image analysis was done with Image Pro 10 (Media Cybernetics). At the beginning the target areas (cortex and hippocampus) were identified by drawing regions of interest (ROI) on the images. Additional ROIs excluded wrinkles, air bubbles, or any other artifacts interfering with the measurement. Afterwards, signals of the following markers were quantitatively evaluated within the identified areas: beta-Amyloid (4G8), GFAP, pSer202/Thr205-tau, U1-70K. Different object features were quantified, among them the percentage of cumulative object area based on ROI size (immunoreactive area; this is the most comprehensive parameter indicating whether there are differences in immunoreactivity), the number of objects normalized to ROI size (object density), the mean signal intensity of identified objects (mean intensity; this indicates if there are differences in the cellular expression level of target proteins), and the size of above-threshold objects.

All raw data is analyzed in GraphPad Prism™ 9 (GraphPad Software Inc., USA). Basic statistical analysis is performed. If appropriate, data are presented as mean±standard error of mean (SEM) and group differences are evaluated by T-test (mean object size) or Mann-Whitney.

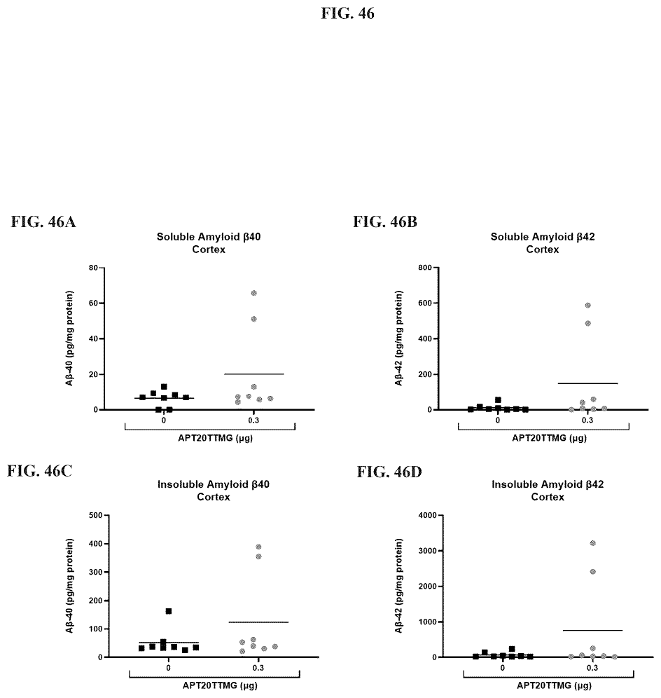

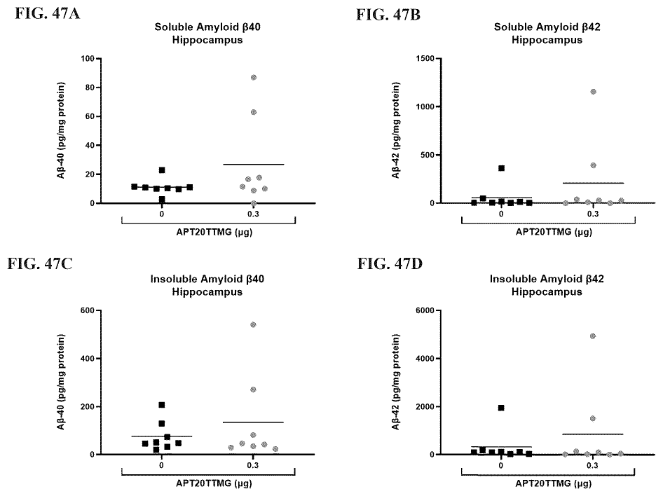

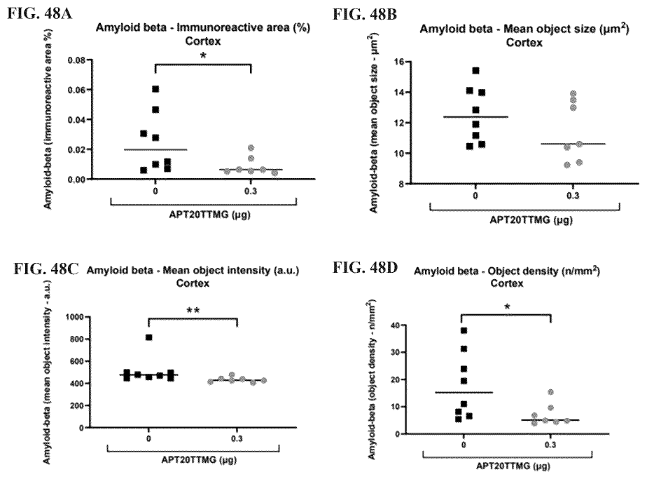

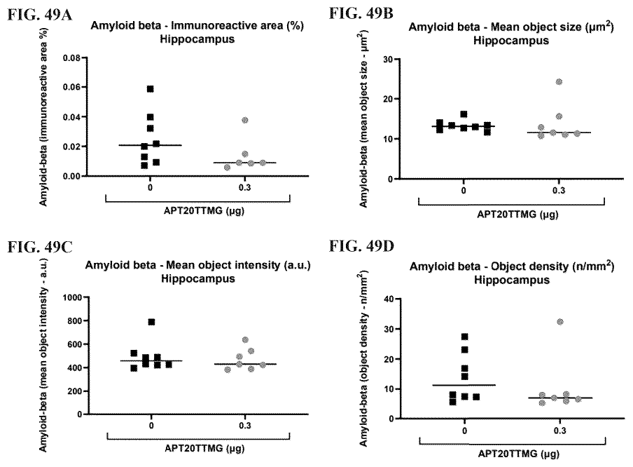

In the biochemical assays, sarkosyl soluble and insoluble protein fractions from cortex and hippocampus samples were analyzed for Aβ load using Mesoscale Discovery technology. No statistically significant differences (Mann-Whitney test) for both soluble and insoluble Aβ40 and Aβ42 levels in cortex and hippocampus (FIGS. 46 and 47). On the other hand, immunofluorescence quantification of Aβ in the cerebral cortex (FIG. 48) and hippocampus (FIG. 49) indicated a statistical reduction of about 70% of Aβ in the cortex (reduction of the immunoreactive area, object intensity, and object density parameters). Although SAMP8 animals do not present plaque formation at around 20 weeks old, it is known that senescent animals show an increase in Aβ monomers, oligomers, and fibrils. Therefore, this result could indicate that, upon a longer treatment. APT20TTMG would avoid amyloid-beta plaques formation.

As shown in FIGS. 48A-D and 49A-D, graphs present the means of immunofluorescent signal measured within the regions of interest (ROI) on 5 brain sections per mouse (n=7). Biological outliers were excluded from the analysis. Statistics: T-test (mean object size and mean object density) or Mann-Whitney test (immunoreactive area and mean object intensity) were performed, comparing all groups to vehicle-treated control animals. *p<0.05. Immunoreactive area [%]: The percentage of the ROI that is covered by above-threshold immunoreactive objects (for example: cell somata, neurites, plaques); this is the most comprehensive parameter indicating whether there are overall differences in immunoreactivity; Object density [number of objects per mm²]: The number of above-threshold immunoreactive objects normalized to the size of the target area; this is especially useful to detect changes in neuronal density; Object intensity [a.u.]: The average brightness of pixels of above-threshold immunoreactive objects; this indicates if there are differences in the cellular expression level of target proteins. Object size [μm²]: The size of above-threshold immunoreactive objects; this is useful to detect differences in activation of microglia or growth of plaques.

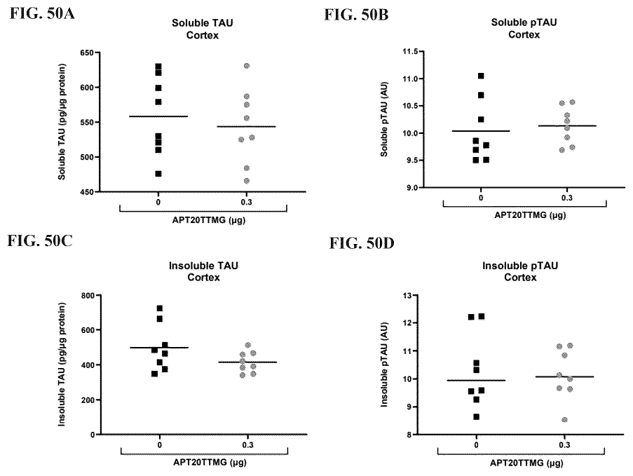

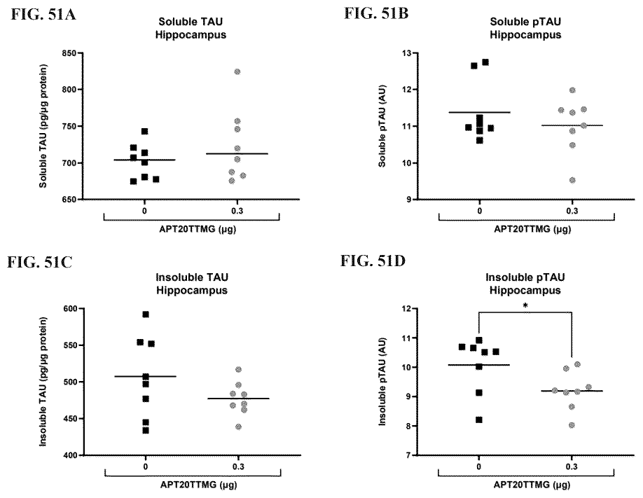

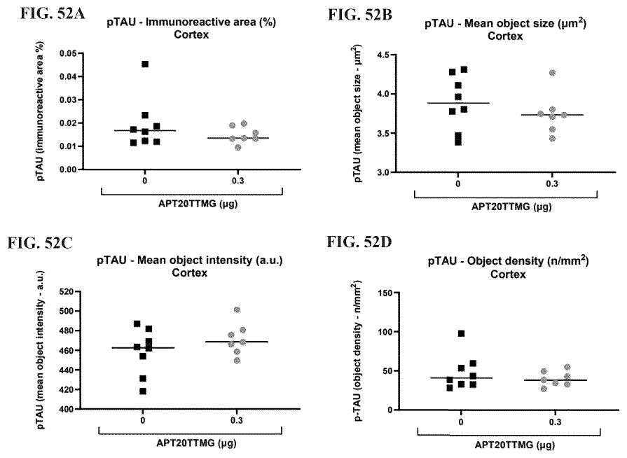

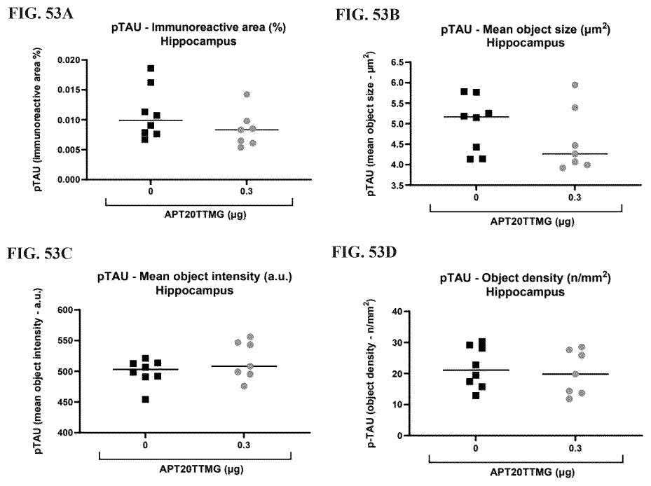

The assessment of levels of total tau and phosphorylated tau (ptau (T231)) (FIGS. 50 and 51) were determined in sarkosyl soluble and insoluble fractions of cortex and hippocampus of animals. Although no differences were observed in the cortex, a significant decrease in insoluble hyperphosphorylated tau levels in the hippocampus was observed (FIG. 51). The importance of this result lies in the potential implications of ptau in neurofibrillary tangles formation mainly in the hippocampus and its impact on neuron function. In the immunofluorescence assay, pSer202/pThr205-Tau levels (detected using AT8 mouse monoclonal antibody) were evaluated, but no significant changes were detected in the cortex and hippocampus regions through this methodology (FIGS. 52 and 53). Moreover, these data highlight the potential of APT20TTMG for reducing only insoluble ptau, without changing normal levels of tau in both cortex and hippocampus.

As shown in FIG. 50A-D, graphs depict levels of (A) soluble tau, (B) soluble tau (T231), (C) insoluble tau (D) insoluble tau (T231) in cortex samples from n=7-8 animals per treatment. Biological outliers as identified by histology were excluded from analysis. Data are given as pg/μg protein (tau) or arbitrary units (ptau).

As shown in FIG. 51A-D, graphs depict levels of (A) soluble tau, (B) soluble tau (T231), (C) insoluble tau (D) insoluble tau (T231) in cortex samples from n=7-8 animals per treatment. Biological outliers as identified by histology were excluded from analysis. Data are given as pg/μg protein (tau) or arbitrary units (ptau).

As shown in FIG. 52A-D, graphs present the means of immunofluorescent signal measured within the ROI on 5 brain sections per mouse (n=7-8). Biological outliers were excluded from the analysis. Immunoreactive area [%]: The percentage of the ROI that is covered by above-threshold immunoreactive objects (for example: cell somata, neurites, plaques); this is the most comprehensive parameter indicating whether there are overall differences in immunoreactivity; Object density [number of objects per mm²]: The number of above-threshold immunoreactive objects normalized to the size of the target area; this is especially useful to detect changes in neuronal density; Object intensity [a.u.]: The average brightness of pixels of above-threshold immunoreactive objects; this indicates if there are differences in the cellular expression level of target proteins. Object size [μm²]: The size of above-threshold immunoreactive objects; this is useful to detect differences in activation of microglia or growth of plaques.

As shown in FIG. 53A-D, graphs present the means of immunofluorescent signal measured within the ROI on 5 brain sections per mouse (n=7-8). Biological outliers were excluded from the analysis. Statistics: T-test was performed, comparing all groups to vehicle-treated control animals. *p<0.05. Immunoreactive area [%]: The percentage of the ROI that is covered by above-threshold immunoreactive objects (for example: cell somata, neurites, plaques); this is the most comprehensive parameter indicating whether there are overall differences in immunoreactivity; Object density [number of objects per mm²]: The number of above-threshold immunoreactive objects normalized to the size of the target area; this is especially useful to detect changes in neuronal density; Object intensity [a.u.]: The average brightness of pixels of above-threshold immunoreactive objects; this indicates if there are differences in the cellular expression level of target proteins. Object size [μm²]: The size of above-threshold immunoreactive objects; this is useful to detect differences in activation of microglia or growth of plaques.

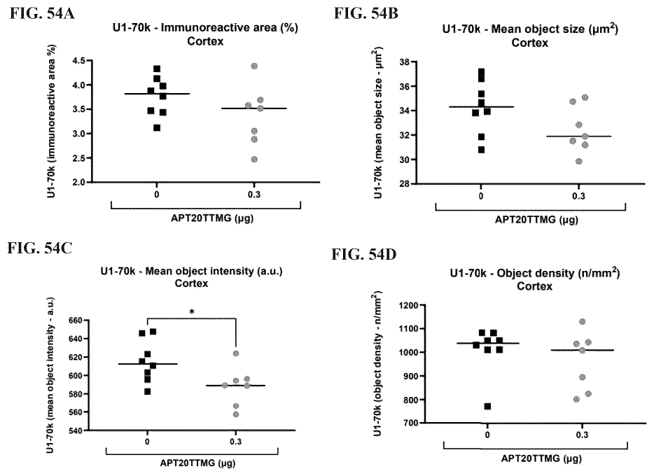

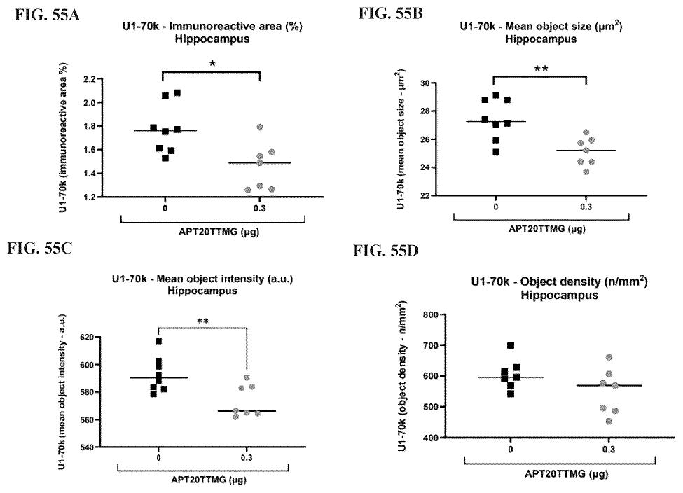

The immunofluorescence of U1-70K was detected with rabbit polyclonal antibody, and the signal was quantified in the cerebral cortex (FIG. 54), and hippocampus (FIG. 55). In the cortex, the treatment (0.3 μg/day) reduced U1-70K object intensity (FIG. 54), while the hippocampus, it significantly decreased U1-70K immunoreactive area, object intensity, and object size parameters (FIG. 55). Notably, the U1-70K reduction, particularly evident in the hippocampus, suggests a potential decrease in the cytoplasmic localization of this snRNP, as it is supported by both the reduction of the immunoreactive area and the mean object size of the U1-70K immunofluorescent signals. This is an impressive result as a cytoplasmic mislocalization and aggregation of U1 snRNPs, mainly of U1-70K, are observed in AD, with scientific evidence that this could be an upstream event that leads to the development of this neurodegenerative disease.

As shown in FIG. 54A-D, graphs present the means of immunofluorescent signal measured within the ROI on 5 brain sections per mouse (n=7-8). Biological outliers were excluded from the analysis. Statistics: T-test (immunoreactive area, mean object size and mean object intensity) or Mann-Whitney test (object density) were performed, comparing all groups to vehicle-treated control animals. *p<0.05. Immunoreactive area [%]: The percentage of the ROI that is covered by above-threshold immunoreactive objects (for example: cell somata, neurites, plaques); this is the most comprehensive parameter indicating whether there are overall differences in immunoreactivity; Object density [number of objects per mm²]: The number of above-threshold immunoreactive objects normalized to the size of the target area; this is especially useful to detect changes in neuronal density; Object intensity [a.u.]: The average brightness of pixels of above-threshold immunoreactive objects; this indicates if there are differences in the cellular expression level of target proteins. Object size [μm²]: The size of above-threshold immunoreactive objects; this is useful to detect differences in activation of microglia or growth of plaques.

As shown in FIG. 55A-D, graphs present the means of immunofluorescent signal measured within the ROI on 5 brain sections per mouse (n=7). Biological outliers were excluded from the analysis. Statistics: T-test was performed, comparing all groups to vehicle-treated control animals. *p<0.05. Immunoreactive area [%]: The percentage of the ROI that is covered by above-threshold immunoreactive objects (for example: cell somata, neurites, plaques); this is the most comprehensive parameter indicating whether there are overall differences in immunoreactivity; Object density [number of objects per mm²]: The number of above-threshold immunoreactive objects normalized to the size of the target area; this is especially useful to detect changes in neuronal density; Object intensity [a.u.]: The average brightness of pixels of above-threshold immunoreactive objects; this indicates if there are differences in the cellular expression level of target proteins. Object size [μm²]: The size of above-threshold immunoreactive objects; this is useful to detect differences in activation of microglia or growth of plaques.

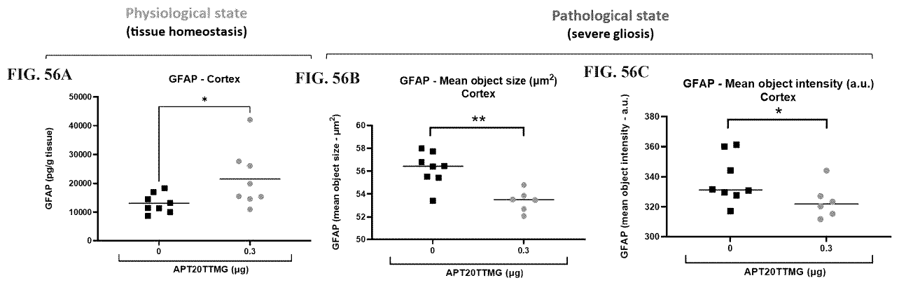

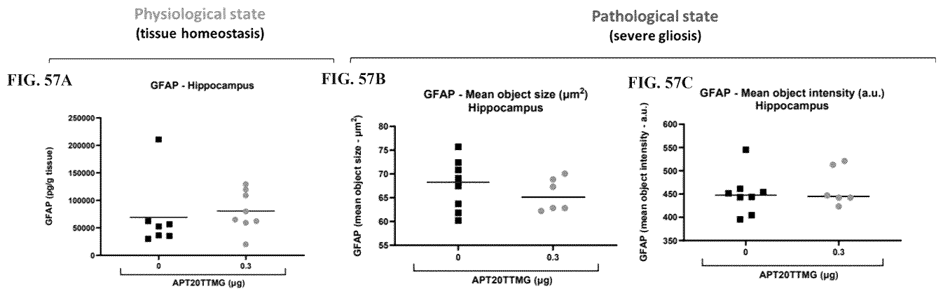

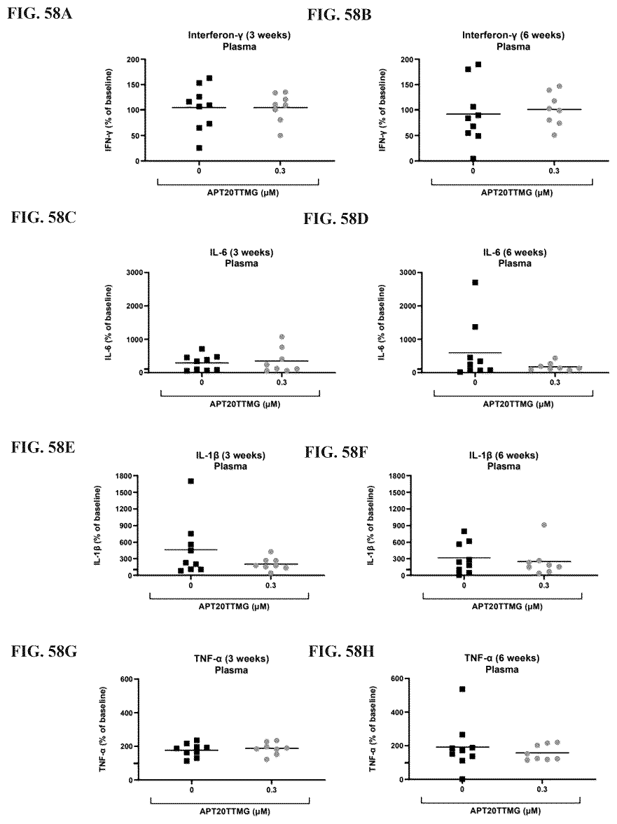

As AD pathology is also associated with morphologic, molecular, and functional remodeling of astrocytes, a process termed reactive astrogliosis, GFAP (an astrocyte marker) was also assessed through homogenate samples using ELISA and immunofluorescence (FIGS. 56 and 57). Astrogliosis is triggered by some type of pathological conditions in the surroundings of the astrocytes microenvironment, e.g., the presence of misfolded proteins. It is also believed that low-grade astrocyte activation (mild astrogliosis) is a key factor to promote Aβ and tau clearance at the early stages of AD progression. As observed in FIG. 56A, APT20TTMG activated astrocytes in the cortex (mild astrogliosis) without increasing pro-inflammatory cytokines-IFN-γ, IL-6, IL-1β, TNF-α—(FIG. 58). It suggests a physiological effect of neuronal repair through functional remodeling. In addition, the results show a significant reduction in gliosis (observed by immunofluorescence analysis, FIG. 56D and FIG. 56C) also in cortex, related to the reduction of pathological gliosis. No significant changes were observed in the hippocampus in any of the assays (FIG. 57).

As shown in FIG. 56A-C, GFAP was measured 5 weeks after the beginning of the treatments in (A) cortical homogenate samples using an immunosorbent assay (Meso Scale Discovery), and (B) and (C) Immunofluorescence of GFAP was detected with rabbit polyclonal antibody and the signal was quantified in the cerebral cortex. (A) Data are given as pg/g tissue and depict group means+SEM of n=8 animals per group. (B) and (C).

As shown in FIG. 57A-C. GFAP was measured 5 weeks after the beginning of the treatments in (A) cortical homogenate samples using an immunosorbent assay (Meso Scale Discovery), and (B) and (C) Immunofluorescence of GFAP was detected with rabbit polyclonal antibody and the signal was quantified in the cerebral cortex. (A) Data are given as pg/g tissue and depict group means+SEM of n=7-8 animals per group. (B) and (C).

As shown in FIG. 58A-H, proinflammatory cytokines were measured in in vivo (3 weeks) and terminal blood plasma (6 weeks) of all Data are shown as pg/ml plasma for (A) IFN-γ (3 weeks), (B) IFN-γ (6 weeks), (C) IL-6 (3 weeks), (D) IL-6 (6 weeks), (E) IL-1β (3 weeks), (F) IL-1β (6 weeks), (G) TNF-α (3 weeks), (H) TNF-α (6 weeks). Biological outliers as identified by histology were excluded from the analysis. Statistics: T-test (IFN-γ, IL-6-3 weeks. IL-1β—3 weeks, and TNF-α—3 weeks) or Mann-Whitney (IL-6-6 weeks. IL-1β—6 weeks, and TNF-α-6 weeks) were performed.

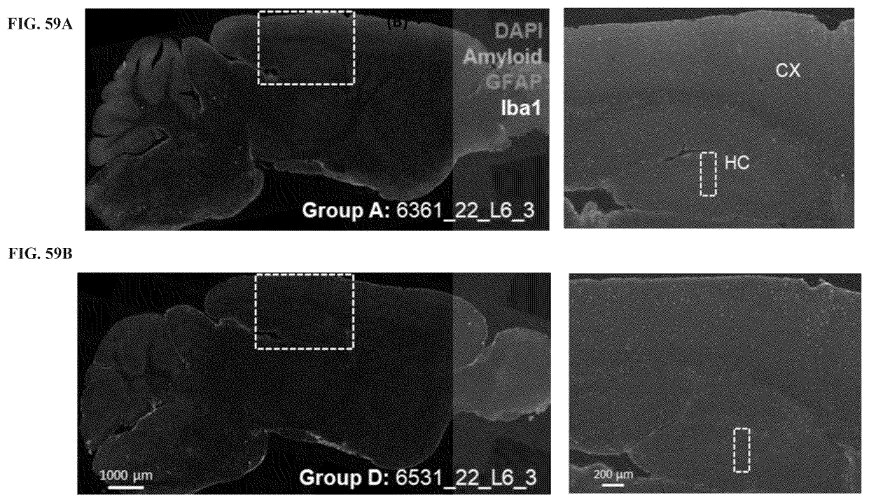

FIG. 59A-B depicts the representative images obtained from immunofluorescence signals of Aβ, Iba1, GFAP on sagittal sections of a senescence animal model (SAMP-8) in the vehicle and APT20TTMG groups.

As shown in FIG. 59A-B, representative images were obtained from vehicle group (A). Representative images obtained from APT20TTMG group (B). Images show examples of immunofluorescent labeling of Amyloid+Iba1+GFAP on sagittal sections: nuclei are labeled with DAPI. Single channel magnifications show labeling in hippocampal dentate gyrus: images were taken at the position indicated by the rectangle in the overview image.

The SAMP-8 study demonstrated the effects of 42 days of treatment with APT20TTMG on important biomarkers associated with AD pathology. Immunofluorescence assays have shown a promising 70% reduction in Aβ in the cerebral cortex, suggesting that APT20TTMG may play a role in decreasing amyloid burden, potentially avoiding plaque formation with prolonged treatment. Furthermore, a noteworthy reduction in hyperphosphorylated tau in the hippocampus observed through ELISA assays suggests the therapeutic potential of APT20TTMG in also mitigating formation of neurofibrillary tangles, one of the critical hallmarks of AD.

The significant decrease in U1-70K, especially in the hippocampus, suggests an attenuation of snRNP mislocalization and aggregation, a proposed early event in neurodegeneration progression. In the cortex. APT20TTMG beneficially modulated astrogliosis, without increasing pro-inflammatory cytokines, which could be an important factor to promote Aβ and tau clearance at early stages of AD. Overall, the reduction in Aβ and hyperphosphorylated tau, alongside the modulation of astrogliosis and U1-70K in this senescence animal model, provide an indication of APT20TTMG therapeutic potential for AD.

### Example 13. Evaluation of mRNA Length and Quality of TDP-43 (TARDP Gene) and Stathmin-2 (STMN2 Gene) in iPS Human Neurons from Alzheimer's Disease Patients

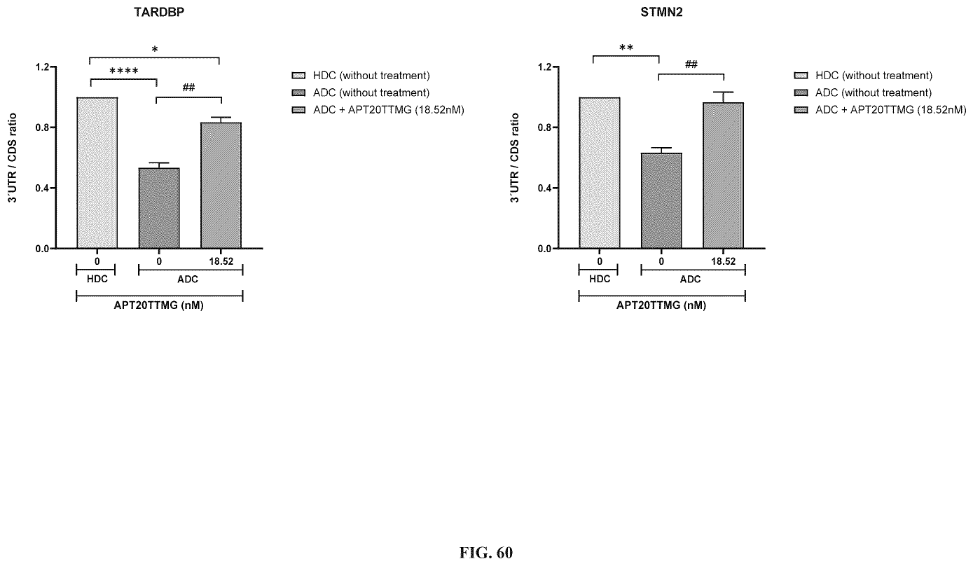

Assays were performed to analyze the effect of the engineered polynucleotides on mRNA related to genes that are associated with FTD and ALS. RNA was extracted from iPSC-derived neurons from ADC and HDCs were cultured for 6 days and incubated with 1 concentration of APT20TTMG (18.52 nM), for 7 days, using the RNeasy Micro Kit. Libraries were generated from 250 ng of total RNA and sequenced. The FASTQ reads, paired with a read length of 101 to achieve 100 million reads per sample, were aligned to the human reference genome. Reads counts of splicing junctions, quantified from sashimi plots generated from the RNA-seq assemblies, were normalized and used to calculate the 3′ UTR/CDS (untranslated regions/coding sequence) ratios of the TARDBP and STMN2 genes. FIG. 60 shows the 3′UTR/CDS ratio after administration with APT20TTMG for TARDBP and STMN2. The CDS expression was calculated based on the average read densities at the last three junctions of the constitutive exons, while the 3′ UTR value was calculated based on the read density of the last junction. Data are represented as mean+standard error of the mean. Graphs show data from three independent experiments performed in three technical replicates, analyzed using ANOVA, followed by Dunnett's post hoc test. ***p<0.05 and ****p<0.01). The analysis of 3′ UTR/CDS ratios for TARDBP and STMN2 genes (mainly related with FTD and ALS) suggests that APT20TTMG treatment may also prevent premature polyadenylation events in neurons from ADC in these genes. The AD correlation with TDP-43 dysfunction is corroborated by some studies linking TDP-43 pathology and accumulation of STMN2 to AD. This reinforces some similarities between both diseases and the potential of our molecule also for FTD and ALS.

While the foregoing disclosure has been described in some detail for purposes of clarity and understanding, it will be clear to one skilled in the art from a reading of this disclosure that various changes in form and detail can be made without departing from the true scope of the disclosure. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually and separately indicated to be incorporated by reference for all purposes.

1. A method of treating a neurodegenerative disease in a subject in need thereof comprising administering to the subject a pharmaceutical composition comprising an engineered polynucleotide comprising:
(i) one or more targeting moieties wherein a targeting moiety of the one or more targeting moieties comprises a nucleic acid sequence complementary to a target sequence, or a consensus sequence thereof, of a pre-messenger ribonucleic acid (pre-mRNA); and
(ii) a recruiting moiety for recruitment of a spliceosomal moiety, wherein the recruiting moiety comprises a nucleic acid sequence comprises (i) one or more nucleotides complementary to a sequence of a portion of a spliceosomal moiety or (ii) one or more chemically modified nucleotides,
wherein, when associated with said pre-mRNA and said engineered polynucleotide, said spliceosomal moiety alters said pre-mRNA in or in proximity to said target sequence.

2. The method of claim 1, wherein the neurodegenerative disease is selected from the group consisting of: Alzheimer's disease, Pick's disease, chronic traumatic encephalopathy, progressive supranuclear palsy, argyrophilic grain disease, Amyotrophic Lateral Sclerosis (ALS), and Frontotemporal Dementia (FTD).

3. The method of claim 1, wherein the neurodegenerative disease is a neurodegenerative disease associated with presence of Tau aggregation or dysfunction or U1 snRNP aggregation or dysfunction in a subject's brain.

4.-10. (canceled)

11. The method of claim 1, wherein the method (i) reduces the total amount of one or more protein aggregates in a subject's brain, wherein the one or mor protein aggregates comprise U1-70K protein, Tau protein, or amyloid β; (ii) generates differentially expressed genes associated with the neurogenesis, neuron differentiation, and regulation of synapsis assembly; or (iii) decreases premature polyadenylation of one or more transcripts of the neuron (iv).

12.-47. (canceled)

48. The method of claim 1, wherein the engineered polynucleotide is administered intrathecally.

49. The method of claim 1, wherein the engineered polynucleotide is administered via subcutaneous injection, intravenous injection, intramuscular injection, intradermal injection, transdermal injection percutaneous administration, intranasal administration, intralymphatic injection, intrathecal administration, intracerebroventricular injection, pulmonary administration, rectal administration intragastric administration, or any other suitable parenteral administration.

50.-52. (canceled)

53. The method of claim 1, wherein the target sequence comprises a splice site.

54. (canceled)

55. The method of claim 53, wherein said splice site comprises 5′-GU-3′

56. (canceled)

57. The method of claim 1, wherein said method alters an expression or activity of a target gene, wherein said target gene comprise said target sequence.

58. The method of claim 1, wherein said one or more targeting moieties comprise (1) a first targeting moiety complementary or identical to a first targeted sequence in said target sequence of said pre-mRNA, and (2) a second targeting moiety complementary or identical to a second targeted sequence in said target sequence of said pre-mRNA.

59.-60. (canceled)

61. The method of claim 58, wherein said first and second targeted sequences are apart in said target sequence by a spacing sequence of no more than five nucleotides (e.g., one or two nucleotides).

62.-68. (canceled)

69. The method of claim 58, wherein: (i) said first targeting moiety comprises a sequence identical or complementary to a sequence selected from the exon sequence column of Table 1; and wherein said second targeting moiety comprises a sequence identical or complementary to a sequence set forth in the intron sequence column of Table 1 or (ii) said first targeting moiety comprises a sequence identical or complementary to a sequence set forth in the intron sequence column of Table 1; and wherein said second targeting moiety comprises a sequence identical or complementary to a sequence set forth in the exon sequence column of Table 1.

70.-74. (canceled)

75. The method of claim 1, wherein said first targeting moiety comprises a sequence at least 90% identical or complementary to a ribosome binding site of a spliceosome snRNA.

76.-77. (canceled)

78. The method of claim 1, wherein said spliceosomal moiety is selected from a spliceosomal ribonucleoprotein complex, a spliceosomal small nuclear ribonucleic acid (snRNA), a spliceosomal protein, a functional variant thereof, or a functional fragment thereof.

79. (canceled)

80. The method of claim 78, wherein (i) said spliceosomal snRNA is selected from U1, U2, U4, U5, U6, U11, U12, U14atac, U6atac, and combinations thereof or (ii) wherein said spliceosomal protein is selected from Sm, U1-70k, U1A, U1C, and combinations thereof.

81. (canceled)

82. The method of claim 1, wherein said recruiting moiety comprises a nucleotide sequence that is at least 90% identical or complementary to any one of SEQ ID Nos: 1-2.

83.-84. (canceled)

85. The method of claim 1, wherein said engineered polynucleotide comprises an apical loop, an upper stem, an internal loop, a lower stem, or a combination thereof

86.-93. (canceled)

94. The method of claim 1, wherein, when associated with said engineered polynucleotide and said spliceosomal moiety, said pre-mRNA exhibits substantially no base pairing with an RNA binding domain (RBD) of U1 snRNA.

95. (canceled)

96. The method of claim 1, wherein said engineered polynucleotide is configured to specifically interact with zinc-finger of U1-C protein.

97.-99. (canceled)

100. The method of claim 1, wherein said recruiting moiety comprises (i) a nucleotide sequence comprising a phosphorothioate internucleotide linkage that binds to a U1-C zinc finger, or (ii) a nucleotide sequence complementary to a partial sequence of Stem-Loop II (SL2) of U1 snRNA.

101.-118. (canceled)

119. The method of claim 1, wherein one or more nucleotides of said engineered polynucleotide are 2′-modified nucleotides.

120. The method of claim 119, wherein said 2′-modified nucleotide comprises a 2′-methoxy, 2′-methoxymethyl, 2′-methoxyethyl, 2′ fluoro, or 2′-aminoethyl nucleotide.

121. The method of claim 1, said engineered polynucleotide comprises nucleotides connected by internucleotide linkages and at least one of said internucleotide linkages does not comprise a phosphate.

122.-125. (canceled)

126. The method of claim 1, wherein said internucleotide linkages comprises a phosphothiolate, methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo, or methyleneoxymethylimino.

127.-132. (canceled)

133. The method of claim 1, wherein said engineered polynucleotide comprises a nucleotide sequence that is at least 70%, 80%, 85%, or 90% identical or complementary to any one of SEQ ID NOs: 1-4.

134. (canceled)

135. The method of claim 1, wherein said engineered polynucleotide comprises:
(i) a first targeting moiety configured to specifically bind a pre-messenger ribonucleic acid (pre-mRNA) at a first targeted sequence therein, wherein the first targeting moiety comprises a sequence identical or complementary to 5′-GTCCA-3′,
(ii) a recruiting moiety comprising a sequence that is at least 90% similar or complementary to SEQ ID NO. 1 and is configured to recruit a spliceosomal moiety that comprises U1 snRNA and a U1-C protein, wherein said recruiting moiety comprises an apical loop, an upper stem adjacent to said apical loop, a lower stem, and an internal loop situated between said upper stem and said lower stem, and
(iii) a second targeting moiety configured to specifically bind the pre-mRNA at a second targeted sequence therein, wherein the second targeting moiety comprises a sequence identical or complementary to 5′-CG-3′.

136-139. (canceled)


SEQUENCE LISTING (22 sequences)

SEQ ID NO: 1 - DNA, 17 bp, synthetic construct
  source 1..17: mol_type = other DNA
ctaacctttcaggccag

SEQ ID NO: 2 - RNA, 17 nt, synthetic construct
  source 1..17: mol_type = other RNA
ctaacctttcaggccag

SEQ ID NO: 3 - DNA, 24 bp, synthetic construct
  source 1..24: mol_type = other DNA
gtccactaacctttcaggccagcg

SEQ ID NO: 4 - RNA, 24 nt, synthetic construct
  source 1..24: mol_type = other RNA
gtccactaacctttcaggccagcg

SEQ ID NO: 5 - protein, 33 aa, synthetic construct
YYCDYCDTYLTHDSPSVRKTHCTGRKHRDNVKF

SEQ ID NO: 6 - RNA, 11 nt, unidentified
  source 1..11: mol_type = unassigned RNA
atacttacctg

SEQ ID NO: 7 - RNA, 12 nt, unidentified
  source 1..12: mol_type = unassigned RNA
ggcctcacgttc

SEQ ID NO: 8 - DNA, 52 bp, unidentified
  source 1..52: mol_type = unassigned DNA
ctcccgtcctcgcctctgtcgactatcaggtaagcgccgcggctccgaaatc

SEQ ID NO: 9 - DNA, 64 bp, synthetic construct
  source 1..64: mol_type = other DNA
gaggcgacaggagagggcaggagcggagacagctgatagtccattcgcggcgccgaggctttag

SEQ ID NO: 10 - RNA, 164 nt, unidentified
  source 1..164: mol_type = unassigned RNA
  modified_base 6: mod_base = OTHER; note = 2'-O-methyl uridine
  modified_base 7: mod_base = OTHER; note = 2'-O-methyl adenosine
  modified_base 70: mod_base = OTHER; note = 2'-O-methyl Adenosine
atacttacctggcaggggagataccatgatcacgaaggtggttttcccagggcgaggcttatccattgcactccggatgtgctgacccctgcgatttccccaaatgtgggaaactcgactgcataatttgtggtagtgggggactgcgttcgcgctttcccctg

SEQ ID NO: 11 - RNA, 51 nt, Homo sapiens
  source 1..51: mol_type = genomic RNA
ccgtcctcgcctctgtcgactatcaggtaagcgccgcggctccgaaatctg

SEQ ID NO: 12 - RNA, 55 nt, unidentified
  source 1..55: mol_type = unassigned RNA
ggatccattgcactccggatccaggagataccatgatcacgaaggtggttttcct

SEQ ID NO: 13 - protein, 279 aa, unidentified
GVPETRPNHTIYINNLNEKIKKDELKKSLYAIFSQFGQILDILVSRSLKMRGQAFVIFKEVSSATNALRSMQGFPFYDKPMRIQYAKTDSDIIAKMKGTFVERDRKREKRGSGSGSGSGSGSGAETREERMERKRREKIERRQQEVETELKMWDPHNDPNAQGDAFKTLFVARVNYDTTESKLRREFEVYGPIKRIHMVYSKRSGKPRGYAFIEYEHERDMHSAYKHADGKKIDGRRVLVDVERGRTVKGWRPRRLGGGLGGTRRGGADVNIRHSGRDD

SEQ ID NO: 14 - RNA, 51 nt, unidentified; identical to SEQ ID NO: 11
  source 1..51: mol_type = unassigned RNA

SEQ ID NO: 15 - RNA, 55 nt, unidentified; identical to SEQ ID NO: 12
  source 1..55: mol_type = unassigned RNA

SEQ ID NO: 16 - protein, 279 aa, unidentified; identical to SEQ ID NO: 13

SEQ ID NO: 17 - protein, 145 aa, unidentified
MPKYYCDYCDTYLTHDSPSVRKTHCTGRKHRDNVKFYYQKWMEEQAQHLIDATTAAFKAGKITNNPFAGGPGGAPPKPAGVSIPPPNMGAPPRPGMPGMPYMPPLMNPMMGMRPPPIMNPMAMMGPPPPLGTIPGVRPGIMNGPK

SEQ ID NO: 18 - DNA, 24 bp, synthetic construct
  source 1..24: mol_type = other DNA
  modified_base 1: mod_base = OTHER; note = 2'-O-methyl Phosphorothioate Guanosine
  modified_base 2: mod_base = OTHER; note = 2'-O-methyl Phosphorothioate Thymidine
  modified_base 3: mod_base = OTHER; note = 2'-O-methyl Phosphorothioate Cytosine
  modified_base 4: mod_base = OTHER; note = 2'-O-methyl Phosphorothioate Cytosine
  modified_base 5: mod_base = OTHER; note = 2'-O-methyl Phosphorothioate (Thiol) adenosine
  modified_base 6: mod_base = OTHER; note = Phosphorothioate Cytosine
  modified_base 7: mod_base = OTHER; note = Phosphorothioate Thymidine
  modified_base 8: mod_base = OTHER; note = Phosphorothioate Adenosine
  modified_base 9: mod_base = OTHER; note = Phosphorothioate Adenosine
  modified_base 10: mod_base = OTHER; note = Phosphorothioate Cytosine
  modified_base 11: mod_base = OTHER; note = Phosphorothioate Cytosine
  modified_base 12: mod_base = OTHER; note = Phosphorothioate Thymidine
  modified_base 13: mod_base = OTHER; note = Phosphorothioate Thymidine
  modified_base 14: mod_base = OTHER; note = Phosphorothioate Thymidine
  modified_base 15: mod_base = OTHER; note = Phosphorothioate Cytosine
  modified_base 16: mod_base = OTHER; note = Phosphorothioate Adenosine
  modified_base 17: mod_base = OTHER; note = Phosphorothioate Guanosine
  modified_base 18: mod_base = OTHER; note = Phosphorothioate Guanosine
  modified_base 19: mod_base = OTHER; note = Phosphorothioate Cytosine
  modified_base 20: mod_base = OTHER; note = 2'-O-methyl Phosphorothioate Cytosine
  modified_base 21: mod_base = OTHER; note = 2'-O-methyl Phosphorothioate Adenosine
  modified_base 22: mod_base = OTHER; note = 2'-O-methyl Phosphorothioate Guanosine
  modified_base 23: mod_base = OTHER; note = 2'-O-methyl Phosphorothioate Cytosine
  modified_base 24: mod_base = OTHER; note = 2'-O-methyl Phosphorothioate Guanosine
gtccactaacctttcaggccagcg

SEQ ID NO: 19 - RNA, 20 nt, synthetic construct
  source 1..20: mol_type = other RNA
  modified_base 11: mod_base = OTHER; note = 2'-O-methyl Adenosine
tatccattgcactccggatg

SEQ ID NO: 20 - DNA, 24 bp, synthetic construct
  source 1..24: mol_type = other DNA
  modified_base 1: mod_base = OTHER; note = 2'-O-methyl Phosphorothioate Guanosine
  modified_base 2: mod_base = OTHER; note = 2'-O-methyl Phosphorothioate Thymidine
  modified_base 3: mod_base = OTHER; note = 2'-O-methyl Phosphorothioate Cytosine
  modified_base 4: mod_base = OTHER; note = 2'-O-methyl Phosphorothioate (Thiol) cytosine
  modified_base 5: mod_base = OTHER; note = 2'-O-methyl Phosphorothioate Adenosine
  modified_base 6: mod_base = OTHER; note = Phosphorothioate Cytosine
  modified_base 7: mod_base = OTHER; note = Phosphorothioate Thymidine
  modified_base 8: mod_base = OTHER; note = Phosphorothioate Adenosine
  modified_base 9: mod_base = OTHER; note = Phosphorothioate Adenosine
  modified_base 10: mod_base = OTHER; note = Phosphorothioate Cytosine
  modified_base 11: mod_base = OTHER; note = Phosphorothioate Cytosine
  modified_base 12: mod_base = OTHER; note = Phosphorothioate Thymidine
  modified_base 13: mod_base = OTHER; note = Phosphorothioate Thymidine
  modified_base 14: mod_base = OTHER; note = Phosphorothioate Thymidine
  modified_base 15: mod_base = OTHER; note = Phosphorothioate Cytosine
  modified_base 16: mod_base = OTHER; note = Phosphorothioate Adenosine
  modified_base 17: mod_base = OTHER; note = Phosphorothioate Guanosine
  modified_base 18: mod_base = OTHER; note = Phosphorothioate Guanosine
  modified_base 19: mod_base = OTHER; note = Phosphorothioate Cytosine
  modified_base 20: mod_base = OTHER; note = 2'-O-methyl Phosphorothioate Cytosine
  modified_base 21: mod_base = OTHER; note = 2'-O-methyl Phosphorothioate Adenosine
  modified_base 22: mod_base = OTHER; note = 2'-O-methyl Phosphorothioate Guanosine
  modified_base 23: mod_base = OTHER; note = 2'-O-methyl Cytosine
  modified_base 24: mod_base = OTHER; note = 2'-O-methyl Guanosine
gtccactaacctttcaggccagcg

SEQ ID NO: 21 - RNA, 16 nt, synthetic construct
  source 1..16: mol_type = other RNA
  misc_difference 11: note = a, c, u or g
yyyyyyyyyyncaggr

SEQ ID NO: 22 - RNA, 165 nt, unidentified
  source 1..165: mol_type = unassigned RNA
  modified_base 6: mod_base = OTHER; note = 2'-O-methyl uridine
  modified_base 7: mod_base = OTHER; note = 2'-O-methyl adenosine
  modified_base 71: mod_base = OTHER; note = 2'-O-methyl adenosine
atacttacctggcagggggagataccatgatcacgaaggtggttttcccagggcgaggcttatccattgcactccggatgtgctgacccctgcgatttccccaaatgtgggaaactcgactgcataatttgtggtagtgggggactgcgttcgcgctttcccctg